(19) United States
(12) Patent Application Publication
(10) Pub. No.: US 2012/0058541 A1
(43) Pub. Date: Mar. 8, 2012

(54) ENGINEERING RESISTANCE TO ALIPHATIC ALCOHOLS

(75) Inventors: Keith Alsaker, Wilmington, DE (US); Daniel Grenfell-Lee, Wilmington, DE (US); Michael Hudson, Wilmington, DE (US); Adam Lawrence, Wilmington, DE (US); Jessica McGrath, Wilmington, DE (US); David Young, Wilmington, DE (US)

(73) Assignee: BUTAMAX(TM) ADVANCED BIOFUELS LLC, Wilmington, DE (US)

(21) Appl. No.: 13/214,310

(22) Filed: Aug. 22, 2011

Related U.S. Application Data
(63) Continuation of application No. PCT/US09/45031, filed on May 22, 2009.

(51) Int. Cl. C12N 1/21; C12N 1/19

(52) U.S. Cl. 435/252.31; 435/252.3; 435/252.33; 435/252.34; 435/252.32; 435/254.23; 435/254.22; 435/254.21

(57) ABSTRACT

The present disclosure provides improved systems for the biological production of certain aliphatic alcohol compounds. In particular, the present disclosure provides biological systems that show improved resistance to aliphatic alcohol toxicity; in sonic embodiments, such improved resistance allows for increased levels of aliphatic alcohol production. Accordingly, the present disclosure provides, inter alia, engineered microorganisms that both produce an aliphatic alcohol compound and show resistance to that compound as measured by an ability to grow to predetermined levels in the presence of a given concentration of the compound.

### CROSS REFERENCE TO RELATED APPLICATIONS

The present application is copending with, shares at least one common inventor with and claims priority to U.S. provisional patent application Ser. No. 61/055,330, filed May 22, 2008, the entire contents of which are incorporated herein by reference.

### BACKGROUND

Aliphatic alcohols, such as butanol, are important industrial chemicals, useful among other things as fuel additives, as chemical feedstocks in the plastics industry, and as food-grade extractants in the food and flavor industry. For example, each year at least 10-12 billion pounds of butanol are produced by petrochemical means, and the need for this commodity chemical will likely increase.

There is a need for the development of new technologies for the production of aliphatic alcohols. Methods of chemical synthesis (typically starting from petrochemical by-products) are expensive and utilize or produce environmentally damaging agents. Efforts have been made to develop biotransformation and fermentation processes that employ microorganisms for some or all of the steps in aliphatic alcohol production. However, reported protocols are typically complicated. Moreover, such efforts often are hampered by toxicity of produced compounds toward the utilized microorganisms.

### SUMMARY

The present disclosure provides improved systems for the biological production of certain aliphatic alcohol compounds. In particular, the present disclosure provides biological systems that show improved resistance to aliphatic alcohol toxicity; in some embodiments, such improved resistance allows for increased levels of aliphatic alcohol production.

Accordingly, the present disclosure provides, inter alia, engineered microorganisms that both produce an aliphatic alcohol compound and show resistance to that compound as measured by an ability to grow to predetermined levels in the presence of a given concentration of the compound.

In one aspect, the present disclosure provides a recombinant microbial cell, characterized in that the recombinant microbial cell comprises at least one alcohol tolerance modification as compared with a parent cell.

In some embodiments, an alcohol tolerance modification comprises introduction of a nucleic acid molecule comprising a 3′ region of a gene encoding a CAAX protease polypeptide. In some embodiments, a 3′ region of the gene is a 3′ untranslated region (UTR). A 3′ region of the gene can be a region sufficient to adjust susceptibility of the cell to one or more toxic effects of one or more aliphatic alcohol compounds. In some embodiments, a 3′ region comprises at least 10 nucleotides, e.g., at least 20, 30, 40, 50, 60, 70, 80, 90, 100, 150, 200, 250, 300, 350, 400, 450, 500, 550, 600, or 650 nucleotides. A 3′ region of the gene can include nucleotides within 500, 200, 100, 50, or fewer nucleotides, or immediately downstream of, sequence encoding the CAAX protease polypeptide. In some embodiments, a nucleic acid molecule comprises a 3′ region of a gene encoding a CAAX protease polypeptide in Lactobacillus, e.g., a 3′ region of a gene encoding a CAAX protease polypeptide in Lactobacillus plantarum. In some embodiments, a 3′ region of the gene comprises 655 nucleotides immediately downstream of a sequence encoding a Lactobacillus plantarum CAAX protease polypeptide. Exemplary sequences from a 3′ region of a gene encoding a CAAX protease polypeptide are shown, e.g., in Table 1B. In some embodiments, a 3′ region of the gene comprises at least 10 consecutive nucleotides (e.g., at least 20, 30, 40, 50, 60, 70, 80, 90, 100, 150, 200, 250, 300, 350, 400, 450, 500, 550, 600, or 650 nucleotides) of the nucleotide sequence shown in Table 1B, row 42, or a homologous sequence thereof (e.g., a sequence having at least 60%, 70%, 80%, 85%, 90%, 95%, 97%, 99% identity).

A recombinant microbial cell can include a nucleic acid molecule comprising a 5′ region of a gene encoding a CAAX protease polypeptide. In some embodiments, an alcohol tolerance modification comprises introduction of both a 3′ region and a 5′ region of a gene encoding a CAAX protease polypeptide. A 3′ region and 5′ regions may be from the same or from different organisms. A 5′ region of the gene can include a 5′ UTR. In some embodiments, a 5′ region of the gene comprises at least 10, 20, 30, 40, 50, 60, 70, 80, 90, or 100 nucleotides. A 5′ region of the gene can include nucleotides within 500, 200, 100, 50, or fewer nucleotides, or immediately upstream of, sequence encoding the CAAX protease polypeptide.

In some embodiments, a nucleic acid molecule includes a 5′ region of a gene encoding a CAAX protease polypeptide in Lactobacillus, e.g., Lactobacillus plantarum. In one embodiment, a 5′ region of the gene includes 111 nucleotides immediately upstream of sequence encoding a Lactobacillus plantarum CAAX protease polypeptide. Exemplary sequences from a 5′ region of a gene encoding a CAAX protease polypeptide are shown, e.g., in Table 1B. In some embodiments, a 5′ region of the gene comprises at least 10 consecutive nucleotides (e.g., at least 20, 30, 40, 50, 60, 70, 80, 90, or 100 nucleotides) of the nucleotide sequence shown in Table 1B, row 40, or a homologous sequence thereof (e.g., a sequence having at least 60%, 70%, 80%, 85%, 90%, 95%, 97%, 99% identity).

In some embodiments, a recombinant microbial cell provided herein exhibits increased tolerance to at least one aliphatic alcohol compound as compared with the parent cell. Increased tolerance to at least one aliphatic alcohol compound can include an increased aliphatic alcohol compound IC₅₀, wherein the IC₅₀ is increased at least 5%, 10%, 20%, 30%, 40%, 50%, 60%, 70%, 80%, 90%, 100%, or more. Increased tolerance to at least one aliphatic alcohol compound can include an increased aliphatic alcohol compound IC₅₀ of at least 10%, 50%, or 100%.

In some embodiments, the increased tolerance to at least one aliphatic alcohol compound comprises increased carbohydrate utilization (e.g., glucose and/or lignocellulosic-based carbohydrate utilization) as compared to the parent cell when grown in same amount of alcohol, e.g., wherein the carbohydrate utilization is increased at least 5%, 10%, 20%, 30%, 40%, 50%, 60%, 70%, 80%, 90%, 100%, or more.

In some embodiments, a recombinant microbial cell provided herein produces at least one aliphatic alcohol compound. In some embodiments, a cell provided herein exhibits increased alcohol production as compared with the parent cell. In some embodiments, alcohol production is increased by the at least one alcohol tolerance modification. Increased alcohol production can be determined by measuring a characteristic selected from the group consisting of: broth titer (grams aliphatic alcohol produced per liter broth (g l-1)), aliphatic alcohol yield (grams aliphatic alcohol produced per gram substrate consumed (g g-1), volumetric productivity (grams aliphatic alcohol produced per liter per hour (g l-1 h-1)), and specific productivity (grams aliphatic alcohol produced per gram host cell biomass per hour (g/g cells h-1)), and combinations thereof. In some embodiments, broth titer is increased at least 10%, 25%, 50%, 75%, 100%, or more. In some embodiments, aliphatic alcohol yield is increased at least 10%, 25%, 50%, 75%, 100%, or more. In some embodiments, volumetric productivity is increased at least 10%, 25%, 50%, 75%, 100%, or more. In some embodiments, specific productivity is increased at least 10%, 25%, 50%, 75%, 100%, or more.

In some embodiments, a parent cell of a cell provided herein naturally produces at least one aliphatic alcohol compound. In some embodiments, a parent cell of a cell provided herein does not naturally produce an aliphatic alcohol compound.

In some embodiments, a cell provided herein is a member of a genus selected from the group consisting of Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Acinetobacter, Pichia, Candida, Hansenula and Saccharomyces. In some embodiments, a cell species is selected from the group consisting of Clostridium acetobutylicum, Clostridium beijerinckii, and Clostridium saccaharoperbuylacetonicum. In some embodiments, a cell species is Escherichia coli. In some embodiments, a cell species is Alcaligenes eutrophus. In some embodiments, a cell species is Bacillus licheniformis. In some embodiments, a cell species is Paenibacillus macerans. In some embodiments, a cell species is Rhodococcus erythropolis. In some embodiments, a cell species is Pseudomonas putida. In some embodiments, a cell species is Bacillus subtilis. In some embodiments, a cell species is Lactobacillus plantarum. In some embodiments, a cell species is Enterococcus faecium. In some embodiments, a cell species is Enterococcus gallinarum. In some embodiments, a cell species is Enterococcus faecalis. In some embodiments, a cell species is Saccharomyces cerevisiae.

In various embodiments, an alcohol tolerance modification increases expression or activity of at least one alcohol tolerance polypeptide in a recombinant microbial cell. For example, the alcohol tolerance modification decreases expression or activity of at least one alcohol tolerance polypeptide, or increases expression or activity of at least one alcohol tolerance polypeptide and decreases expression or activity of at least one other alcohol tolerance polypeptide.

In some embodiments, the at least one alcohol tolerance polypeptide is either encoded by or homologous to a polypeptide encoded by the genome of a parent cell. In some embodiments, the at least one alcohol tolerance polypeptide is at least one polypeptide selected from the group consisting of those encoded by determinant sequences in Table 1A, and combinations thereof. In some embodiments, the at least one alcohol tolerance polypeptide is at least one polypeptide selected from the group consisting of those encoded by determinant sequences in Table 1A, homologs thereof, and combinations thereof. In some embodiments, the at least one alcohol tolerance polypeptide is at least one polypeptide selected from the group consisting of those encoded by determinant sequences in Table 2, and combinations thereof. For example, in some embodiments, the at least one alcohol tolerance polypeptide is a polypeptide selected from the group consisting of a calcineurin-like phosphoesterase polypeptide, a cation transport protein (mntH3 related) polypeptide, a transcription regulator (1p_{—}2159 related) polypeptide, a lp_{—}2160 related polypeptide, a lp_{—}2169 related polypeptide, a phosphoglycerate mutase polypeptide, a CAAX protease polypeptide, and a peptidylprolyl isomerase (prs2A related polypeptide).

The at least one alcohol tolerance modification can include introduction of an alcohol tolerance determinant found within the alcohol tolerance determinant sequences in Tables 1 and 2. In some embodiments, the at least one alcohol tolerance modification comprises introduction of an alcohol tolerance determinant selected from the group consisting of those found within a Table 1A row selected from the group consisting of row 20 (lp_{—}1293), row 21 (lp_{—}1295 [nmtH3]), row 34 (lp_{—}2159), row 35 (lp_{—}2160), row 36 (lp_{—}2169), row 37 (lp_{—}2170), row 44 (lp_{—}2911), row 50 (lp_{—}3193), homologs thereof, and combinations of any of the foregoing. In some embodiments, homologs thereof are selected from among those found in one or more of Tables 2T, 2U, 2AH, 2AI, 2AJ, 2AK, 2AR, 2AX, and combinations thereof.

The at least one alcohol tolerance modification can further include introduction of an alcohol tolerance determinant selected from the group consisting of those found in Tables 3 and 4. In some embodiments, an alcohol tolerance determinant found in Tables 3 and 4 is selected from the group consisting of those found in sequences present in rows 2 (groES), 3 (groEL), 5 (cfa1) and/or 8 (cfa2) of Table 3, or in any of Tables 4B, 4C, 4E and 4H.

In some embodiments, the at least one alcohol tolerance modification further includes disruption or inhibition of an alcohol tolerance determinant selected from the group consisting of those found in Tables 3 and 4. In some embodiments, an alcohol tolerance determinant found in Tables 3 and 4 is selected from the group consisting of those found in sequences present in rows 1 (htrA), 4 (clpP), 6 (relA/spoT), 7 (hrcA), and/or 8 (cfa2) of Table 3, or in any of Tables 4A, 4D, 4F, 4G and 4H.

In some embodiments, the at least one alcohol tolerance polypeptide whose activity or expression is altered in a recombinant microbial cell provided herein comprises a CAAX protease. In some embodiments, the at least one alcohol tolerance polypeptide comprises a prs2A related polypeptide. In some embodiments, the at least one alcohol tolerance polypeptide comprises a calcineurin-like phosphoesterase. In some embodiments, the at least one alcohol tolerance polypeptide comprises a cation transport protein (mntH3 related).

In some embodiments, an alcohol tolerance modification comprises introduction of an alcohol determinant sequence found within a DNA insert sequence depicted in Table 1B. In some embodiments, an alcohol tolerance modification comprises introduction of an alcohol determinant sequence found within a DNA insert of p5AE4-1 depicted in Table 1B. In some embodiments, an alcohol tolerance modification comprises introduction of an alcohol determinant sequence found within a DNA insert of p5AE0-4, p5AE0-14, or p5AE0-24, depicted in Table 1B.

The at least one alcohol tolerance polypeptide whose activity or expression is altered in a recombinant microbial cell provided herein can be heterologous to the host cell. In some embodiments, the at least one alcohol tolerance polypeptide is at least one polypeptide selected from the group consisting of a polypeptide in Table 1, Table 2, or a homolog thereof. In some embodiments, a host cell is a L. plantarum cell, or a C. acetobutylicum cell. In some embodiments, the at least one alcohol tolerance polypeptide is selected from the group consisting of those presented in Table 2. In some embodiments, the at least one alcohol tolerance polypeptide is selected from the group consisting of those presented in Table 1 and Table 2. In some embodiments, the at least one alcohol tolerance polypeptide is at least one polypeptide selected from the group consisting of those encoded by determinant sequences in Table 1A, homologs thereof, and combinations thereof. In some embodiments, the at least one alcohol tolerance polypeptide is a polypeptide selected from the group consisting of a calcineurin-like phosphoesterase polypeptide, a cation transport protein (mntH3 related) polypeptide, a transcription regulator (lp_{—}2159 related) polypeptide, a lp_{—}2160 related polypeptide, a lp_{—}2169 related polypeptide, a phosphoglycerate mutase polypeptide, a CAAX protease polypeptide, and a peptidylprolyl isomerase (prs2A related polypeptide).

In some embodiments, the at least one alcohol tolerance modification includes introduction of an alcohol tolerance determinant which is heterologous to the host cell, and which is found within the alcohol tolerance determinant sequences in Tables 1 and 2. The at least one alcohol tolerance modification can include introduction of an alcohol tolerance determinant selected from the group consisting of those found within a Table 1A row selected from the group consisting of row 20 (lp_{—}1293), row 21 (lp_{—}1295 [nmtH3]), row 34 (lp_{—}2159), row 35 (lp_{—}2160), row 36 (lp_{—}2169), row 37 (lp_{—}2170), row 44 (lp_{—}2911), row 50 (lp_{—}3193), homologs thereof, and combinations of any of the foregoing. The homologs thereof can be selected from among those found in one or more of Tables 2T, 2U, 2AH, 2AI, 2AJ, 2AK, 2AR, 2AX, and combinations thereof. The at least one alcohol tolerance modification can further include introduction of an alcohol tolerance determinant selected from the group consisting of those found in Tables 3 and 4, e.g., wherein the alcohol tolerance determinant found in Tables 3 and 4 is selected from the group consisting of those found in sequences present in rows 2 (groES), 3 (groEL), 5 (cfa1) and/or 8 (cfa2) of Table 3, or in any of Tables 4B, 4C, 4E and 4H. The at least one alcohol tolerance modification can further include disruption or inhibition of an alcohol tolerance determinant selected from the group consisting of those found in Tables 3 and 4, e.g., wherein the alcohol tolerance determinant found in Tables 3 and 4 is selected from the group consisting of those found in sequences present in rows 1 (htrA), 4 (clpP), 6 (relA/spoT), 7 (hrcA), and/or 8 (cfa2) of Table 3, or in any of Tables 4A, 4D, 4F, 4G and 4H.

In some embodiments, an alcohol tolerance modification comprises expression of at least one heterologous alcohol tolerance polypeptide in a recombinant microbial cell. In some embodiments, an alcohol tolerance modification comprises expression of at least one heterologous gene encoding the at least one heterologous alcohol tolerance polypeptide. In some embodiments, the at least one heterologous alcohol tolerance polypeptide is a butanol tolerance polypeptide. In some embodiments, a butanol tolerance polypeptide is selected from the group consisting of a polypeptide in Table 1, or a homolog thereof. The at least one heterologous alcohol tolerance polypeptide can include at least two heterologous alcohol tolerance polypeptides.

In some embodiments, an alcohol tolerance modification comprises increased expression or activity of at least one endogenous alcohol tolerance polypeptide in a recombinant microbial cell, which endogenous alcohol tolerance polypeptide is endogenous to a parental cell. In some embodiments, the alcohol tolerance modification comprises increased expression or activity of at least one endogenous gene encoding the at least one endogenous alcohol tolerance polypeptide. In some embodiments, the at least one endogenous alcohol tolerance polypeptide is a butanol tolerance polypeptide, e.g., a butanol tolerance polypeptide selected from the group consisting of a polypeptide in Table 1, or a homolog thereof. In some embodiments, the at least one endogenous alcohol tolerance polypeptide comprises at least two endogenous alcohol tolerance polypeptides. In some embodiments, the at least two endogenous alcohol tolerance polypeptides are each butanol tolerance polypeptides. In some embodiments, butanol tolerance polypeptides are each selected from the group consisting of a polypeptide in Table 1, or a homolog thereof.

In some embodiments, an alcohol tolerance modification comprises decreased expression or activity of at least one endogenous alcohol tolerance polypeptide in a recombinant microbial cell, which endogenous alcohol tolerance polypeptide is endogenous to a parental cell. In some embodiments, an alcohol tolerance modification comprises decreased expression or activity of at least one endogenous gene encoding the at least one endogenous alcohol tolerance polypeptide. In some embodiments, the at least one endogenous alcohol tolerance polypeptide is a butanol tolerance polypeptide. In some embodiments, a butanol tolerance polypeptide is selected from the group consisting of a polypeptide in Table 1, or a homolog thereof. In some embodiments, the at least one endogenous alcohol tolerance polypeptide comprises at least two endogenous alcohol tolerance polypeptides. In some embodiments, the at least two endogenous alcohol tolerance polypeptides are each butanol tolerance polypeptides. In some embodiments, butanol tolerance polypeptides are each selected from the group consisting of a polypeptide in Table 1, or a homolog thereof.

A recombinant microbial cell provided herein can further include at least one alcohologenic modification. In some embodiments, an alcohologenic modification increases expression or activity of at least one alcohologenic polypeptide. In some embodiments, an alcohologenic modification decreases expression or activity of at least one alcohologenic polypeptide. In some embodiments, an alcohologenic modification increases expression or activity of at least one alcohologenic polypeptide and decreases expression or activity of at least one other alcohologenic polypeptide. In some embodiments, an alcohologenic modification comprises expression of at least one heterologous alcohologenic polypeptide in a recombinant microbial cell. In some embodiments, an alcohologenic modification comprises expression of at least one heterologous gene encoding the at least one heterologous alcohologenic polypeptide.

The at least one alcohologenic polypeptide can be a polypeptide that increases production of an aliphatic alcohol selected from the group consisting of: methanol, ethanol, 1-propanol, 2-propanol, iso-propanol, 1-butanol, 2-butanol, iso-butanol, tert-butanol, 1-pentanol, 2-pentanol, 3-pentanol, iso-pentanol, 1-hexanol, 2-hexanol, 3-hexanol, 2-ethyl hexanol, iso-hexanol, 1-heptanol, 2-heptanol, 3-heptanol, 4-heptanol, iso-heptanol, 1-octanol, 2-octanol, 3-octanol, iso-octanol, and 4-octanol, and combinations thereof. In some embodiments, the at least one alcohologenic polypeptide is a polypeptide that increases production of an aliphatic alcohol selected from the group consisting of: 1-butanol, 2-butanol, iso-butanol, tert-butanol, and combinations thereof. In some embodiments, the at least one alcohologenic polypeptide is a polypeptide that increases production of 1-butanol, 2-butanol, or iso-butanol.

In some embodiments, the at least one alcohologenic polypeptide catalyzes a substrate to product conversion selected from the group consisting of: a) acetyl-CoA to acetoacetyl-CoA; b) acetoacetyl-CoA to 3-hydroxybutyryl-CoA; c) 3-hydroxybutyryl-CoA to crotonyl-CoA; d) crotonyl-CoA to butyryl-CoA; e) butyryl-CoA to butyraldehyde; f) butyraldehyde to 1-butanol; and combinations thereof. In some embodiments, the polypeptide that catalyzes a substrate to product conversion of acetyl-CoA to acetoacetyl-CoA is acetyl-CoA acetyltransferase. In some embodiments, a polypeptide that catalyzes a substrate to product conversion of acetoacetyl-CoA to 3-hydroxybutyryl-CoA is 3-hydroxybutyryl-CoA dehydrogenase. In some embodiments, a polypeptide that catalyzes a substrate to product conversion of 3-hydroxybutyryl-CoA to crotonyl-CoA is crotonase. In some embodiments, a polypeptide that catalyzes a substrate to product conversion of crotonyl-CoA to butyryl-CoA is butyryl-CoA dehydrogenase. In some embodiments, a polypeptide that catalyzes a substrate to product conversion of butyryl-CoA to butyraldehyde is butyraldehyde dehydrogenase. In some embodiments, a polypeptide that catalyzes a substrate to product conversion of butyraldehyde to 1-butanol is butanol dehydrogenase.

In some embodiments, the at least one alcohologenic polypeptide catalyzes a substrate to product conversion selected from the group consisting of: a) pyruvate to acetolactate; b) acetolactate to 2,3-dihydroxyisovalerate; c) alpha-ketoisovalerate to isobutyraldehyde; d) isobutyraldehyde to isobutanol; e) 2,3-dihydroxyisovalerate to alpha-ketoisovalerate; f) alpha-ketoisovalerate to isobutyraldehyde; g) alpha-ketoisovalerate to isobutyryl-CoA; h) isobutyryl-CoA to isobutyraldehyde; i) alpha-ketoisovalerate to L-valine; j) L-valine to isobutylamine; k) isobutylamine to isobutyraldehyde; 1) butyryl-CoA to isobutyryl-CoA; and combinations thereof.

In some embodiments, a polypeptide that catalyzes a substrate to product conversion of pyruvate to acetolactate is acetolactate synthase. In some embodiments, a polypeptide that catalyzes a substrate to product conversion of acetolactate to 2,3-dihydroxyisovalerate is acetohydroxy acid reductoisomerase. In some embodiments, a polypeptide that catalyzes a substrate to product conversion of acetolactate to 2,3-dihydroxyisovalerate is acetohydroxy acid isomeroreductase. In some embodiments, a polypeptide that catalyzes a substrate to product conversion of alpha-ketoisovalerate to isobutyraldehyde is branched-chain alpha-keto acid decarboxylase. In some embodiments, a polypeptide that catalyzes a substrate to product conversion of isobutyraldehyde to isobutanol is branched-chain alcohol dehydrogenase. In some embodiments, a polypeptide that catalyzes a substrate to product conversion of 2,3-dihydroxyisovalerate to alpha-ketoisovalerate is acetohydroxy acid dehydratase. In some embodiments, a polypeptide that catalyzes a substrate to product conversion of alpha-ketoisovalerate to isobutyraldehyde is branched-chain alpha-keto acid decarboxylase. In some embodiments, a polypeptide that catalyzes a substrate to product conversion of alpha-ketoisovalerate to isobutyryl-CoA is branched-chain keto acid dehydrogenase. In some embodiments, a polypeptide that catalyzes a substrate to product conversion of isobutyryl-CoA to isobutyraldehyde is acylating aldehyde dehydrogenase. In some embodiments, a polypeptide that catalyzes a substrate to product conversion of alpha-ketoisovalerate to L-valine is transaminase. In some embodiments, a polypeptide that catalyzes a substrate to product conversion of alpha-ketoisovalerate to L-valine is valine dehydrogenase. In some embodiments, a polypeptide that catalyzes a substrate to product conversion of L-valine to isobutylamine is valine decarboxylase. In some embodiments, a polypeptide that catalyzes a substrate to product conversion of isobutylamine to isobutyraldehyde is omega transaminase. In some embodiments, a polypeptide that catalyzes a substrate to product conversion of butyryl-CoA to isobutyryl-CoA is isobutyryl-CoA mutase.

In some embodiments, the at least one alcohologenic polypeptide catalyzes a substrate to product conversion selected from the group consisting of: a) pyruvic acid to alpha-acetolactate; b) alpha-acetolactate to acetoin; c) acetoin to 2,3-butanediol; d) 2,3-butanediol to 2-butanone; e) 2-butanone to 2-butanol; and combinations thereof.

In some embodiments, a polypeptide that catalyzes a substrate to product conversion of alpha-acetolactate to acetoin is acetolactate decarboxylase. In some embodiments, a polypeptide that catalyzes a substrate to product conversion of pyruvic acid to alpha-acetolactate is acetolactate synthase. In some embodiments, a polypeptide that catalyzes a substrate to product conversion of acetoin to 2,3-butanediol is butanediol dehydrogenase. In some embodiments, a polypeptide that catalyzes a substrate to product conversion of 2,3-butanediol to 2-butanone is butanediol dehydratase. In some embodiments, a polypeptide that catalyzes a substrate to product conversion of 2-butanone to 2-butanol is butanol dehydrogenase.

In some embodiments, a recombinant microbial cell provided herein exhibits increased alcohol production as compared with the parent cell. In some embodiments, alcohol production is increased by the at least one alcohologenic modification. In some embodiments, increased alcohol production is determined by measuring a characteristic selected from the group consisting of: broth titer (grams aliphatic alcohol produced per liter broth (g l-1)), aliphatic alcohol yield (grams aliphatic alcohol produced per gram substrate consumed (g g-1), volumetric productivity (grams aliphatic alcohol produced per liter per hour (g l-1 h-1)), and specific productivity (grams aliphatic alcohol produced per gram recombinant cell biomass per hour (g/g cells h-1)), and combinations thereof.

In some embodiments, broth titer is increased at least 10%, 25%, 50%, 75%, 100%, or more. In some embodiments, yield is increased at least 10%, 25%, 50%, 75%, 100%, or more. In some embodiments, volumetric productivity is increased at least 10%, 25%, 50%, 75%, 100%, or more. In some embodiments, specific productivity is increased at least 10%, 25%, 50%, 75%, 100%, or more.

In some embodiments, an aliphatic alcohol compound comprises a compound selected from the group consisting of: methanol, ethanol, 1-propanol, 2-propanol, iso-propanol, 1-butanol, 2-butanol, iso-butanol, tert-butanol, 1-pentanol, 2-pentanol, 3-pentanol, iso-pentanol, 1-hexanol, 2-hexanol, 3-hexanol, 2-ethyl hexanol, iso-hexanol, 1-heptanol, 2-heptanol, 3-heptanol, 4-heptanol, iso-heptanol, 1-octanol, 2-octanol, 3-octanol, iso-octanol, and 4-octanol, and combinations thereof.

In some embodiments, an aliphatic alcohol compound comprises a compound selected from the group consisting of: 1-butanol, 2-butanol, iso-butanol, tert-butanol, and combinations thereof. In particular embodiments, the aliphatic alcohol compound comprises 1-butanol.

In another aspect, the present disclosure provides a recombinant cell engineered to contain or express an alcohol tolerance determinant selected from the group consisting of: a) a determinant sequence set forth in Table 1A; b) a determinant sequence set forth in Table 1B; c) a determinant sequence set forth in Table 2; and combinations thereof. In some embodiments, a recombinant cell is engineered to contain or express an alcohol tolerance determinant which is a determinant sequence set forth in Table 1A. In some embodiments, a cell is engineered to contain or express a determinant sequence selected from the group consisting of those found within a Table 1A row selected from the group consisting of row 20 (lp_{—}1293), row 21 (lp_{—}1295 [nmtH3]), row 34 (lp_{—}2159), row 35 (lp_{—}2160), row 36 (lp_{—}2169), row 37 (lp_{—}2170), row 44 (lp_{—}2911), and row 50 (lp_{—}3193).

In some embodiments, a cell is engineered to contain or express an alcohol tolerance determinant which is a determinant sequence set forth in Table 1B. In some embodiments, a cell is engineered to contain or express an alcohol tolerance determinant which is a determinant sequence set forth in Table 2. In some embodiments, a cell is engineered to contain or express a determinant sequence selected from the group consisting of those found within Tables 2T, 2U, 2AH, 2AI, 2AJ, 2AK, 2AR, and 2AX.

In another aspect, the present disclosure provides a recombinant cell that includes an alcohol tolerance modification (e.g., as compared with a parent cell), wherein the alcohol tolerance modification comprises introduction of alcohol tolerance determinant sequences selected from the group consisting of: a) a determinant sequence set forth in Table 1A; b) a determinant sequence set forth in Table 1B; c) determinant sequence set forth in Table 2; and combinations thereof. In some embodiments, a recombinant cell includes an alcohol tolerance determinant which is a determinant sequence set forth in Table 1A. In some embodiments, a cell includes a determinant sequence selected from the group consisting of those found within a Table 1A row selected from the group consisting of row 20 (lp_{—}1293), row 21 (lp_{—}1295 [nmtH3]), row 34 (lp_{—}2159), row 35 (lp_{—}2160), row 36 (lp_{—}2169), row 37 (lp_{—}2170), row 44 (lp_{—}2911), and row 50 (lp_{—}3193). In some embodiments, a cell includes an alcohol tolerance determinant which is a determinant sequence set forth in Table 1B. In some embodiments, a cell includes an alcohol tolerance determinant which is a determinant sequence set forth in Table 2. In some embodiments, a cell includes a determinant sequence selected from the group consisting of those found within Tables 2T, 2U, 2AH, 2AI, 2AJ, 2AK, 2AR, and 2AX.

In another aspect, the present disclosure provides a recombinant cell that includes an alcohol tolerance modification (e.g., as compared with a parent cell), which alcohol tolerance modification comprises introduction of a first alcohol tolerance determinant sequence and introduction of a second alcohol tolerance determinant sequence, wherein the first alcohol tolerance determinant sequence is selected from the group consisting of those found in Tables 1 and 2, and the second alcohol tolerance determinant sequence is selected from the group consisting of those found in Tables 3 and 4.

In some embodiments, a first alcohol tolerance determinant sequence is an alcohol tolerance determinant sequence selected from the group consisting of: a) a determinant sequence set forth in Table 1A; b) a determinant sequence set forth in Table 1B; c) determinant sequence set forth in Table 2; and combinations thereof. In some embodiments, a recombinant cell includes an alcohol tolerance determinant which is a determinant sequence set forth in Table 1A. In some embodiments, a cell includes a determinant sequence selected from the group consisting of those found within a Table 1A row selected from the group consisting of row 20 (lp_{—}1293), row 21 (lp_{—}1295 [nmtH3]), row 34 (lp_{—}2159), row 35 (lp_{—}2160), row 36 (lp_{—}2169), row 37 (lp_{—}2170), row 44 (lp_{—}2911), and row 50 (lp_{—}3193). In some embodiments, a cell includes an alcohol tolerance determinant which is a determinant sequence set forth in Table 1B.

In some embodiments, a cell includes an alcohol tolerance determinant which is a determinant sequence set forth in Table 2. In some embodiments, a cell includes a determinant sequence selected from the group consisting of those found within Tables 2T, 2U, 2AH, 2AI, 2AJ, 2AK, 2AR, and 2AX.

In some embodiments, a second alcohol tolerance determinant sequence is an alcohol tolerance determinant sequence found in Tables 3 and 4 which is selected from the group consisting of those found in sequences present in rows 2 (groES), 3 (groEL), 5 (cfa1) and/or 8 (cfa2) of Table 3, or in any of Tables 4B, 4C, 4E and 4H.

In some embodiments, the at least one alcohol tolerance modification further comprises disruption or inhibition of an alcohol tolerance determinant selected from the group consisting of those found in sequences present in rows 1 (htrA), 4 (clpP), 6 (relA/spoT), 7 (hrcA), and/or 8 (cfa2) of Table 3, or in any of Tables 4A, 4D, 4F, 4G and 4H.

In some embodiments, a recombinant microbial cell further comprises at least one alcohologenic modification. In some embodiments, an alcohologenic modification increases expression or activity of at least one alcohologenic polypeptide. In some embodiments, an alcohologenic modification decreases expression or activity of at least one alcohologenic polypeptide. In some embodiments, an alcohologenic modification increases expression or activity of at least one alcohologenic polypeptide and decreases expression or activity of at least one other alcohologenic polypeptide. In some embodiments, an alcohologenic modification comprises expression of at least one heterologous alcohologenic polypeptide in the recombinant microbial cell. In some embodiments, an alcohologenic modification comprises expression of at least one heterologous gene encoding the at least one heterologous alcohologenic polypeptide. In some embodiments, the at least one alcohologenic polypeptide is a polypeptide that increases production of an aliphatic alcohol selected from the group consisting of: methanol, ethanol, 1-propanol, 2-propanol, iso-propanol, 1-butanol, 2-butanol, iso-butanol, tert-butanol, 1-pentanol, 2-pentanol, 3-pentanol, iso-pentanol, 1-hexanol, 2-hexanol, 3-hexanol, 2-ethyl hexanol, iso-hexanol, 1-heptanol, 2-heptanol, 3-heptanol, 4-heptanol, iso-heptanol, 1-octanol, 2-octanol, 3-octanol, iso-octanol, and 4-octanol, and combinations thereof. In some embodiments, the at least one alcohologenic polypeptide is a polypeptide that increases production of an aliphatic alcohol selected from the group consisting of: 1-butanol, 2-butanol, iso-butanol, tert-butanol, and combinations thereof. In some embodiments, the at least one alcohologenic polypeptide is a polypeptide that increases production of 1-butanol, 2-butanol, or iso-butanol.

In some embodiments, the at least one alcohologenic polypeptide catalyzes a substrate to product conversion selected from the group consisting of: a) acetyl-CoA to acetoacetyl-CoA; b) acetoacetyl-CoA to 3-hydroxybutyryl-CoA; c) 3-hydroxybutyryl-CoA to crotonyl-CoA; d) crotonyl-CoA to butyryl-CoA; e) butyryl-CoA to butyraldehyde; f) butyraldehyde to 1-butanol; and combinations thereof.

In some embodiments, a polypeptide that catalyzes a substrate to product conversion of acetyl-CoA to acetoacetyl-CoA is acetyl-CoA acetyltransferase. In some embodiments, a polypeptide that catalyzes a substrate to product conversion of acetoacetyl-CoA to 3-hydroxybutyryl-CoA is 3-hydroxybutyryl-CoA dehydrogenase. In some embodiments, a polypeptide that catalyzes a substrate to product conversion of 3-hydroxybutyryl-CoA to crotonyl-CoA is crotonase. In some embodiments, a polypeptide that catalyzes a substrate to product conversion of crotonyl-CoA to butyryl-CoA is butyryl-CoA dehydrogenase. In some embodiments, a polypeptide that catalyzes a substrate to product conversion of butyryl-CoA to butyraldehyde is butyraldehyde dehydrogenase. In some embodiments, a polypeptide that catalyzes a substrate to product conversion of butyraldehyde to 1-butanol is butanol dehydrogenase.

In some embodiments, the at least one alcohologenic polypeptide catalyzes a substrate to product conversion selected from the group consisting of: a) pyruvate to acetolactate; b) acetolactate to 2,3-dihydroxyisovalerate; c) alpha-ketoisovalerate to isobutyraldehyde; d) isobutyraldehyde to isobutanol; e) 2,3-dihydroxyisovalerate to alpha-ketoisovalerate; f) alpha-ketoisovalerate to isobutyraldehyde; g) alpha-ketoisovalerate to isobutyryl-CoA; h) isobutyryl-CoA to isobutyraldehyde; i) alpha-ketoisovalerate to L-valine; j) L-valine to isobutylamine; k) isobutylamine to isobutyraldehyde; 1) butyryl-CoA to isobutyryl-CoA; and combinations thereof.

In some embodiments, a polypeptide that catalyzes a substrate to product conversion of pyruvate to acetolactate is acetolactate synthase. In some embodiments, a polypeptide that catalyzes a substrate to product conversion of acetolactate to 2,3-dihydroxyisovalerate is acetohydroxy acid reductoisomerase. In some embodiments, a polypeptide that catalyzes a substrate to product conversion of acetolactate to 2,3-dihydroxyisovalerate is acetohydroxy acid isomeroreductase. In some embodiments, a polypeptide that catalyzes a substrate to product conversion of alpha-ketoisovalerate to isobutyraldehyde is branched-chain alpha-keto acid decarboxylase. In some embodiments, a polypeptide that catalyzes a substrate to product conversion of isobutyraldehyde to isobutanol is branched-chain alcohol dehydrogenase. In some embodiments, a polypeptide that catalyzes a substrate to product conversion of 2,3-dihydroxyisovalerate to alpha-ketoisovalerate is acetohydroxy acid dehydratase. In some embodiments, a polypeptide that catalyzes a substrate to product conversion of alpha-ketoisovalerate to isobutyraldehyde is branched-chain alpha-keto acid decarboxylase. In some embodiments, a polypeptide that catalyzes a substrate to product conversion of alpha-ketoisovalerate to isobutyryl-CoA is branched-chain keto acid dehydrogenase. In some embodiments, a polypeptide that catalyzes a substrate to product conversion of isobutyryl-CoA to isobutyraldehyde is acylating aldehyde dehydrogenase. In some embodiments, a polypeptide that catalyzes a substrate to product conversion of alpha-ketoisovalerate to L-valine is transaminase. In some embodiments, a polypeptide that catalyzes a substrate to product conversion of alpha-ketoisovalerate to L-valine is valine dehydrogenase. In some embodiments, a polypeptide that catalyzes a substrate to product conversion of L-valine to isobutylamine is valine decarboxylase. In some embodiments, a polypeptide that catalyzes a substrate to product conversion of isobutylamine to isobutyraldehyde is omega transaminase. In some embodiments, a polypeptide that catalyzes a substrate to product conversion of butyryl-CoA to isobutyryl-CoA is isobutyryl-CoA mutase.

In some embodiments, the at least one alcohologenic polypeptide catalyzes a substrate to product conversion selected from the group consisting of: a) pyruvic acid to alpha-acetolactate; b) alpha-acetolactate to acetoin; c) acetoin to 2,3-butanediol; d) 2,3-butanediol to 2-butanone; e) 2-butanone to 2-butanol; and combinations thereof.

In some embodiments, a polypeptide that catalyzes a substrate to product conversion of alpha-acetolactate to acetoin is acetolactate decarboxylase. In some embodiments, a polypeptide that catalyzes a substrate to product conversion of pyruvic acid to alpha-acetolactate is acetolactate synthase. In some embodiments, a polypeptide that catalyzes a substrate to product conversion of acetoin to 2,3-butanediol is butanediol dehydrogenase. In some embodiments, a polypeptide that catalyzes a substrate to product conversion of 2,3-butanediol to 2-butanone is butanediol dehydratase. In some embodiments, a polypeptide that catalyzes a substrate to product conversion of 2-butanone to 2-butanol is butanol dehydrogenase.

In another aspect, the present disclosure features an engineered bacterial cell comprising one or more alcohol tolerance modifications, wherein the one or more alcohol tolerance modifications comprises introduction of an alcohol tolerance determinant sequence encoding a calcineurin-like phosphoesterase polypeptide.

In another aspect, the present disclosure features an engineered bacterial cell comprising one or more alcohol tolerance modifications, wherein the one or more alcohol tolerance modifications comprises introduction of an alcohol tolerance determinant sequence encoding a cation transport protein (mntH3 related) polypeptide.

In another aspect, the present disclosure features an engineered bacterial cell comprising one or more alcohol tolerance modifications, wherein the one or more alcohol tolerance modifications comprises introduction of an alcohol tolerance determinant sequence encoding a transcription regulator (lp_{—}2159 related) polypeptide.

In another aspect, the present disclosure features an engineered bacterial cell comprising one or more alcohol tolerance modifications, wherein the one or more alcohol tolerance modifications comprises introduction of an alcohol tolerance determinant sequence encoding an lp_{—}2160 related polypeptide.

In another aspect, the present disclosure features an engineered bacterial cell comprising one or more alcohol tolerance modifications, wherein the one or more alcohol tolerance modifications comprises introduction of an alcohol tolerance determinant sequence encoding a lp_{—}2169 related polypeptide.

In another aspect, the present disclosure features an engineered bacterial cell comprising one or more alcohol tolerance modifications, wherein the one or more alcohol tolerance modifications comprises introduction of an alcohol tolerance determinant sequence encoding a phosphoglycerate mutase polypeptide.

In another aspect, the present disclosure features an engineered bacterial cell comprising one or more alcohol tolerance modifications, wherein the one or more alcohol tolerance modifications comprises introduction of an alcohol tolerance determinant sequence encoding a CAAX protease polypeptide.

In another aspect, the present disclosure features an engineered bacterial cell comprising one or more alcohol tolerance modifications, wherein the one or more alcohol tolerance modifications comprises introduction of an alcohol tolerance determinant sequence encoding a peptidylprolyl isomerase (prs2A related polypeptide).

In another aspect, the present disclosure features an engineered bacterial cell comprising one or more alcohol tolerance modifications, wherein the one or more alcohol tolerance modifications comprises introduction of an alcohol tolerance determinant sequence found within Table 1A row 20 (lp_{—}1293).

In another aspect, the present disclosure features an engineered bacterial cell comprising one or more alcohol tolerance modifications, wherein the one or more alcohol tolerance modifications comprises introduction of an alcohol tolerance determinant sequence found within Table 1A, row 21 (lp_{—}1295 [nmtH3]).

In another aspect, the present disclosure features an engineered bacterial cell comprising one or more alcohol tolerance modifications, wherein the one or more alcohol tolerance modifications comprises introduction of an alcohol tolerance determinant sequence found within Table 1A, row 34 (lp_{—}2159).

In another aspect, the present disclosure features an engineered bacterial cell comprising one or more alcohol tolerance modifications, wherein the one or more alcohol tolerance modifications comprises introduction of an alcohol tolerance determinant sequence found within Table 1A, row 35 (lp_{—}2160).

In another aspect, the present disclosure features an engineered bacterial cell comprising one or more alcohol tolerance modifications, wherein the one or more alcohol tolerance modifications comprises introduction of an alcohol tolerance determinant sequence found within Table 1A, row 36 (lp_{—}2169).

In another aspect, the present disclosure features an engineered bacterial cell comprising one or more alcohol tolerance modifications, wherein the one or more alcohol tolerance modifications comprises introduction of an alcohol tolerance determinant sequence found within Table 1A, row 37 (lp_{—}2170).

In another aspect, the present disclosure features an engineered bacterial cell comprising one or more alcohol tolerance modifications, wherein the one or more alcohol tolerance modifications comprises introduction of an alcohol tolerance determinant sequence found within Table 1A, row 44 (lp_{—}2911).

In another aspect, the present disclosure features an engineered bacterial cell comprising one or more alcohol tolerance modifications, wherein the one or more alcohol tolerance modifications comprises introduction of an alcohol tolerance determinant sequence found within Table 1A, row 50 (lp_{—}3193).

In another aspect, the present disclosure provides a method of engineering a cell to include an alcohol tolerance modification, the method comprising: obtaining a parent cell, introducing into the parent cell at least one alcohol tolerance determinant sequence found within the alcohol tolerance determinant sequences in Tables 1 and 2, thereby engineering a cell to include an alcohol tolerance modification. In some embodiments, an alcohol tolerance determinant sequence comprises an alcohol tolerance determinant sequence in Table 1A. In some embodiments, an alcohol tolerance determinant sequence comprises an alcohol determinant sequence selected from the group consisting of those found within a Table 1A row selected from the group consisting of row 20 (lp_{—}1293), row 21 (lp_{—}1295 [nmtH3]), row 34 (lp_{—}2159), row 35 (lp_{—}2160), row 36 (lp_{—}2169), row 37 (lp_{—}2170), row 44 (lp_{—}2911), and row 50 (lp_{—}3193). In some embodiments, an alcohol tolerance determinant sequence comprises an alcohol tolerance determinant sequence in Table 2. In some embodiments, an alcohol tolerance determinant sequence comprises an alcohol tolerance determinant sequence in one or more of Tables 2T, 2U, 2AH, 2AI, 2AJ, 2AK, 2AR, 2AX.

In some embodiments, a method further includes introducing a second alcohol tolerance determinant sequence selected from the group consisting of those found in Tables 3 and 4. In some embodiments, an alcohol tolerance determinant sequence found in Tables 3 and 4 is selected from the group consisting of those found in sequences present in rows 2 (groES), 3 (groEL), 5 (cfa1) and/or 8 (cfa2) of Table 3, or in any of Tables 4B, 4C, 4E and 4H. In some embodiments, the at least one alcohol tolerance modification further comprises disruption or inhibition of an alcohol tolerance determinant selected from the group consisting of those found in Tables 3 and 4. In some embodiments, an alcohol tolerance determinant found in Tables 3 and 4 is selected from the group consisting of those found in sequences present in rows 1 (htrA), 4 (clpP), 6 (relA/spoT), 7 (hrcA), and/or 8 (cfa2) of Table 3, or in any of Tables 4A, 4D, 4F, 4G and 4H.

In another aspect, the present disclosure features a method of producing an aliphatic alcohol compound, comprising steps of: a) cultivating the recombinant microbial cell provided herein under conditions and for a time sufficient that the aliphatic alcohol compound is produced; and b) isolating the produced aliphatic alcohol compound. In some embodiments, an aliphatic alcohol compound is selected from the group consisting of: methanol, 1-propanol, 2-propanol, iso-propanol, 1-butanol, 2-butanol, iso-butanol, tert-butanol, 1-pentanol, 2-pentanol, 3-pentanol, iso-pentanol, 1-hexanol, 2-hexanol, 3-hexanol, 2-ethyl hexanol, iso-hexanol, 1-heptanol, 2-heptanol, 3-heptanol, 4-heptanol, iso-heptanol, 1-octanol, 2-octanol, 3-octanol, iso-octanol, and 4-octanol, and combinations thereof.

In another aspect, the present disclosure features a method of producing a butanol compound, comprising steps of: a) cultivating a recombinant microbial cell provided herein under conditions that allow production of the aliphatic alcohol compound under conditions and for a time sufficient that the butanol compound accumulates to greater than 30 grams per liter; and b) isolating the produced butanol compound. In some embodiments, a butanol compound is selected from the group consisting of: 1-butanol, 2-butanol, iso-butanol, tert-butanol, and combinations thereof. In some embodiments, a butanol compound is 1-butanol.

In another aspect, the present disclosure provides a method of producing an aliphatic alcohol compound, comprising steps of: a) cultivating a recombinant microbial cell provided herein under conditions and for a time sufficient that the aliphatic alcohol compound is produced; and b) isolating the produced aliphatic alcohol compound. In some embodiments, an aliphatic alcohol compound is selected from the group consisting of: methanol, ethanol, 1-propanol, 2-propanol, iso-propanol, 1-butanol, 2-butanol, iso-butanol, tert-butanol, 1-pentanol, 2-pentanol, 3-pentanol, iso-pentanol, 1-hexanol, 2-hexanol, 3-hexanol, 2-ethyl hexanol, iso-hexanol, 1-heptanol, 2-heptanol, 3-heptanol, 4-heptanol, iso-heptanol, 1-octanol, 2-octanol, 3-octanol, iso-octanol, and 4-octanol, and combinations thereof.

In another aspect, the present disclosure provides a method of producing a butanol compound, comprising steps of: a) cultivating the recombinant microbial cell described herein under conditions and for a time sufficient that the butanol compound accumulates to greater than 30 grams per liter; and b) isolating the produced butanol compound. In some embodiments, a butanol compound is selected from the group consisting of: 1-butanol, 2-butanol, iso-butanol, tert-butanol, and combinations thereof. In some embodiments, a butanol compound is 1-butanol.

In another aspect, the present disclosure provides an aliphatic alcohol compound composition, prepared by a method comprising steps of: a) cultivating a recombinant microbial cell provided herein under conditions and for a time sufficient that the aliphatic alcohol compound is produced; and b) isolating the produced aliphatic alcohol compound. In some embodiments, an aliphatic alcohol compound is selected from the group consisting of: methanol, 1-propanol, 2-propanol, iso-propanol, 1-butanol, 2-butanol, iso-butanol, tert-butanol, 1-pentanol, 2-pentanol, 3-pentanol, iso-pentanol, 1-hexanol, 2-hexanol, 3-hexanol, 2-ethyl hexanol, iso-hexanol, 1-heptanol, 2-heptanol, 3-heptanol, 4-heptanol, iso-heptanol, 1-octanol, 2-octanol, 3-octanol, iso-octanol, and 4-octanol, and combinations thereof.

In another aspect, the present disclosure provides an aliphatic alcohol compound composition, prepared by a method comprising steps of: a) cultivating a recombinant microbial cell provided herein under conditions and for a time sufficient that the aliphatic alcohol compound is produced; and b) isolating the produced aliphatic alcohol compound. In some embodiments, an aliphatic alcohol compound is selected from the group consisting of: methanol, ethanol, 1-propanol, 2-propanol, iso-propanol, 1-butanol, 2-butanol, iso-butanol, tert-butanol, 1-pentanol, 2-pentanol, 3-pentanol, iso-pentanol, 1-hexanol, 2-hexanol, 3-hexanol, 2-ethyl hexanol, iso-hexanol, 1-heptanol, 2-heptanol, 3-heptanol, 4-heptanol, iso-heptanol, 1-octanol, 2-octanol, 3-octanol, iso-octanol, and 4-octanol, and combinations thereof.

In another aspect, the present disclosure provides a butanol compound composition, prepared by a method comprising steps of: a) cultivating a recombinant microbial cell provided herein under conditions and for a time sufficient that the butanol compound is produced; and b) isolating the produced butanol compound. In some embodiments, a butanol compound is selected from the group consisting of: 1-butanol, 2-butanol, iso-butanol, tert-butanol, and combinations thereof. In some embodiments, the butanol compound is 1-butanol.

In another aspect, the present disclosure provides a butanol compound composition, prepared by a method comprising steps of: a) cultivating a recombinant microbial cell provided herein under conditions and for a time sufficient that the butanol compound is produced; and b) isolating the produced butanol compound. In some embodiments, a butanol compound is selected from the group consisting of: 1-butanol, 2-butanol, iso-butanol, tert-butanol, and combinations thereof. In some embodiments, a butanol compound is 1-butanol.

In another aspect, the present disclosure provides a method of preparing an aliphatic alcohol compound-containing product, comprising steps of: a) cultivating a recombinant microbial cell provided herein under conditions and for a time sufficient that the aliphatic alcohol compound is produced; b) isolating the aliphatic alcohol compound; and c) combining the aliphatic alcohol compound with one or more other additive components. In some embodiments, an aliphatic alcohol compound is a butanol compound selected from the group consisting of 1-butanol, 2-butanol, iso-butanol, tert-butanol, and combinations thereof. In some embodiments, a butanol compound is 1-butanol. In some embodiments, a product comprises transport fuel. In some embodiments, a product comprises a solvent. In some embodiments, a product comprises a swelling agent. In some embodiments, a product comprises a brake fluid. In some embodiments, a product comprises an extractant. In some embodiments, a product comprises a cement additive. In some embodiments, a product comprises an ore flotation agent. In some embodiments, a product comprises a melamine formaldehyde resin.

The present disclosure also provides isolated nucleic acid molecules that include, or consist of, a nucleic acid molecule having a sequence disclosed herein. Thus, in one aspect, the disclosure provides an isolated nucleic acid molecule comprising a 3′ region of a gene encoding a CAAX protease polypeptide. In some embodiments, a nucleic acid molecule comprises a nucleotide sequence at least 80% identical to the nucleotide sequence shown in Table 1B, row 42, a homolog thereof, or a portion thereof. In some embodiments, a nucleic acid molecule, when introduced into a host cell (e.g., a microbial host cell, e.g., a bacterial host cell), is sufficient to adjust susceptibility of the cell to a toxic effect of an alcohol compound. In some embodiments, a nucleic acid molecule lacks a nucleotide sequence encoding a CAAX protease polypeptide. In some embodiments, a nucleic acid molecule further includes a 5′ region of a gene encoding a CAAX protease polypeptide, e.g., wherein the 5′ region comprises a nucleotide sequence at least 80% identical to the nucleotide sequence shown in Table 1B, row 40, a homolog thereof, or a portion thereof.

In another aspect, the present disclosure provides an isolated nucleic acid molecule comprising a 5′ region of a gene encoding a CAAX protease polypeptide. In some embodiments, a 5′ region comprises a nucleotide sequence at least 80% identical to the nucleotide sequence shown in Table 1B, row 40, a homolog thereof, or a portion thereof. In some embodiments, a nucleic acid molecule lacks a nucleotide sequence encoding a CAAX protease polypeptide.

Vectors comprising the nucleic acid molecules are also provided herein.

### BRIEF DESCRIPTION OF THE DRAWING

FIG. 1 presents a representative metabolic pathway that produces aliphatic alcohol compounds such as ethanol and 1-butanol. The depicted pathway is utilized, for example, in many C. acetobutylicum strains. Names of certain particular enzymes known to perform indicated steps in such strains are indicated, with their corresponding gene names indicated in parentheses.

FIG. 2 illustrates different growth conditions that promote different metabolic states in certain microorganisms such as, for example, C. acetobutylicum. Panel A illustrates the solventogenesis state; Panel B illustrates the alcohologenesis state. Abbreviations: Fd, ferredoxin; Fdred, reduced ferredoxin; Fdox, oxidized ferredoxin; dep, dependent; AYDH, acetaldehyde dehydrogenase; ETDH, ethanol dehydrogenase; ADC, acetoacetate decarboxylase; BYDH, butyraldehyde dehydrogenase; BUDH, butanol dehydrogenase; +, high level of in vitro enzyme activity; −, low level of in vitro enzyme activity.

FIG. 3 illustrates certain metabolic pathways that operate to produce a particular aliphatic alcohol compound, isobutanol. In particular, FIG. 3 shows four different isobutanol biosynthetic pathways. The steps labeled “a”, “b”, “c”, “d”, “e”, “f”, “g”, “h”, “i”, “j” and “k” represent the substrate to product conversions

FIG. 4, Panels A-D, show metabolic tolerance assay results for Lactobacillus plantarum strains comprising alcohol tolerant determinant sequences. Graphs depict OD₆₀₀ and HPLC measurements of lactate, glucose, and 1-butanol levels. The strains were grown in the presence of either 0% (panel A), 1.7% (panel B), 2.0% (panel C), or 2.3% (panel D) 1-butanol.

### TABLES

The tables referenced in the description exceed more than 100 pages and are submitted electronically. The tables themselves and each reference and information designated by each of the Genbank Accession and GI numbers are hereby incorporated by reference in their entirety.

### DEFINITIONS

Acetohydroxy acid dehydratase: The term “acetohydroxy acid dehydratase” refers to an enzyme that catalyzes the conversion of 2,3-dihydroxyisovalerate to α-ketoisovalerate. Preferred acetohydroxy acid dehydratases are known by the EC number 4.2.1.9 (Enzyme Nomenclature 1992, Academic Press, San Diego). These enzymes are available from a vast array of microorganisms, including, but not limited to, Escherichia coli (GenBank Nos: YP_{—}026248 (SEQ ID NO:6 in US patent application 2007/0092957), NC_{—}000913 (SEQ ID NO:5 in US patent application 2007/0092957) NCBI (National Center for Biotechnology Information) amino acid sequence and NCBI nucleotide sequences), Saccharomyces cerevisiae (GenBank Nos: NP_{—}012550 (SEQ ID NO:186 in US patent application 2007/0092957), NC_{—}001142 (SEQ ID NO:83 in US patent application 2007/0092957)), Methanococcus maripaludis (GenBank Nos: CAF29874 (SEQ ID NO:188 in US patent application 2007/0092957), BX957219 (SEQ ID NO:187 in US patent application 2007/0092957)), and Bacillus subtilis (GenBank Nos: CAB14105 (SEQ ID NO:190 in US patent application 2007/0092957), Z99115 (SEQ ID NO:189 in US patent application 2007/0092957)).

Acetohydroxy acid isomeroreductase: The terms “acetohydroxy acid isomeroreductase” and “acetohydroxy acid reductoisomerase” are used interchangeably herein to refer to an enzyme that catalyzes the conversion of acetolactate to 2,3-dihydroxyisovalerate using NADPH (reduced nicotinamide adenine dinucleotide phosphate) as an electron donor. Preferred acetohydroxy acid isomeroreductases are known by the EC number 1.1.1.86 and sequences are available from a vast array of microorganisms, including, but not limited to, E. coli (GenBank Nos: NP_{—}418222 (SEQ ID NO:4 in US patent application 2007/0092957), NC_{—}000913 (SEQ ID NO:3 in US patent application 2007/0092957)), S. cerevisiae (GenBank Nos: NP_{—}013459 (SEQ ID NO:181 in US patent application 2007/0092957), NC_{—}001144 (SEQ ID NO:80 in US patent application 2007/0092957)), Methanococcus maripaludis (GenBank Nos: CAF30210 (SEQ ID NO:183 in US patent application 2007/0092957), BX957220 (SEQ ID NO:182 in US patent application 2007/0092957)), and B. subtilis (GenBank Nos: CAB14789 (SEQ ID NO:185 in US patent application 2007/0092957), Z99118 (SEQ ID NO:184 in US patent application 2007/0092957)).

Acetolactate synthase: The terms “acetolactate synthase” and “acetolactate synthetase” are used interchangeably herein to refer to an enzyme that catalyzes the conversion of pyruvate to acetolactate and CO₂. Exemplary acetolactate synthases are known by the EC number 2.2.1.6. These enzymes are available from a number of sources, including, but not limited to, B. subtilis (GenBank Nos: CAB15618 (SEQ ID NO:178 of US patent application 2007/0092957), Z99122 (SEQ ID NO:78 of US patent application 2007/0092957)), Klebsiella pneumoniae (GenBank Nos: AAA25079 (SEQ ID NO:2 of US patent application 2007/0092957), M73842 (SEQ ID NO:1 of US patent application 2007/0092957)), and Lactococcus lactis (GenBank Nos: AAA25161 (SEQ ID NO:180), L16975 (SEQ ID NO:179)).

Acetyl-CoA acetyltransferase: The term “acetyl-CoA acetyltransferase” refers to an enzyme that catalyzes the conversion of two molecules of acetyl-CoA to acetoacetyl-CoA and coenzyme A (CoA). Preferred acetyl-CoA acetyltransferases are acetyltransferases with substrate preferences (reaction in the forward direction) for a short chain acyl-CoA and acetyl-CoA and are classified as E.C.2.3.1.9.; although, enzymes with a broader substrate range (E.C.2.3.1.16) will be functional as well. Acetyl-CoA acetyltransferases are available from a number of sources, for example, E. coli (GenBank Nos: NP_{—}416728 (SEQ ID NO:129 in WO 2007/041269), NC_{—}000913 (SEQ ID NO:128 in WO 2007/041269)), Clostridium acetobutylicum (GenBank Nos: NP_{—}349476.1 (SEQ ID NO:2 in WO 2007/041269), NC_{—}003030 (SEQ ID NO:1 in WO 2007/041269), NP_{—}149242 (SEQ ID NO:4 in WO 2007/041269), NC_{—}001988 (SEQ ID NO:3 in WO 2007/041269)), B. subtilis (GenBank Nos: NP_{—}390297 (SEQ ID NO:131 in WO 2007/041269), NC_{—}000964 (SEQ ID NO:130 in WO 2007/041269)), and S. cerevisiae (GenBank Nos: NP_{—}015297 (SEQ ID NO:133 in WO 2007/041269), NC_{—}001148 (SEQ ID NO:132 in WO 2007/041269)).

Acylating aldehyde dehydrogenase: The term “acylating aldehyde dehydrogenase” refers to an enzyme that catalyzes the conversion of isobutyryl-CoA to isobutyraldehyde, using either NADH or NADPH as electron donor. Preferred acylating aldehyde dehydrogenases are known by the EC numbers 1.2.1.10 and 1.2.1.57. These enzymes are available from multiple sources, including, but not limited to, Clostridium beijerinckii (GenBank Nos: AAD31841 (SEQ ID NO:222 in US patent application 2007/0092957), AF157306 (SEQ ID NO:221 in US patent application 2007/0092957)), C. acetobutylicum (GenBank Nos: NP_{—}149325 (SEQ ID NO:224 in US patent application 2007/0092957), NC_{—}001988 (SEQ ID NO:223 in US patent application 2007/0092957) NP_{—}149199 (SEQ ID NO:226 in US patent application 2007/0092957), NC_{—}001988 (SEQ ID NO:225 in US patent application 2007/0092957)), Pseudomonas putida (GenBank Nos: AAA89106 (SEQ ID NO:228 in US patent application 2007/0092957), U13232 (SEQ ID NO:227 in US patent application 2007/0092957)), and Thermus thermophilus (GenBank Nos: YP_{—}145486 (SEQ ID NO:230 in US patent application 2007/0092957), NC_{—}006461 (SEQ ID NO:229 in US patent application 2007/0092957)).

Alcohol tolerance determinant: The term “alcohol tolerance determinant”, as used herein, refers to a nucleic acid that, when introduced into an organism, alters its susceptibility to toxic effects of one or more aliphatic alcohol compounds, as described herein. Thus, introduction of an alcohol tolerance determinant into an organism constitutes applying an alcohol tolerance modification to that organism. In some embodiments, an alcohol tolerance determinant includes sequences that encode one or more polypeptides; in some embodiments such polypeptides may be alcohol tolerance polypeptides. In some embodiments, an alcohol tolerance determinant includes sequences that do not encode one or more polypeptides; in some embodiments, an alcohol tolerance determinant does not encode a polypeptide. In some embodiments, an alcohol tolerance determinant is found among determinant sequences presented in one or more of Tables 1-4 (i.e., in one or more of Tables 1A, 1B, 2A-2BE, 3, and 4A-4H). In some embodiments, introduction of an alcohol tolerance determinant into (or expression of an alcohol tolerance determinant in) a cell increases tolerance to one or more toxic effects of one or more aliphatic alcohols; in some embodiments, inactivation or inhibition of an alcohol tolerance determinant in a cell increases tolerance. To give but a few examples, alcohol tolerance determinants whose introduction into or expression in a cell increases alcohol tolerance may include, e.g., determinants in Table 1A in row 20 (lp_{—}1293), row 21 (lp_{—}1295 [nmtH3]), row 34 (lp_{—}2159), row 35 (lp_{—}2160), row 36 (lp_{—}2169), row 37 (lp_{—}2170), row 44 (lp_{—}2911), and row 50 (lp_{—}3193); determinants in rows 2 (groES), 3 (groEL), 5 (cfa1) and/or 8 (cfa2) of Table 3, or determinants in any of Tables 2T, 2U, 2AH, 2AI, 2AJ, 2AK, 2AR, 2AX, 4B, 4C, 4E and/or 4H; alcohol tolerance determinants whose inactivation or inhibition in a cell increases tolerance may include, e.g., those in rows 1 (htrA), 4 (clpP), 6 (relA/spoT), 7 (hrcA), and/or 8 (cfa2) of Table 3, or in any of Tables 4A, 4D, 4F, 4G and/or 4H.

Alcohol tolerance modification: The term “alcohol tolerance modification” refers to a modification of a host organism that adjusts its susceptibility to one or more toxic effects of one or more aliphatic alcohol compounds, as described herein. For example, in some embodiments, an organism containing an alcohol tolerance modification exhibits an increased aliphatic alcohol compound IC₅₀ as compared with an otherwise identical organism lacking the modification; in some embodiments, the aliphatic alcohol compound IC₅₀ is increased 5%, 10%, 20%, 30%, 40%, 50%, 60%, 70%, 80%, 90%, 100% or more. In some embodiments, an organism containing an alcohol tolerance modification exhibits increased carbohydrate utilization as compared with an otherwise identical organism lacking the modification when grown in the presence of the same amount of aliphatic alcohol compound; in some cases the carbohydrate utilization is increased by 5%, 10%, 20%, 30%, 40%, 50%, 60%, 70%, 80%, 90%, 100% or more. In some embodiments, an organism containing an alcohol tolerance modification exhibits increased production of at least one aliphatic alcohol compound as compared with an otherwise identical organism lacking the modification; in some embodiments, such increased production results in a broth titer of the produced at least one aliphatic alcohol compound that is 10%, 25%, 50%, 75%, 100% or more higher than that observed with an otherwise identical organism lacking the modification under comparable conditions, and/or such increased production results in a yield that is 10%, 25%, 50%, 75%, 100% or more of that of an otherwise identical organism lacking the modification, under comparable conditions, and/or such increased production results in volumetric productivity that is 10%, 25%, 50%, 75%, 100% or more of that of an otherwise identical organism lacking the modification, under comparable conditions, and/or such increased production results in a specific productivity increase of 10%, 25%, 50%, 75%, 100% or more as compared with an otherwise identical organism lacking the modification under comparable conditions. In some embodiments, an alcohol tolerance modification comprises introduction and/or expression of an alcohol tolerance determinant (so that a modified cell has an increased amount and/or level of expression or activity of an alcohol tolerance determinant as compared with a parental cell); in some embodiments, an alcohol tolerance modification comprises inactivation and/or inhibition of an alcohol tolerance determinant (so that a modified cell has a decreased amount and/or level of expression or activity of the alcohol tolerance determinant) as compared with a parental cell. In some embodiments, an alcohol tolerance modification achieves (or enhances, or inhibits) expression of one or more alcohol tolerance polypeptides in a cell.

Alcohol tolerance polypeptide: An alcohol tolerance polypeptide, as that term is used herein is any polypeptide that, when expressed in a cell, contributes to the cell's tolerance (e.g., as measured by IC₅₀, carbohydrate utilization, etc.) to at least one aliphatic alcohol compound. For example, a butanol tolerance polypeptide is a polypeptide whose expression in a cell contributes to that cell's resistance to butanol, etc. In some embodiments, alcohol tolerance polypeptides are selected from the group consisting of calcineurin-like phosphoesterase polypeptides, cation transport (mntH3 related) polypeptides, transcription regulator (lp_{—}2159 related) polypeptides, lp_{—}2160 related polypeptides, lp_{—}2169 related polypeptides, phosphoglycerate mutase polypeptides, CAAX protease polypeptides, peptidylprolyl isomerase (prs2A related) polypeptides, and combinations thereof. In some embodiments, certain stress-response polypeptides are alcohol tolerance polypeptides. For example, GroES chaperonin polypeptides, GroEL chaperonin polypeptides, and combinations thereof, are alcohol tolerance polypeptides in accordance with certain embodiments of the present disclosure. Alternatively or additionally, in some embodiments, alcohol tolerance polypeptides include serine protease HtrA polypeptides, GroES chaperonin polypeptides, GroEL chaperonin polypeptides, ATP-dependent Clp protease proteolytic subunit polypeptides, cyclopropane-fatty-acyl-phospholipid synthase #1 (cfa1) polypeptides, GTP pyrophosphokinase (relA/spoT) polypeptides, heat-inducible transcription repressor (hrca) polypeptides, cyclopropane-fatty-acyl-phospholipid synthase #2 (cfa2) polypeptides and combinations thereof. In some embodiments, alcohol tolerance polypeptides are encoded by an alcohol tolerance determinant, for example as set forth in any one or more of Tables 1-4. In some particular embodiments, alcohol tolerance polypeptides are encoded by an alcohol tolerance determinant, for example, included in determinant sequences set forth in any of rows 20 (lp_{—}1293), 210p_{—}1295 [nmtH3], 34 (lp_{—}2159), 35 (lp_{—}2160), 36 (lp_{—}2169), 37 (lp_{—}2170), 44 (lp_{—}2911), and/or 50 (lp_{—}3193 [prs2A]) of Table 1A, and/or in any of Tables 2T, 2U, 2AH, 2AI, 2AJ, 2AK, 2AR, and/or 2AX. In some embodiments, alcohol tolerance polypeptides are encoded by an alcohol tolerance determinant, for example, set forth in Table 3 or Table 4. An alcohol tolerance modification may alter production and/or activity of any such alcohol tolerance polypeptide, or combination thereof. In some embodiments, increased expression or activity of an alcohol tolerance polypeptide increases tolerance; in some embodiments, decreased expression or activity of an alcohol tolerance polypeptide increases tolerance. To give but a few examples, alcohol tolerance polypeptides whose increased expression or activity in a cell increases alcohol tolerance may include, e.g., those encoded by alcohol tolerance determinants included in the determinant sequences found in Table 1A, row 20 (lp_{—}1293), row 21 (lp_{—}1295 [nmtH3]), row 34 (lp_{—}2159), row 35 (lp_{—}2160), row 36 (lp_{—}2169), row 37 (lp_{—}2170), row 44 (lp_{—}2911), row 50 (lp_{—}3193); those encoded by alcohol tolerance determinants included in determinant sequences found in rows 2 (groES), 3 (groEL), 5 (cfa1) and/or 8 (cfa2) of Table 3, or in any of Tables 2T, 2U, 2AH, 2AI, 2AJ, 2AK, 2AR, 2AX, 4B, 4C, 4E and/or 4H; alcohol tolerance polypeptides whose decreased expression or activity in a cell increases tolerance may include, e.g., those encoded by alcohol tolerance determinants included in determinant sequences found in rows 1 (htrA), 4 (clpP), 6 (relA/spoT), 7 (hrcA), and/or 8 (cfa2) of Table 3, or in any of Tables 4A, 4D, 4F, 4G and/or 4H.

Alcohologenic modification: The term “alcohologenic modification” refers to a modification of a host organism that increases its production of at least one aliphatic alcohol compound. For example, in some embodiments, such increased production results in a broth titer of the produced at least one aliphatic alcohol compound that is 10%, 25%, 50%, 75%, 100% or more higher than that observed with an otherwise identical organism lacking the modification under comparable conditions, and/or such increased production results in a yield that is 10%, 25%, 50%, 75%, 100% or more higher than that observed with an otherwise identical organism lacking the modification under comparable conditions, and/or such increased production results in volumetric productivity that is 10%, 25%, 50%, 75%, 100% or more higher than that observed with an otherwise identical organism lacking the modification under comparable conditions, and/or such increased production results in a specific productivity increase of 10%, 25%, 50%, 75%, 100% or more higher than that observed with an otherwise identical organism lacking the modification under comparable conditions. In some embodiments, an alcohologenic modification is also an alcohol tolerance modification. In some embodiments, an alcohologenic modification comprises expression of an aliphatic alcohol biosynthesis polypeptide and/or inhibition of an aliphatic alcohol biosynthesis competitor polypeptide. In some embodiments, an alcohologenic modification increases expression of an alcohologenic polypeptide which is a homologous polypeptide (e.g., the alcohologenic modification increases expression of a polypeptide that naturally occurs in the organism in which it is being expressed). In some embodiments, an alcohologenic modification comprises expression of a heterologous alcohologenic polypeptide.

Aliphatic alcohol biosynthesis polypeptide: An “aliphatic alcohol biosynthesis polypeptide”, as that term is used herein, refers to any polypeptide that is involved in the synthesis of an aliphatic alcohol compound. In some embodiments, an aliphatic alcohol compound catalyzes at least one synthetic step in production of at least one aliphatic alcohol compound. An aliphatic alcohol biosynthesis polypeptide involved in the synthesis of a particular aliphatic alcohol compound may be referred to by reference to that compound (e.g., ethanol biosynthesis polypeptide, 1-butanol biosynthesis polypeptide, butanol biosynthesis peptide, isobutanol biosynthesis polypeptide, etc.). Thus, in some embodiments, a butanol biosynthesis polypeptide catalyzes at least one step in the synthesis of butanol. In some embodiments, an aliphatic alcohol biosynthesis polypeptide catalyzes a substrate to product conversion selected from the group consisting of a) acetyl-CoA to acetoacetyl-CoA; b) acetoacetyl-CoA to 3-hydroxybutyryl-CoA; c) 3-hydroxybutyryl-CoA to crotonyl-CoA; d) crotonyl-CoA to butyryl-CoA; e) butyryl-CoA to butyraldehyde; f) butyraldehyde to 1-butanol; and combinations thereof. In some embodiments, an aliphatic alcohol biosynthesis polypeptide is an acetyl-CoA acetyltransferase polypeptide, a 3-hydroxybutyryl-CoA dehydrogenase polypeptide, a crotonase polypeptide, a butyryl-CoA dehydrogenase polypeptide, a butyraldehyde dehydrogenase polypeptide, a butanol dehydrogenase polypeptide, or a combination thereof. In some embodiments, an isobutanol biosynthesis polypeptide catalyzes at least one step in the synthesis of isobutanol. Thus, in some embodiments, an aliphatic alcohol biosynthesis polypeptide catalyzes a substrate to product conversion selected from the group consisting of a) pyruvate to acetolactate; b) acetolactate to 2,3-dihydroxyisovalerate; c) 2,3-dihydroxyisovalerate to α-ketoisovalerate; d) α-ketoisovalerate to isobutyraldehyde; e) isobutyraldehyde to isobutanol; f) α-ketoisovalerate to isobutyryl-CoA; g) isobutyryl-CoA to isobutyraldehyde; h) α-ketoisovalerate to valine; i) valine to isobutylamine; j) isobutylamine to isobutyraldehyde; k) butyryl-CoA to isobutyryl-CoA; and combinations thereof. In some embodiments, an aliphatic alcohol biosynthesis polypeptide is an acetolactate synthase polypeptide, an acetohydroxy acid isomeroreductase polypeptide, an acetohydroxy acid dehydratase polypeptide, a branched-chain keto acid decarboxylase polypeptide, a branched-chain alcohol dehydrogenase polypeptide, a branched-chain keto acid dehydrogenase polypeptide, an acylating aldehyde dehydrogenase polypeptide, a valine dehydrogenase polypeptide, a transaminase polypeptide, a valine decarboxylase polypeptide, an omega transaminase polypeptide, an isobutyryl-CoA mutase polypeptide, or a combination thereof. Representative examples of some such aliphatic alcohol biosynthesis polypeptides are presented in Tables 5 and 6.

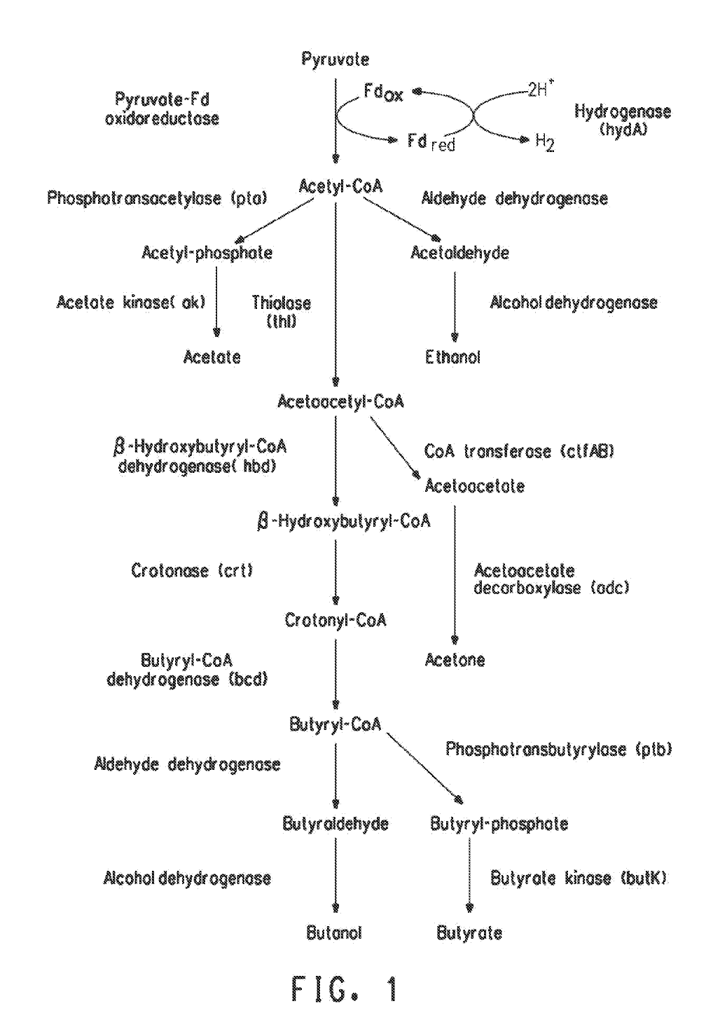

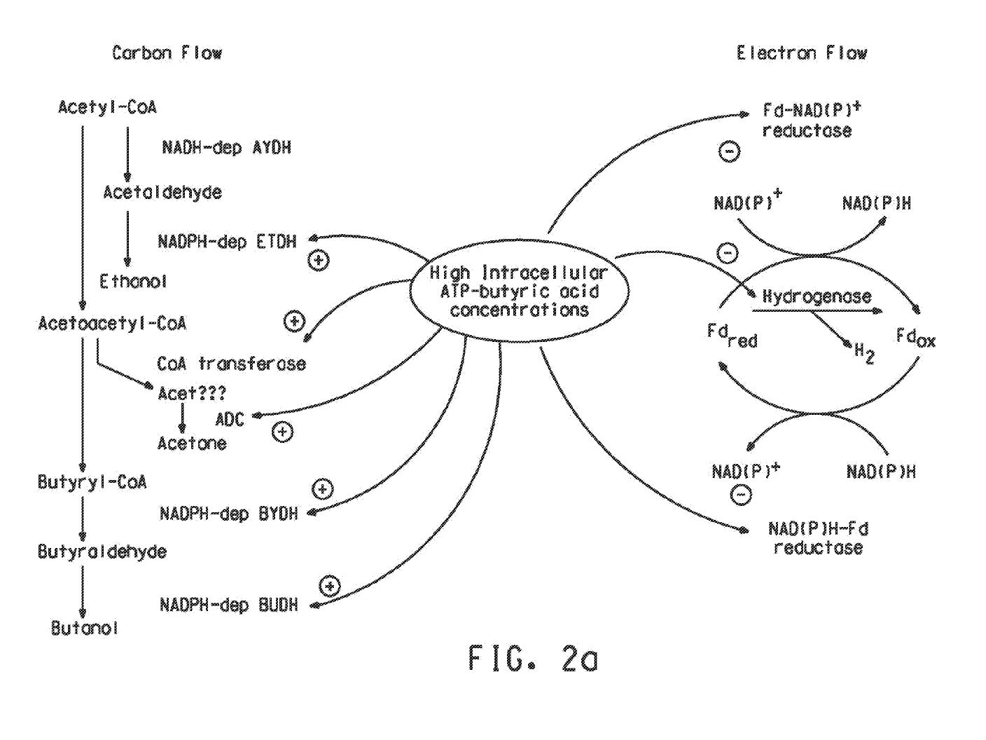

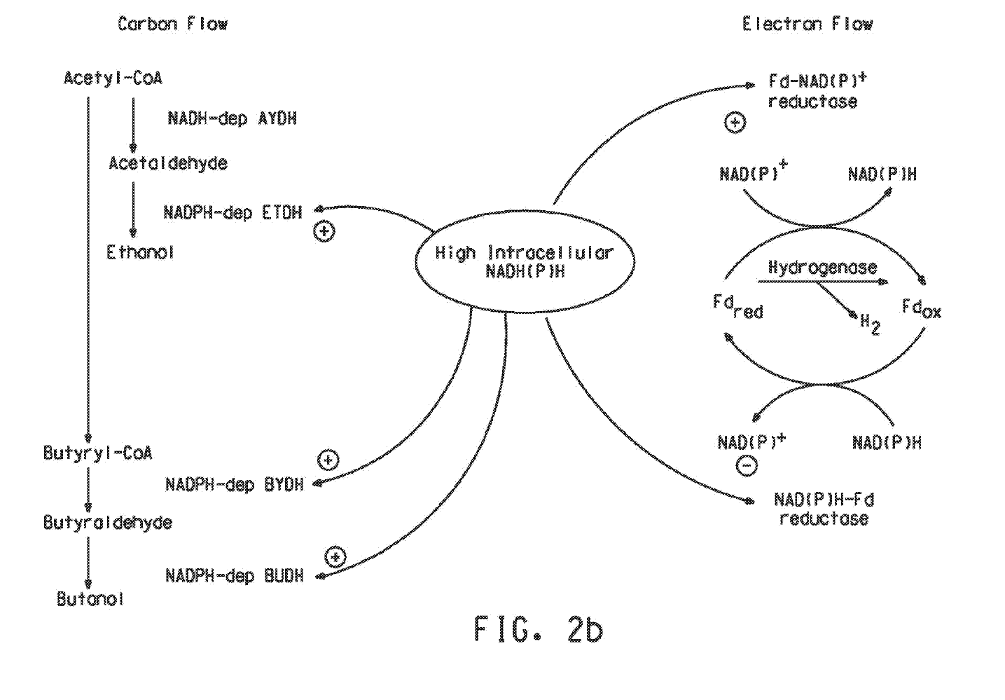

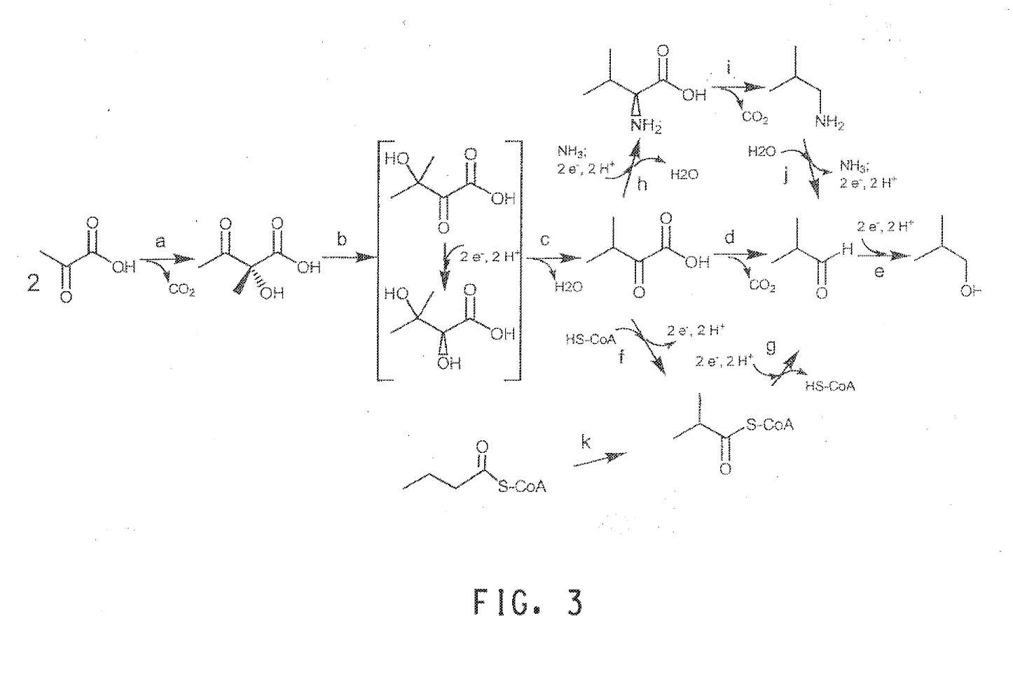

Aliphatic alcohol biosynthesis competitor polypeptide: An “aliphatic alcohol biosynthesis competitor polypeptide”, as that term is used here, is a polypeptide whose expression in a cell results in diversion of one or more metabolic intermediates away from a pathway that would otherwise produce one or more aliphatic alcohol compounds. In some embodiments of the present disclosure, aliphatic alcohol biosynthesis competitor polypeptides catalyze a metabolic reaction in a pathway that intersects an aliphatic alcohol biosynthesis pathway. In some embodiments of the present disclosure, expression of an aliphatic alcohol biosynthesis competitor polypeptide in a cell reduces levels of aliphatic alcohol compounds generally. In some embodiments of the present disclosure, expression of an aliphatic alcohol biosynthesis competitor polypeptide reduces levels of a particular aliphatic alcohol compound. In some embodiments of the present disclosure, expression of an aliphatic alcohol biosynthesis competitor polypeptide alters relative production levels of different aliphatic alcohol compounds. To give but one example, in some embodiments a butanol biosynthesis competitor polypeptide catalyzes the diversion of butanol metabolic intermediates to alternative pathways, such as those that promote the production of lactate, ethanol, butyrate, acetone, or acetoin. Thus, for example (see, for example as in FIG. 1), aliphatic alcohol biosynthesis competitor polypeptides may include but are not limited to phosphotransbutyrylase polypeptides, butyrate kinase polypeptides, CoA tranferase polypeptides, acetoacetate decarboxylase polypeptides, phosphotransacetylase polypeptides, acetate kinase polypeptides, aldehyde dehydrogenase polypeptides, alcohol dehydrogenase polypeptides, and combinations thereof.

Aliphatic alcohol compound: An “aliphatic alcohol compound” is a compound in which one or more hydroxyl groups is attached to an alkyl radical. Aliphatic alcohol compounds of particular interest in accordance with the present disclosure are those with fewer than 10 carbon atoms. For example, aliphatic alcohol compounds include 1-butanol, 2-butanol, iso-butanol, tert-butanol, ethanol, 1-heptanol, 2-heptanol, 3-heptanol, 4-heptanol, iso-heptanol, 1-hexanol, 2-hexanol, 3-hexanol, 2-ethyl hexanol, iso-hexanol, methanol, 1-octanol, 2-octanol, 3-octanol, iso-octanol, and 4-octanol, 1-pentanol, 2-pentanol, 3-pentanol, iso-pentanol, 1-propanol, 2-propanol, iso-propanol, and combinations thereof.

Biosynthesis polypeptide: The term “biosynthesis polypeptide” as used herein (typically in reference to a particular compound or class of compounds), refers to polypeptides involved in the production of the compound or class of compounds. In some embodiments of the disclosure, biosynthesis polypeptides are synthetic enzymes that catalyze particular steps in a synthesis pathway that ultimately produce a relevant compound. In some embodiments, the term “biosynthesis polypeptide” may also encompass polypeptides that do not themselves catalyze synthetic reactions, but that regulate expression and/or activity of other polypeptides that do so.

Branched chain α-keto acid decarboxylase: The term “branched-chain α-keto acid decarboxylase” (also referred to herein as branched-chain keto acid decarboxylase keto acid decarboxylase) refers to an enzyme that catalyzes the conversion of α-ketoisovalerate to isobutyraldehyde and CO₂. Preferred branched-chain α-keto acid decarboxylases are known by the EC number 4.1.1.72 and are available from a number of sources, including, but not limited to, L. lactis (GenBank Nos: AAS49166 (SEQ ID NO:193 in US patent application 2007/0092957), AY548760 (SEQ ID NO:192), CAG34226 (SEQ ID NO:8 in US patent application 2007/0092957), AJ746364 (SEQ ID NO:191 in US patent application 2007/0092957)), Salmonella typhimurium (GenBank Nos: NP_{—}461346 (SEQ ID NO:195 in US patent application 2007/0092957), NC_{—}003197 (SEQ ID NO:194 in US patent application 2007/0092957)), and C. acetobutylicum (GenBank Nos: NP_{—}149189 (SEQ ID NO:197 in US patent application 2007/0092957), NC_{—}001988 (SEQ ID NO:196 in US patent application 2007/0092957)).

Branched-chain alcohol dehydrogenase: The term “branched-chain alcohol dehydrogenase” refers to an enzyme that catalyzes the conversion of isobutyraldehyde to isobutanol. Preferred branched-chain alcohol dehydrogenases are known by the EC number 1.1.1.265, but may also be classified under other alcohol dehydrogenases (specifically, EC 1.1.1.1 or 1.1.1.2). These enzymes utilize NADH (reduced nicotinamide adenine dinucleotide) and/or NADPH as an electron donor and are available from a number of sources, including, but not limited to, S. cerevisiae (GenBank Nos: NP_{—}010656 (SEQ ID NO:199 in US patent application 2007/0092957), NC_{—}001136 (SEQ ID NO:198 in US patent application 2007/0092957), NP_{—}014051 (SEQ ID NO:201 in US patent application 2007/0092957), NC_{—}001145 (SEQ ID NO:200 in US patent application 2007/0092957)), E. coli (GenBank Nos: NP_{—}417484 (SEQ ID NO:10 in US patent application 2007/0092957), NC_{—}000913 (SEQ ID NO:9 in US patent application 2007/0092957)), and C. acetobutylicum (GenBank Nos: NP_{—}349892 (SEQ ID NO:203 in US patent application 2007/0092957), NC_{—}003030 (SEQ ID NO:202 in US patent application 2007/0092957), NP_{—}349891 (SEQ ID NO:204 in US patent application 2007/0092957), NC_{—}003030 (SEQ ID NO:158 in US patent application 2007/0092957)).

Branched-chain keto acid dehydrogenase: The term “branched-chain keto acid dehydrogenase” refers to an enzyme that catalyzes the conversion of α-ketoisovalerate to isobutyryl-CoA (isobutyryl-coenzyme A), using NAD (nicotinamide adenine dinucleotide) as electron acceptor. Preferred branched-chain keto acid dehydrogenases are known by the EC number 1.2.4.4. These branched-chain keto acid dehydrogenases are comprised of four subunits and sequences from all subunits are available from a vast array of microorganisms, including, but not limited to, B. subtilis (GenBank Nos: CAB14336 (SEQ ID NO:206 in US patent application 2007/0092957), Z99116 (SEQ ID NO:205 in US patent application 2007/0092957), CAB14335 (SEQ ID NO:208 in US patent application 2007/0092957), Z99116 (SEQ ID NO:207 in US patent application 2007/0092957), CAB14334 (SEQ ID NO:210 in US patent application 2007/0092957), Z99116 (SEQ ID NO:209 in US patent application 2007/0092957), CAB14337 (SEQ ID NO:212 in US patent application 2007/0092957), Z99116 (SEQ ID NO:211 in US patent application 2007/0092957)) and P. putida (GenBank Nos: AAA65614 (SEQ ID NO:214 in US patent application 2007/0092957), M57613 (SEQ ID NO:213 in US patent application 2007/0092957), AAA65615 (SEQ ID NO:216 in US patent application 2007/0092957), M57613 (SEQ ID NO:215 in US patent application 2007/0092957), AAA65617 (SEQ ID NO:218 in US patent application 2007/0092957), M57613 (SEQ ID NO:217 in US patent application 2007/0092957), AAA65618 (SEQ ID NO:220 in US patent application 2007/0092957), M57613 (SEQ ID NO:219 in US patent application 2007/0092957)).

Butanol: The term “butanol”, as used herein, refers to a material that consists of 1-butanol, 2-butanol, iso-butanol, and/or tert-butanol. In some embodiments, “butanol” is 1-butanol.

Butanol biosynthesis polypeptide: As used herein, the term “butanol biosynthesis polyepeptide” refers to an aliphatic alcohol biosynthesis polypeptide that participates in the synthesis of butanol. In some embodiments, a butanol biosynthesis polypeptide participates in the synthesis of 1-butanol. In some embodiments, a butanol biosynthesis polypeptide catalyzes at least one step in the synthesis of butanol (e.g., 1-butanol). In some embodiments, a butanol biosynthesis polypeptide catalyzes a substrate to product conversion selected from the group consisting of a) acetyl-CoA to acetoacetyl-CoA; b) acetoacetyl-CoA to 3-hydroxybutyryl-CoA; c) 3-hydroxybutyryl-CoA to crotonyl-CoA; d) crotonyl-CoA to butyryl-CoA; e) butyryl-CoA to butyraldehyde; f) butyraldehyde to 1-butanol; and combinations thereof. In some embodiments, a butanol biosynthesis polypeptide is an acetyl-CoA acetyltransferase polypeptide, a 3-hydroxybutyryl-CoA dehydrogenase polypeptide, a crotonase polypeptide, a butyryl-CoA dehydrogenase polypeptide, a butyraldehyde dehydrogenase polypeptide, a butanol dehydrogenase polypeptide, or a combination thereof. In some embodiments, a butanol biosynthesis polypeptide catalyzes at least one step in the synthesis of isobutanol (i.e., is an isobutanol biosynthesis polypeptide). In some such embodiments, a butanol biosynthesis polypeptide catalyzes a substrate to product conversion selected from the group consisting of i) pyruvate to acetolactate (isobutanol biosynthesis pathway step a); ii) acetolactate to 2,3-dihydroxyisovalerate (isobutanol biosynthesis pathway step b); iii) 2,3-dihydroxyisovalerate to α-ketoisovalerate (isobutanol biosynthesis pathway step c); iv) α-ketoisovalerate to isobutyraldehyde, (isobutanol biosynthesis pathway step d); v) isobutyraldehyde to isobutanol (isobutanol biosynthesis pathway step e); vi) α-ketoisovalerate to isobutyryl-CoA, (isobutanol biosynthesispathway step f); vii) isobutyryl-CoA to isobutyraldehyde (isobutanol biosynthesis pathway step g); viii) α-ketoisovalerate to valine, (isobutanol biosynthesis pathway step h); ix) valine to isobutylamine (isobutanol biosynthesis pathway step i); x) isobutylamine to isobutyraldehyde (isobutanol biosynthesis pathway step j); xi) butyryl-CoA to isobutyryl-CoA (isobutanol biosynthesis pathway step k); and combinations thereof. For example, in some embodiments, a butanol biosynthesis polypeptide catalyzes a substrate to product conversion selected from the group consisting of i) pyruvate to acetolactate (isobutanol biosynthesis pathway step a) ii) acetolactate to 2,3-dihydroxyisovalerate (isobutanol biosynthesis pathway step b); iii) 2,3-dihydroxyisovalerate to α-ketoisovalerate (isobutanol biosynthesis pathway step c); iv) α-ketoisovalerate to isobutyraldehyde, (isobutanol biosynthesis pathway step d); and v) isobutyraldehyde to isobutanol (isobutanol biosynthesis pathway step e); and combinations thereof. In some embodiments, a butanol biosynthesis polypeptide catalyzes a substrate to product conversion selected from the group consisting of i) pyruvate to acetolactate, (isobutanol biosynthesis pathway step a); ii) acetolactate to 2,3-dihydroxyisovalerate, (isobutanol biosynthesis pathway step b); iii) 2,3-dihydroxyisovalerate to α-ketoisovalerate, (isobutanol biosynthesis pathway step c); iv) α-ketoisovalerate to isobutyryl-CoA, (isobutanol biosynthesis pathway step f); v) isobutyryl-CoA to isobutyraldehyde, (isobutanol biosynthesis pathway step g); and vi) isobutyraldehyde to isobutanol; (isobutanol biosynthesis pathway step e); and combinations thereof. In some embodiments, a butanol biosynthesis polypeptide catalyzes a substrate to product conversion selected from the group consisting of i) pyruvate to acetolactate (isobutanol biosynthesis pathway step a); ii) acetolactate to 2,3-dihydroxyisovalerate (isobutanol biosynthesis pathway step b); iii) 2,3-dihydroxyisovalerate to α-ketoisovalerate (isobutanol biosynthesis pathway step c); iv) α-ketoisovalerate to valine (isobutanol biosynthesis pathway step h); v) valine to isobutylamine (isobutanol biosynthesis pathway step i); vi) isobutylamine to isobutyraldehyde (isobutanol biosynthesis pathway step j); vii) isobutyraldehyde to isobutanol (isobutanol biosynthesis pathway step e) and combinations thereof. In some embodiments, a butanol biosynthesis polypeptide catalyzes a substrate to product conversion selected from the group consisting of i) butyryl-CoA to isobutyryl-CoA (isobutanol biosynthesis pathway step k); ii) isobutyryl-CoA to isobutyraldehyde, (isobutanol biosynthesis pathway step g); iii) isobutyraldehyde to isobutanol (isobutanol biosynthesis pathway step e); and combinations thereof. Butanol dehydrogenase: The term “butanol dehydrogenase” refers to an enzyme that catalyzes the conversion of butyraldehyde to 1-butanol, using either NADH or NADPH as cofactor. Butanol dehydrogenases are available from, for example C. acetobutylicum (GenBank Nos: NP_{—}149325 (SEQ ID NO:153 in WO 2007/041269), NC_{—}001988 SEQ ID NO:152 in WO 2007/041269; note: this enzyme possesses both aldehyde and alcohol dehydrogenase activity), NP_{—}349891 (SEQ ID NO:14 in WO 2007/041269), NC_{—}003030 (SEQ ID NO:13 in WO 2007/041269), NP_{—}349892 (SEQ ID NO: 16 in WO 2007/041269), NC_{—}003030 (SEQ ID NO:15 in WO 2007/041269)) and E. coli (GenBank Nos: NP_{—}417484 (SEQ ID NO: 155 in WO 2007/041269), NC_{—}000913 (SEQ ID NO:154 in WO 2007/041269)).

Butyraldehyde dehydrogenase: The term “butyraldehyde dehydrogenase” refers to an enzyme that catalyzes the conversion of butyryl-CoA to butyraldehyde, using NADH or NADPH as cofactor. Butyraldehyde dehydrogenases with a preference for NADH are known as E.C. 1.1.1.57 and are available from, for example, C. beijerinckii (Genbank Nos: AAD31841 (SEQ ID NO:12 in WO 2007/041269), AF157306 (SEQ ID NO:11 in WO 2007/041269)) and C. acetobutylicum (GenBank Nos: NP_{—}149325 (SEQ ID NO:153 in WO 2007/041269), NC_{—}001988 (SEQ ID NO:152 in WO 2007/041269)).

Butyryl-CoA dehydrogenase: The term “butyryl-CoA dehydrogenase” refers to an enzyme that catalyzes the conversion of crotonyl-CoA to butyryl-CoA. Butyryl-CoA dehydrogenases may be either NADH-dependent or NADPH-dependent and are classified as E.C. 1.3.1.44 and E.C. 1.3.1.38, respectively. Butyryl-CoA dehydrogenases are available from a number of sources, for example, C. acetobutylicum (GenBank Nos: NP_{—}347102 (SEQ ID NO:10 in WO 2007/041269), NC_{—}003030 (SEQ ID NO:9 in WO 2007/041269)), Euglena gracilis (GenBank Nos: □5EU90 (SEQ ID NO:147 in WO 2007/041269), AY741582 SEQ ID NO:146 in WO 2007/041269)), Streptomyces collinus (GenBank Nos: AAA92890 (SEQ ID NO:149 in WO 2007/041269), U37135 (SEQ ID NO: 148 in WO 2007/041269)), and Streptomyces coelicolor (GenBank Nos: CAA22721 (SEQ ID NO:151 in WO 2007/041269), AL939127 (SEQ ID NO:150 in WO 2007/041269)).

Crotonase: The term “crotonase” refers to an enzyme that catalyzes the conversion of 3-hydroxybutyryl-CoA to crotonyl-CoA and H₂O. Crotonases may have a substrate preference for (S)-3-hydroxybutyryl-CoA or (R)-3-hydroxybutyryl-CoA and are classified as E.C. 4.2.1.17 and E.C. 4.2.1.55, respectively. Crotonases are available from a number of sources, for example, E. coli (GenBank Nos: NP_{—}415911 (SEQ ID NO:141 in WO 2007/041269), NC_{—}000913 (SEQ ID NO:140 in WO 2007/041269)), C. acetobutylicum (GenBank Nos: NP_{—}349318 (SEQ ID NO:8 in WO 2007/041269), NC_{—}003030 (SEQ ID NO:6 in WO 2007/041269)), B. subtilis (GenBank Nos: CAB13705 (SEQ ID NO:143 in WO 2007/041269), Z99113 (SEQ ID NO: 142 in WO 2007/041269)), and Aeromonas caviae (GenBank Nos: BAA21816 (SEQ ID NO:145 in WO 2007/041269), D88825 (SEQ ID NO:144 in WO 2007/041269)).

Engineered microorganism: An “engineered microorganism”, as that term is used herein, is one that contains a modification introduced by the hand of man, so that the engineered microorganism differs from a parent organism to which it is otherwise identical. Progeny of a microorganism that also contain the modification are encompassed by the term “engineered microorganism”.

Gene: The term “gene”, as used herein, generally refers to a nucleic acid encoding a polypeptide, optionally including certain regulatory elements that may affect expression of one or more gene products (i.e., RNA or protein).

Genetic compatibility: The phrase “genetic compatibility” is used herein to refer to pairs (or sets) of organisms for which genetic elements from cells of one organism operate (and/or are expressed) in the other organism. Those of ordinary skill in the art will appreciate, of course, that two organisms may be genetically compatible even though one or more particular genetic elements, and particularly genetic regulatory sequences, may not function in both organisms. The techniques of molecular biology may readily be applied, for example, to adjust and/or substitute expression control sequences, to account for codon bias preferences, etc. in order to increase expression of heterologous sequences from a source organism in cells of a host organism. Those of ordinary skill in the art will further appreciate that genetic compatibility can be determined by any of a variety of modes of assessment. In some embodiments, for example, genetic compatibility is determined by experimental success in achieving expression of source organism genetic elements in host recipient cells. In some embodiments, genetic compatibility is determined (or at least predicted) based on taxonomical relationship between source and host organisms. For example, there is a reasonable expectation of genetic compatibility between multiple members of the gram-positive, low G+C firmicutes group of bacteria (e.g. Lactobacillus plantarum and Clostridium acetobutylicum).

Heterologous: The term “heterologous”, as used herein to refer to genes or polypeptides, refers to a gene or polypeptide that does not naturally occur in the organism in which it is being expressed. It will be understood that, in general, when a heterologous gene or polypeptide is selected for introduction into and/or expression by a host cell, the particular source organism from which the heterologous gene or polypeptide may be selected is not essential to the practice of the present disclosure. Relevant considerations may include, for example, how closely related the potential source and host organisms are in evolution, or how related the source organism is with other source organisms from which sequences of other relevant polypeptides have been selected. Where a plurality of different heterologous polypeptides are to be introduced into and/or expressed by a host cell, different polypeptides may be from different source organisms, or from the same source organism. To give but one example, in some cases, individual polypeptides may represent individual subunits of a complex protein activity and/or may be required to work in concert or in a sequential order with other polypeptides in order to achieve the goals of the present disclosure. In some embodiments, it will often be desirable for such polypeptides to be from the same source organism, and/or to be sufficiently related to function appropriately when expressed together in a host cell. In some embodiments, such polypeptides may be from different, even unrelated source organisms. It will further be understood that, where a heterologous polypeptide is to be expressed in a host cell, it will often be desirable to utilize nucleic acid sequences encoding the polypeptide that have been adjusted to accommodate codon preferences of the host cell and/or to link the encoding sequences with regulatory elements active in the host cell.

Homolog: A “homolog” is a polypeptide, gene, or portion thereof (e.g., a 3′ region of a gene, e.g., a 3′ untranslated region (UTR) of a gene, e.g., a 5′ region of a gene, e.g., a 5′ UTR) that shows a designated degree of sequence identity (and/or similarity) with another polypeptide, gene, or portion thereof. For example, any polypeptide that shows at least about 30-40% overall sequence identity with another polypeptide, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide is a homolog of that polypeptide. In many embodiments, a homolog of a polypeptide further shares sequence similarity with and/or at least one functional attribute or activity of the polypeptide. With regard to genes or nucleotide sequences, any gene or nucleotide sequence that (i) shows at least about 60% overall sequence identity with another gene or nucleotide sequence; and or (ii) has a same function as, and/or encodes a homolog of a polypeptide encoded by, the other gene or nucleotide sequence is a homolog of that gene or nucleotide sequence. With regard to a 3′ region of a gene (e.g., 3′ UTR) that adjusts susceptibility of an organism to one or more toxic effects of one or more aliphatic alcohol compounds, any nucleotide sequence that (i) either shows at least 60% overall sequence identity, and/or is a 3′ region of a gene that is a homolog of the gene as defined above; and (ii) also adjusts susceptibility of an organism to toxic effects of alcohol compounds is a homolog of that 3′ region. As is known by those of ordinary skill in the art, a variety of strategies are known, and tools are available, for performing comparisons of amino acid or nucleotide sequences in order to assess degrees of identity and/or similarity. These strategies include, for example, manual alignment, computer assisted sequence alignment and combinations thereof. A number of algorithms (which are generally computer implemented) for performing sequence alignment are widely available, or can be produced by one of skill in the art. Representative algorithms include, e.g., the local homology algorithm of Smith and Waterman (Adv. Appl. Math., 1981, 2: 482); the homology alignment algorithm of Needleman and Wunsch (J. Mol. Biol., 1970, 48: 443); the search for similarity method of Pearson and Lipman (Proc. Natl. Acad. Sci. (USA), 1988, 85: 2444); and/or by computerized implementations of these algorithms (e.g., GAP, BESTFIT, FASTA, and TFASTA in the Wisconsin Genetics Software Package Release 7.0, Genetics Computer Group, 575 Science Dr., Madison, Wis.). Readily available computer programs incorporating such algorithms include, for example, BLASTN, BLASTP, Gapped BLAST, PILEUP, CLUSTALW, etc. When utilizing BLAST and Gapped BLAST programs, default parameters of the respective programs may be used. Alternatively, the practitioner may use non-default parameters depending on his or her experimental and/or other requirements (see for example, the Web site having URL www.ncbi.nlm.nih.gov).

Host cell: As used herein, the “host cell” is a microbial cell that is manipulated according to the present disclosure. For example, in some embodiments, a host cell is manipulated such that its tolerance for one or more aliphatic alcohol compounds is increased (e.g., via an alcohol tolerance modification); in some embodiments, a host cell is manipulated such that its production of one or more aliphatic alcohol compounds is increased (e.g., via an alcohologenic modification). A “modified host cell”, as used herein, is any host cell which has been modified, engineered, or manipulated in accordance with the present disclosure as compared with an otherwise identical parental cell. In some embodiments, the modified host cell has at least one alcohol tolerance modification and/or at least one (and optionally more than one as compared with the parental cell) alcohologenic modification. In some embodiments, the parental cell is a naturally occurring parental cell. In some embodiments, the parental cell produces at least one aliphatic alcohol.

3-Hydroxybutyryl-CoA dehydrogenase: The term “3-hydroxybutyryl-CoA dehydrogenase” refers to an enzyme that catalyzes the conversion of acetoacetyl-CoA to 3-hydroxybutyryl-CoA. 3-Hydroxybutyryl-CoA dehydrogenases may be reduced nicotinamide adenine dinucleotide (NADH)-dependent, with a substrate preference for (S)-3-hydroxybutyryl-CoA or (R)-3hydroxybutyryl-CoA and are classified as E.C. 1.1.1.35 and E.C. 1.1.1.30, respectively. Additionally, 3-hydroxybutyryl-CoA dehydrogenases may be reduced nicotinamide adenine dinucleotide phosphate (NADPH)-dependent, with a substrate preference for (S)-3-hydroxybutyryl-CoA or (R)-3-hydroxybutyryl-CoA and are classified as E.C. 1.1.1.157 and E.C. 1.1.1.36, respectively. 3-hydroxybutyryl-CoA dehydrogenases are available from a number of sources, for example, C. acetobutylicum (GenBank Nos: NP_{—}349314 (SEQ ID NO:6 in WO 2007/041269), NC_{—}003030 (SEQ ID NO:5 in WO 2007/041269)), B. subtilis (GenBank Nos: AAB09614 (SEQ ID NO:135 in WO 2007/041269), U29084 (SEQ ID NO:134 in WO 2007/041269)), Ralstonia eutropha (GenBank Nos: YP 294481 (SEQ ID NO:137 in WO 2007/041269), NC_{—}007347 (SEQ ID NO:136 in WO 2007/041269)), and Alcaligenes eutrophus (GenBank Nos: AAA21973 (SEQ ID NO:139 in WO 2007/041269), J04987 (SEQ ID NO:138 in WO 2007/041269)).

Introduce: The term “introduce”, as used herein with reference to introduction of a nucleic acid into a cell or organism is intended to have its broadest meaning and to encompass introduction, for example by transformation methods (e.g., calcium-chloride-mediated transformation, electroporation, particle bombardment), and also introduction by other methods including transduction, conjugation, and mating. In some embodiments, a vector is utilized to introduce a nucleic acid into a cell or organism.

Isobutyryl-CoA mutase: The term “isobutyryl-CoA mutase” refers to an enzyme that catalyzes the conversion of butyryl-CoA to isobutyryl-CoA. This enzyme uses coenzyme B₁₂ as a cofactor. Preferred isobutyryl-CoA mutases are known by the EC number 5.4.99.13. These enzymes are found in a number of Streptomycetes, including, but not limited to, Streptomyces cinnamonensis (GenBank Nos: AAC08713 (SEQ ID NO:256 in US patent application 2007/0092957), U67612 (SEQ ID NO:255 in US patent application 2007/0092957), CAB59633 (SEQ ID NO:258 in US patent application 2007/0092957), AJ246005 (SEQ ID NO:257 in US patent application 2007/0092957)), S. coelicolor (GenBank Nos: CAB70645 (SEQ ID NO:260 in US patent application 2007/0092957), AL939123 (SEQ ID NO:259 in US patent application 2007/0092957), CAB92663 (SEQ ID NO:262 in US patent application 2007/0092957), AL939121 (SEQ ID NO:261 in US patent application 2007/0092957)), and Streptomyces avermitilis (GenBank Nos: NP_{—}824008 (SEQ ID NO:264 in US patent application 2007/0092957), NC_{—}003155 (SEQ ID NO:263 in US patent application 2007/0092957), NP_{—}824637 (SEQ ID NO:266 in US patent application 2007/0092957), NC_{—}003155 (SEQ ID NO:265 in US patent application 2007/0092957)).

Isolated: The term “isolated”, as used herein, means that the isolated entity has been separated from at least one component with which it was previously associated. When most other components have been removed, the isolated entity is “purified” or “concentrated”. Isolation and/or purification and/or concentration may be performed using any techniques known in the art including, for example, distillation, fractionation, gas stripping, extraction, precipitation, or other separation.

Modification: In principle, “modification”, as that term is used herein, may be any chemical, physiological, genetic, or other modification of an organism that appropriately alters a designated feature of a host organism (e.g., an alcohologenic modification alters production of at least one aliphatic alcohol compound, an alcohol tolerance modification alters susceptibility to one or more aliphatic alcohol compounds, etc.) as compared with an otherwise identical organism not subject to the same modification. In most embodiments, however, the modification will comprise a genetic modification, typically resulting in decreased susceptibility to one or more selected aliphatic alcohol compounds (e.g., butanol). In some embodiments, the modification comprises at least one chemical, physiological, genetic, or other modification; in other embodiments, the modification comprises more than one chemical, physiological, genetic, or other modification. In certain embodiments where more than one modification is utilized, such modifications can comprise any combination of chemical, physiological, genetic, or other modification (e.g., one or more genetic, chemical and/or physiological modification(s)).

Omega transaminase: The term “omega transaminase” refers to an enzyme that catalyzes the conversion of isobutylamine to isobutyraldehyde using a suitable amino acid as an amine donor. Preferred omega transaminases are known by the EC number 2.6.1.18 and are available from a number of sources, including, but not limited to, Alcaligenes denitrificans (GenBank Nos: AAP92672 (SEQ ID NO:248 in US patent application 2007/0092957), AY330220 (SEQ ID NO:247 in US patent application 2007/0092957)), Ralstonia eutropha (GenBank Nos: YP 294474 (SEQ ID NO:250 in US patent application 2007/0092957), NC_{—}007347 (SEQ ID NO:249 in US patent application 2007/0092957)), Shewanella oneidensis (GenBank Nos: NP_{—}719046 (SEQ ID NO:252 in US patent application 2007/0092957), NC_{—}004347 (SEQ ID NO:251 in US patent application 2007/0092957)), and P. putida (GenBank Nos: AAN66223 (SEQ ID NO:254 in US patent application 2007/0092957), AE016776 (SEQ ID NO:253 in US patent application 2007/0092957)).

Polypeptide: The term “polypeptide”, as used herein, generally has its art-recognized meaning of a polymer of at least three amino acids. However, the term is also used to refer to specific functional classes of polypeptides, such as, for example, biosynthesis polypeptides, competitor polypeptides, alcohol tolerance polypeptides, etc. For each such class, the present specification provides several examples of known sequences of such polypeptides. Those of ordinary skill in the art will appreciate, however, that the term “polypeptide” is intended to be sufficiently general as to encompass not only polypeptides having the complete sequence recited herein (or referred to by specific reference to a description in publication or database, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Other regions of similarity and/or identity can be determined by those of ordinary skill in the art by analysis of the sequences of various polypeptides presented in the Tables herein.

Small Molecule: In general, a small molecule is understood in the art to be an organic molecule that is less than about 5 kilodaltons (Kd) in size. In some embodiments, the small molecule is less than about 3 Kd, 2 Kd, or 1 Kd. In some embodiments, the small molecule is less than about 800 daltons (D), 600 D, 500 D, 400 D, 300 D, 200 D, or 100 D. In some embodiments, small molecules are non-polymeric. In some embodiments, small molecules are not proteins, peptides, or amino acids. In some embodiments, small molecules are not nucleic acids or nucleotides. In some embodiments, small molecules are not saccharides or polysaccharides.

Source organism: The term “source organism”, as used herein, refers to the organism in which a particular polypeptide or nucleotide (e.g., gene) is found in nature. Thus, for example, if one or more heterologous polypeptides is/are being expressed in a host organism, the organism in which the polypeptides are expressed in nature (and/or from which their genes were originally cloned) is referred to as the “source organism”. Where multiple heterologous polypeptides are being expressed in a host organism, one or more source organism(s) may be utilized for independent selection of each of the heterologous polypeptide(s). It will be appreciated that any and all organisms that naturally contain relevant polypeptide sequences may be used as source organisms in accordance with the present disclosure. Representative source organisms include, for example, animal, mammalian, insect, plant, fungal, yeast, algal, bacterial, archaebacterial, cyanobacterial, and protozoal source organisms.

Transaminase: The term “transaminase” refers to an enzyme that catalyzes the conversion of α-ketoisovalerate to valine, using either alanine or glutamate as amine donor. Preferred transaminases are known by the EC numbers 2.6.1.42 and 2.6.1.66. These enzymes are available from a number of sources. Examples of sources for alanine-dependent enzymes include, but are not limited to, E. coli (GenBank Nos: YP_{—}026231 (SEQ ID NO:232 in US patent application 2007/0092957), NC_{—}000913 (SEQ ID NO:231 in US patent application 2007/0092957)) and Bacillus lichenifonnis (GenBank Nos: YP_{—}093743 (SEQ ID NO:234 in US patent application 2007/0092957), NC_{—}006322 (SEQ ID NO:233 in US patent application 2007/0092957)). Examples of sources for glutamate-dependent enzymes include, but are not limited to, E. coli (GenBank Nos: YP_{—}026247 (SEQ ID NO:236 in US patent application 2007/0092957), NC_{—}000913 (SEQ ID NO:235 in US patent application 2007/0092957)), S. cerevisiae (GenBank Nos: NP_{—}012682 (SEQ ID NO:238), NC_{—}001142 (SEQ ID NO:237 in US patent application 2007/0092957)) and Methanobacterium thermoautotrophicum (GenBank Nos: NP_{—}276546 (SEQ ID NO:240 in US patent application 2007/0092957), NC_{—}000916 (SEQ ID NO:239 in US patent application 2007/0092957)).

Valine decarboxylase: The term “valine decarboxylase” refers to an enzyme that catalyzes the conversion of valine to isobutylamine and CO₂. Preferred valine decarboxylases are known by the EC number 4.1.1.14. These enzymes are found in Streptomycetes, such as for example, Streptomyces viridifaciens (GenBank Nos: AAN10242 (SEQ ID NO:246 in US patent application 2007/0092957), AY116644 (SEQ ID NO:245 in US patent application 2007/0092957)).

Valine dehydrogenase: The term “valine dehydrogenase” refers to an enzyme that catalyzes the conversion of α-ketoisovalerate to valine, using NAD(P)H as electron donor and ammonia as amine donor. Preferred valine dehydrogenases are known by the EC numbers 1.4.1.8 and 1.4.1.9 and are available from a number of sources, including, but not limited to, Streptomyces coelicolor (GenBank Nos: NP_{—}628270 (SEQ ID NO:242 in US patent application 2007/0092957), NC_{—}003888 (SEQ ID NO:241 in US patent application 2007/0092957)) and B. subtilis (GenBank Nos: CAB14339 (SEQ ID NO:244 in US patent application 2007/0092957), Z99116 (SEQ ID NO:243 in US patent application 2007/0092957)).

### Detailed Description of Certain Embodiments

The present disclosure embraces the reasoning that microbial strains can be engineered to have increased tolerance to aliphatic alcohols. According to the present disclosure, microbial strains are engineered to contain one or more modifications that increase their tolerance to one or more aliphatic alcohol compounds. Alternatively or additionally, microbial strains for use in accordance with the present disclosure may be engineered to contain one or more modifications that increase their ability to produce one or more aliphatic alcohol compounds. In some embodiments, a modification that increases a cell's tolerance to one or more aliphatic alcohol compounds will also allow higher production of one or more aliphatic alcohol compounds.

In certain embodiments, engineered microbial cells show an increased aliphatic alcohol IC₅₀ as compared with parental cells. In certain embodiments, the aliphatic alcohol IC₅₀ is increased at least 5%, 10%, 20%, 30%, 40%, 50%, 60%, 70%, 80%, 90%, 100%, or more. In certain embodiments, engineered microbial cells show increased carbohydrate utilization as compared to parental cells when grown in same amount of alcohol. For example, in some embodiments, carbohydrate utilization is increased at least 5%, 10%, 20%, 30%, 40%, 50%, 60%, 70%, 80%, 90%, 100%, or more. In some embodiments, the carbohydrate whose utilization is increased is glucose.

Engineered cells and processes of using them as described herein may provide one or more advantages as compared with parental cells. Such advantages may include, but are not limited to: increased yield (gram of aliphatic alcohol compound produced per gram of carbohydrate substrate consumed), increased titer (gram(s) of aliphatic alcohol compound produced per liter of broth), increased specific productivity (gram(s) of aliphatic alcohol compound produced per gram of host cell biomass per unit of time (e.g. hour)), and/or increased volumetric productivity (gram(s) of aliphatic alcohol compound produced per liter of broth per unit of time (e.g. hour) of the desired aliphatic alcohol compound (and/or intermediates thereof), and/or decreased formation of undesirable side products (for example, undesirable intermediates).

Thus, for example, the yield for one or more desired aliphatic alcohol compounds (or total aliphatic alcohol compound content) produced from a glucose-containing substrate, may be increased at least about 5%, 10%, 25%, 50%, 75%, 100% or more as compared with a parental cell. In some embodiments, the yield (g aliphatic alcohol/g glucose substrate) for one or more desired aliphatic alcohol compounds, or total aliphatic alcohol compound content, may be at or about 0.01, at or about 0.05, at or about 0.10, at or about 0.15, at or about 0.20, at or about 0.25, at or about 0.28, at or about 0.30, at or about 0.32, at or about 0.34, at or about 0.36, at or about 0.38, at or about 0.40 or more.

In some embodiments, aliphatic alcohol compound production is assessed by measuring broth titer (g aliphatic alcohol/liter broth). In some embodiments, broth titer for a particular aliphatic alcohol compound, or combination of compounds, is increased at least about 5%, 10%, 25%, 50%, 75%, 100% or more in cells engineered according to the present disclosure as compared with parental cells. In some embodiments, such broth titer achieves levels as high as at or about 1, at or about 5, at or about 10, at or about 15, at or about 20, at or about 25, at or about 30, at or about 35, at or about 40, at or about 50, at or about 55, at or about 60, at or about 65, at or about 70, at or about 75, at or about 80 or more.

Various aspects and features of certain embodiments of the disclosure are discussed in more detail below.

### Host Cells

Inventive modifications may be applied to any of a variety of host cells in accordance with the present disclosure. For example, in some embodiments, parental cells already produce one or more aliphatic alcohol compounds before being engineered in accordance with the present disclosure. In other words, in some embodiments, modifications are applied to cells that already produce one or more aliphatic alcohol compounds. In some embodiments, however, parental cells do not produce one or more aliphatic alcohol compounds before being engineered in accordance with the present disclosure. In some embodiments of the present disclosure, cells are engineered to increase (whether from zero or from a base level) production of one or more aliphatic alcohol compounds, and/or to alter relative production levels of different aliphatic alcohol compounds. In some embodiments of the present disclosure, parental cells do not produce a particular aliphatic alcohol compound of interest (e.g., butanol, for example 1-butanol) prior to application of one or more modifications of the present disclosure. In some embodiments, cells are therefore engineered to produce the one or more particular aliphatic alcohol compounds. In some such embodiments, the cells are engineered to express (and/or activate) a plurality of biosynthesis polypeptides (e.g. aliphatic alcohol biosynthesis polypeptides), such that synthesis is achieved. In some embodiments, cells engineered to produce at least one aliphatic alcohol compound lack one or more aliphatic alcohol biosynthesis competitor polypeptides. Indeed, in some embodiments, it is desirable to engineer cells that lack one or more aliphatic alcohol biosynthesis competitor polypeptides such that diversion of carbon flow away from one or more desired aliphatic alcohol biosynthesis pathways is minimized

In some embodiments of the present disclosure, parental cells already show some degree of tolerance to one or more aliphatic alcohol compounds before being engineered in accordance with the present disclosure. In other words, in some embodiments, modifications are applied to cells that already show tolerance to one or more aliphatic alcohol compounds. In some embodiments, however, parental cells do not show tolerance to one or more aliphatic alcohol compounds before being engineered in accordance with the present disclosure. In some embodiments of the present disclosure, cells are engineered to increase (whether from zero or from a base level) tolerance to one or more aliphatic alcohol compounds, and/or to alter relative tolerance levels to different aliphatic alcohol compounds.

In some embodiments, desirable cells or organisms to which modifications are applied in accordance with the present disclosure are characterized by one or more attributes such as (i) intrinsic tolerance to one or more aliphatic alcohol compounds; (ii) evidence of an ability to adapt to or be modified to (e.g. through chemical mutagenesis) enhanced tolerance to one or more aliphatic alcohol compounds; (iii) availability of genomic sequence information, or at least sequence information of relevant genetic elements (e.g., genes encoding polypeptides that contribute to tolerance); (iv) availability of tools to achieve molecular manipulation (e.g., of genetic sequences); (v) genetic stability; (vi) metabolic tendencies such as the ability to metabolize particular carbon sources (e.g., lignocellulosic biomass); (vii) potential for anaerobic growth; (viii) environmental niche (e.g., exposure to butane, butanol, etc); (ix) ability to biosynthesize one or more aliphatic alcohol compounds (e.g., butanol, and particularly 1-butanol); (x) minimal biosafety issues (e.g., infectious potential, etc.); and/or (xi) adaptability to growth under cost-effective, large scale commercial conditions (e.g., temperatures that do not require significant cooling of the fermentation vessel).

In some embodiments, host cells engineered in accordance with the present disclosure are members of a genus selected from the group consisting of Clostridium, Zymomonas, Escherichia, Salmonella, Rhodococcus, Pseudomonas, Bacillus, Lactobacillus, Lactococcus, Enterococcus, Alcaligenes, Klebsiella, Paenibacillus, Arthrobacter, Corynebacterium, Brevibacterium, Acinetobacter, Pichia, Candida, Hansenula and Saccharomyces.

In some embodiments, where host cells engineered in accordance with the present disclosure are members of the genus Clostridium, they are members of a species selected from the group consisting of Clostridium acetobutylicum, Clostridium beijerinckii, and Clostridium saccaharoperbuylacetonicum. Natural strains of each of these organisms have some ability to produce aliphatic alcohol compounds (e.g., butanol), maximally on the order of about 12 g/L. In some embodiments, host cells engineered in accordance with the present disclosure are members of the species Clostridium acetobutylicum.

In some embodiments, where host cells engineered in accordance with the present disclosure are members of the genus Escherichia, they are members of the species Escherichia coli.

In some embodiments, where host cells engineered in accordance with the present disclosure are members of the genus Alcaligenes, they are members of the species Alcaligenes eutrophus.

In some embodiments, where host cells engineered in accordance with the present disclosure are members of the genus Bacillus, they are members of the species Bacillus licheniformis or Bacillus subtilis.

In some embodiments, where host cells engineered in accordance with the present disclosure are members of the genus Paenibacillus, they are members of the species Paenibacillus macerans.

In some embodiments, where host cells engineered in accordance with the present disclosure are members of the genus Rhodococcus, they are members of the species Rhodococcus erythropolis.

In some embodiments, where host cells engineered in accordance with the present disclosure are members of the genus Pseudomonas, they are members of the species Pseudomonas putida.

In some embodiments, where host cells engineered in accordance with the present disclosure are members of the genus Lactobacillus, they are members of the species Lactobacillus plantarum.

In some embodiments, where host cells engineered in accordance with the present disclosure are members of the genus Enterococcus, they are members of the species Enterococcus faecium, Enterococcus gallinarum, or Enterococcus faecalis.

In some embodiments, where host cells engineered in accordance with the present disclosure are members of the genus Saccharomyces, they are members of the species Saccharomyces cerevisiae.

Those of ordinary skill in the art will appreciate that the selection of a particular host cell for use in accordance with the present disclosure will also affect, for example, the selection of expression sequences utilized with any heterologous polypeptide to be introduced into the cell, codon bias that can optionally be engineered into any nucleic acid to be expressed in the cell and will also influence various aspects of culture conditions, etc. Much is known about the different gene regulatory requirements and cultivation requirements of different host cells to be utilized in accordance with the present disclosure.

To give but a few examples, vectors or cassettes useful for the modification (e.g. transformation) of a variety of host cells are common and commercially available from companies such as EPICENTRE® (Madison, Wis.), Invitrogen Corp. (Carlsbad, Calif.), Stratagene (La Jolla, Calif.), and New England Biolabs, Inc. (Beverly, Mass.). Typically, the vector or cassette contains sequences directing transcription and translation of the relevant gene, a detectable or selectable marker and sequences allowing autonomous replication or chromosomal integration. Suitable vectors often comprise a region 5′ of a gene coding sequence which harbors transcriptional initiation controls and a region 3′ of the gene coding sequence which controls transcriptional termination. Both control regions may be derived from genes endogenous or homologous to the transformed host cell, although it is to be understood that such control regions may also be derived from genes that are not native to the specific species chosen as a production host.

Initiation control regions or promoters, which are useful to drive expression of genetic elements in a host cell are numerous and familiar to those skilled in the art. Virtually any promoter capable of driving these genetic elements is suitable for the present invention including, but not limited to, CYC1, HIS3, GAL1, GAL10, ADH1, PGK, PHO5, GAPDH, ADC1, TRP1, URA3, LEU2, ENO1, ENO2, TPI, CUP1, FBA, GPD, and GPM (useful for expression in Saccharomyces); AOX1 (useful for expression in Pichia); and lac, ara, tet, trp, IP_{L}, IP_{R}, T7, tac, and trc (useful for expression in Escherichia coli, Alcaligenes, and Pseudomonas); the amy, apr, npr promoters and various phage promoters useful for expression in Bacillus subtilis, Bacillus licheniformis, and Paenibacillus macerans; nisA (useful for expression in Gram-positive bacteria, Eichenbaum et al. Appl. Environ. Microbiol. 64(8):2763-2769 (1998)); and the synthetic P11 promoter (useful for expression in Lactobacillus plantarum, Rud et al., Microbiology 152:1011-1019 (2006)).

Termination control regions may also be derived from various genes native or heterologous to the relevant host. Termination control regions are not required, but are often utilized in accordance with the present invention.

Certain vectors are capable of replicating in a broad range of host bacteria and can be transferred by conjugation. Many such vectors are well known. For example, the complete and annotated sequence of pRK404 and three related vectors-pRK437, pRK442, and pRK442(H) are available. These derivatives have proven to be valuable tools for genetic manipulation in Gram-negative bacteria (Scott et al., Plasmid 50(1):74, 2003). Several plasmid derivatives of broad-host-range Inc P4 plasmid RSF1010 are also available with promoters that can function in a range of Gram-negative bacteria. Plasmid pAYC36 and pAYC37, have active promoters along with multiple cloning sites to allow for the heterologous gene expression in Gram-negative bacteria.

Chromosomal gene replacement tools are also widely available. For example, a thermosensitive variant of the broad-host-range replicon pWV101 has been modified to construct a plasmid pVE6002 which can be used to create gene replacement in a range of Gram-positive bacteria (Maguin et al., J. Bacteriol. 174(17):5633, 1992). Additionally, in vitro transposomes are available to create random mutations in a variety of genomes from commercial sources such as EPICENTRE®. Particular non-limiting examples of suitable vectors for use in transformation of Lactobacillus (e.g., L. plantarum) include pAMβ1 and derivatives thereof (Renault et al., Gene 183:175, 1996) and O'Sullivan et al., Gene 137:227, 1993); pMBB1 and pHW800, a derivative of pMBB1 (Wyckoff et al. Appl. Environ. Microbiol. 62:1481, 1996); pMG1, a conjugative plasmid (Tanimoto et al., J. Bacteriol. 184:5800, 2002); pNZ9520 (Kleerebezem et al., Appl. Environ. Microbiol. 63:4581, 1997); pAM401 (Fujimoto et al., Appl. Environ. Microbiol. 67:1262, 2001); and pAT392 (Arthur et al., Antimicrob. Agents Chemother. 38:1899, 1994). Several plasmids from Lactobacillus plantarum have also been reported (e.g., van Kranenburg et al., Appl. Environ. Microbiol. 71: 1223, 2005). For example, expression of the 1-butanol biosynthetic pathway in Lactobacillus plantarum is described in WO 2007/041269.

### Engineering Alcohol Tolerance

As already noted herein, attempts to produce aliphatic alcohol compounds by fermentation of producing organisms have generally been limited by the toxicity of the produced compounds. Butanol is particularly toxic; it is generally reported that concentrations of butanol above about 12-13 g/L result in profound cellular degradation. Thus, butanol production levels above 12-13 g/L have rarely been achieved through fermentation. Moreover, butanol production levels at or about this level have never been reported for a modified organism that does not naturally produce butanol.

Without wishing to be bound by any particular theory, we note that it has been proposed that aliphatic alcohols exert their toxicity through effects on cell membranes, on metabolism, and/or on the stability and/or conformation of cellular proteins (see, for example, Bowles et al., Appl Environ. Microbiol. 50:1165, 1985; Huang et al., Appl. Environ. Microbiol. 50:1043, 1985; Baer et al., Appl. Environ Microbiol. 55:2854, 1987; Lepage et al., J. Gen. Microbiol. 133:103, 1987; Tomas et al., Appl. Environ. Microbiol. 69:4951, 2003). For example, it has been proposed that aliphatic alcohols can permeabilize the cell membrane. Such permeabilization may, among others things, allow leakage and/or passive flux of solutes (e.g., ATP, protons, ions, even macromolecules) across the membrane. Permeabilization may also disrupt the proton and/or electrical potential gradients across cell membranes. Alternatively or additionally, aliphatic alcohols may alter membrane fluidity and/or affect the three-dimensional structure and/or activity of membrane proteins.

Among other strategies, cells may achieve increased tolerance to aliphatic alcohols through altered membrane composition, increased efflux of toxic compounds (particularly aliphatic alcohols and/or their metabolites), altered metabolism to toxic compounds into non-toxic compounds, and/or induction of systems that counteract effects of toxic compounds (e.g., of stress response systems).

According to the present disclosure, in some embodiments, cells are engineered to show increased alcohol tolerance through application of a modification that alters expression and/or activity of one or more membrane components that participates in aliphatic alcohol resistance. In some embodiments, cells are engineered to show increased alcohol tolerance through application of a modification that alters expression and/or activity of one or more membrane components that participates in achieving efflux (i.e., out-transport) of one or more toxic compounds. In some embodiments, cells are engineered to show increased alcohol tolerance through application of a modification that alters expression or activity of one or more components that participates in metabolism of toxic compounds into less toxic (or non-toxic) compounds. In some embodiments, cells are engineered to show increased alcohol tolerance through application of a modification that alters expression or activity of one or more components of a stress response system.

According to the present disclosure, tolerance to aliphatic alcohols may be assessed through any of a variety of means. For example, in some embodiments, aliphatic alcohol compound IC₅₀ is determined (expected to increase with increasing tolerance). In some embodiments, carbohydrate utilization is monitored (expected to increase with increasing tolerance). In some embodiments, broth titer of a produced aliphatic alcohol is measured (expected to increase with increasing tolerance). In some embodiments, aliphatic alcohol yield is measured (expected to increase with increasing tolerance). In some embodiments, specific or volumetric productivity of a one or more aliphatic alcohols is evaluated (both expected to increase with increasing tolerance). Alternatively or additionally, attributes such as, for example, membrane fluidity (expected to decrease with increasing tolerance), ratio of saturated to unsaturated lipids in a membrane (expected to decrease with increasing tolerance), activity of membrane-bound ATP-ases (expected to increase with increasing tolerance), internal cellular pH (expected to increase with increasing tolerance), presence of a pH gradient across a cellular membrane (expected to be present under conditions of tolerance), activity of certain membrane proteins (expected to increase with increasing tolerance), presence of membrane potential (expected to be present under conditions of tolerance), degree of active transport across cellular membrane (expected to increase with increasing tolerance), etc. may be assessed. Those of ordinary skill in the art will readily appreciate a wide range of assays that can be employed and/or parameters that can be assessed in order to evaluate the aliphatic alcohol tolerance of a given cell (e.g., an engineered cell and/or a parental cell or other comparitor cell) in accordance with the present disclosure.

In some particular embodiments of the present disclosure, a cell or organism to which an alcohol tolerance modification has been applied exhibits an increased aliphatic alcohol compound IC₅₀ as compared with an otherwise identical organism lacking the modification; in some embodiments, the aliphatic alcohol compound IC₅₀ is increased 5%, 10%, 20%, 30%, 40%, 50%, 60%, 70%, 80%, 90%, 100% or more. In some embodiments, a cell or organism to which an alcohol tolerance modification has been applied exhibits increased carbohydrate utilization as compared with an otherwise identical organism lacking the modification when grown in the presence of the same amount of aliphatic alcohol compound; in some cases the carbohydrate utilization is increased by 5%, 10%, 20%, 30%, 40%, 50%, 60%, 70%, 80%, 90%, 100% or more. In some embodiments, a cell or organism to which an alcohol tolerance modification has been applied exhibits increased production of at least one aliphatic alcohol compound as compared with an otherwise identical organism lacking the modification; in some embodiments, such increased production results in a broth titer of the produced at least one aliphatic alcohol compound that is 10%, 25%, 50%, 75%, 100% or more higher than that observed with an otherwise identical organism lacking the modification under comparable conditions, and/or such increased production results in a yield that is 10%, 25%, 50%, 75%, 100% or more higher than that observed with an otherwise identical organism lacking the modification under comparable conditions, and/or such increased production results in volumetric productivity that is 10%, 25%, 50%, 75%, 100% or more higher than that observed with an otherwise identical organism lacking the modification under comparable conditions, and/or such increased production results in a specific productivity increase of 10%, 25%, 50%, 75%, 100% or more higher than that observed with an otherwise identical organism lacking the modification under comparable conditions.

In some embodiments of the present disclosure, application of an alcohol tolerance modification involves application of a genetic modification—i.e., a change in the genetic information content in a cell. In some embodiments, such a genetic modification comprises altering (i.e., increasing or decreasing) expression or activity of one or more genetic elements (e.g., alcohol tolerance determinants) already present in the cell; in some embodiments, such a genetic modification comprises introducing one or more genetic elements into a cell, and/or removing one or more genetic elements from a cell. In some embodiments, such genetic elements encode or regulate one or more alcohol tolerance polypeptides; in some embodiments, such genetic elements consist of or comprise non-coding sequences. Representative examples of particular alcohol tolerance determinants for use in accordance with certain embodiments of the present invention include sequences found within the determinant sequences presented Tables 1-4, and particularly in Tables 1 and/or 2.

In some embodiments of the present disclosure, at least two alcohol tolerance determinants are utilized. For example, in some embodiments, at least two alcohol tolerance determinants from the determinant sequences in Tables 1 and/or 2 are utilized; in some embodiments, at least two alcohol tolerance determinants from the determinant sequences in Tables 3 and/or 4 are utilized; in some embodiments, at least one alcohol tolerance determinant from the determinant sequences in Tables 1 and/or 2 and at least one alcohol tolerance determinant from the determinant sequences in Tables 3 and/or 4 are utilized.

In some embodiments of the present disclosure, at least one alcohol tolerance determinant from the determinant sequences in Table 1A or Table 1B is utilized in combination with at least one determinant from the determinant sequences in Table 3.

In some embodiments, the present invention utilizes one or more alcohol tolerance determinants found within determinant sequences selected from determinant sequences provided in rows 20 (lp_{—}1293), 21 (lp_{—}1295 [nmtH3], 34 (lp_{—}2159), 35 (lp_{—}2160), 36 (lp_{—}2169), 37 (lp_{—}2170), 44 (lp_{—}2911), and/or 50 (lp_{—}3193 [prs2A]) of Table 1A, and/or in any of Tables 2T, 2U, 2AH, 2AI, 2AJ, 2AK, 2AR, and/or 2AX and combinations thereof. In one or more of these embodiments, an alcohol tolerance determinant is utilized in combination with one or more alcohol tolerance determinants found in the determinant sequences of Tables 3 and/or 4. In some embodiments, the determinants found in determinant sequences of Tables 3 and/or 4 are determinants whose introduction into or activity in a cell increases alcohol tolerance (e.g., those found in determinant sequences found in rows 2 (groES), 3 (groEL), 5 (cfa1) and/or 8 (cfa2) of Table 3, or in any of Tables 4B, 4C, 4E and/or 4H). In some embodiments, the determinant found in determinant sequences of Tables 3 and/or 4 are determinants whose disruption or inhibition in a cell increases alcohol tolerance (e.g., those found in determinant sequences found in rows 1 (htrA), 4 (clpP), 6 (relA/spoT), 7 (hrcA), and/or 8 (cfa2) of Table 3, or in any of Tables 4A, 4D, 4F, 4G and/or 4H).

In some embodiments of the present disclosure, a genetic modification that increases tolerance to one or more aliphatic alcohol compounds comprises introduction of heterologous genetic sequences (e.g., alcohol tolerance determinant sequences) into a cell. In some embodiments, such heterologous genetic sequences are ones that are found in source cells (e.g., in nature or in other engineered cells) that show tolerance to one or more aliphatic alcohol compounds.

In some embodiments, desirable source cells or organisms from which genetic sequences are obtained for introduction into host cells applied in accordance with the present disclosure are characterized by one or more attributes such as (i) intrinsic tolerance to one or more aliphatic alcohol compounds; (ii) environmental niche (e.g., exposure to butane, butanol, etc); (iii) potential to biosynthesize one or more aliphatic alcohol compounds (e.g., butanol); (iv) availability of genomic sequence information, or at least sequence information of relevant genetic elements (e.g., genes encoding polypeptides that contribute to tolerance); (v) taxonomic proximity to host cells; and/or (vi) minimal biosafety issues (e.g., infectious potential, etc).

In some embodiments, a source cell or organism is characterized by genetic compatibility with the intended host organism (i.e., the intended recipient of the source organism genetic information).

In some embodiments of the present disclosure, application of an alcohol tolerance modification involves increasing expression or activity of one or more alcohol tolerance polypeptides. In some embodiments, the alcohol tolerance polypeptide is heterologous to the host cell; in some embodiments, it is endogenous to the host cell.

As described herein, suitable alcohol tolerance polypeptides for use in accordance with the present disclosure include, among others, polypeptides that alter membrane composition, that participate in transport of undesirable factors (e.g., toxic compounds) out of the cell or desirable factors into the cell, that participate in metabolism of toxic compounds within the cell, and/or that otherwise protect cells from toxicity of aliphatic alcohol compounds. In some embodiments, such alcohol tolerance polypeptides are encoded by one or more alcohol tolerance determinants sequences presented in Tables 1 and/or 3. Alternatively or additionally, such alcohol tolerance polypeptides are homologs of those encoded by one or more alcohol tolerance determinants presented in Tables 1 and/or 3 and/or are encoded by determinant sequence presented in Tables 2 and/or 4.

In some embodiments of the present disclosure, cells are engineered to alter expression and/or activity of at least two alcohol tolerance polypeptides. For example, in some embodiments, a cell is separately or simultaneously engineered (e.g., by introduction of genetic elements [e.g., genes] encoding relevant polypeptides) to express (and/or activate) at least one alcohol tolerance polypeptide encoded by an alcohol tolerance determinant in Table 1 or 2 and also at least one alcohol tolerance polypeptide encoded by an alcohol tolerance determinant in determinant sequences of Tables 3 or 4. References to a Table herein include all subparts of the Table, unless otherwise noted. For example, “Table 1” includes both Table 1A and Table 1B.

One particular class of alcohol tolerance polypeptides whose expression or activity may desirably be altered by application of an alcohol tolerance modification in accordance with the present disclosure includes polypeptides (e.g., heat shock proteins) that participate in stress responses. In certain embodiments of the disclosure, modifications that alter expression or activity of such heat shock proteins are combined with one or more other alcohol tolerance modifications. Stress response polypeptides typically bind normative states of other proteins and assist in proper folding by recognizing exposed hydrophobic surfaces on normative protein species, which ultimately end up buried when the protein is in its properly folded, functional state. Stress response polypeptides typically form noncovalent interactions with the hydrophobic regions of misfolded proteins, thereby stabilizing them from irreversible multimeric aggregation, misfolding of nascent polypeptides, unfolding during exposure to stress and eventual degradation. The stabilized and properly folded proteins are therefore available to perform their cellular function(s).

The major established classes of heat shock proteins are the 90-kDa heat shock protein (HSP90), the 60-kDa heat shock protein (HSP60; including GroEL), the 70-kDa heat shock protein (HSP70; DnaK in E. coli) and 40-kDa heat shock protein (HSP40 or the DnaJ family). Another important protein involved in the heat shock response is a co-chaperone of HSP60 called chaperonin 10 (cpn10; GroES in E. coli).

DnaK operates by binding to nascent polypeptide chains on ribosomes, preventing premature folding, misfolding, or aggregation. DnaK is composed of two major functional domains. The NH₂-terminal ATPase domain and the COOH-terminal domain. The NH₂-terminal ATPase domain binds ADP and ATP and hydrolyzes ATP, whereas the COOH-terminal domain is responsible for polypeptide binding. DnaJ is a co-chaperone for DnaK. GrpE, another chaperone involved in the DnaKJ folding pathway, facilitates exchange between ADP and ATP. In many organisms, the genes for DnaK, DnaJ and GrpE are organized as an operon (the dnaK operon).

The GroEL/ES family of proteins binds to partially folded intermediates, preventing their aggregation, and facilitating folding and assembly. In addition, it has been suggested that GroEL, with the assistance of its co-chaperonin GroES, may allow misfolded structures to unfold and refold. The GroEL of E. coli consists of 14 identical subunits in two-stacked heptameric rings, each containing a central cavity. The size of the GroEL/ES complex cavity suggests that proteins of up to 50-60 kDa can be handled by this chaperone system. The genes for GroEL/ES are also typically organized as an operon (the groE operon). In B. subtilis, expression of the dnaK and groE operons is negatively regulated by a repressor protein through a CIRCE DNA element (a palindromic sequence between the promoter and the initiation codon). For example, in B. subtilis, inactivation of this repressor protein (HrcA), whose activity is modulated by GroEL/ES, results in constitutive expression of the two HSP operons, and this enhances the folding and secretory production of proteins which are difficult to fold.

In some embodiments, heat shock proteins, or other stress-related polypeptides are useful alcohol tolerance polypeptides. In certain embodiments, stress-related polypeptides that are heterologous to the host cell are employed; in some embodiments, stress-related polypeptides are utilized that are found in a source cell other than E. coli, B. subtilis, and/or C. acetobutylicum.

In some particular embodiments of the present disclosure, application of an alcohol tolerance modification involves increasing expression or activity of one or more alcohol tolerance polypeptides selected from the group consisting of a calcineurin-like phosphoesterase polypeptide, a cation transport protein (mntH3 related) polypeptide, a transcription regulator (lp_{—}2159 related) polypeptide, an lp_{—}2160 related polypeptide, an lp_{—}2169 related polypeptide, a phosphoglycerate mutase polypeptide, a CAAX protease polypeptide, a peptidylprolyl isomerase (prs2A related) polypeptide, and combinations thereof, optionally in combination with increasing expression or activity of one or more of a GroES polypeptide, a GroEL polypeptide, a cyclopropane-fatty-acyl-phospholipid synthase #1 (cfa1) polypeptide, a cyclopropane-fatty-acyl-phospholipid synthase #2 (cfa2) polypeptide, and combinations thereof and/or with decreasing expression and/or activity of one or more of a serine protease HtrA polypeptide, an ATP-dependent Clp protease proteolytic subunit polypeptide, a GTP pyrophosphokinase (relA/spoT) polypeptide, a heat-inducible transcription repressor (hrca) polypeptide, and/or a cyclopropane-fatty-acyl-phospholipid synthase #2 (cfa2) polypeptide.

### Biosynthesis of Aliphatic Alcohols

As discussed herein, a variety of organisms are known that produce one or more aliphatic alcohol compounds; metabolic pathways that operate in such organisms are well understood.

For example, FIG. 1 presents a schematic of a representative metabolic process that produces certain aliphatic alcohol compounds (e.g., ethanol, 1-butanol). This particular pathway is utilized, for example, in many strains of C. acetobutylicum.

In the pathway illustrated in FIG. 1, aliphatic alcohol compounds are produced from acetyl-CoA. In the production of ethanol, acetyl-CoA is reduced to acetaldehyde by the action of an aldehyde dehydrogenase, and then acetaldehyde is reduced to ethanol through the action of an alcohol dehydrogenase. In the production of butanol (in this case, 1-butanol), acetyl-CoA is first converted to acetoacetyl-CoA through action of acetyl-CoA acetyltransferase (a thiolase) that catalyzes the condensation of two acetyl-CoA molecules to produce aceotacetyl-CoA. Acetoacetyl-CoA is then converted to 3-hydroxybutyryl-CoA by 3-hydroxybutyryl-CoA dehydrogenase; 3-hydroxybutyryl-CoA is converted to crotonyl-CoA by crotonase; crotonyl-CoA is converted to butyryl-CoA by butyryl-CoA dehydrogenase; butyryl-CoA is converted to butyraldehyde by (butyrl)aldehyde dehydrogenase; and butyraldehyde is converted to 1-butanol by the alcohol dehydrogenase, butanol dehydrogenase. Each of the enzymes depicted in FIG. 1 as participating in production of ethanol and/or 1-butanol is an aliphatic alcohol biosynthesis polypeptide according to the present disclosure.

FIG. 1 also illustrates certain “competing reactions” that can occur and can divert carbon flow away from production of one or more aliphatic alcohol compounds. For example, butyryl-CoA can be diverted away from butanol production (and toward butyrate production) by action of phosphotransbutyrylase (optionally followed by butyrate kinase); acetoacetyl-CoA can be diverted away from production of butanol (and toward production of acetone) by action of CoA tranferase (optionally followed by acetoacetate decarboxylase); and acetyl-CoA can be diverted away from production of either butanol or ethanol (and toward production of acetate) by action of phosphotransacetylase (optionally followed by acetate kinase), and acetyl-CoA can be diverted away from production of butanol (and toward production of ethanol) by action of aldehyde dehydrogenase (optionally followed by alcohol dehydrogenase). Indeed, FIG. 1 is intended only as a representative illustration and not as an exhaustive depiction of all relevant metabolic pathways in a cell.

Those of ordinary skill will readily appreciate any of a variety of other competing reactions that may occur in any particular cell. Enzymes that participate in such competing reactions are considered aliphatic alcohol biosynthesis competitor polypeptides as described herein. In some embodiments of the present disclosure, a modification is applied that reduces level or activity of one or more aliphatic alcohol biosynthesis competitor polypeptides, such that higher levels of aliphatic alcohol compounds, or of a particular aliphatic alcohol compound (e.g., relative to other compounds, for example other aliphatic alcohol compounds) are produced.

Polypeptides that catalyze different steps of the pathway illustrated in FIG. 1 have been identified in a variety of source organisms; in many cases, their genes have been cloned. For example, international patent application number PCT/US2006/038001 (publication number WO 2007/041269) describes a variety of polypeptides and genes, from a number of source organisms that catalyze steps involved in butanol synthesis. Representative particular genes are presented in Table 2 of PCT/US2006/038001 (WO 2007/041269).

FIG. 3 illustrates certain metabolic pathways that operate to produce a particular aliphatic alcohol compound, isobutanol. In particular, FIG. 3 shows four different isobutanol biosynthetic pathways. The steps labeled “a”, “b”, “c”, “d”, “e”, “f”, “g”, “h”, “i”, “j” and “k” represent the substrate to product conversions described below.

Three of the isobutanol biosynthetic pathways depicted in FIG. 3 comprise conversion of pyruvate to isobutanol via a series of enzymatic steps. The preferred isobutanol pathway (FIG. 3, steps a to e), comprises the following substrate to product conversions:

a) pyruvate to acetolactate, as catalyzed for example by acetolactate synthase,

b) acetolactate to 2,3-dihydroxyisovalerate, as catalyzed for example by acetohydroxy acid isomeroreductase,

c) 2,3-dihydroxyisovalerate to α-ketoisovalerate, as catalyzed for example by acetohydroxy acid dehydratase,

d) α-ketoisovalerate to isobutyraldehyde, as catalyzed for example by a branched-chain keto acid decarboxylase, and

e) isobutyraldehyde to isobutanol, as catalyzed for example by, a branched-chain alcohol dehydrogenase.

This pathway combines enzymes known to be involved in well-characterized pathways for valine biosynthesis (pyruvate to α-ketoisovalerate) and valine catabolism (α-ketoisovalerate to isobutanol). Since many valine biosynthetic enzymes also catalyze analogous reactions in the isoleucine biosynthetic pathway, substrate specificity can be an important consideration in selecting the gene sources. In some embodiments, genes of interest for the acetolactate synthase enzyme are those from Bacillus (alsS) and Klebsiella (budB). These particular acetolactate synthases are known to participate in butanediol fermentation in these organisms and show increased affinity for pyruvate over ketobutyrate (Gollop et al., J. Bacteriol. 172(6):3444, 1990; Holtzclaw et al., J. Bacteriol. 121(3):917, 1975).

The second and third steps are catalyzed by acetohydroxy acid reductoisomerase and dehydratase, respectively. These enzymes have been characterized from a number of sources, such as for example, E. coli (Chunduru et al., Biochemistry 28(2):486, 1989; Flint et al., J. Biol. Chem. 268(29):14732, 1993).

The final two steps of this isobutanol pathway are known to occur in yeast, which can use valine as a nitrogen source and, in the process, secrete isobutanol. α-ketoisovalerate can be converted to isobutyraldehyde by a number of keto acid decarboxylase enzymes, such as for example pyruvate decarboxylase. In some embodiments, a decarboxylase with decreased affinity for pyruvate is utilized in order to reduce or prevent routing of pyruvate away from isobutanol production. At least two such enzymes are known in the art (Smit et al., Appl. Environ. Microbiol. 71(1):303, 2005; de la Plaza et al., FEMS Microbiol. Lett. 238(2):367, 2004). Both enzymes are from strains of Lactococcus lactis and have a 50-200-fold preference for ketoisovalerate over pyruvate. Also, a number of aldehyde reductases have been identified in yeast, many with overlapping substrate specificity. Those known to prefer branched-chain substrates over acetaldehyde include, but are not limited to, alcohol dehydrogenase VI (ADH6) and Ypr1p (Larroy et al., Biochem. J. 361 (Pt 1):163, 2002; Ford et al., Yeast 19(12):1087, 2002), both of which use NADPH as an electron donor. An NADPH-dependent reductase, YqhD, active with branched-chain substrates has also been identified in E. coli (Sulzenbacher et al., J. Mol. Biol. 342(2):489, 2004).

Another pathway for converting pyruvate to isobutanol comprises the following substrate to product conversions (FIG. 3, steps a, b, c, f, g, e):

a) pyruvate to acetolactate, as catalyzed for example by acetolactate synthase,

b) acetolactate to 2,3-dihydroxyisovalerate, as catalyzed for example by acetohydroxy acid isomeroreductase,

c) 2,3-dihydroxyisovalerate to α-ketoisovalerate, as catalyzed for example by acetohydroxy acid dehydratase,

f) α-ketoisovalerate to isobutyryl-CoA, as catalyzed for example by a branched-chain keto acid dehydrogenase,

g) isobutyryl-CoA to isobutyraldehyde, as catalyzed for example by an acylating aldehyde dehydrogenase, and

e) isobutyraldehyde to isobutanol, as catalyzed for example by, a branched-chain alcohol dehydrogenase.

The first three steps in this pathway (a,b,c) are the same as those described above. The α-ketoisovalerate is converted to isobutyryl-CoA by the action of a branched-chain keto acid dehydrogenase. While yeast typically can only use valine as a nitrogen source, many other organisms (both eukaryotes and prokaryotes) can use valine as the carbon source as well. These organisms have branched-chain keto acid dehydrogenase (Sokatch et al. J. Bacteriol. 148(2):647, 1981), which generates isobutyryl-CoA. Isobutyryl-CoA may be converted to isobutyraldehyde by an acylating aldehyde dehydrogenase. Dehydrogenases active with the branched-chain substrate have been described in at least Leuconostoc and Propionibacterium (Kazahaya et al., J. Gen. Appl. Microbiol. 18:43, 1972; Hosoi et al., J. Ferment. Technol. 57:418, 1979). However, it is also possible that acylating aldehyde dehydrogenases known to function with straight-chain acyl-CoAs (i.e. butyryl-CoA), may also work with isobutyryl-CoA. The isobutyraldehyde is then converted to isobutanol by a branched-chain alcohol dehydrogenase, as described above for the first pathway.

Another pathway for converting pyruvate to isobutanol comprises the following substrate to product conversions (FIG. 3, steps a, b, c, h, i, j, e):

a) pyruvate to acetolactate, as catalyzed for example by acetolactate synthase,

b) acetolactate to 2,3-dihydroxyisovalerate, as catalyzed for example by acetohydroxy acid isomeroreductase,

c) 2,3-dihydroxyisovalerate to α-ketoisovalerate, as catalyzed for example by acetohydroxy acid dehydratase,

h) α-ketoisovalerate to valine, as catalyzed for example by valine dehydrogenase or transaminase,

i) valine to isobutylamine, as catalyzed for example by valine decarboxylase,

j) isobutylamine to isobutyraldehyde, as catalyzed for example by omega transaminase, and

e) isobutyraldehyde to isobutanol, as catalyzed for example by, a branched-chain alcohol dehydrogenase.

The first three steps in this pathway (a, b, c) are the same as those described above.

This pathway involves the addition of a valine dehydrogenase or a suitable transaminase. Valine (and or leucine) dehydrogenase catalyzes reductive amination and uses ammonia; Kₘ values for ammonia are in the millimolar range (Priestly et al., Biochem J. 261(3):853, 1989; Vancura et al., J. Gen. Microbiol. 134(12):3213, 1988; Zink et al., Arch. Biochem. Biophys. 99:72, 1962; Sekimoto et al. J. Biochem (Japan) 116(1):176, 1994). Transaminases typically use either glutamate or alanine as amino donors and have been characterized from a number of organisms (Lee-Peng et al., J. Bacteriol. 139(2):339, 1979; Berg et al., J. Bacteriol. 155(3):1009, 1983). An alanine-specific enzyme may be desirable, since the generation of pyruvate from this step could be coupled to the consumption of pyruvate later in the pathway when the amine group is removed (see below).

The next step is decarboxylation of valine, a reaction that occurs in valanimycin biosynthesis in Streptomyces (Garg et al., Mol. Microbiol. 46(2):505, 2002). The resulting isobutylamine may be converted to isobutyraldehyde in a pyridoxal 5′-phosphate-dependent reaction by, for example, an enzyme of the omega-aminotransferase family. Such an enzyme from Vibrio fluvialis has demonstrated activity with isobutylamine (Shin et al., Biotechnol. Bioeng. 65(2):206, 1999). Another omega-aminotransferase from Alcaligenes denitrificans has been cloned and has some activity with butylamine (Yun et al., Appl. Environ. Microbiol. 70(4):2529, 2004). In this direction, these enzymes use pyruvate as the amino acceptor, yielding alanine. As mentioned above, adverse affects on the pyruvate pool may be offset by using a pyruvate-producing transaminase earlier in the pathway. The isobutyraldehyde is then converted to isobutanol by a branched-chain alcohol dehydrogenase, as described above for the first pathway.

The fourth isobutanol biosynthetic pathway depicted in FIG. 3 comprises the substrate to product conversions shown as steps k, g, e of that Figure. A number of organisms are known to produce butyrate and/or butanol via a butyryl-CoA intermediate (Dune et al., FEMS Microbiol. Rev. 17(3):251, 1995; Abbad-Andaloussi et al., Microbiology 142(5):1149, 1996). Isobutanol production may be engineered in these organisms by addition of a mutase able to convert butyryl-CoA to isobutyryl-CoA (FIG. 3, step k). Genes for both subunits of isobutyryl-CoA mutase, a coenzyme B₁₂-dependent enzyme, have been cloned from a Streptomycete (Ratnatilleke et al., J. Biol. Chem. 274(44):31679, 1999). The isobutyryl-CoA is converted to isobutyraldehyde (step g in FIG. 3), which is converted to isobutanol (step e in FIG. 3).

Those of ordinary skill are therefore aware of a variety of biosynthetic pathways that may be employed and/or engineered for the production of isobutanol according to the present disclosure. Furthermore, those of ordinary skill will be able to utilize publicly available sequences to construct and/or otherwise utilize such pathways. Representative such sequences (gene sequences) can be found, for example, in Table 2 of US patent application 2007/0092957.

### Production of Aliphatic Alcohol Compounds

Aliphatic alcohol compounds can be produced by cultivating engineered microorganisms as described herein.

In general, cells engineered as described herein are grown in the presence of a suitable carbon source and other nutrients, under appropriate growth conditions. In some embodiments, modified cells are grown under aerobic conditions; in some embodiments, modified cells are grown under anaerobic conditions. As is known in the art, conditions under which cells having the ability to produce a particular compound are grown can often influence the amount of compound produced and/or the timing of its production. For example, it is known that factors such as temperature, pH, carbon source, availability of certain cofactors, growth rate, etc. can affect the metabolic state of cultured microorganisms, and therefore can alter production of particular compounds of interest.

For example, it is known that certain C. acetobutylicum strains have three different basic metabolic states that can be induced by changes in culture conditions. In particular, these strains have an “acidogenic” state characterized by production of acetic and butyric acids, a “solventogenic” state characterized by production of acetone, butanol, and ethanol, and an “alcohologenic” state characterized by production of butanol and ethanol. The acidogenic state is observed when these strains are grown under conditions of neutral pH with glucose as a carbon source and/or when these strains are grown under conditions of low ATP availability (e.g., under carbon limitation); the solventogenic state is observed when these strains are grown at low pH with glucose as a carbon source and/or when strains are grown under conditions of high ATP availability (e.g., under carbon-sufficient conditions and/or at low growth rates); and the alcohologenic state is observed when these strains are grown at neutral pH under conditions of high NAD(P)H availability (e.g., due to lowered electron flow toward molecular-hydrogen production, for example by decreasing hydrogenase activity as occurs under conditions of iron limitation, in the presence of carbon monoxide, and/or in the presence of artificial electron carriers such as viologen or neutral red; due to use of a more reduced substrate than glucose [e.g., use of glycerol]; etc). In some embodiments of the present disclosure, one or more aliphatic alcohol compounds is produced by growth of a C. acetobutylicum strain in its solventogenic or alcohologenic state.

In general, appropriate carbon sources for use in accordance with the present disclosure include, but are not limited to monosaccharides (e.g., fructose, glucose, etc), oligosaccharides (e.g., lactose, sucrose, etc), polysaccharides (e.g., cellulose, starch, etc), single carbon substrates (e.g., carbon dioxide, methanol, etc) and mixtures thereof. Particular sugar carbon sources of interest include, for example, fructose, glycerol, glucose, galactose, dextrose, and sucrose. Those of ordinary skill in the art will appreciate that the source of carbon may be provided by way of pure material or through complex or crude mixtures including, for example, cheese whey permeate, cornsteep liquor, sugar beet molasses, barley malt, etc.

Those of ordinary skill in the art are also aware of a variety of different nitrogen sources (e.g., ammonium sulfate, proline, sodium glutamate, soy acid hydrolysate, yeast extract-peptone, yeast nitrogen base, corn steep liquor, etc, and combinations thereof) that can be utilized in accordance with the present disclosure.

In some embodiments, cells are grown via batch or fed-batch fermentation; in some embodiments, cells are grown via continuous feed fermentation.

In general, classical batch fermentation typically utilizes a closed system where the composition of the medium is set at the beginning of the fermentation and is not subject to artificial alterations during the fermentation. Thus, at the beginning of the fermentation the medium is inoculated with the desired organism or organisms, and fermentation is permitted to occur. In some embodiments, no additions are made to the system once fermentation has begun. In some embodiments, additions are made, for example, of salts, etc., and/or of factors that modulate pH and/or oxygen concentration. In many embodiments, however, no carbon source additions are made.

In many batch fed fermentation embodiments, the metabolite and biomass compositions of the system change constantly up to the time that fermentation is stopped. Within batch cultures, cells often pass through a static lag phase to a high growth log phase and finally to a stationary phase where growth rate is diminished or halted. If untreated, cells in the stationary phase will eventually die. Cells in log phase are often responsible for the bulk of production of relevant compounds (e.g., aliphatic alcohol compounds and/or intermediates).

One variation on the standard batch system is the fed-batch system. In fed-batch fermentations, substrate is added in increments as the fermentation progresses. Fed-batch systems are particularly useful when catabolite repression is apt to inhibit metabolism of the cells and/or where it is desirable to have limited amounts of substrate in the medium. Measurement of actual amounts of a particular substrate can be performed, often by indirect assessment, for example through measurement of changes in factors such as pH, dissolved oxygen, and the partial pressure of gases such as CO₂. Batch and fed-batch fermentation protocols are well known in the art.

Continuous fermentation processes typically utilize an open system where a defined fermentation medium is added continuously to a bioreactor, and an equal amount of conditioned medium is removed for processing. Continuous fermentation can maintain cultures at a constant high density where cells are primarily in log phase growth.

Continuous fermentation allows for the modulation of one factor or any number of factors that affect cell growth and/or production of desired product (e.g., aliphatic alcohol compound). For example, in some embodiments, a particular nutrient is maintained at limiting levels and other factors are permitted to fluctuate. In some embodiments, a number of factors affecting growth can be simultaneously or sequentially altered continuously; cell concentration (e.g., measured by medium turbidity) may optionally be kept constant.

In some embodiments, a continuous system is operated at steady state growth, so that cell loss that is concomitant with medium removal is balanced against the cell growth rate. Methods of continuous fermentation, including methods of modulating nutrients and growth factors, and for maximizing rate and/or extent of production of a desired product (e.g., one or more aliphatic alcohol compounds) are known in the art.

In some embodiments, inventive modified cells are grown in a multi-phase feeding protocol, for example in which different phases are designed to induce different metabolic states. In some embodiments, inventive modified cells are grown in a multi-phase feeding protocol, for example in which some phases are continuous and some are batch fed (see, for example, U.S. Pat. No. 5,063,156).

In some embodiments, inventive modified cells are cultivated at constant temperature (e.g., between about 20-40, or 20-30 degrees, including for example at about 20, 20.5, 21, 21.5, 22, 22.5, 23, 23.5, 24, 24.5, 25, 25.5, 26, 26.5, 27, 27.5, 28, 28.5, 29, 29.5, 30° C. or above, typically within the range of about 35-40° C.) and/or pH (e.g., within a range of about 4-7.5, or 4-6.5, including at about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5 or above, typically within the range of about 5.0-9.0, and often with the range of about 6.0-8.0); in other embodiments, temperature and/or pH may be varied during the culture period, either gradually or in a stepwise fashion.

In some embodiments of the present disclosure, asporogenic strains are utilized, particularly of C. acetobutylicum, for example as described in U.S. Pat. No. 5,063,156.

### Isolation of Aliphatic Alcohol Compounds

Aliphatic alcohol compounds produced as described herein may be isolated using any of a variety of known techniques. For example, solids may be removed from the fermentation medium, e.g., by centrifugation, filtration, decantation, etc. Techniques such as distillation, gas stripping, liquid-liquid extraction, membrane-based separation, etc. may be employed to isolate one or more aliphatic alcohol compounds.

Those of ordinary skill in the art will be well aware of the advantages and disadvantages of different techniques in different situations. To give one particular example, 1-butanol forms a low boiling point, azeotropic mixture with water, so that distillation generally can only be used to separate the mixture up to its azeotrophic composition. However, distillation may be used in combination with one or more other separation techniques to obtain separation around the azeptrope. Exemplary such techniques include, for example, decantation, liquid-liquid extraction, adsorption, pervaporation, membrane-based techniques, etc. Alternatively or additionally, 1-butanol may be isolated using azeotropic distillation with an entrainer (see, for example, Doherty & Malone, Conceptual Design of Distillation Systems, McGraw Hill, New York, 2001).

### Uses of Aliphatic Alcohol Compounds

Aliphatic alcohol compounds produced and/or isolated as described herein may be utilized as and/or incorporated into any of a variety of commercial products. To give but a few examples, such aliphatic alcohol compounds may be employed as or in transport fuels, solvents, swelling agents, brake fluid, extractants, cement additives, ore flotation agents, melamine formaldehyde resins, etc.

In certain embodiments, the aliphatic alcohol compound is butanol (e.g., 1-butanol). Butanol may be employed as a transport fuel or fuel additive, bulk chemical precursor for production of acrylate and methacrylate esters, glycol ethers, butyl acetate, butylamines, and amino resins. It may also be useful for the production of adhesives/scalants, alkaloids, antibiotics, camphor, deicing fluid, dental products, detergents, elastomers, electronics, emulsifiers, eye makeup, fibers, flocculants, flotation aids (e butyl xanthate), hard-surface cleaners, hormones and vitamins, hydraulic and brake fluids, industrial coatings, lipsticks, nail care products, paints, paint thinners, perfumes, pesticides, plastics, printing ink, resins, safety glass, shaving and personal hygiene products, surface coatings, super absorbents, synthetic fruit flavoring, textiles, as mobile phases in paper and thin-layer chromatography, as oil additive, as well as for leather and paper finishing,

### EXEMPLIFICATION

All basic molecular biology and DNA manipulation procedures described herein are generally performed according to Sambrook et al., or Ausubel et al., (Sambrook J, Fritsch E F, Maniatis T (ed.) 1989, Molecular Cloning: A Laboratory Manual, Cold Spring Harbor Laboratory Press: New York; Ausubel F M, Brent R, Kingston R E, Moore D D, Seidman J G, Smith J A, Struhl K (ed.) 1998 Current Protocols in Molecular Biology, Wiley: New York).

### Example 1

### Oligonucleotide Primers Used in Plasmid Construction

Table 7 lists oligonucleotide sequences used in the plasmid contruction described in the examples below.

TABLE 7

| Primer | Sequence |
|---|---|
| 5174 | 5′-TTTGGATCCGATTCTATTGTTAGCTATTTTGGGTGG-3′ |
| 5175 | 5′-AAAGTCGACATCGTGGTATTAGTGATGCAAAGAAAGG-3′ |
| 5433 | 5′-AACGGCCGAAGGATTATTCGGCTGGTTGAGACGTTAAA-3′ |
| 5434 | 5′-AACGGCCGAAATAACACCTTAAGTCTAGCACCACCCGC-3′ |
| 5435 | 5′-CTCCAGTAAGAATATTTGCATTGTGTATC-3′ |
| 5436 | 5′-AAAAAGGCCCTTATAACTTACAAATAACCCC-3′ |
| 5437 | 5′-AAAAAAGCGGCCGCTCTTTATTCTTCAACTAAAGCACC-3′ |
| 5438 | 5′- |
| | AAAAAAGCGGCCGCAATGTATTTAGAAAAATAAACAAATAGG-3′ |
| 5450 | 5′- |
| | AAAAAGCGGAAGAGCGAGGGCGGAGTTGTTGACAGCCGAGGTAC |
| | CATGTGGTATAATCCCGAGTGTGGAATTGTGAGCGGATAACA-3′ |
| 5451 | 5′-GTTTTCGAGGGGTTATTTGTAAGTTATAAGGCCCT-3′ |
| 5452 | 5′-AAAAAGCGGAAGAGCGAGGGCGGAGTTG-3′ |

### Example 2

### Description of L. Plantarum Strains and Plasmids

Table 8 describes some of the L. plantarum strains (including the plasmids they contain) used in subsequent examples.

TABLE 8

| L. plantarum | | |
|---|---|---|
| Strain | Plasmid | Plasmid Description |
| PNO512 | None | Wild Type Host CONTROL |
| MS362 | pMPE6 | Vector Only CONTROL |
| MS379 | pMPE41 | A plasmid construct for overexpression of open reading frame |
| | | (1p_3193) from L. plantarum BAA-793. This ORF encodes the Prs2A |
| | | protein foldase. |
| MS354 | p5AE4-1 | Randomly cloned, 1960-bp fragment of L. plantarum BAA-793 |
| | | genomic DNA cloned in vector pMPE6. Fragment includes open |
| | | reading frame (ORF) 1p_2911 which encodes a putative membrane |
| | | bound protein that is predicted to be a member of the CAAX protease |
| | | family. Also included on the fragment is 104 bp of DNA upstream of |
| | | 1p_2911 and 655 bp of downstream sequence. The p5AE4-1 plasmid |
| | | insert sequence is shown in Table 1B. |
| MS356 | p5AE0-4 | A randomly cloned, 3240-bp fragment of L. plantarum BAA-793 |
| | | genomic DNA cloned in vector pMPE6. Fragment includes 2 |
| | | complete ORFs; 1p_1295 which encodes a putative cation transport |
| | | protein and 1p_1293 which encodes a conserved hypothetical protein |
| | | that shares similarity to a group of phosphoesterases. The p5AE0-4 |
| | | plasmid insert sequence is shown in Table 1B. |
| MS359 | p5AE0-14 | A randomly cloned, 2341-bp fragment of L. plantarum BAA-793 |
| | | genomic DNA cloned in vector pMPE6. Fragment includes 2 |
| | | complete ORFs; 1p_2159 which encodes a predicted transcriptional |
| | | regulator that shows homology to a group of sphingosine kinases, and |
| | | 1p_2160 which encodes a small, 66 amino acids, hypothetical protein |
| | | that shows no significant homology with proteins of known function. |
| | | The p5AE0-14 plasmid insert sequence is shown in Table 1B. |
| MS364 | p5AE4-24 | A randomly cloned, 2477-bp fragment of L. plantarum BAA-793 |
| | | genomic DNA cloned in vector pMPE6. Fragment includes 2 |
| | | complete open reading frames (ORFs); 1p_2170 which encodes a |
| | | predicted phosphoglycerate mutase and 1p_2169 which encodes a |
| | | hypothetical protein of unknown function. Also included on the insert |
| | | is a portion (1001 bp) of the 5′-end of recD. The p5AE4-24 plasmid |
| | | insert sequence is shown in Table 1B. |

### Example 3

### Construction of Lactobacillus plantarum Plasmid Cloning Vector

The purpose of this example is to describe the construction a cloning vector that can be stably propagated in both Escherichia coli and Lactobacillus plantarum. Such a vector allows genes cloned as “inserts” within the vector multiple cloning site (MCS) to be expressed via an upstream promoter. Genes cloned as inserts will also be subject to increased expression as a result of their increased copy number which is due to the fact that multiple copies of a plasmid are replicated within each host cell.

A series of shuttle vectors that replicate in both E. coli and L. plantarum were constructed. pMPE1 was constructed by amplifying a 0.7 kb fragment of pLF1, a naturally occurring plasmid in L. plantarum strain ATCC 14917. The 0.7 kb fragment containing the predicted minimum sequence required for replication of pLF1 was amplified using primers 5436 and 5435. This sequence was identified by sequence comparisons with p256 a closely related plasmid for which the minimum required sequence for replication had been experimentally determined (Sorvig et al. 2005. Microbiology 151:421-431). The pLF1 PCR product was digested with EagI and ligated to a 2.6 kb DraI fragment obtained from pMK4 (Sullivan et al. 1984. Gene 29:21-26), a Bacillus shuttle vector obtained from the Bacillus Genetic Stock Center (Ohio State University). The DraI fragment contained the pMK4 E. coli origin of replication (pUC-ori), its multiple cloning site (MCS) and its chloramphenicol resistance gene. The resulting plasmid, designated pMPE1, was tested by transforming it into L. plantarum strain BAA-793 by electroporation where it was found to replicate and to allow selection by demanding resistance to chloramphenicol.

pMPE1 was modified to improve its stability in L. plantarum by the addition of a toxin/antitoxin stability cassette. The cassette was PCR amplified from pLF1 using the primers 5437 and 5438. The resulting 0.8 kb PCR product was digested with Eco01091 and then ligated with pMPE1 which had been digested with Eco01091 and DraI (a 2904 by fragment). The resulting plasmid was designated pMPE5. Stability tests subsequently showed that pMPE5 was significantly more stable than pMPE1 in L. plantarum (100% vs. ˜75% after 80 generations of growth without selection respectively).

pMPE5 was modified to replace the lacZ promoter upstream of its MCS with a promoter that more closely resembled an L. plantarum native promoter. To do this, pMPE5 was cut with Eco01091 and SapI which removed the 5′ end of the lacZa and the lacZ promoter from the vector. Using the PCR primers 5451, 5450, and 5452 in a 2 step PCR protocol, a PCR product containing the 5′ portion of lacZa, a ribosomal binding site (RBS) and an L. plantarum rRNA promoter was generated using pMPE5 as a PCR template. The PCR product was digested with Eco01091 and SapI and ligated into the previously digested pMPE5 fragment producing pMPE6.

### Example 4

### Construction of a Lactobacillus plantarum Knock-Out Vector

The purpose of this example is to describe the construction of a plasmid vector that allows the inactivation of L. plantarum chromosomal genes via single crossover recombination. This technique has been described for many bacteria including L. plantarum (Leer et al. 1993. Mol. Gen. Genet. 239:269-272).

pMPE3 was constructed as a knockout vector for use in L. plantarum. pMPE3 was constructed by amplifying a 2.45 kb fragment of pMK4 using the primers 5433 and 5434. The amplified fragment contained the multiple cloning site, lacZa, pUC-ori and chloramphenicol resistance gene from pMK4. The amplified fragment was digested with NotI and then circularized by ligation, generating pMPE3. Because pMPE3 does not replicate in L. plantarum, portions of genes can be cloned into the MCS of the plasmid and the resulting recombinant plasmid can then be transformed into L. plantarum by electroporation. Selection for transformants that are resistant to chloramphenicol results in strains in which a single crossover recombination event has occurred between the cloned gene fragment and its analogous chromosomal gene. Such a recombination event results in the integration of the vector sequence into the chromosome and an insertional mutation in the target gene.

### Example 5

### Quantification of Glucose, Lactose, and 1-Butanol

The present example describes a particular protocol used to quantify glucose, lactate, and butanol levels in broth samples.

Glucose, lactate, and butanol levels were quantified from broth samples using HPLC analysis. The instrumentation for detection was comprised of a Waters 717 Plus auto sampler fronting a Waters 515 pump, which was coupled to a Waters 2414 refractive index (RI) detector. An Aminex Fast Acid ion exclusion column (100-mm×7.8-mm, Bio-Rad), with Aminex HPX-87H guard column (20-mm×7.8-mm guard column, Bio-Rad), was used for separation.

Samples were prepared for HPLC analysis by first centrifuging (30,000×g) harvested shake flask cultures and transferring supernatant to a fresh Eppendorf tube. Samples were diluted 10-fold into mobile phase, and the resulting preparations were loaded onto the 96 vial autosampler carousel, which is maintained at 15° C. 20 μL of diluted sample is used for instrument injection.

An isocratic separation was performed at 30° C. using 0.05% trifluoracetic acid as the mobile phase at a flow rate of 0.6 mL/min (1400 PSI as high pressure limit).

### Example 6

### Metabolic Assay to Determine Relative Levels of 1-Butanol Tolerance

The purpose of this example is to describe a quantitative tolerance assay for L. plantarum strains growing in liquid cultures. The assay uses metabolic activity as a tolerance metric. Individual strains were grown in cultures containing varying 1-butanol concentrations and metabolic activity levels were determined by HPLC measurements of the levels of lactate produced and/or glucose removed by each strain. Strains with higher tolerance were identified by their ability to produce higher levels of lactate and/or to remove more glucose from the culture supernatant in the presence of 1-butanol.

10-ml De Man, Rogosa and Sharpe (MRS; J. Appl. Bact., 23; 130-135 (1960)) broth cultures containing 0%, 1.7%, 2.0% and 2.3% (w/v) 1-BuOH were grown (3 replicates for each strain at each BuOH concentration) in 15-ml tubes (1% stationary phase inoculum) at 30° C. Samples of individual cultures were removed after 48 hr for analysis by HPLC to determine concentrations of glucose, lactate and 1-butanol. Cell density was also determined at each time point by measuring OD₆₀₀.

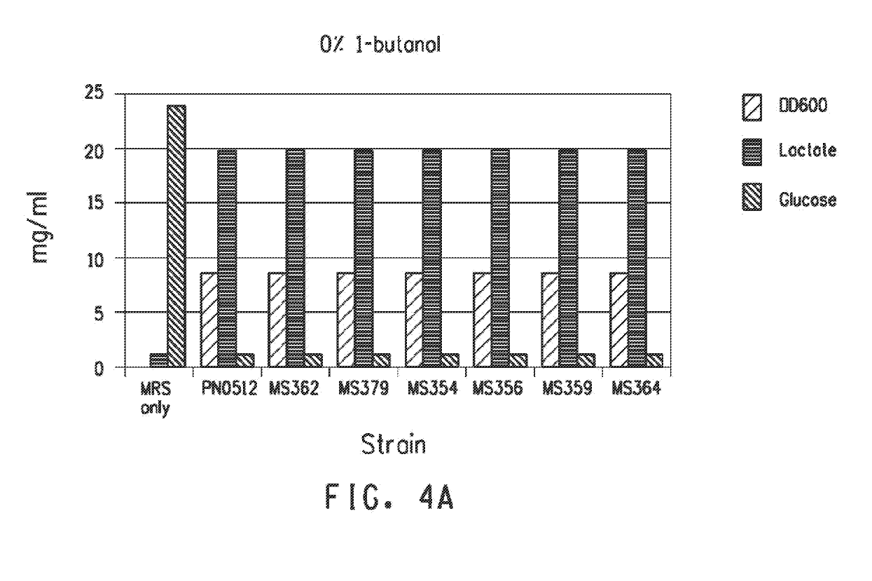

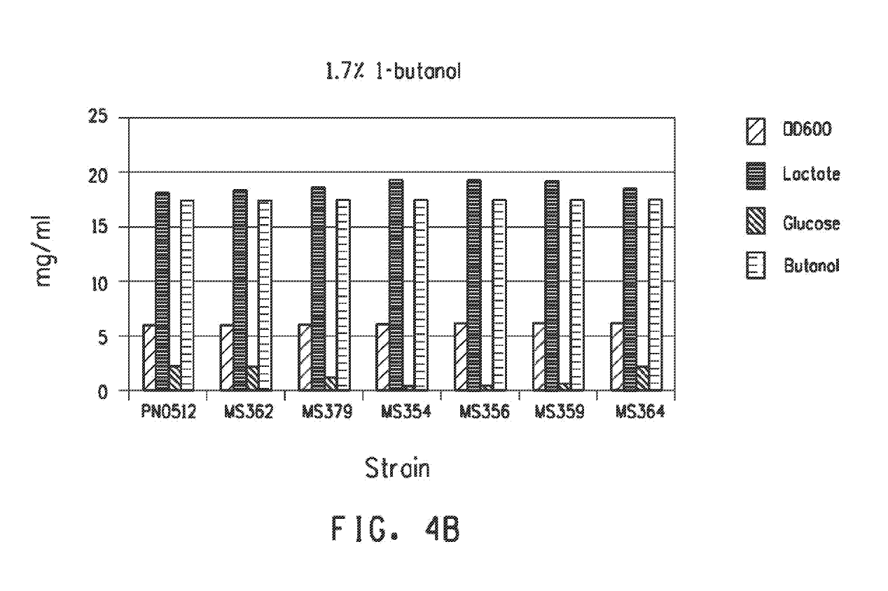

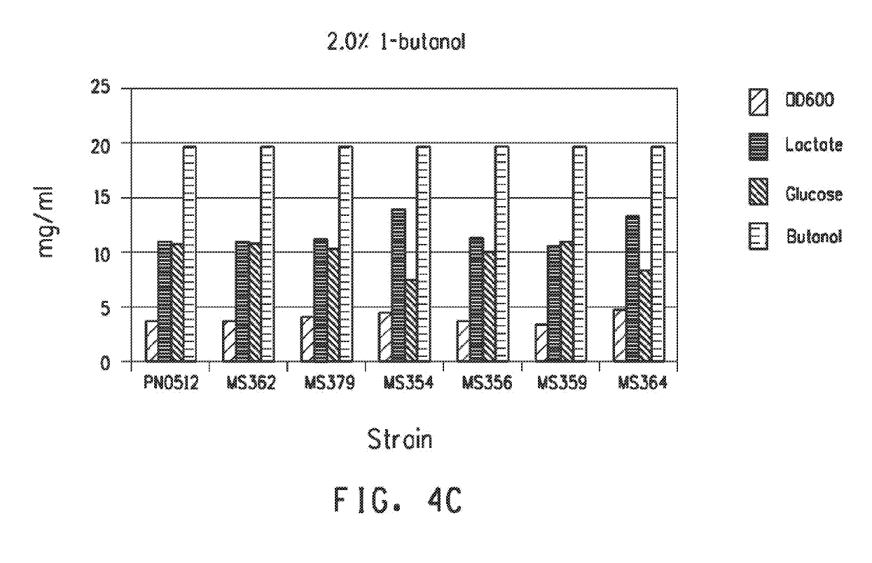

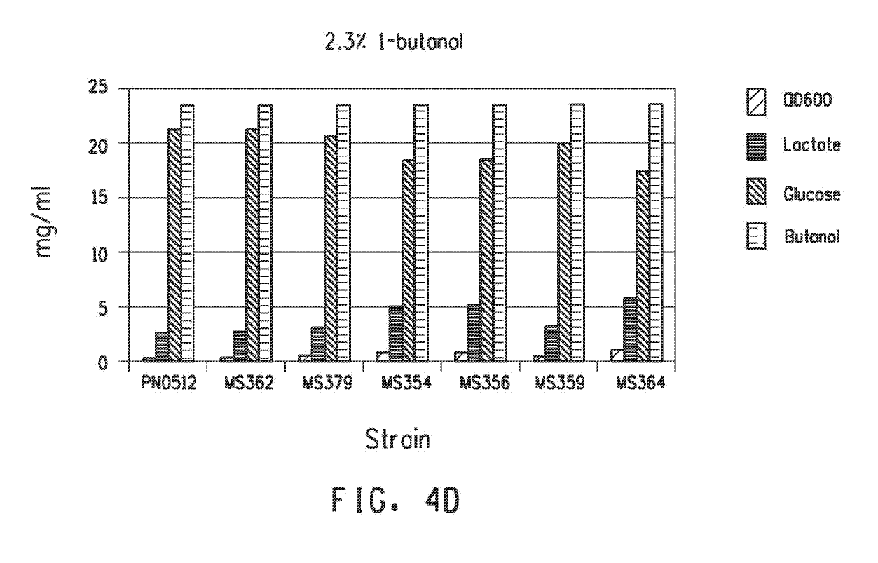

FIG. 4 shows graphic depictions of OD₆₀₀ and HPLC lactate, glucose and butanol measurements for 7 strains grown for 48 hours in MRS broth in the presence of predetermined concentrations of 1-butanol (w/v) (0.1% (panel 4A), 1.7% (panel 4B), 2.0% (panel 4C) and 2.3% (panel 4D)). Five of the strains contained plasmids encoding different alcohol tolerance determinant sequences. Data are the averages of 3 replicates. Descriptions of the strains and plasmids are given in Table 8 in Example 2 above. All 5 of the strains that carried plasmids which contained alcohol tolerance determinant sequences exhibited increased metabolic activity (and thus, butanol tolerance) in the presence of butanol as demonstrated by higher glucose consumption and lactate production relative to the controls. All five strains showed significant increases in glucose uptake (107% for MS354, 106% for MS356, 40% for MS359, 156% for MS364, 27% for MS379) as well as lactate production (124% for MS354, 122% for MS356, 45% for MS359, 182% for MS364, 27% for MS379) relative to the vector only control strain in the presence of 2.3% butanol, the 1-butanol IC₉₀ of L. plantarum PN0512.

### MRS Medium

| | | |
|---|---|---|
| Yeast extract | 5 | g |
| Beef extract | 10 | g |
| Peptone | 10 | g |
| Glucose | 20 | g |
| Tween 80 | 5 | ml |
| K₂HPO₄ | 2 | g |
| Sodium acetate | 5 | g |
| Diamonium citrate | 2 | g |
| MgSO₄•7H₂O | 0.2 | g |
| MnSO₄•4H₂O | 0.05 | g |
| Distilled water | 1000 | ml |

Dissolve the ingredients in water and adjust pH to 6.2 - 6.6.

### Example 7

### Assessment of Aliphatic Alcohol IC₅₀

The present example described methods used to determine IC₅₀ values for aliphatic alcohol compounds (with regard to particular microorganism strains or cultures).

IC₅₀ values were determined in shake flasks as follows. Duplicate glass test tubes containing 5 ml MRS medium plus 10 μg/ml chloramphenicol (MRS-Cm) were each inoculated with a single colony and grown for 24 h at 30° C. in a rollerdrum. 500 μl of each of these cultures were used to inoculate 25 ml MRS-Cm, which was grown for 16 h at 30° C. with shaking at 110 rpm. These precultures were then used to inoculate duplicate flasks containing 40 ml MRS plus aliphatic alcohol compound (e.g., butanol) at 0, 1.4%, 1.7%, 2.0% and 2.3% (w/v). Cultures were grown at 30° C. with shaking at 110 rpm, and growth was measured by monitoring OD₆₀₀. OD₆₀₀ values that were clearly off the growth curve were discarded (less than 1% of values were discarded).

For each flask, growth rates (μ) and doubling times (T_{d}) were determined by linear regression of the natural log of the OD using the set of points (minimum 3, usually 4 or 5) that gave the highest μ and lowest T_{d} Inhibition in each shake flask was determined relative to 0% butanol flasks inoculated from the same preculture.

IC₅₀ was calculated using the formula (C₂−C₁)(I₂−I₁)*(50%−I₁)+C₁ where C₁ and C₂ are the compound (e.g., butanol) concentrations that exhibited just under and just over 50% inhibition, respectively, and I₁ and I₂ were the % inhibition at C₁ and C₂, respectively. This is mathematically equivalent to drawing a line between (C₁,I₁) and (C₂,I₂) and finding the concentration at which that line intersects I=50%. An IC₅₀ was calculated for each set of flasks inoculated from the same preculture. An average IC₅₀ was also calculated.

Finally, the change in IC₅₀ (ΔIC₅₀) was determined by subtracting the simultaneously determined IC₅₀ of the vector only strain from the IC₅₀ of the strain being tested, and % ΔIC₅₀ was determined by dividing ΔIC₅₀ by the simultaneously determined IC₅₀ of the vector only strain.

### Example 8

### Identification of Butanol Responsive Genes by Whole-Genome Microarray Transcription Analysis

The purpose of this example is to describe how potential tolerance genes can be identified using whole genome oligonucleotide microarrays.

Bacteria undergo changes in genomic expression patterns when faced with environmental challenges. The most significant changes are often observed for genes whose products are involved in protecting the cell from a given stress. Therefore, such stress-responsive genes can often be identified by comparing global transcription patterns in the presence and absence of the stress. Once the putative stress responsive genes are identified, they can be engineered to optimize their putative protective effect on the cell. In most cases this involves, but is not limited to, over-expressing the genes.

An oligonucleotide microarray was designed and constructed based on the published (Kleerebezem M. et al. 2003. PNAS 100(4) 1990-1995) genome sequence (NCIB Accession NC_{—}004567) for Lactobacillus plantarum strain BAA-793 (ATCC). The microarray contains 3195 unique targets which consist of oligonucleotides that are 70 nucleotides in length. The targets were spotted on each microarray in triplicate and included:

| | |
|---|---|
| Chromosome ORFs: | 3002 |
| Plasmid ORFs: | 50 |
| Pseudogenes: | 42 |
| rRNA Genes: | 15 |
| Negative Controls: | 60 |
| 5′ → 3′ Controls: | 11 |
| Opposite Strand Controls: | 15 |

Microarrays were used to identify L. plantarum genes that demonstrated significantly different levels of expression when the cell was challenged with 1-butanol. Such butanol responsive genes were considered to be potential butanol-tolerance determinants and were targeted for genetic modification in an attempt to increase the overall butanol tolerance of L. plantarum.

In a typical experiment, a culture of L. plantarum BAA-793 was grown at 30° C. in MRS to early exponential phase (Abs₆₀₀ ˜1.0). Equal aliquots of the culture were divided into separate 50 ml tubes. One half of the tubes were placed in a 30° C. water bath (controls) and the other half had prewarmed 1-butanol added, to a final concentration of 1.0% (w/v), and were then placed in the 30° C. water bath. The tubes were incubated for 30 minutes, and then each culture was removed and RNA was isolated using a hot phenol/chloroform method (Chuang et al. 1993 JBact. April; 175(7): 2026-36). For each butanol-treated culture, two cDNA probes are made by reverse transcription and indirect labeling (one Cy3-labeled and one Cy5-labeled for each treatment) and two microarray hybridizations were performed against Cy-labeled probe made from RNA isolated from the control culture. Microarray spot intensities were quantitated using GenePix software and the data was then analyzed using the TM4 Microarray Software Suite (www.tm4.org).

One example of the utility of this approach was seen in the identification of lp_{—}3193, an L. plantarum butanol responsive gene encoding a predicted protein foldase. Microarray experiments indicated that lp_{—}3193 was up-expressed in L. plantarum BAA-793 when the strain was exposed to 1% (w/v) 1-butanol in MRS broth. The gene was subsequently isolated by PCR amplification using primers 5174 and 5175. The PCR product was then digested with BamHI and SalI and ligated into vector pMPE6, which had previously been digested with BamHI and SalI. The resulting plasmid was designated, pMPE41 (Table 8; Example 2 above). Plasmid pMPE41 was transformed into L. plantarum PN0512. The resulting strain was shown to possess increased tolerance to 1-butanol on the basis of its improved ability (relative to controls strains) to remove glucose and produce higher levels of lactate in broth cultures containing 1-butanol (FIG. 4).

### Example 9

### Enrichment of L. plantarum Populations Containing Random Genomic Libraries to Identify Plasmids Encoding Potential Tolerance Determinants

The present example describes a method for identifying potential alcohol tolerance determinant sequences, in the form of plasmid inserts that contribute to increased tolerance within a recombinant L. plantarum strain.

A random genomic library was constructed using purified gDNA from L. plantarum strain BAA-793 and the shuttle vector pMPE6 using techniques that are well known to those practiced in the art. The gDNA from BAA-793 was partially digested with Sau3AI and size fractionated on an agarose gel. DNA fragments with an average size ranging from 1- to 6-kb were purified and used in ligation reactions with BamHI digested pMPE6. Multiple ligation reactions were performed and transformed into E. coli DH5α by electroporation. The E. coli transformation cultures were grown in 5-ml LB cultures containing chloramphenicol for 24 hours at which time the plasmids from each culture were isolated and pooled. The pooled plasmids were then transformed into L. plantarum via electroporation. Multiple transformations were performed. Following grow out in MRS for 4 hours the individual transformation cultures were used to inoculate 5 ml MRS plus chloramphenicol cultures which were grown for 24 hours and then pooled into a single library population. The library was aliquoted and frozen as 20% glycerol stocks at −80° C.

Serial enrichment cultures were used to isolate L. plantarum library strains that possessed increased levels of butanol tolerance relative to other members of a mixed population. Frozen stocks of L. plantarum containing the random gDNA plasmid library were thawed and used to inoculate MRS plus chloramphenicol broth overnight cultures. These cells were then used to inoculate MRS broth cultures containing 1.8% (w/v) 1-butanol. This culture was incubated at 30° C. (100 rpm) until it reached an OD₆₀₀ of ˜4.0. This culture was then used to inoculate a new MRS culture containing 2.0% 1-butanol to starting OD₆₀₀ between 0.25 and 0.3. This culture was again grown to a final OD₆₀₀ of ˜4.0 and subsequently passed to a third MRS 2.0% 1-butanol culture (starting OD₆₀₀ 0.25-0.3). This serial passaging was continued for a specified period of time (usually about 14 days or 6 transfers).

Two methods were used to identify genes that were enriched relative to others in the serially passaged population. The first was a phenotypic selection that involved spreading aliquots of the enriched cultures onto MRS plates containing 1-butanol and isolating single colonies. Strains that were more tolerant to butanol produced single colonies on the selective medium more quickly than others. The isolated strains were streak-purified, their plasmids isolated and the corresponding L. plantarum plasmid insert DNAs were sequenced. The plasmids were also re-transformed into a wild type L. plantarum host strain and the butanol tolerance of the resulting transformants was compared to control strains (either the host strain or the host strain containing the vector only). This step was performed to ensure that increases in tolerance seen in the enriched population were encoded by the plasmids and were not the result of background chromosomal mutations in the enriched host strains.

A second approach used to identify genes that were enriched within the selected population utilized L. plantarum microarrays and a procedure known as Parallel Gene Trait Mapping (Gill et al. 2002. PNAS 99(10) 7033-7038). In this procedure, the plasmids were isolated en masse from the cells remaining in the enrichment population. The isolated plasmid DNA was then labeled and hybridized to L. plantarum microarrays. The relative numbers of L. plantarum genes present in the plasmid sample were then analyzed by comparing their signal intensities on the hybridized arrays. Genes shown to be enriched were cloned and over expressed to test their influence on tolerance.

The utility of the library enrichment method was shown by the isolation of 4 separate plasmids. Plasmids p5AE4-1, p5AE0-4, p5AE0-14 and p5AE4-24 were all isolated by MRS enrichments followed by phenotypic selection for growth on MRS plates containing 3.2% (w/v) 1-butanol. Plasmids were isolated from enrichment strains. DNA sequence analysis identified the portion of L. plantarum BAA-793 chromosome contained within each of the plasmid inserts (Table 1B). The plasmids were transformed into L. plantarum PN0512 and their ability to increase tolerance was confirmed by demonstrating that strains containing the plasmids were able to produce higher levels of lactate than either the wild type parent strain or the parent strain containing vector pMPE6 only in the presence of 1-butanol with concomitant removal of glucose (FIG. 4).

### Example 10

### Batch Fed Fermentation of C. beijerinckii Under Conditions that Produce One or More Aliphatic Alcohol Compounds

The present example provides a description of conditions that can be utilized to grow C. beijerinckii in batch fed cultures under conditions that produce one or more aliphatic alcohol compounds.

C. beijerinckii strains can be maintained under anaerobic conditions as spore suspensions in doubled distilled water (ddH₂O) at room temperature. Spores can be heat shocked at 80° C. for 10 minutes and inoculated into Tryptone, Glucose, Yeast (TGY) medium (Annous et al., Appl. Environ. Microbiol. 56:2559, 1990, herein incorporated by reference).

After overnight growth, cultures can be plated out on TGY agar plates and single colony isolates picked and inoculated into 10 ml TGY medium. The culture can be incubated anaerobically overnight at 37° C. until an optical density at 600 nm of 1.0 to 1.5 is achieved when using a Spectronic 20 spectrophotometer (Bausch and Lomb, Rochester, N.Y.).

P-2 medium (Annous et al., Appl. Environ. Microbiol. 56:2559, 1990, herein incorporated by reference) containing 0.1% yeast extract can be prepared with either 6% glucose or 6% maltodextrin (STAR-DR15™; A. E. Staley Manufacturing Co., Decatur, Ill.) as a carbohydrate source. Semi-defined P2 medium (pH=6.5; 100 ml) can be inoculated with 5 ml of TGY medium culture and incubated anaerobically 18-20 hours at approximately 30° C. The culture can be decanted into 1 liter of semi-defined P2 medium and incubated anaerobically for 16-18 hours at approximately 30° C. until the optical density at 600 nm is 1.0 to 1.5. Batch fermentations can be performed using a 421 Braun fermentor (B. Braun Biotech International GMBH, Melsungen, Germany). Semi-defined P2 medium can be sterilized in the fermentor and agitated and sparged with nitrogen overnight prior to inoculation. Note that, in some embodiments, medium containing acetate is utilized in order to enhance solvent production by C. beijerinckii.

A 5% inoculum of C. beijerinckii can be used for the batch fermentation experiments. 20 liter batch fermentations can be performed at 33° C. in the absence of agitation and pH control. Sterilized nitrogen gas can be sparged (1950 ml/min) through the fermentor to aid mixing and to exclude oxygen. During the course of the fermentation, temperature, pH, and percent oxygen can be measured continuously. Optical density can be monitored by spectrophotometric analysis of culture broth as described above.

### Example 11

### Continuous Feed Fermentation of C. beijerinckii Under Conditions that Produce One or More Aliphatic Alcohol Compounds

The present example provides a description of conditions that can be utilized to grow C. beijerinckii in continuous feed cultures under conditions that produce one or more aliphatic alcohol compounds.

Continuous cultivation of C. beijerinckii strains can be carried out in P2 medium plus 6% glucose using a Braun Biostat 2 liter continuous culture apparatus (B. Braun Biotech International GMBH, Melsungen, Germany) set at 35° C. and 50 rpm stirring rate with no pH control. P2 medium containing 6% glucose can be flushed with nitrogen and inoculated with 100 ml of 18-20 hours old culture. The dilution rate can be set at 0.05 (h.sup.-1) or 0.20 (h.sup.-1). Samples (1 ml) can be routinely removed for solvent analysis. Volumetric solvent production rate can be calculated as g/L/h. Note that, in some embodiments, medium containing acetate is utilized in order to enhance solvent production by C. beijerinckii.

Production of relevant compounds (e.g., acetone and/or aliphatic alcohol compounds such as butanol, and ethanol) can be measured by using a gas chromatograph (5710A; Hewlett-Packard Co., Avondale, Pa.) equipped with a flame ionization detector and a glass column (1.83 m by 2 mm [inner diameter]) packed with 90/100 Carbopack C-0.1% SP-1000 (Supelco, Inc., Bellefonte, Pa.). Butyric and acetic acids can be determined using a Hewlett Packard 5890 series II gas chromatograph and a column packed with Supelco GP 10% SP-1200/1% H₃ PO₄ on chromosorb WAW. Run conditions consisted of 175° C. injector temperature, 180° C. detector temperature and 125° C. oven temperature and a nitrogen carrier gas set at a flow rate of 72 mL/min. Total residual carbohydrate can be determined by using the phenol-sulfuric acid method (Dubois et al., Anal. Chem. 28:350, 1956). Product yield can be calculated by dividing the grams of solvent produced by the grams of carbohydrate consumed. Carbon recovery following fermentation by C. beijerinckii when grown in semi-defined P2 medium containing 6% carbohydrate can be examined by determining the moles of carbon substrate utilized and the moles of carbon product produced as described by Gottschalk (Gottschalk, Butyrate and butanol-acetone fermentation. pp. 231-232. In: Bacterial Metabolism, 2nd edition, 1986) for the ABE fermentation.

### Example 12

### Mapping the Butanol Tolerance Activity of the CAAX Gene

Plasmid p5AE4-1 insert sequence is as follows:

GATCTTTATTAGTTAGTCGTGGAATCCGATAAATCTAAACAAAATCACGT
GTGAGCGTCCCCAATCTGGTATGATTAATGCATATCAGATTGGGGGATTT
TTTT (CAAX protease (lp_2911) upstream (5′)
intergenic sequence)
ATGACGCCGGAAACCGAACAATTATTACGACGCTGGTACATGGGGCAGCT
CATCGTGTTATTTGGCGCGGCCTTTATTCAACTATTTACGTTTGATGGTG
GTGTGTTTTTCCCAGTTGGTGGTATGCAGTTGCTGATATGGGGACTGTTA
GCCTGGTGGCCAGCTGCCGAGGAGGACCAAGCACAGTGGCGGCGTTTGCG
ACATGTTAATTATTATGTCCAAACAGTACTGCAGTTCACACTCTTGCCGA
TTTTACTGGCGAACCTCGTGGCTTGGTTAAGTCAGCTGTCATGGTTAGAC
GAGCAGGGATTGATTGCTGTGGGGATGGCTTATTTAATGGTCGCATTCGT
ACCGGTGGCAGTGGTGGTCACTAAACCGATCGAATCTGTGATTGGCCGGA
TTGCGGTCCTAATTACGGCTATTTTTAGTGGTGTCGTCAGTGCGCAGCAG
ACTTTTTTGATTTTACCGAATCTGCAAGCACCATCAGTATTCGAGATGGT
CAGTGATACTGGTATTTTAGGCGCCCTGGGCTTTGTGATTGCTGTTGGGG
TCTTACTGCGGGGATGGGGATTGACGGGCCCATCGTGGCGGTTTAATCGT
CAGGCCCAAACTAGTTTAGTGGTTGGGCTGATCGTGGTGGGAACGGCTTT
TAGTCTATGGAATGCCTTTAGTGCGGGTGGTTCATGGGCGACAACGTTCA
CACATTGGGACTTCCAGCTACGGTCAGCGACTTGGAAAATGTTTTTGAGT
GGGTTAGAACCGGGAATCGCAGAGGAATGGTTGTATCGTTTTGCCGTTTT
AACCTTGTTATTACAAGCTTTTCGGCATCGGCGTCACCAACTCGACTTGG
CAGTGTGGCTAAGCGGTGGCCTATTTGGAATGTGGCATATTACAAACGTT
TTTGCGGGCCAACCCTTGTCAGCCACGGTTGAGCAAATCATTTTTGCAGC
GACACTAGGCTGGTTTTTAGCCTCGACGTACCTGTACTCAGGTAGTATCT
TGCTGCCGATGGTGATCCATGCTGCTATTGATATTTTGAGCATGATGGCA
TCAGGTAGCCAGACAATGGTTAAGCCGGATGCGTTCGAATGGCAAACAAT
CGGTGCTACCGTCATTATTTTTGTTGGCATAACGATTTATTTCTTGACCG
GTTCTCGGCGACAAGTTATTCAAGCACATGTCAATCAACGGCTTTCAGTT
CAATAA (CAAX protease (lp_2911) coding sequence)
AGGCCGACTGTTAAGACCATAGTGGGCGACTTTGTTCGTTAAAGATAAAC
TGGGTGTCCGTAGCCAGAGACGATTAAGCAATACCAGGCTAACTTTTAGT
TGGTTTAGACCAGTTGTAACATTTTTGTAATCTTCGTGTTATCTAAACGC
AATGCTGGCTCGCTATACTAAAGACAAAGTTATGAAGCAATACATACGCT
TTGTCAGCGGATTTAGGTTGGGAGCCGGATCGATTTACTTTGTCAGGACA
TTGTTAATAAGCAATTATTGATAGTGATAAGTAGCTCAGTTAGCTGAATC
ATAACGTTTGACAAGCATTTATACCTCTCGGGATGGGCTGGGTCCATGAC
GAGGCACATACACAATGGCAAGCTTGGGGTTTGCAAGTCGATCAGAGAAA
GGGACGGTTGGTTACCGGCCCTTTTATTGTGGTTAAAATTTGCGAGAATT
GGATTTAGAACTGCGCCCGATTTGAAGCGGTAGGAACTGCGATGCTGGCA
CAGGTGACTTTGCCAAATCATTGAGAGTGGAACGAAATAATTTACATTTG
CCAGTAGATTATTATAATTAACGAATCAATAATAATTTGGAGATGGCAAT
TTGACTCAGTTTGAAACGGAACGGTTGATATTACGACCAATGACAGCGGC
GGATC-3′ (CAAX protease(1p_2911) downstream (3′)
intergenic sequence).

To determine which region of p5AE4-1 is responsible for the butanol tolerance phenotype, 2 deletion constructs were created. Plasmid pMPE73 has a precise deletion that removes only the CAAX ORF DNA, leaving the upstream and downstream intergenic DNA intact. Plasmid pMPE74 has only the downstream intergenic DNA deleted, leaving the DNA upstream of the CAAX ORF and the CAAX ORF itself intact. Plasmids pMPE73, pMPE74, and the parent plasmid, p5AE4-1 were tested for their ability to confer butanol tolerance by spotting L. plantarum strains transformed with these individual plasmids onto MRS agar plates containing 1-butanol. The strains carrying either p5AE4-1 or pMPE73 (CAAX deletion) showed similar, increased levels of tolerance, whereas the strain carrying pMPE74 (deleted for the DNA downstream of CAAX) showed the same level of tolerance as a control carrying just the vector. Thus, it appears as though the tolerance increase conferred by p5AE4-1 is due to the DNA downstream of the CAAX ORF (lp_{—}2911).

### EQUIVALENTS

Those skilled in the art will recognize, or be able to understand that the foregoing description and examples are illustrative of practicing the provided disclosure. Those skilled in the art will be able to ascertain using no more than routine experimentation, many variations of the detail presented herein may be made to the specific embodiments of the disclosure described herein without departing from the spirit and scope of the present disclosure.

Lengthy table referenced here
US20120058541A1-20120308-T00001
Please refer to the end of the specification for access instructions.

Lengthy table referenced here
US20120058541A1-20120308-T00002
Please refer to the end of the specification for access instructions.

Lengthy table referenced here
US20120058541A1-20120308-T00003
Please refer to the end of the specification for access instructions.

Lengthy table referenced here
US20120058541A1-20120308-T00004
Please refer to the end of the specification for access instructions.

Lengthy table referenced here
US20120058541A1-20120308-T00005
Please refer to the end of the specification for access instructions.

Lengthy table referenced here
US20120058541A1-20120308-T00006
Please refer to the end of the specification for access instructions.

Lengthy table referenced here
US20120058541A1-20120308-T00007
Please refer to the end of the specification for access instructions.

Lengthy table referenced here
US20120058541A1-20120308-T00008
Please refer to the end of the specification for access instructions.

Lengthy table referenced here
US20120058541A1-20120308-T00009
Please refer to the end of the specification for access instructions.

Lengthy table referenced here
US20120058541A1-20120308-T00010
Please refer to the end of the specification for access instructions.

Lengthy table referenced here
US20120058541A1-20120308-T00011
Please refer to the end of the specification for access instructions.

Lengthy table referenced here
US20120058541A1-20120308-T00012
Please refer to the end of the specification for access instructions.

Lengthy table referenced here
US20120058541A1-20120308-T00013
Please refer to the end of the specification for access instructions.

Lengthy table referenced here
US20120058541A1-20120308-T00014
Please refer to the end of the specification for access instructions.

Lengthy table referenced here
US20120058541A1-20120308-T00015
Please refer to the end of the specification for access instructions.

Lengthy table referenced here
US20120058541A1-20120308-T00016
Please refer to the end of the specification for access instructions.

Lengthy table referenced here
US20120058541A1-20120308-T00017
Please refer to the end of the specification for access instructions.

Lengthy table referenced here
US20120058541A1-20120308-T00018
Please refer to the end of the specification for access instructions.

Lengthy table referenced here
US20120058541A1-20120308-T00019
Please refer to the end of the specification for access instructions.

Lengthy table referenced here
US20120058541A1-20120308-T00020
Please refer to the end of the specification for access instructions.

Lengthy table referenced here
US20120058541A1-20120308-T00021
Please refer to the end of the specification for access instructions.

Lengthy table referenced here
US20120058541A1-20120308-T00022
Please refer to the end of the specification for access instructions.

Lengthy table referenced here
US20120058541A1-20120308-T00023
Please refer to the end of the specification for access instructions.

Lengthy table referenced here
US20120058541A1-20120308-T00024
Please refer to the end of the specification for access instructions.

Lengthy table referenced here
US20120058541A1-20120308-T00025
Please refer to the end of the specification for access instructions.

Lengthy table referenced here
US20120058541A1-20120308-T00026
Please refer to the end of the specification for access instructions.

Lengthy table referenced here
US20120058541A1-20120308-T00027
Please refer to the end of the specification for access instructions.

Lengthy table referenced here
US20120058541A1-20120308-T00028
Please refer to the end of the specification for access instructions.

Lengthy table referenced here
US20120058541A1-20120308-T00029
Please refer to the end of the specification for access instructions.

Lengthy table referenced here
US20120058541A1-20120308-T00030
Please refer to the end of the specification for access instructions.

Lengthy table referenced here
US20120058541A1-20120308-T00031
Please refer to the end of the specification for access instructions.

Lengthy table referenced here
US20120058541A1-20120308-T00032
Please refer to the end of the specification for access instructions.

Lengthy table referenced here
US20120058541A1-20120308-T00033
Please refer to the end of the specification for access instructions.

Lengthy table referenced here
US20120058541A1-20120308-T00034
Please refer to the end of the specification for access instructions.

Lengthy table referenced here
US20120058541A1-20120308-T00035
Please refer to the end of the specification for access instructions.

Lengthy table referenced here
US20120058541A1-20120308-T00036
Please refer to the end of the specification for access instructions.

Lengthy table referenced here
US20120058541A1-20120308-T00037
Please refer to the end of the specification for access instructions.

Lengthy table referenced here
US20120058541A1-20120308-T00038
Please refer to the end of the specification for access instructions.

Lengthy table referenced here
US20120058541A1-20120308-T00039
Please refer to the end of the specification for access instructions.

Lengthy table referenced here
US20120058541A1-20120308-T00040
Please refer to the end of the specification for access instructions.

Lengthy table referenced here
US20120058541A1-20120308-T00041
Please refer to the end of the specification for access instructions.

Lengthy table referenced here
US20120058541A1-20120308-T00042
Please refer to the end of the specification for access instructions.

Lengthy table referenced here
US20120058541A1-20120308-T00043
Please refer to the end of the specification for access instructions.

Lengthy table referenced here
US20120058541A1-20120308-T00044
Please refer to the end of the specification for access instructions.

Lengthy table referenced here
US20120058541A1-20120308-T00045
Please refer to the end of the specification for access instructions.

Lengthy table referenced here
US20120058541A1-20120308-T00046
Please refer to the end of the specification for access instructions.

Lengthy table referenced here
US20120058541A1-20120308-T00047
Please refer to the end of the specification for access instructions.

Lengthy table referenced here
US20120058541A1-20120308-T00048
Please refer to the end of the specification for access instructions.

Lengthy table referenced here
US20120058541A1-20120308-T00049
Please refer to the end of the specification for access instructions.

Lengthy table referenced here
US20120058541A1-20120308-T00050
Please refer to the end of the specification for access instructions.

Lengthy table referenced here
US20120058541A1-20120308-T00051
Please refer to the end of the specification for access instructions.

Lengthy table referenced here
US20120058541A1-20120308-T00052
Please refer to the end of the specification for access instructions.

Lengthy table referenced here
US20120058541A1-20120308-T00053
Please refer to the end of the specification for access instructions.

Lengthy table referenced here
US20120058541A1-20120308-T00054
Please refer to the end of the specification for access instructions.

Lengthy table referenced here
US20120058541A1-20120308-T00055
Please refer to the end of the specification for access instructions.

Lengthy table referenced here
US20120058541A1-20120308-T00056
Please refer to the end of the specification for access instructions.

Lengthy table referenced here
US20120058541A1-20120308-T00057
Please refer to the end of the specification for access instructions.

Lengthy table referenced here
US20120058541A1-20120308-T00058
Please refer to the end of the specification for access instructions.

Lengthy table referenced here
US20120058541A1-20120308-T00059
Please refer to the end of the specification for access instructions.

Lengthy table referenced here
US20120058541A1-20120308-T00060
Please refer to the end of the specification for access instructions.

Lengthy table referenced here
US20120058541A1-20120308-T00061
Please refer to the end of the specification for access instructions.

Lengthy table referenced here
US20120058541A1-20120308-T00062
Please refer to the end of the specification for access instructions.

Lengthy table referenced here
US20120058541A1-20120308-T00063
Please refer to the end of the specification for access instructions.

Lengthy table referenced here
US20120058541A1-20120308-T00064
Please refer to the end of the specification for access instructions.

Lengthy table referenced here
US20120058541A1-20120308-T00065
Please refer to the end of the specification for access instructions.

Lengthy table referenced here
US20120058541A1-20120308-T00066
Please refer to the end of the specification for access instructions.

Lengthy table referenced here
US20120058541A1-20120308-T00067
Please refer to the end of the specification for access instructions.

Lengthy table referenced here
US20120058541A1-20120308-T00068
Please refer to the end of the specification for access instructions.

Lengthy table referenced here
US20120058541A1-20120308-T00069
Please refer to the end of the specification for access instructions.

Lengthy table referenced here
US20120058541A1-20120308-T00070
Please refer to the end of the specification for access instructions.

LENGTHY TABLES
The patent application contains a lengthy table section. A copy of the table is available in electronic form from the USPTO web site (http://seqdata.uspto.gov/?pageRequest=docDetail&DocID=US20120058541A1). An electronic copy of the table will also be available from the USPTO upon request and payment of the fee set forth in 37 CFR 1.19(b)(3).

1-294. (canceled)

295. A recombinant microbial cell, characterized in that the recombinant microbial cell comprises at least one alcohol tolerance modification as compared with a parent cell.

296. The recombinant microbial cell of claim 1, wherein the alcohol tolerance modification comprises introduction of a nucleic acid molecule comprising a 3′ region of a gene encoding a CAAX protease polypeptide.

297. The recombinant microbial cell of claim 296, wherein the 3′ region of the gene comprises a 3′ untranslated region (UTR).

298. The recombinant microbial cell of claim 296, wherein the 3′ region of the gene comprises nucleotides immediately downstream of sequence encoding the CAAX protease polypeptide.

299. The recombinant microbial cell of claim 296, wherein the nucleic acid molecule comprises a 3′ region of a gene encoding a CAAX protease polypeptide in Lactobacillus.

300. The recombinant microbial cell of claim 299, wherein the 3′ region of the gene comprises at least 10 consecutive nucleotides of the nucleotide sequence shown in Table 1B, row 42.

301. The recombinant microbial cell of any of claim 295, wherein the alcohol tolerance modification comprises introduction of a nucleic acid molecule comprising a 5′ region of a gene encoding a CAAX protease polypeptide.

302. The recombinant microbial cell of claim 301, wherein the 5′ region of the gene comprises a 5′ UTR.

303. The recombinant microbial cell of claim 301, wherein the 5′ region of the gene comprises nucleotides immediately upstream of sequence encoding the CAAX protease polypeptide.

304. The recombinant microbial cell of claim 301, wherein the nucleic acid molecule comprises a 5′ region of a gene encoding a CAAX protease polypeptide in Lactobacillus.

305. The recombinant microbial cell of claim 304, wherein the 5′ region of the gene comprises at least 10 consecutive nucleotides of the nucleotide sequence shown in Table 1B, row 40.

306. The recombinant microbial cell of any of claim 295, wherein the cell exhibits increased tolerance to at least one aliphatic alcohol compound as compared with the parent cell.

307. The recombinant microbial cell of claim 306, wherein the increased tolerance to at least one aliphatic alcohol compound comprises an increased aliphatic alcohol compound IC₅₀, wherein the IC₅₀ is increased at least 5%, 10%, 20%, 30%, 40%, 50%, 60%, 70%, 80%, 90%, 100%, or more.

308. The recombinant microbial cell of claim 306 or 307, wherein the increased tolerance to at least one aliphatic alcohol compound comprises increased carbohydrate utilization as compared to the parent cell when grown in same amount of alcohol.

309. The recombinant microbial cell of claim 308, wherein the carbohydrate utilization is increased at least 5%, 10%, 20%, 30%, 40%, 50%, 60%, 70%, 80%, 90%, 100%, or more.

310. The recombinant cell of any of claim 295, wherein the cell exhibits increased alcohol production as compared with the parent cell.

311. The recombinant microbial cell of any one of claim 295, wherein the cell is a member of a genus selected from the group consisting of Clostridium, Zymomonas, Escherichia, Salmonella, Rhodococcus, Pseudomonas, Bacillus, Lactobacillus, Enterococcus, Alcaligenes, Klebsiella, Paenibacillus, Arthrobacter, Corynebacterium, Brevibacterium, Acinetobacter, Pichia, Candida, Hansenula and Saccharomyces.


SEQUENCE LISTING (203 sequences)

SEQ ID NO: 1 - DNA, 36 bp, Artificial Sequence
  Primer
tttggatccgattctattgttagctattttgggtgg

SEQ ID NO: 2 - DNA, 37 bp, Artificial Sequence
  Primer
aaagtcgacatcgtggtattagtgatgcaaagaaagg

SEQ ID NO: 3 - DNA, 38 bp, Artificial Sequence
  Primer
aacggccgaaggattattcggctggttgagacgttaaa

SEQ ID NO: 4 - DNA, 38 bp, Artificial Sequence
  Primer
aacggccgaaataacaccttaagtctagcaccacccgc

SEQ ID NO: 5 - DNA, 29 bp, Artificial Sequence
  Primer
ctccagtaagaatatttgcattgtgtatc

SEQ ID NO: 6 - DNA, 31 bp, Artificial Sequence
  Primer
aaaaaggcccttataacttacaaataacccc

SEQ ID NO: 7 - DNA, 38 bp, Artificial Sequence
  Primer
aaaaaagcggccgctctttattcttcaactaaagcacc

SEQ ID NO: 8 - DNA, 42 bp, Artificial Sequence
  Primer
aaaaaagcggccgcaatgtatttagaaaaataaacaaatagg

SEQ ID NO: 9 - DNA, 86 bp, Artificial Sequence
  Primer
aaaaagcggaagagcgagggcggagttgttgacagccgaggtaccatgtggtataatcccgagtgtggaattgtgagcggataaca

SEQ ID NO: 10 - DNA, 35 bp, Artificial Sequence
  Primer
gttttcgaggggttatttgtaagttataaggccct

SEQ ID NO: 11 - DNA, 28 bp, Artificial Sequence
  Primer
aaaaagcggaagagcgagggcggagttg

SEQ ID NO: 12 - DNA, 104 bp, Artificial Sequence
  CAAX protease (lp_2911) upstream (5′) intergenic sequence
gatctttattagttagtcgtggaatccgataaatctaaacaaaatcacgtgtgagcgtccccaatctggtatgattaatgcatatcagattgggggattttttt

SEQ ID NO: 13 - DNA, 1206 bp, Artificial Sequence
  CAAX protease (lp_2911) coding sequence
atgacgccggaaaccgaacaattattacgacgctggtacatggggcagctcatcgtgttatttggcgcggcctttattcaactatttacgtttgatggtggtgtgtttttcccagttggtggtatgcagttgctgatatggggactgttagcctggtggccagctgccgaggaggaccaagcacagtggcggcgtttgcgacatgttaattattatgtccaaacagtactgcagttcacactcttgccgattttactggcgaacctcgtggcttggttaagtcagctgtcatggttagacgagcagggattgattgctgtggggatggcttatttaatggtcgcattcgtaccggtggcagtggtggtcactaaaccgatcgaatctgtgattggccggattgcggtcctaattacggctatttttagtggtgtcgtcagtgcgcagcagacttttttgattttaccgaatctgcaagcaccatcagtattcgagatggtcagtgatactggtattttaggcgccctgggctttgtgattgctgttggggtcttactgcggggatggggattgacgggcccatcgtggcggtttaatcgtcaggcccaaactagtttagtggttgggctgatcgtggtgggaacggcttttagtctatggaatgcctttagtgcgggtggttcatgggcgacaacgttcacacattgggacttccagctacggtcagcgacttggaaaatgtttttgagtgggttagaaccgggaatcgcagaggaatggttgtatcgttttgccgttttaaccttgttattacaagcttttcggcatcggcgtcaccaactcgacttggcagtgtggctaagcggtggcctatttggaatgtggcatattacaaacgtttttgcgggccaacccttgtcagccacggttgagcaaatcatttttgcagcgacactaggctggtttttagcctcgacgtacctgtactcaggtagtatcttgctgccgatggtgatccatgctgctattgatattttgagcatgatggcatcaggtagccagacaatggttaagccggatgcgttcgaatggcaaacaatcggtgctaccgtcattatttttgttggcataacgatttatttcttgaccggttctcggcgacaagttattcaagcacatgtcaatcaacggctttcagttcaataa

SEQ ID NO: 14 - DNA, 655 bp, Artificial Sequence
  CAAX protease(lp_2911) downstream (3′) intergenic sequence
aggccgactgttaagaccatagtgggcgactttgttcgttaaagataaactgggtgtccgtagccagagacgattaagcaataccaggctaacttttagttggtttagaccagttgtaacatttttgtaatcttcgtgttatctaaacgcaatgctggctcgctatactaaagacaaagttatgaagcaatacatacgctttgtcagcggatttaggttgggagccggatcgatttactttgtcaggacattgttaataagcaattattgatagtgataagtagctcagttagctgaatcataacgtttgacaagcatttatacctctcgggatgggctgggtccatgacgaggcacatacacaatggcaagcttggggtttgcaagtcgatcagagaaagggacggttggttaccggcccttttattgtggttaaaatttgcgagaattggatttagaactgcgcccgatttgaagcggtaggaactgcgatgctggcacaggtgactttgccaaatcattgagagtggaacgaaataatttacatttgccagtagattattataattaacgaatcaataataatttggagatggcaatttgactcagtttgaaacggaacggttgatattacgaccaatgacagcggcggatc

SEQ ID NO: 15 - DNA, 423 bp, Lactobacillus plantarum
atggctaatactttaatgaatcggaacgatttcggcatgttggatccgtttgaacggatggcacgctccttctgggcaccattagaaaacatggatcaagtattgaagaccgacattaacgaaactgatgatcagtatcaagtgaaggttgatgtccctggtattgataagcaagatgtgaagttggattatcgtgacaatgtgttgtctatcaaggttcaaaaagatagctttgtggatcatgaagatcaagaccaaaacattgtgatgaatgagcgtcatactggcaccttgcaacggcagtatatgttaccaaacgttgcggcgaataaaattacggcatcccaagctgacggtgtcttaacgattacgttacctaagacccagccgagcgcgaatgacggtcaaatcgaaattcaataa

SEQ ID NO: 16 - protein, 140 aa, Lactobacillus plantarum
MANTLMNRNDFGMLDPFERMARSFWAPLENMDQVLKTDINETDDQYQVKVDVPGIDKQDVKLDYRDNVLSIKVQKDSFVDHEDQDQNIVMNERHTGTLQRQYMLPNVAANKITASQADGVLTITLPKTQPSANDGQIEIQ

SEQ ID NO: 17 - DNA, 273 bp, Lactobacillus plantarum
gtggatcccaagcgagttaaatatctgaaattgattagtaagttagcgacatttacgtgtattgcgatgtacgtgtcatatattccgcaaatcatttcgaatttctcaggtgacccagtatcgccgctacaaccactcgtggcaatgattaatgggatactatggactggttacggctggttcaagacttataaggattggcccgttattatttcaaatgttcccggggtgatttttggatttatcactgttttaaccgtatatattcattaa

SEQ ID NO: 18 - protein, 90 aa, Lactobacillus plantarum
MDPKRVKYLKLISKLATFTCIAMYVSYIPQIISNFSGDPVSPLQPLVAMINGILWTGYGWFKTYKDWPVIISNVPGVIFGFITVLTVYIH

SEQ ID NO: 19 - DNA, 1179 bp, Lactobacillus plantarum
atgtatcaagttaaaacctataacgccatcgccccagccggcctcaacacgtttactgctgattacacgctcaatcaatctgagcatccggatgcttacttaattcgctcggtcaacctacataccgagacattaccgtcatcgttgaaagtcattgtgcgcgctggtgccggcgttaacaacattcctatcgatcaggcaaccgccaacgggactgcagttttcaacaccccgggaagtaacgctaatgccgttaaggaactcatcatcggcctgctcattatggcatcccgtaatctaatagctgcaacgacctattcggcccagcataccgaagctgatatttctcaacgcacagaacacgacaagacgcaatttaatggtattgaattaacgggtaagaccttggccgtcatcggactcggccatgttggcgctctcgttgccaatgcagcattgagtctaggcatgaatgtaattggttacgacccctatctatctgcagatgccgcttggaacattgctaaacaagtccagcgagcggccacgctgccagatgcagtcaaacaagctgattttgtcaccgtccacgttcctaaaaatgccgacacacttcatctgattaataaagatgcgttagccgccatgccaacaggcgttcaattatttaattattcacggctgggcatcgttgacaatactgccgtcatgaatgcgttagccacgggacaagttgcccactactacaccgattttggcgaaccccagcttgccaaccaatccgcggttaccgtgacaccccatatcggcggctcgactatcgaggctgaaatcaacggtgccacacaagctgcgcgcactatcatgacttatttggaaaccggtaacgttcatgcggccatcaatctgccagacttaaacgtcccgttcaacgcggcttaccgctttacagtcattcacgaaaatgtgcctaacatggtgagtcaaatcacggccaaactagcagcggccaacctcaacatcactaccatggctaacgccgctaagcaccagattgcttacaccatcattgacgtcgacgacctacagcagccacaacaagccgacctaatagctgaactgtctaaaattccggcagtgagtcgcgttcgactattaaaacggggtagcgtcgaatga

SEQ ID NO: 20 - protein, 392 aa, Lactobacillus plantarum
MYQVKTYNAIAPAGLNTFTADYTLNQSEHPDAYLIRSVNLHTETLPSSLKVIVRAGAGVNNIPIDQATANGTAVFNTPGSNANAVKELIIGLLIMASRNLIAATTYSAQHTEADISQRTEHDKTQFNGIELTGKTLAVIGLGHVGALVANAALSLGMNVIGYDPYLSADAAWNIAKQVQRAATLPDAVKQADFVTVHVPKNADTLHLINKDALAAMPTGVQLFNYSRLGIVDNTAVMNALATGQVAHYYTDFGEPQLANQSAVTVTPHIGGSTIEAEINGATQAARTIMTYLETGNVHAAINLPDLNVPFNAAYRFTVIHENVPNMVSQITAKLAAANLNITTMANAAKHQIAYTIIDVDDLQQPQQADLIAELSKIPAVSRVRLLKRGSVE

SEQ ID NO: 21 - DNA, 1074 bp, Lactobacillus plantarum
atgccaatttataatttttctgctgggccagccgttctaccacaaccagtcatcactcaaattcaagcggagctaccatcatttcgagactccggcatgagcattttagagatctcgcatcgctccgatttatttgcgcaagtccttcaagatgccgaacaagatcttcgcgatttaatggccattcctgacaactatcacgtgctcttctttcaaggcgggggcacgctacagttcacagctgcgccactaaatctggcgcctcatcatcgtatcgggttgcttgacagcggtcactgggcacaacgcgccgccgatgaagctaaacgggtcggtactaaagtcacgatactggggagtagcgctgccaaccattttaaccaactgccaacggtcgtccagcccatcgatcaatccctcgattatattcatcttacaactaataatactattgaaggaaccatgatgacgcgcctgccagttacgggtcaagtaccactggtagccgacatgtcatcaaactttttaggtgaaccttaccaagtcagcgattttgggctcatctttgctggtgctcagaagaatctgggtcccgctggtttgacaatcgtcattgtccgtgatgatttaattggtcaagtcgccaacctgccaagcatgctggattaccagctattcgcggctaaagattcgatgttcaacacgccgcctgtttttgctatttacgccgcgggtctcgtactcaagtggctaaaggcccaaggcgggctcagcacaatgactgctcgcaatcacgctaaagccgccttactctatgatttcttagaccagtcacaactatttactaatccagtcaagaccagcgaccgttcgaccatgaacgttccattcgtcacaggtcaggccgacctcgatgccgcagtcattcaaggcgcccgtgagcacgggttattaaacctaaagggtcaccgcttagttggcggtatgcgtgccagcctctataacgccatgccgttagccggtgttcaggcattagttgactatctagccgcttttgaagcacaccatcgttaa

SEQ ID NO: 22 - protein, 357 aa, Lactobacillus plantarum
MPIYNFSAGPAVLPQPVITQIQAELPSFRDSGMSILEISHRSDLFAQVLQDAEQDLRDLMAIPDNYHVLFFQGGGTLQFTAAPLNLAPHHRIGLLDSGHWAQRAADEAKRVGTKVTILGSSAANHFNQLPTVVQPIDQSLDYIHLTTNNTIEGTMMTRLPVTGQVPLVADMSSNFLGEPYQVSDFGLIFAGAQKNLGPAGLTIVIVRDDLIGQVANLPSMLDYQLFAAKDSMFNTPPVFAIYAAGLVLKWLKAQGGLSTMTARNHAKAALLYDFLDQSQLFTNPVKTSDRSTMNVPFVTGQADLDAAVIQGAREHGLLNLKGHRLVGGMRASLYNAMPLAGVQALVDYLAAFEAHHR

SEQ ID NO: 23 - DNA, 1368 bp, Lactobacillus plantarum
atgctgaaagaaatggaagaaacaaccgtatcacgttcaatcgatcggttagtcttaaatgcttcgttagctgccaaccgtcttgaagtcatggaccaaagtcaagttgatcaggctgtcgctgccatggcccgcgctgcccacgctgctcgtggcatgctggccgctatggccgtcgaagaaacgggtcgcggaaattatcgtgataaagttgcgaagaacgactttgcagccaaaaacgtttataactacatcaaggatgacaagacggtcggtatcattaatgacgatccagtcagtggcgtgatgaaagttgctgaaccagttggaattattgcgggggtcaccccagttaccaacccaacatcaaccgtcattttcaatgccatgttagcattaaagactcgcaatcccattatttttggtttccatccctttgcacaaaaatcttgtgttgaaactggccgaatcatccgcgatgctgctattgcctctggcgctcctaaggattggattcagtggatcaagacgcctagccttgaagcaaccaacaccttgatgaaccatccgggcgtcgctaccattattgcaactggcggtgccggcatggtcaagaccgcgtattcaactggtaaaccggcactcggtgttggccctggtaacgtgccatgcttcatcgagcaaaccgcagacattcaacaggcagtcagtgatgtcgtcacttccaagtcattcgacaacggcatgatctgtgcttccgaatcaaacttaatcgttgctgatcaaatctatgatcaagttaaacgtgaattaagtcacaacggtgtgtactttgtcggtaccgagaacttcaaggccttagaagcaactgtcatgaacctggataaacaggctgttgacccgaaagtagctgggcaaacgccatggcaaatcgctcagtgggctggctttgatgtcccatccgataccaaagtattagcagttgagttgcctagcatcggtggtgaccaagtcttatcacgagaaaagttatcaccagtcctcgccgtcgttcatgccaaggatactgaggccggcttcaacctgatgaaacgcagcctagcacttggcggactgggacatacggccgccttgcatacgactgacgaagctgttatgaacaagtttgccttagaaatgactgcttgtcgagcattgatcaacgtgccgtcttcacaaggtgccattggttataaatatgataacgtcgcaccatccttaacactcggttgtggaacatgggggcataactcgatttcacacaacttggaagattgggatctactaaatattaagaccgttgcaaaacgcttaactaagattcgctaa

SEQ ID NO: 24 - protein, 455 aa, Lactobacillus plantarum
MLKEMEETTVSRSIDRLVLNASLAANRLEVMDQSQVDQAVAAMARAAHAARGMLAAMAVEETGRGNYRDKVAKNDFAAKNVYNYIKDDKTVGIINDDPVSGVMKVAEPVGIIAGVTPVTNPTSTVIFNAMLALKTRNPIIFGFHPFAQKSCVETGRIIRDAAIASGAPKDWIQWIKTPSLEATNTLMNHPGVATIIATGGAGMVKTAYSTGKPALGVGPGNVPCFIEQTADIQQAVSDVVTSKSFDNGMICASESNLIVADQIYDQVKRELSHNGVYFVGTENFKALEATVMNLDKQAVDPKVAGQTPWQIAQWAGFDVPSDTKVLAVELPSIGGDQVLSREKLSPVLAVVHAKDTEAGFNLMKRSLALGGLGHTAALHTTDEAVMNKFALEMTACRALINVPSSQGAIGYKYDNVAPSLTLGCGTWGHNSISHNLEDWDLLNIKTVAKRLTKIR

SEQ ID NO: 25 - DNA, 1527 bp, Lactobacillus plantarum
atgcagacattaatacagacgtttatagatcgacgcggggatttgctgactgcgctatggcaacacttggggatttcattagcatcattagtcatcgcaatggtaattgcgattccgttggctatttgggtcgttcgacgaccacggtgggccgagggattgttacagctcacgagtgtcctacagacgattccgtctttggcactgttagggttattgattccgttagttgggattggaacggtgccagcagtaattgcgctggtgatctatgctttactgccgatttttcaaaatacttacttgggtatctcagaaatcgatgcctcaattgaagaggccgccgatgcctttgggatgtcacgaatgcgtaagttgtttaaagttgaactacccattgccctaccacagatcattgctgggattcggaccgcgctcgttttaatcattgggacggctactttggccgctttgattggtgctgggggtctcgggacctttatcatgctcggtattgaccgtaatgatacttcgttattattgattggggccatctcatcagcattgttagcaattctgctgagtgcgctcgttcggtggtttcaaacggctaaaccacgccacgccttaatcgtctttgtcggtattttagctttacttggtggtggcggggcttatagtgtctatgccaatcgagttgaaacaattacgattgcaggtaaacttggttccgaaccagaaatcttgattaatatgtataagcagctgattgaagctgaagatgaacacgttcatgtgacgctcaagcctaactttggcaagaccacgttcttattcagcgcgttaaagaataatcaggttgatatttatcctgaatttactggctcggtgctggagaccttagttaagggaaataacccagctggtcaaacagctaaccagacctatcagctcgccaaacagcgcctcgctaagcaggaacaaatgacttacttgaagccgatgcagtataacaatacgtacgcattggcagtgactaagaaatttcaacaagaacatcatttgaagacaatcagcgacttaacgcaagttgaatcgattctgaaacccggaatgaccctagagtttattgatcgtaatgatggcttaaaaggaatcaagaagacttatgggttagacgtgactgccaagtcgatggagccggcgctacgttatgaagccatcagtaaggggaaaatcaacttggtagatgcctatgcgacggatagtgaattacggcagtatcacttggccttattgaaggataacaagcacttcttcccaacgtatcaaggggcaccgttgatgaagacgagctttgccaacaaacatcctaaggtcgttaaagcgttgaataagttagcaggaaagatttcagaaactgatatgcaagaaatgaactatgaagtcaatgttaagaagcagtccgcttcgacggttgcacatcgctatcttgtgaagcacggtttattgaaggagggacgttaa

SEQ ID NO: 26 - protein, 508 aa, Lactobacillus plantarum
MQTLIQTFIDRRGDLLTALWQHLGISLASLVIAMVIAIPLAIWVVRRPRWAEGLLQLTSVLQTIPSLALLGLLIPLVGIGTVPAVIALVIYALLPIFQNTYLGISEIDASIEEAADAFGMSRMRKLFKVELPIALPQIIAGIRTALVLIIGTATLAALIGAGGLGTFIMLGIDRNDTSLLLIGAISSALLAILLSALVRWFQTAKPRHALIVFVGILALLGGGGAYSVYANRVETITIAGKLGSEPEILINMYKQLIEAEDEHVHVTLKPNFGKTTFLFSALKNNQVDIYPEFTGSVLETLVKGNNPAGQTANQTYQLAKQRLAKQEQMTYLKPMQYNNTYALAVTKKFQQEHHLKTISDLTQVESILKPGMTLEFIDRNDGLKGIKKTYGLDVTAKSMEPALRYEAISKGKINLVDAYATDSELRQYHLALLKDNKHFFPTYQGAPLMKTSFANKHPKVVKALNKLAGKISETDMQEMNYEVNVKKQSASTVAHRYLVKHGLLKEGR

SEQ ID NO: 27 - DNA, 972 bp, Lactobacillus plantarum
atgacaacggcaattgaatttcaacacgtccagaaagactttaatgggcagaccgtgattcccgaccttaatttaacgattgaccagggtgagctatttgttttggtagggacttctgggagtggcaaaacgacgtcacttaaaatgatcaactgcttagagccactgacggctggtaaaattctagttaatggtactgatacaaccacgataccagtccgaagtctacggtggcaaatggggtatgtcttacagcaaattgccttgttcccaacgatgacggtggcgcaaaatatcgccgtgattccggaaatgaaagggacagctaagaaggaaattaatcaaacgattgatgagctattggcggaagttggcctcgatccaaaggaataccgtgaccggatgccgtcagaattatccggtggtgagcagcaacgcatcggtatcttacgggcgattgcggcgcaaccagatattgttttgatggatgaaccatttagtgcgttagaccccatctcgcggcaacaattgcaagacttggtcttacggctacacgcccgctatcacaacacgatcgtcttcgtgacgcatgatatgaatgaggcgttgaagttgggtgaccggatcggtgtcatgcaacacggtcagttaatacaagtcgatacgccggctgctctggctcagcatccagtgaacgactttgtgcggaacttctttggtgcgagccgagctaaaaatgtctatgatgtctacgttgggcgtgtagggcttattcagggttatctcacagaagaacccagtgttgcgagtggtcggattcaatcgttagacgttcaagccacgttacgcaccgcctttacggcattgacagatcacgattatgtggcggtcacggaagaaaatcgggttgttggctatttggatcgccaacgaatcgtggcttacttgagtcaacatgaagaagtatcttaa

SEQ ID NO: 28 - protein, 323 aa, Lactobacillus plantarum
MTTAIEFQHVQKDFNGQTVIPDLNLTIDQGELFVLVGTSGSGKTTSLKMINCLEPLTAGKILVNGTDTTTIPVRSLRWQMGYVLQQIALFPTMTVAQNIAVIPEMKGTAKKEINQTIDELLAEVGLDPKEYRDRMPSELSGGEQQRIGILRAIAAQPDIVLMDEPFSALDPISRQQLQDLVLRLHARYHNTIVFVTHDMNEALKLGDRIGVMQHGQLIQVDTPAALAQHPVNDFVRNFFGASRAKNVYDVYVGRVGLIQGYLTEEPSVASGRIQSLDVQATLRTAFTALTDHDYVAVTEENRVVGYLDRQRIVAYLSQHEEVS

SEQ ID NO: 29 - DNA, 1332 bp, Lactobacillus plantarum
atggcggaacagtacgatgttgttgtgattggtggcggaccagccggcaatgccatggctagcggattaaaggcccagggcaagacagtgttgatcgttgaagcggatctgtggggcggcacttgtcctaaccgcggttgtgaccctaagaaaatcctgttaagcgccgtcgaagcgcgacaagcggcgcaacatttacaagggcagggcctgattggcgcgcccaaaattgattggccagcactgatggcgcataaacgaggctatacggatggcatcaacgatgggacgttgaacggactaacggggcaagatattgcgacgttacatggccaagcacactttcaatccgacaatcagttagcggtcggggatcgagtagtcagtgccactgattacgtgattgctactggtcagcgtccagcgattctaccgattaccgggcacgaatactttaagacgagcactgacttcttagatttggaccagatgcctaaacgcgtgacatttgtaggtggtggctacgtaggctttgaattggcgacgattgcgaatgccgctggcgctgatgtgcacgtgattcatcataatgaccgcccgttaaaagcttttgatgcagatttggttaaggatttgatggccgcaatgacggctgatggaatcacgtttgacttgaatacggatgtccaagcaattactaaaacggcgaccggtctacaattgacagctgataatttcgagctgacaacggatctggtcatcagctcagcgggacggattccgaacgcggaccagttaggtctagccaacgtgggcgttacctttgatcggcatgggattcaagtcaacgatcatttgcagacggccaacccgcacatttatgccattggggatgtcagcgatacaccggtaccgaagttaacgccagttgcaggttttgaagcgcgttatctggtcggtgagttgacgcatcctggcgcagccataaagtatcccgttgtgccaacgcaggtttttgcagcgcccaagttagcgcaagtcgggatcagcgcggccgtggcgactgagcatccagatgagtatcgtgtcaatacacttgatatgacgaagtggttcacttattaccgctttggcgcacaacaagcccaagctaaagtagtggttgctaaagcgagtgggcaggttgtgggtgctacccttctaagtgatgttgccgacgagatgattaactacttcacgttgttaattgaaaaacacgtgactttaccagatttacaacggttggtattggcttacccaacgccggctagtgacttacaatatttgtattaa

SEQ ID NO: 30 - protein, 443 aa, Lactobacillus plantarum
MAEQYDVVVIGGGPAGNAMASGLKAQGKTVLIVEADLWGGTCPNRGCDPKKILLSAVEARQAAQHLQGQGLIGAPKIDWPALMAHKRGYTDGINDGTLNGLTGQDIATLHGQAHFQSDNQLAVGDRVVSATDYVIATGQRPAILPITGHEYFKTSTDFLDLDQMPKRVTFVGGGYVGFELATIANAAGADVHVIHHNDRPLKAFDADLVKDLMAAMTADGITFDLNTDVQAITKTATGLQLTADNFELTTDLVISSAGRIPNADQLGLANVGVTFDRHGIQVNDHLQTANPHIYAIGDVSDTPVPKLTPVAGFEARYLVGELTHPGAAIKYPVVPTQVFAAPKLAQVGISAAVATEHPDEYRVNTLDMTKWFTYYRFGAQQAQAKVVVAKASGQVVGATLLSDVADEMINYFTLLIEKHVTLPDLQRLVLAYPTPASDLQYLY

SEQ ID NO: 31 - DNA, 1614 bp, Lactobacillus plantarum
atgaccaaatatatttttgtaactggtggcgttgtgtcatccattggtaaaggtatcgtcgctgcttcgctagggcgtttattgaagaaccggggcttaaaggtcacgattcaaaagtttgatccctatatcaacgttgatcctggtacgatgagtccttatcaacacggtgaagtcttcgtgaccgatgatgggaccgaaactgacttagaccttggacattatgaacggtttatcgacattaaccttaataaatattcaaatgttaccaccggtaagatttattcagaagttctgcaaaaggaacggcggggcgattatttaggcgccacggtgcaagtgattccgcatatcacgaacgctatcaaggaaaaaatcatgcgtgcgggtacgacgacggattccgatatcgtgattactgaaatcggtgggacggtcggtgatatcgaatccttgccatttattgaagcgctacggcaaatgaagagtgatttaggttccgacaatgttttctatatccataccacattgatcccatatttacgggcagctggtgaaatgaagacgaagccaacgcaacattctgttaaggaattgcggagttatgggattcagccgaacatgttagttgtccggactgaacaaccaattacgcgggaaatgcggaataagattgcgtccttctgtgacgtggaaccagaagcagtcattgaatccttagacgttaagacgatttattcaattccgttgaatgttcagaaacaaaacatggaccaaatcgtccttgaccattttgatgtacaggcacctaaggccgacatgagtgaatggattgacttagaacatcatgttcagaacttatcacggaccatcaagattgcgctagtcggaaaatacgtcgctttacaggatgcttatatctcagtgacggaagcattgaagcatgctggctatacggatgatgccgacattgatttgaagaagatttctgctgaagatgttacgccagaaaatgtcgaagaactactcggcgatgctgacggaatcttagttcctggtggctttggtgatcggggaattgaaggtaagattacggcaatcaagtatgcccgtgaaaacgacgtgccattcttaggtatctgcttgggaatgcaaatggccagtgtcgaatttgcacgtaacgtacttggattgaaggatgctaactctgctgaaatcgatccgaagacgccggacaatattattgatttgatggccgaccaagaagacgttgaagacatgggtggaacgcaacgtttaggcgcttacccttgcaagctgaagccgggaactgtggcggctaaagcctatcacaatgaagaagttgtgatggaacgtcatcgccaccgttatgaattcaataataagtatcgtgaagcaatggctgctaagggcatggtcttctccggaacttcgcctgacaaccggctcgtcgaagtgattgaattaccaaagaagcgcttcttcgtggcctcacaataccatccagaattcttatcacggcctaaccgtccagaagggttattcaaggcattcatcgatgccgctaaccagactggtaaggtgaaggcataa

SEQ ID NO: 32 - protein, 537 aa, Lactobacillus plantarum
MTKYIFVTGGVVSSIGKGIVAASLGRLLKNRGLKVTIQKFDPYINVDPGTMSPYQHGEVFVTDDGTETDLDLGHYERFIDINLNKYSNVTTGKIYSEVLQKERRGDYLGATVQVIPHITNAIKEKIMRAGTTTDSDIVITEIGGTVGDIESLPFIEALRQMKSDLGSDNVFYIHTTLIPYLRAAGEMKTKPTQHSVKELRSYGIQPNMLVVRTEQPITREMRNKIASFCDVEPEAVIESLDVKTIYSIPLNVQKQNMDQIVLDHFDVQAPKADMSEWIDLEHHVQNLSRTIKIALVGKYVALQDAYISVTEALKHAGYTDDADIDLKKISAEDVTPENVEELLGDADGILVPGGFGDRGIEGKITAIKYARENDVPFLGICLGMQMASVEFARNVLGLKDANSAEIDPKTPDNIIDLMADQEDVEDMGGTQRLGAYPCKLKPGTVAAKAYHNEEVVMERHRHRYEFNNKYREAMAAKGMVFSGTSPDNRLVEVIELPKKRFFVASQYHPEFLSRPNRPEGLFKAFIDAANQTGKVKA

SEQ ID NO: 33 - DNA, 870 bp, Lactobacillus plantarum
ttgacacatggacaactaatcagaaaattacgaaaagaacgtggcctaactcaggcacaattagcagaaggaatttctagtcgcactaccctttccacattagaaaatagtaaaactgacgttaatataaataccctttttagctatttggatcgtttaaatgtatctattcaggaatatatgttttatttcaacgacagttctaataccgaaaaggaattagcaaccaaatacttttacgataacattgtgaaaaagcgtgatattgaaattgaacaacgaattttagattatcagtctaaatataaagattctaaggatttctattactgctgtttgtctattgagctaaaactcttcttgaataaaaagaaagataaaactgtctttgacgtaagggaagatacagagattataaaaaagtatttggaacgtgttactcaatggggacattttgagatgtctatttttgccaactgtctatacattttcaccagtgattatattcgagccacctttacaatcctattgaaaagaactaaaattctcagcaaaattgatacttatcaaaatgatatttctatttttctaaataattgcattgtactggcacttgaaagaaagaattaccaaaatgcacgcttctatattcaacagctttaccaaatatctgagaaaacacctcgtaaagcttatgacagaatgatgtgtgcttattacctagcactactcaaacaacttaagggtgttaacgcgaacgttgatagtacgattagtcattttaaagaactaggtttttctgagcacgctgaaatgcttgaaaatttacgggatagattactgtcttcgagtaaacaatccatagcttaa

SEQ ID NO: 34 - protein, 289 aa, Lactobacillus plantarum
MTHGQLIRKLRKERGLTQAQLAEGISSRTTLSTLENSKTDVNINTLFSYLDRLNVSIQEYMFYFNDSSNTEKELATKYFYDNIVKKRDIEIEQRILDYQSKYKDSKDFYYCCLSIELKLFLNKKKDKTVFDVREDTEIIKKYLERVTQWGHFEMSIFANCLYIFTSDYIRATFTILLKRTKILSKIDTYQNDISIFLNNCIVLALERKNYQNARFYIQQLYQISEKTPRKAYDRMMCAYYLALLKQLKGVNANVDSTISHFKELGFSEHAEMLENLRDRLLSSSKQSIA

SEQ ID NO: 35 - DNA, 678 bp, Lactobacillus plantarum
atggcagaaacaaaaattccacgggcaacggcaaaacggttaccgatttattaccgctatttaaatatcttgctagatgcagataagaagcgggtctcatcgaccgagttgtccgaggcggttaaagtagattcagcaacgattcggcgagatttctcgtattttggggcgctcggcaaacgagggtatggatacgatgttgaaacgttacttgcatttttcaaaaagattttaaatcaagacaccttaacgaatgttgctttaattggggtcggtaatttgggccacgccctactgaactttaattttcacaaaaacagtaatgtccgcatttcagcagcatttgatgtcaacgaggcgattgccaatacagtccaaagtggggttccagtgtacccaatgacggagctcaaaaagcaattgatcgaacaacagattgagattgctatcttaacggtgccaaccacggttgttcagaaaattaccgatgacttggttgatgcaaacgtcaaaggaatcatgaactttacgccgttacgaatctccgttcctgagacagtacgggttcagaacgttgatttgaccaacgaattacaaacattgatctacttcattgaacattacggtcagcaattaggtgacaatggtaatgacgatgaaaatgagactgaagattaa

SEQ ID NO: 36 - protein, 225 aa, Lactobacillus plantarum
MAETKIPRATAKRLPIYYRYLNILLDADKKRVSSTELSEAVKVDSATIRRDFSYFGALGKRGYGYDVETLLAFFKKILNQDTLTNVALIGVGNLGHALLNFNFHKNSNVRISAAFDVNEAIANTVQSGVPVYPMTELKKQLIEQQIEIAILTVPTTVVQKITDDLVDANVKGIMNFTPLRISVPETVRVQNVDLTNELQTLIYFIEHYGQQLGDNGNDDENETED

SEQ ID NO: 37 - DNA, 561 bp, Lactobacillus plantarum
atgcgaaaggatgcacaaattaaccaacagaaaattttgactgctgcgcgacaactctttgccgcgcgttcaatcgaaaccgttagtatgaaagatattgcgacggccgctggtatcggtcccggaacgttgtatcgtcactatgcccataaaagtacactatgtttggcattggtaacggaccgagtcgcaacttttattaaaaccaatcaagtctacttgaccacgacctcggtaggtgcagcagcacgttttgatcatgttattggggaatatttagcgattcgtgagcacaacatggcgttattaatgaatgtcgaggccggtgaacctggtcgccgtcaattttatcagagcgaactttatcaacaattatgtgacctattaacgcaactggttcgtgatttaaagccaacgctttcgaaaccggcatgtgagtttcaagctgatatgttaattgccatgctgaaggggactagttatgcttttcaacgtcaatggcgaggacggtcgcagtccgaactattggcgcaattgcacgcgttaatggtgactgaatga

SEQ ID NO: 38 - protein, 186 aa, Lactobacillus plantarum
MRKDAQINQQKILTAARQLFAARSIETVSMKDIATAAGIGPGTLYRHYAHKSTLCLALVTDRVATFIKTNQVYLTTTSVGAAARFDHVIGEYLAIREHNMALLMNVEAGEPGRRQFYQSELYQQLCDLLTQLVRDLKPTLSKPACEFQADMLIAMLKGTSYAFQRQWRGRSQSELLAQLHALMVTE

SEQ ID NO: 39 - DNA, 882 bp, Lactobacillus plantarum
atggaatttacttggcaacaccaggggcaaccaatggcaatgaaacgattcttgacgacgcatgccatcagtatgcgaacaatcaaggcaatcaagcatggcaccggcgcgtttcttgtcaataatcaagttcaaacgggcgttattaccattcatgatggcgatattgcgggaattcaactaccagacgaggcaccggatacagcggtagccgtcagcgaacaaccaatccaaattgagtatgaagacgctaattggctcgttcttaataaaacagccgggttaaccagcgtgccggggcctagtaatcggaccgatacgttggtcaatcgaatcaagggttatttgatggccagtcatgccagtaatcaacgaccgcacctgatcacgcggttggaccgggatacgagtggccttgtgttagttgctaaacatcgggtggcgcaggggatgttgacagagccccgaattgcggcgcaattagtgaagacgtatcaagcttggatcgaagggaccattacgccggctagtggcacaattgatcgcccgattggccgggtggctgacagtcctcggcgagtggtcaccacggcgggccaacgcgccattacgacgtatcaagtggaggcggaccaattgcagcataacgtgagtcggttacggttggaacttgtgactggacggacgcatcaaattcgggtccatctaacgacgcttgggcaccccttattaggtgatgcgctgtatggcggtaacttggggtggattcaacggcaagccttacacgccgctagtttacagttctttgaccccttttcggaacagactttacactttgaggcggcattgccagctgatctgcaagccttgaatcacgactaa

SEQ ID NO: 40 - protein, 293 aa, Lactobacillus plantarum
MEFTWQHQGQPMAMKRFLTTHAISMRTIKAIKHGTGAFLVNNQVQTGVITIHDGDIAGIQLPDEAPDTAVAVSEQPIQIEYEDANWLVLNKTAGLTSVPGPSNRTDTLVNRIKGYLMASHASNQRPHLITRLDRDTSGLVLVAKHRVAQGMLTEPRIAAQLVKTYQAWIEGTITPASGTIDRPIGRVADSPRRVVTTAGQRAITTYQVEADQLQHNVSRLRLELVTGRTHQIRVHLTTLGHPLLGDALYGGNLGWIQRQALHAASLQFFDPFSEQTLHFEAALPADLQALNHD

SEQ ID NO: 41 - DNA, 327 bp, Lactobacillus plantarum
atggctaattttcaacaatccgaaacggcacttattgaaagtgccacactaattaatatcttcgccgaaaaaattcggcgtcagattatcattgctctggggaacagtgataatggtctcaacgttactgatattaccgccttggtcaatatttcacgccccgccgtttcacatcatctacgcttaatgcgtgaagctggggtcattgatatgcgcagtaacggagtcgagcatatttattttctcacgttaaccgcaccattacagcaattacaggcaacttttgcgacgctaacggctgataacgcgccacttagtcagtcgtaa

SEQ ID NO: 42 - protein, 108 aa, Lactobacillus plantarum
MANFQQSETALIESATLINIFAEKIRRQIIIALGNSDNGLNVTDITALVNISRPAVSHHLRLMREAGVIDMRSNGVEHIYFLTLTAPLQQLQATFATLTADNAPLSQS

SEQ ID NO: 43 - DNA, 783 bp, Lactobacillus plantarum
atgatgggagtggaaaatgtgaaagtcttaggaatattaggtgcgcatcgcgctgatggcgtgactgcccagctactgcaatccgtcttaaagggggccgcggccagcgctgacacggaactagtcaacctcaacgattatgagttgcgaccagatcacgatagtcaaccgaatgctgacttagacgcgctggaagcaaaattaatggcggcggatgtctgggtattagctgcaccaacctatttggggagcttatcgggggtaatgaaaaacttctgtgactgttttcgggggcggatcgcacggtttaattccgtgggtgaagcagtacctgatcgctttaagaacaagcattatgtgacgatcacggattgttacgcgggtggtattgaaaattatttgaccggcgtgactgacgcaacgtttaaaacacttgataaatttttgacgatgggtggtctcatcaaattacgggagattgtcgtaactaaaacgtggggtatgcaaaccatcacagctgctaagcaagcagaatgtgaacgggtcggcgcgcgggctgcacataaaaaggaaagggatgacagtacggtgaaacggtatattcaattattcttcatgattgcggtgatggcactactaacaatgggaatcgaagcggggattcaacaattgattccgctgaacaatttttgggcctactacggcgtctttgtcgtcgtcttttatgttcttttagcaatgattttacatttcttcactgttgttaaacaccggcgtcgttaa

SEQ ID NO: 44 - protein, 260 aa, Lactobacillus plantarum
MMGVENVKVLGILGAHRADGVTAQLLQSVLKGAAASADTELVNLNDYELRPDHDSQPNADLDALEAKLMAADVWVLAAPTYLGSLSGVMKNFCDCFRGRIARFNSVGEAVPDRFKNKHYVTITDCYAGGIENYLTGVTDATFKTLDKFLTMGGLIKLREIVVTKTWGMQTITAAKQAECERVGARAAHKKERDDSTVKRYIQLFFMIAVMALLTMGIEAGIQQLIPLNNFWAYYGVFVVVFYVLLAMILHFFTVVKHRRR

SEQ ID NO: 45 - DNA, 1002 bp, Lactobacillus plantarum
atgcaagtttttggacaatttattgcaacagtcggttggctaggattggcactagtcgccagcgaactaggtgcgacgttaatccattggctcggtcagtgggtcggatttcgattaattggtgctcgaattgtccggattaccggttttcgacttcaattaagtcgggttcgtggtcattggaaattagaacgaccgctgacgcgtcatccacatatcgtggcagcaccctcggcggatgccaaacggttcaatcacgccatttattgttttggcggtggcctgttcaacttactgacggtcatgctcagtttaataactctgaatcaatttaagtttagtttcgatttatggttgtttgcgttcattatttggatctgggtcaatacgttgaaagccgcccaattattaccaatgaacttgcacggttatcccacggcgggacaggaatttcggcaggcacgcgaatcaacggcggcgatgaccgccgcgtatgtcactgcgtgtgctgcggccgttaaggttcagaccggtagtgtccgtgaccttgatgcaagtatgattgttatgccgcgcgatggtggcaatcggaattatttagtcgtccggcaagcctggttgattctggaatggggacttcaacatgggctggacacccccgaactgttagcggggttgagtcgcttggagccaagtttcaatacgttgccgccagctgatttggcgaagtatttagatgcaacattgtactggaacttggtcaccaatcatcgtgacccccagatcatagcctggtatcaagatgatggtgttcaacagttattacgtcgctaccaacctttggctcattataagttaactgccgtttatgaatggcgggtccatcagcagcctgaacaggccctagcattgattgaaaagggactaaaaattgctcagcgcctccacgatgaggaagaaattgcttggctgaaagctttacgcgttcaagtaacggcctag

SEQ ID NO: 46 - protein, 333 aa, Lactobacillus plantarum
MQVFGQFIATVGWLGLALVASELGATLIHWLGQWVGFRLIGARIVRITGFRLQLSRVRGHWKLERPLTRHPHIVAAPSADAKRFNHAIYCFGGGLFNLLTVMLSLITLNQFKFSFDLWLFAFIIWIWVNTLKAAQLLPMNLHGYPTAGQEFRQARESTAAMTAAYVTACAAAVKVQTGSVRDLDASMIVMPRDGGNRNYLVVRQAWLILEWGLQHGLDTPELLAGLSRLEPSFNTLPPADLAKYLDATLYWNLVTNHRDPQIIAWYQDDGVQQLLRRYQPLAHYKLTAVYEWRVHQQPEQALALIEKGLKIAQRLHDEEEIAWLKALRVQVTA

SEQ ID NO: 47 - DNA, 1134 bp, Lactobacillus plantarum
ttgaagatgagaaaacggctagcaattgtgtgggggagcctagcattgctcgcactattattgggatatgcttgctacgccctgagtattcagcgcggtcaagacacggtcacgcggatttatcaaactgatcaaaatgggacgccgattatttcacccggaccaattaccttagtaggtaaggtcaatcaccgcaatttatttcaatctggcattaatggctatgtcttaacgaatcgcgatccattgtcgactttgttgccgcgccgtaatcaaacagtgcatctaaagtatcgttctgcacagacgacggcggagctacgcaagacgctacgtcaagcacggtatttacaggccggtactcagaataccgccacgccggtctttcaaaatcgacagcagcgaggtgatgcgacaacgtacggtcgtatcagtaccagccaagacggccggatatggacgaaactacccattagttatccgcatgtgcaattgtcacggccgagtgtctggtacgcgaatggccgcttgacgttgatagatgggaaagaccgttactggacgactaattttaaagattggcaacatcaacggttgaactttaacggggctgattttaagcaaggtcgggttcaggccgtctttccaggtacgactcgttcagcggttgttgtggttcgcggcattgatcgccaaagcagtcgcgccaaactctattatggacagctcacgaagactggacgggtcaaagcttggcacgcgttacaactaggaaagctcccagcgcgccaagtcgctggaatgagcttgattgatcaacacttatacctgtttcttcagcgcggtacgcagttggccatttatcgtgccaatcggttgacgcgtccggtcaggttggttggtcgcgttaagctaaatcatgcgcagtcacaacgagtgaccgcggtgaatttgataccgaccaccaagcatcgctaccggttaatatttgacttgacgacagctgaaaaagttcagaaacagccacgttatcggttacttgatcggcgatttaaagcagtggggcagcagcatctattggtcactgattatctctggagccaatttcaaattagtctacgtgggagtgagtga

SEQ ID NO: 48 - protein, 377 aa, Lactobacillus plantarum
MKMRKRLAIVWGSLALLALLLGYACYALSIQRGQDTVTRIYQTDQNGTPIISPGPITLVGKVNHRNLFQSGINGYVLTNRDPLSTLLPRRNQTVHLKYRSAQTTAELRKTLRQARYLQAGTQNTATPVFQNRQQRGDATTYGRISTSQDGRIWTKLPISYPHVQLSRPSVWYANGRLTLIDGKDRYWTTNFKDWQHQRLNFNGADFKQGRVQAVFPGTTRSAVVVVRGIDRQSSRAKLYYGQLTKTGRVKAWHALQLGKLPARQVAGMSLIDQHLYLFLQRGTQLAIYRANRLTRPVRLVGRVKLNHAQSQRVTAVNLIPTTKHRYRLIFDLTTAEKVQKQPRYRLLDRRFKAVGQQHLLVTDYLWSQFQISLRGSE

SEQ ID NO: 49 - DNA, 633 bp, Lactobacillus plantarum
gtgaggcacatgaaggtacagccaaaggaacgctttagtctagcgtggcggtggttgccgctcgaattgctgatcattatgctaagcgtcggccttggatgggcgggcaatcgatggctacctaagccggtgtatcaagcatctgttgatattcagattgcgcaaacgccgcgttcagggctgtcaacagcccgtctaaaacgtcagcgacgccaggatatcaaagctatcacgcagttcaacgtgatgccacaccagagtgcagtgctgactcaagccagcacttatgcctatgcgcattatggcatttggcaaccgattcaggaactgagtgagtcggtccaagcggcaccagttgcgcggcgaccggtcttacgggtgacagcaacgagtagttcacggcaagtggcccagcagaatgctcaggcgttcaatgtggcgattaaagctaatctgacgggcttaaaaaattatcgagtgaagacagttaaacgtaccgtaacgcgtgagacgaacgtgattcgcggggcgctttggaagttaatattagttgttgggggcggcttggcgttgctgagtccgtacctcgtgaaatatggtcagggttgggggcggcacgatgatgagacgtag

SEQ ID NO: 50 - protein, 210 aa, Lactobacillus plantarum
MRHMKVQPKERFSLAWRWLPLELLIIMLSVGLGWAGNRWLPKPVYQASVDIQIAQTPRSGLSTARLKRQRRQDIKAITQFNVMPHQSAVLTQASTYAYAHYGIWQPIQELSESVQAAPVARRPVLRVTATSSSRQVAQQNAQAFNVAIKANLTGLKNYRVKTVKRTVTRETNVIRGALWKLILVVGGGLALLSPYLVKYGQGWGRHDDET

SEQ ID NO: 51 - DNA, 891 bp, Lactobacillus plantarum
atgatgagacgtaggggagcaagtatgcagcagcaccgtaatgtgctctatctgattatcttcggaatctacttagcctcagtcacactacagacgacgacctttaacgagatgataccgcatcgagtgggcgttttgattgaattagcgactttggccgcattactgggcctcgtggtttgcttagataccttgacccccggccaaattattggagaagtcagtttacttgtactggtgactgtcgtgacactcacatcgggtgcgcattatttgatgccgacaatcatgttggtgattgcagcccgggaagtttcgtttcggcagatcattcaagtttatctgggcgtcgtggggacgattctcttgttagcgctagttgctgcggaagtcggactgattaaaaatattacgtttgcaactgccgatgggttacgtcagtcgtttggtgtcgtgtataccactgattttgcggcccatattttctatctgtgtgcggcgtatttgtatttattggcccgtcgttttcgattagtagcgctattacccgtgttgtttggcctggcaatgatttaccagtttacgaaaacgatgacggatgtgattgctttactcgttttgatcagcttgtacttggtctatatctatcgccgtcagcttcggtggcttcggccgatgattcggtatagctttttgatgttaccacttgctagtgggctaattattgggttgtcgaatatttttaattatcaagaccggttgctggtagcgctcaataataccttgtccacgcgactcgcgctagggaataacgcgttattagcatatggcgttaaattattcggccaagccccgattccaattaatggttggggcggcgatccggtttga

SEQ ID NO: 52 - protein, 296 aa, Lactobacillus plantarum
MMRRRGASMQQHRNVLYLIIFGIYLASVTLQTTTFNEMIPHRVGVLIELATLAALLGLVVCLDTLTPGQIIGEVSLLVLVTVVTLTSGAHYLMPTIMLVIAAREVSFRQIIQVYLGVVGTILLLALVAAEVGLIKNITFATADGLRQSFGVVYTTDFAAHIFYLCAAYLYLLARRFRLVALLPVLFGLAMIYQFTKTMTDVIALLVLISLYLVYIYRRQLRWLRPMIRYSFLMLPLASGLIIGLSNIFNYQDRLLVALNNTLSTRLALGNNALLAYGVKLFGQAPIPINGWGGDPV

SEQ ID NO: 53 - DNA, 837 bp, Lactobacillus plantarum
atggtaaaaattgcagtcctatctgacgttcacggtaacgctactgcactcgaagcagtcttagcagacgcccagaaacaacacgttgacgaatactggacagtaggcgacatgaccgttcgcgggccagaatcggagcgctgtctcaccttactagaccgcgttcaccccaccgcctacgttctcggaaatcacgaggaaaactaccaaaaagtaatggcagccaatcccaacacgtttactaaacccaaacaaattatggcaacggttctcaccgcttttgatcggcgccagctgagttcgacacactttgaacggttactgaacttaccaatgacagtcatcaaacacgtcggcccgttaaccatccgtctccaacacgttttaccgaccgtcgctagtggacacgcgctcgcaccaactgccagtcaggccaactttgaccaagccgctgaaggcgatgtcgatatcgtcatctacgcgcacacacaccagcccatcatgcgctacgcaaccacgggacagttgattctaaacgccgggacagttggtcttccgactgccattaatccccacctacgccaaccacgagcaaactacttgcttctgacaattgatgagactggcctccagcatgttgattaccgcgccgtagatttcgattggcagcgtgctatcacgattgcgcaaaatacccacttaccctacttcgaattctatgaacaaactttgcagactaatacttaccaatatgcaccgagtgcggtcgctgcttataatacgcaacatgacatggcccgagaagcgcgcaagattttacttgaaaatcgtcactga

SEQ ID NO: 54 - protein, 278 aa, Lactobacillus plantarum
MVKIAVLSDVHGNATALEAVLADAQKQHVDEYWTVGDMTVRGPESERCLTLLDRVHPTAYVLGNHEENYQKVMAANPNTFTKPKQIMATVLTAFDRRQLSSTHFERLLNLPMTVIKHVGPLTIRLQHVLPTVASGHALAPTASQANFDQAAEGDVDIVIYAHTHQPIMRYATTGQLILNAGTVGLPTAINPHLRQPRANYLLLTIDETGLQHVDYRAVDFDWQRAITIAQNTHLPYFEFYEQTLQTNTYQYAPSAVAAYNTQHDMAREARKILLENRH

SEQ ID NO: 55 - DNA, 1326 bp, Lactobacillus plantarum
atgcaatcacatcgtcatcaaagtcttgaagaaatcaatcagagcgtcgcggttcccgacgttcatcagacggccttttggcgcaaatttttagcctatagtggtcccggtgcactagtggcagtcggctatatggatcccggcaactggttgacatccctagccggtggcggtcagtttcagtaccggcttttagccgtgctcgcattagccatcattgtcgccatgttcatgcaaggcctggcaatcaggctaggcgttgtagcccggcaagacttagcacaagccatcgctagcaagctgccccggcccgtgcgttacgccgcgtggattttaaacgaagtcgcgatgatggcgactgatatgacgggcgtaattggaaccgcaatcgccttaaaaatgttattcggcttaccactacttgcaggaattttactgacgattgcggacgtcttagttgtcttgttgtttttgagatttggcattcggcgtgtcgaagtaatcgtcctcgtcgctattttgaccgtcggcattatttttggtatcgaggtgggacgggcccacgttcaatttggcaacgtgttgctcggcttagttccaacaccattgatcgtcaaaaatcataccgcactagtcctcagtctcggaatcttgggcgcaaccatcatgccacataacttatacttacactcatcgcttgcacaaagccggcgttatgattatcataatccagcccaagtcacagaagcactgcgcttcgccaattgggactcaacagtgcacttgattgcggcttttctcatcaacgcacttttgctcgtccttggtgggacgcttttcttcggtcacaccaacgcgttagcgagtctgcaggccgtcttcgatgggttaaaaagtaccaccgtggttggcgcccttgctagcccggtcatgagctggttatttgcattagccctactaattaccggcctaatttcatccatcactagcaccttagctggtcagatcgtcatggaaggttatttacacatccgcttaccgctatggcaacgccggctgcttactcgcgctgtcacgctaattccgattctgattatcggtatgttagtcggctttagtgacgctgcctttgaaaacttgatcatttacgcgcaagtggcactcagcatcgccctcccctttaccttgttgccactagttgcgctgacaaatgacgccagcctgatgaaggcccacgttaatcgcccggcggtaacgtgggtgggatatggactggccggaattattacggtgttgaatatttatttggtgtatagcttgttttga

SEQ ID NO: 56 - protein, 441 aa, Lactobacillus plantarum
MQSHRHQSLEEINQSVAVPDVHQTAFWRKFLAYSGPGALVAVGYMDPGNWLTSLAGGGQFQYRLLAVLALAIIVAMFMQGLAIRLGVVARQDLAQAIASKLPRPVRYAAWILNEVAMMATDMTGVIGTAIALKMLFGLPLLAGILLTIADVLVVLLFLRFGIRRVEVIVLVAILTVGIIFGIEVGRAHVQFGNVLLGLVPTPLIVKNHTALVLSLGILGATIMPHNLYLHSSLAQSRRYDYHNPAQVTEALRFANWDSTVHLIAAFLINALLLVLGGTLFFGHTNALASLQAVFDGLKSTTVVGALASPVMSWLFALALLITGLISSITSTLAGQIVMEGYLHIRLPLWQRRLLTRAVTLIPILIIGMLVGFSDAAFENLIIYAQVALSIALPFTLLPLVALTNDASLMKAHVNRPAVTWVGYGLAGIITVLNIYLVYSLF

SEQ ID NO: 57 - DNA, 897 bp, Lactobacillus plantarum
atgaagaaatggctcattgcccttgctggtgtcttactaaccttcaccttagctggttgtggtagcaagaccgttgcatcaacttccggtggtaagattaccgaaagccaatattacagtagtatgaagggaacctcttcaggtaagcaagtcttgcaacaaatgatcctgaataaggtgctcgaaaaggattatggctcaaaagtttcgactaagcaagtgacgaagcaatataatacttacaagtcacaatatggtagttctttctcaaccgtcttatcgcaaaatggtttgacgaccaagaccttcaaggaacaattacgttctaacttattattgaaggaagccgttaaagacaaggtcaagattactgataaagctttgaagaagcaatggaagtcttacgaacctaaagtcacggttcaacatatcctagttgccaaatcagcaactgctgacaaagtcttagacgctttgaagaaggattctagccaagccaactttacgaagttagccaagaagtattcaactgatacaacgactaagaatgatggtggtaagttatcagcctttgataacactaacacgagctactcatctaaattcttaacggctgctttcaagctgaagaacggtgaatacacgacttccgctgttaagaccagcaacggttatgaaatcatccggatgatcaagaaccctggtaagggtaagatgtctgatcacaccgctgatttgaagaaacaaatttgggacaatgatatgagcgactccactgtcttacaaaacgttgtttctaaagtgcttaagggtgggaacgtttcaatcaaggataacgatttgaaggatatcttatcgtcatacctttcaacctcatcttcatcaagctctaactaa

SEQ ID NO: 58 - protein, 298 aa, Lactobacillus plantarum
MKKWLIALAGVLLTFTLAGCGSKTVASTSGGKITESQYYSSMKGTSSGKQVLQQMILNKVLEKDYGSKVSTKQVTKQYNTYKSQYGSSFSTVLSQNGLTTKTFKEQLRSNLLLKEAVKDKVKITDKALKKQWKSYEPKVTVQHILVAKSATADKVLDALKKDSSQANFTKLAKKYSTDTTTKNDGGKLSAFDNTNTSYSSKFLTAAFKLKNGEYTTSAVKTSNGYEIIRMIKNPGKGKMSDHTADLKKQIWDNDMSDSTVLQNVVSKVLKGGNVSIKDNDLKDILSSYLSTSSSSSSN

SEQ ID NO: 59 - DNA, 891 bp, Lactobacillus plantarum
atgaataagcaattattaactgaatttgaatccaagtggcgtcctcagattaatcagtatttagatgagcagcttcaagcgtgttctgaccaatcaaccctgactgacgccatgcgttattcggtactagcaggtggcaaacggttacggccgttattgacgttagccattttagatacttttgatatcacaacgacggctgcaaacttgcgggcaagtgtggccgttgagttgatgcatacttattcgctaattcacgatgatttaccggcgatggataatgatcagctacgacgtggcgaaccgactaaccatgttaagtttggggaagacgttgccattcttgcaggggatgctttacaaccgttgactttcgaatggattgcggatagtgggttaccggcatcgatcgtcgctaatcaaaccttagcattagcgcaggccaccggacctcgcgggatggtggctggtcaagttgctgatgtccttggagcgggacaacatctggctttgccagccttacaacagctgcatcgcgagaagacgggggcgttaattcactacgctgtccaggcagggttgattcaagctcaagtgcaaccaaccgtgcaggaattgctattacaatatgctgatgcctatggattggcgtttcaaatttacgatgatattttagacgtgacgagtacgcctgctcagttaggaaaagctacgcataaggatgccgatgagcataagaatacgtatccaggtttgctggggcttgcaggcgcacgaacagcgttagaacaagcggtaacagctgctcaaacggcgttagtaaaagctagtgctgctagtcaacgaggcatgggcttgcttgcagcttttctaacgtattttacagattaa

SEQ ID NO: 60 - protein, 296 aa, Lactobacillus plantarum
MNKQLLTEFESKWRPQINQYLDEQLQACSDQSTLTDAMRYSVLAGGKRLRPLLTLAILDTFDITTTAANLRASVAVELMHTYSLIHDDLPAMDNDQLRRGEPTNHVKFGEDVAILAGDALQPLTFEWIADSGLPASIVANQTLALAQATGPRGMVAGQVADVLGAGQHLALPALQQLHREKTGALIHYAVQAGLIQAQVQPTVQELLLQYADAYGLAFQIYDDILDVTSTPAQLGKATHKDADEHKNTYPGLLGLAGARTALEQAVTAAQTALVKASAASQRGMGLLAAFLTYFTD

SEQ ID NO: 61 - DNA, 759 bp, Lactobacillus plantarum
atggatggaattttatcgggtaagaccattgtggtcatgggtgtggccaatcagcgcagtattgcctgggggtgtaccgaggcattaattgcacagggggcccaggttatcttgacttaccaaaatgaccgtttgaagcaaagcttacaacggtttgttgcgccagatgtgccgttaattgcctgtgatgttgctgatgatgacaatgttgagcgggcatttgcaagcattaaacaacagtatggtgccatcgatgggattatccatgcgattgcttatgcggataaagcaaccttagaaggtgattttgtgaataccacgaaagctggatatgatttggcacaaaatattagtgcgtattcgctgattgcagttgcccgagcagctcggccaatgctgaaaccaggagccagtctcgtaacgttgacgtattttggatcagagcgagccgtaccaaattacaatatgatgggggttgctaaggccgcgttggaagcaaatgtgcgttacttggcgcgtgaccttggaccacaacaagtccgcgtgaatgcaatttcagccggagcagtcaaaacgttggcggtaacgggtattcatgagcatcagcaattattaaaattatctcgcagtatgacagttgatggagaaccggtaaaaacgcgtgagatcggcaacgtggctgcctttttattaagcaatctatcgactggaatgaccggggacgtggtatacgtggataaaggggtccacttaagttaa

SEQ ID NO: 62 - protein, 252 aa, Lactobacillus plantarum
MDGILSGKTIVVMGVANQRSIAWGCTEALIAQGAQVILTYQNDRLKQSLQRFVAPDVPLIACDVADDDNVERAFASIKQQYGAIDGIIHAIAYADKATLEGDFVNTTKAGYDLAQNISAYSLIAVARAARPMLKPGASLVTLTYFGSERAVPNYNMMGVAKAALEANVRYLARDLGPQQVRVNAISAGAVKTLAVTGIHEHQQLLKLSRSMTVDGEPVKTREIGNVAAFLLSNLSTGMTGDVVYVDKGVHLS

SEQ ID NO: 63 - DNA, 552 bp, Lactobacillus plantarum
atgccaatcactcaagttgtctttaaacggcagtggcttcagatgccggtcgatgtttctaaaaaaatgcggcgggtcactcagcgaaccgtgagtcgccaattaattcagcaagtgttatcggtaccattagcttatcatcgattgggccagccctattttccaagtcatcctcgattaggtgttagtgttagtcacacgcaccagttagtgatggtagcggttggtccgggacctctggggattgatgttgaacaggtccgtccatatgatgtgactgccattcggcgagcctttacatcggtggaatggcagctattacaggttttatcggtgcaagatcgttatcggttagggtggcaactttggacggctaaagaagcggtattaaagttagtgggctgtggcttgacccatgcgccccgccgtgttgaggttcttgatttagaacgtggactagcgtgctatcaaacacagttataccagttgacgccgttagaattgcctgcgactcacgagggatttttggctcgtcccttgtcggttggctga

SEQ ID NO: 64 - protein, 183 aa, Lactobacillus plantarum
MPITQVVFKRQWLQMPVDVSKKMRRVTQRTVSRQLIQQVLSVPLAYHRLGQPYFPSHPRLGVSVSHTHQLVMVAVGPGPLGIDVEQVRPYDVTAIRRAFTSVEWQLLQVLSVQDRYRLGWQLWTAKEAVLKLVGCGLTHAPRRVEVLDLERGLACYQTQLYQLTPLELPATHEGFLARPLSVG

SEQ ID NO: 65 - DNA, 1662 bp, Lactobacillus plantarum
atgagtaatcatcaaatccgcttgtccttatcaatcatcaccagttgcttgttggcaactctgattatcggcccgttagtcgccctgattggtcaaacactagtcgggcaatcgccaagccagctatggtcacaactgacgcagccaaccaaccgtgtgagcattcaacacagtctgttcctcagtgggggcacggtcgtcgggacaaccctgctagccacccctttggcatggatcatgacgcacacccgtttaacaaagctcgcctggttgcattggctcttgttagtgccattcatgacaccaccatatattaacgcgatgggctggttatatttctttcaaccacacggattactggctcagcttaatccgagttggcaccaccaatttcagtggctattttcaccgttcgggatggtcattatcatgagtctgcatttgtatcccgtggcatacttaggcttacgcgcagccctcatgcaattcaaccagcgctggcttcaagcggccgaagttcatggggtcaacacctggcaacgactagtgcgaatcacattaccaatcatgttagtcccatacttagctgtatggattttagtctttaccaaaaccttggctgaatttggaacgccagccacctttggtcggagcatccacttcgaagttctgacgactacgattcaaagggacctcagtcagtggcccttagatttccaaaacggggtactcaccggcaccctcctactgaccattgccctgattgcctggggtatccagcaatggttgttacgccggccagctgttaagttcaccggacaacggtcagcgtcacaatatcggcagcttggagtgacaacattagcaggcactttcgtcaccctagtcatcagtattgctattgtcctgccattcagtgccatcgtgctccaatcgctactcaaacaacgcagtcttggttggagtccgtctaatttgacacttgtacactatatagacctcttacgctttgatagtcctgcctggcaggccattgttacgaccgtcggattggcattactgattagcagtctcaatgtgatcgttggtttattcttgagcgttgggagtttaacaaaacgttttcccaagtggctgcgacagttatgtcataccttgggcgcattgccactcgcaattccaaacgtcgtcttagcattgagcttaatgatgctcttttcacaggtgctggcgttcaccaaattatacggcaccctaaccatcctcctgatcgcggatgtcaccttatttctaccaacaacggtgcaatacttgacgaccgccctcaaggcctttgactcggaattgctggctagcgcgcgcatcttcgaacctagtttcggccgcattatcctaaaaattgcacttccgattctatggcccgcgctactcaacagctttgtgatggctttcattgccacgagtcgtgaattagtcgttgccctattgttactgccttccggtatgacgaccgtttcaacatttatctatcaatcgttcgaacaaggtgaagcggccgccggtatggccctagcggtattgacggtagcattgacattcattggactgattgcagctaatcacctgcaatcagctaccaagccagtacgccaaccaaactag

SEQ ID NO: 66 - protein, 553 aa, Lactobacillus plantarum
MSNHQIRLSLSIITSCLLATLIIGPLVALIGQTLVGQSPSQLWSQLTQPTNRVSIQHSLFLSGGTVVGTTLLATPLAWIMTHTRLTKLAWLHWLLLVPFMTPPYINAMGWLYFFQPHGLLAQLNPSWHHQFQWLFSPFGMVIIMSLHLYPVAYLGLRAALMQFNQRWLQAAEVHGVNTWQRLVRITLPIMLVPYLAVWILVFTKTLAEFGTPATFGRSIHFEVLTTTIQRDLSQWPLDFQNGVLTGTLLLTIALIAWGIQQWLLRRPAVKFTGQRSASQYRQLGVTTLAGTFVTLVISIAIVLPFSAIVLQSLLKQRSLGWSPSNLTLVHYIDLLRFDSPAWQAIVTTVGLALLISSLNVIVGLFLSVGSLTKRFPKWLRQLCHTLGALPLAIPNVVLALSLMMLFSQVLAFTKLYGTLTILLIADVTLFLPTTVQYLTTALKAFDSELLASARIFEPSFGRIILKIALPILWPALLNSFVMAFIATSRELVVALLLLPSGMTTVSTFIYQSFEQGEAAAGMALAVLTVALTFIGLIAANHLQSATKPVRQPN

SEQ ID NO: 67 - DNA, 1011 bp, Lactobacillus plantarum
atgtctaaagcagcaattatcacattatccacgctcggcgtgctcgcaattggcacgtcgctttatgttaatcaacatcagaaaaaaacgttaaccgccaacgcgcaaaccagtcaacaagtcttaacagtatacgctgctggacctaaacccctttctgatcaaatcatccacggttttgaagccaaaactggcattaaagtcaaaagttttgacggcacgaccgggaaaattttaagtaaggtcaaggccgagcaaggcaatccccaagctgatgtgctgattttagcttcaatggccgctggcgtcgatttacaaaagaatggccagctattaacctatcagccttctcaagctaaacacctgaataaacaatttaaagatactagccaccagttgatcaattacagtgcttcggcagtcggcatcacctacaatacgcggcacatcaaatcggcaccgacagactggtctgacttgacaaccgctccgtatcgcaatcaagtgaccattccggacccccaaacctctggttctagcttggacttcattaacgcttatcaaatgaaacacggtacgcaactacttaaagcccttcaagaaaacggtgccgatatcgggggtgctaacaaggaagtactcgatgcagtcatcactggccaaaaaatcgccgtctttggtggggtcgattacatgagtctaacagctattaaaaaaggcgaaaaaattggtttcgtttatcctaagagtgggactttggtcaatccacgaccggcgatgattttgaaggctagtcgtcatcaagccgccgccaaacaatttattgactatctcttatcagctaaagttcaaagacagattcaaaaaagtaacttaattccaggtaccacgagcactttgaccgatccacgcaatggcgaagccatcaaagcctacacggtcaattggaccagtgccaacgcggccctgaccaaaaacgttgtcgcattcaatcaggtctttagccaatga

SEQ ID NO: 68 - protein, 336 aa, Lactobacillus plantarum
MSKAAIITLSTLGVLAIGTSLYVNQHQKKTLTANAQTSQQVLTVYAAGPKPLSDQIIHGFEAKTGIKVKSFDGTTGKILSKVKAEQGNPQADVLILASMAAGVDLQKNGQLLTYQPSQAKHLNKQFKDTSHQLINYSASAVGITYNTRHIKSAPTDWSDLTTAPYRNQVTIPDPQTSGSSLDFINAYQMKHGTQLLKALQENGADIGGANKEVLDAVITGQKIAVFGGVDYMSLTAIKKGEKIGFVYPKSGTLVNPRPAMILKASRHQAAAKQFIDYLLSAKVQRQIQKSNLIPGTTSTLTDPRNGEAIKAYTVNWTSANAALTKNVVAFNQVFSQ

SEQ ID NO: 69 - DNA, 330 bp, Lactobacillus plantarum
ttgagtaatgaggtgattagcatgtttgatcaagatgaagaacggtttgcaacgctagggttagcggccaagctaccgagcgcggtgattgatggcatttgggatattattgatcaaaatctaaagggggtcgttcgcctgccacgggtcctgcaatttgccctgatcgcacgtaatgggcaagtcaccgtggcttttgatgcgcagcacgatgccatcatggaattcgatttaccagtcaattaccaacgggagtttcccgagacggtggcagtcttagacgatggtcagtatcagaccatgatgttgatggacgaactctccgtctga

SEQ ID NO: 70 - protein, 109 aa, Lactobacillus plantarum
MSNEVISMFDQDEERFATLGLAAKLPSAVIDGIWDIIDQNLKGVVRLPRVLQFALIARNGQVTVAFDAQHDAIMEFDLPVNYQREFPETVAVLDDGQYQTMMLMDELSV

SEQ ID NO: 71 - DNA, 747 bp, Lactobacillus plantarum
gtgtgtctaatggcgaaaacagcagtgtgcattgtcgatcaacaacgttaccaagttgtggacggtatgcgattagaagaattggaaactagtttgcggcaaatgattttaaaagattttccgcaggcccataatagcagtttcatttgtagtgagcatctcgtacattatcgcttagcaaagatggatgcgatgatcgagaacgattatcaacaaaatgataaggtcaatgcgcaattatctaagattctcgctaaccacacgtatcgggtcgtcgatgttaatagcgagctggaaagttcattgacatttggtcaacgggtcgcggatggggtcgcacggttcggggggagctgggcgtttatcatttcgtttgtcgtggtgatgctcgtgtggatgttgctcaacgtcttaccaatttttagccatcattttgacccttatccctttattttattaaatttatttttaagcatggtcgcagcaatccaggcaccattgatcatgatgagtcagaatcgggcagctgagtatgatcggctacaagcggccaatgattttaaagtgaactcgatgtctgaagaggagatccgggtcctgcactcgaaagtcgatcatttaattcaacaagatgaaccaaacatgcttgaaatccagaaaatgcaaacacaaatgttaggtgagattcaagcacaagtcaatgaattacgacgattgcagccgcggcgacgtcgcaatcaaagttaa

SEQ ID NO: 72 - protein, 248 aa, Lactobacillus plantarum
MCLMAKTAVCIVDQQRYQVVDGMRLEELETSLRQMILKDFPQAHNSSFICSEHLVHYRLAKMDAMIENDYQQNDKVNAQLSKILANHTYRVVDVNSELESSLTFGQRVADGVARFGGSWAFIISFVVVMLVWMLLNVLPIFSHHFDPYPFILLNLFLSMVAAIQAPLIMMSQNRAAEYDRLQAANDFKVNSMSEEEIRVLHSKVDHLIQQDEPNMLEIQKMQTQMLGEIQAQVNELRRLQPRRRRNQS

SEQ ID NO: 73 - DNA, 885 bp, Lactobacillus plantarum
atgaattatcgcaacttgctcgtattaccacttgccgtcttgttggtcggctgttcatcaccaacacaccaggattccaagtcaactagtcagacgaccacgagtgccaaggcaacggttagcagtacccaaaagaaggctaaggctaccagtagtactagtagtcggcctcaaaccgcagccacgcgctcgtctagaacagcgcgtgagcgggccgccagcgccgctaacaagtcggtcacccagcccacggctacgacccggctggcagcattgaatcaacaattgactaagacgttgggaaagcaggcgctcgttccacaagtcgatgggttaactagtggcagttcgaagttgaacatgcgctattcaggtgacgcagccaattacaccatcaattatagtgtgggacaacaggcccagccattcaacgcggcggccgtggtggatgaaacggcttatgcgactgtcactaagacgacctatgcgacaactaatgccgcggcccagcaggtgggttatcgtgataataaatccacagctgggctgccaaccgtcgatctcggtcatcaaatcaccgcgcatatcgacgcgggtgctggtcaacgatatatcatgtggaatgagggccgctggtcgttgaccgtgcatgcgaacatgatgcacgaagatgcgggcgtcgcgttagctaaacaggccgtcgctactttcgagcaggtctacttaccagcaccacagtcggtcggcgccatcacttttgacgcgatttcgtcaggctctggacctttagaccaagttatccaatggcaagctggtaaagtggtttatcaagtcaaggctcaagagatggcaacggctatcaaaatggctgccagtatgcaataa

SEQ ID NO: 74 - protein, 294 aa, Lactobacillus plantarum
MNYRNLLVLPLAVLLVGCSSPTHQDSKSTSQTTTSAKATVSSTQKKAKATSSTSSRPQTAATRSSRTARERAASAANKSVTQPTATTRLAALNQQLTKTLGKQALVPQVDGLTSGSSKLNMRYSGDAANYTINYSVGQQAQPFNAAAVVDETAYATVTKTTYATTNAAAQQVGYRDNKSTAGLPTVDLGHQITAHIDAGAGQRYIMWNEGRWSLTVHANMMHEDAGVALAKQAVATFEQVYLPAPQSVGAITFDAISSGSGPLDQVIQWQAGKVVYQVKAQEMATAIKMAASMQ

SEQ ID NO: 75 - DNA, 351 bp, Lactobacillus plantarum
atgcaagtgcgcttagtgccaaacttacaactcggtgaacggattatcgggccgacccccgaccctgaggccaatcgcgcgctttatcaacgttatgcgaaacgattacaggcgcggctaggtatcggctttcaagtctacctagatatgagtgacggttatgatttactgcatgcgcgtgattacgacaccgatacttgttgggtggttgcagcggctgtttaccaagcattaactgattctgccgtgatcacccaccaccgtatcatctcgctgagtgaccaagcacttatcttaaaagcgacgcagcccatcgaacaacaactgcgccaatctccgactgatcaatag

SEQ ID NO: 76 - protein, 116 aa, Lactobacillus plantarum
MQVRLVPNLQLGERIIGPTPDPEANRALYQRYAKRLQARLGIGFQVYLDMSDGYDLLHARDYDTDTCWVVAAAVYQALTDSAVITHHRIISLSDQALILKATQPIEQQLRQSPTDQ

SEQ ID NO: 77 - DNA, 726 bp, Lactobacillus plantarum
atgatagattggattaagctgctcaaaatgcactggcggattgttggtggtgttactgccgtgatcattattttgataacgggatgggcactgagtcaacttaagcagcctcaacccgccggtacggataacttgttggcgcattcgttcaattccacatcaatgggaggcgcgagtcgaacgtctgccaatgctgaccagcccgcaacgagtacccaaccgtcgaacgctacaccgagcccagcccgaccgacaggtgctagttcacccgggtatgtcgatattaagggtgcggttaataaaccagggttgtatcaggttactgctagtatgcgggtcgcggatgtcatccaactggcacaaggcatgcagccacaggcagatgctcagcagatcaacttggctgccaaagtgactgatcagcaagtgatctacgtgccagctaagggcgaacaggccccggctgttgcgccaccagtcgtccagtcaacggggcctactggcggaacaccaactagtgatcatgcggcaacggataaggtcaatctcaacacggctgatgtggccgcgttgcaaacgttgagcggaatcgggcagaagaaggctgaaaaaatcattgattatcgccagcaacatggtaattttaaaacaattgatgatttgaaaaatgtcagcggctttggagaaaagactgtggtcaaatacaaagaccagctcaccgtctag

SEQ ID NO: 78 - protein, 241 aa, Lactobacillus plantarum
MIDWIKLLKMHWRIVGGVTAVIIILITGWALSQLKQPQPAGTDNLLAHSFNSTSMGGASRTSANADQPATSTQPSNATPSPARPTGASSPGYVDIKGAVNKPGLYQVTASMRVADVIQLAQGMQPQADAQQINLAAKVTDQQVIYVPAKGEQAPAVAPPVVQSTGPTGGTPTSDHAATDKVNLNTADVAALQTLSGIGQKKAEKIIDYRQQHGNFKTIDDLKNVSGFGEKTVVKYKDQLTV

SEQ ID NO: 79 - DNA, 1047 bp, Lactobacillus plantarum
atgactaagggccgggagttgttaaaacgttactggggcttgctggtcgtgatactggtggtacttgcactatttctaattcctttaccgtactatattgaaggacccggaagtgcaaacaatttgaagacttttgtgaccgtcaagcggcatccggatcatcaccggggtaagtttatgttgacctcggtcgcagaagctcgagcgacgccgctgatgtggctttacgcacaattgaatccgcactatgacgtggtcagtgctcaggatatgactggcggtcaggatgacgcgacttataatcgggttcagaagttttatatgcgaagtgcaatcaacgaagctatcgcgacggcgtattcggctgcgcatcagcaataccgcaaggtttatcagggtatctacgttttaacggttcagtctaattcgaaatttagaaaccagttaaaagttggcgatacgattacgaaagtcgatggccaccattttaatacagccagtgcgtatcagcattatattggtaagcagggcgtcggacatcgagtgacgatcacgtatcggcgaaagggccatttgaagcaagcaagtgcgcccctaatcaagctgagcacgcaccgcgccgggattggtatcggcctaactgataatattaaagtgacgacgactattccggtcaaggtcgatcccggacaaatcgggggtccctcggcgggcttgatgtttagtttgcaaatttatcagcaattgaccaatcagaacttacgacacggacgcaagattgccgggaccggcaccatcgatcaaaatggacaagttggtgaaattggtggtatcgacaagaaagtgattgctgctaagcgggcaggggcgacaattttctttgcaccgtatgtgaaaccaaccaaagcgcttttggcggttgaagaaaagggtcaaactaactatcaacttgctaaagcgaccgcgaaaaagtacgcgcctaatatgaaagttgttccagtgacctcatttaaacaggccgttcattatttgcagacacaccaatag

SEQ ID NO: 80 - protein, 348 aa, Lactobacillus plantarum
MTKGRELLKRYWGLLVVILVVLALFLIPLPYYIEGPGSANNLKTFVTVKRHPDHHRGKFMLTSVAEARATPLMWLYAQLNPHYDVVSAQDMTGGQDDATYNRVQKFYMRSAINEAIATAYSAAHQQYRKVYQGIYVLTVQSNSKFRNQLKVGDTITKVDGHHFNTASAYQHYIGKQGVGHRVTITYRRKGHLKQASAPLIKLSTHRAGIGIGLTDNIKVTTTIPVKVDPGQIGGPSAGLMFSLQIYQQLTNQNLRHGRKIAGTGTIDQNGQVGEIGGIDKKVIAAKRAGATIFFAPYVKPTKALLAVEEKGQTNYQLAKATAKKYAPNMKVVPVTSFKQAVHYLQTHQ

SEQ ID NO: 81 - DNA, 960 bp, Lactobacillus plantarum
ttgattgctgaatactacattattattaatgaactggcaggatctggtcacggtaaggtcgtttgggaaaccgtcaagccgattctagaacaacgacagattcgatttgaatatcgaatttctgaatatgccggccacacaattcggctcgcaaatgagtacgttaaaaccattcaacgacgaccaaacgtgaccccggtcattctggtcattggtggtgatggcacactgaacgaggccttgaatggtattatgcaggtcccacaagctgaaccgatcccgctcgcctacattcctggaggttcgggcaacgactttgctcgcggtctgggtatggcgactgatccagcaattgcacttgcacaagtactcaacaatatgcggccccgttcgttaaatgttggttatttccatgaaaccttgaaaaacgaacaccggtatttcgtcaacaacgttggtttaggatttgacgctcaaatcgttgatgacacaaaccgtagcaaaaagaagggccgtctgggtcgttgggcttatctcagtaacatgctggccgcatattcccaacaggaaggcttcccgctaaccgtacacgttaaccggaagcgagactattataagcgggctttcctttgtacagtctcgaacattccatactttggtggcggagttaaaattctgcctcaggctaatctgcacgataatcagctcgaattgatcgttgtcgaagagcctcactggtggattatcctctggttgttcgtcttactgctactgggtggccgtcatcttaagtcgcgtttcgttcaccattatcgcaacgctaacttgcacttgttggttaactctgttgaaattggtcagatggatggtcaaattattgggaatcgtaattacgacctctacttgtccacccatccctacccattctggatcgacactagtatccatgaccaccactaa

SEQ ID NO: 82 - protein, 319 aa, Lactobacillus plantarum
MIAEYYIIINELAGSGHGKVVWETVKPILEQRQIRFEYRISEYAGHTIRLANEYVKTIQRRPNVTPVILVIGGDGTLNEALNGIMQVPQAEPIPLAYIPGGSGNDFARGLGMATDPAIALAQVLNNMRPRSLNVGYFHETLKNEHRYFVNNVGLGFDAQIVDDTNRSKKKGRLGRWAYLSNMLAAYSQQEGFPLTVHVNRKRDYYKRAFLCTVSNIPYFGGGVKILPQANLHDNQLELIVVEEPHWWIILWLFVLLLLGGRHLKSRFVHHYRNANLHLLVNSVEIGQMDGQIIGNRNYDLYLSTHPYPFWIDTSIHDHH

SEQ ID NO: 83 - DNA, 204 bp, Lactobacillus plantarum
gtgagtaatcgttttgagattctggaagaatatcaagaggctaataccgaactcgatcatttaaggacgctagccgttcggcaacaggatcgctcacgggttgtgaccatttatccgcatttgaaagaacgggtgagtcacttatctcgtaaatgtgaacaacttgacatgcttctggaagcaatcaacgcttctgaggactaa

SEQ ID NO: 84 - protein, 67 aa, Lactobacillus plantarum
MSNRFEILEEYQEANTELDHLRTLAVRQQDRSRVVTIYPHLKERVSHLSRKCEQLDMLLEAINASED

SEQ ID NO: 85 - DNA, 675 bp, Lactobacillus plantarum
atggcaaaaacatcacgtcctagcaaagctaaacagcaagcactagtccatcaactcattggtaagattgatgcacagcccgaggactaccatgcttattatgaactagtggtattattaacggctggtcaagattttgaacaggcagaggcgttagcaatgaaggcgttgggaaagtttgaccaccaacaacccgcagccgactacctgcgttatgcgttgggaaatgtctattatcaagctcaaacttatgacaaagcgttaccatattatcaacaaattacggatgatcaactaaaacaggatgcttatttaatgagtgcacaagctttaatggcccaacacgattaccaacatgccctagtctgggccattacggctcaagaggcacgtccacaacaacttgacgctaatttgttagtagcagacatattacttgcattaggcaataatcagcaagcatcggattactatcaacgcgcatataagattgattcgcaatctgggcgagctgcttttaaccttggactgaccgcaatggtactgggaaaaccgtatgccacctggtttgaacgcgcgcagaaattggacagtcaatattttaaaagtcatcagcaacagctaacggatattgaaaaaatgttagccgcacaagcagataatcaaaatcactaa

SEQ ID NO: 86 - protein, 224 aa, Lactobacillus plantarum
MAKTSRPSKAKQQALVHQLIGKIDAQPEDYHAYYELVVLLTAGQDFEQAEALAMKALGKFDHQQPAADYLRYALGNVYYQAQTYDKALPYYQQITDDQLKQDAYLMSAQALMAQHDYQHALVWAITAQEARPQQLDANLLVADILLALGNNQQASDYYQRAYKIDSQSGRAAFNLGLTAMVLGKPYATWFERAQKLDSQYFKSHQQQLTDIEKMLAAQADNQNH

SEQ ID NO: 87 - DNA, 666 bp, Lactobacillus plantarum
ttgacaaaactattatttgttcgccatgggaaaacagagtggaatcttgaggggcgctatcaaggctctcagggagattcaccattattaccgactagttatcaagaaattcatgaattggcagcggcgctccaggatattcggtttagtcatatctatgtcagtccgttaaaacgggcgcgtgatacagcgatgacactacgtaatgatttgacacaatcagagttacccataacggtactgagtcgtttacgggagttcaatctcggtaagatggaaggaatggccttcacggatgttgaagctacgtatccggccgaattcgacgcgtttcgaaatcatccggatcagtatgacccgacagcgattcagggggagagctttcaacaactgctgaagcggatgactcccgctattaagcaaattgttcaagcaaatccacgtcgcgatgacaatgttttgatcgttagtcatggtgcggccttgaatgccttggtcaactcattactgggagcgacactggcgacgttacggcaacggggtggcttgtcgaatacgtcaacaacaattttagagacgcgtgaccgtggtcaacattttaagctattagattggaatgacacctcatacctatcacggcggcccgatgcaactgatacgatttaa

SEQ ID NO: 88 - protein, 221 aa, Lactobacillus plantarum
MTKLLFVRHGKTEWNLEGRYQGSQGDSPLLPTSYQEIHELAAALQDIRFSHIYVSPLKRARDTAMTLRNDLTQSELPITVLSRLREFNLGKMEGMAFTDVEATYPAEFDAFRNHPDQYDPTAIQGESFQQLLKRMTPAIKQIVQANPRRDDNVLIVSHGAALNALVNSLLGATLATLRQRGGLSNTSTTILETRDRGQHFKLLDWNDTSYLSRRPDATDTI

SEQ ID NO: 89 - DNA, 1086 bp, Lactobacillus plantarum
atgttgatcgcgatggatgcccagcatcaactcgttaatgcggcgacggctgatcgccaaatagaatattactgtccaggatgtgtgcagccggtgcgattgaaacggggggcagtgatcgtgccacattttgcccacgtccatgccacggattgtgatgctttctctgaaggggaaacaacggaacatcttcgtggtaaacaacaattagcgacctggttcgctgccagtggttatacggtgcgcttagaggctggtttgccagagatacatcagcgcccggatatcttggttcgacgaggtacagcgcaaccactcgcgttagaatttcagtgttcacccttgtcagtggagcgactcgcagctcggacgcagggctatcgtcagcatggctatcaagtgttatggttgttgggacgcccctatcagcgacaattgcacctcaatagcaaggctttgaagtttttgcagtaccagcaacggtggggcctgtttctactttttttggacactcaaagtactagtgttcgtttattgcatcacgtcttgacattggatacggaaccgctgacctatcaaacgattcgactggatacgcgcagccagtcggtgttacattttcgccaattggctccaaaaattaacaccccaactttgccggacacgcatttacggcattattatcaacagctaatgttggcacggcttcgacatcaacgtggttttgatgcgttacaagtggcttgctatcaacgcgggggaacgattgcccagttaccgacgtggacgatgcccactgtgccgcaactaccgctgttatcggtaccatatctggtctggcacgcccacgtcttttttgcactgcggcaacagtccggacggcttgctgggtcacaattagaggcattgatctgggcccaattacggccattacttgcccggcgtgcctgcctgcaagcacgaggcacgttaatacaacaactgataacgaccatgattacgatgcttagcgagcagcaagtgataaactggcagaaaactgagtggcaggtgaattccgaccagcttcgatggcgcaaacattga

SEQ ID NO: 90 - protein, 361 aa, Lactobacillus plantarum
MLIAMDAQHQLVNAATADRQIEYYCPGCVQPVRLKRGAVIVPHFAHVHATDCDAFSEGETTEHLRGKQQLATWFAASGYTVRLEAGLPEIHQRPDILVRRGTAQPLALEFQCSPLSVERLAARTQGYRQHGYQVLWLLGRPYQRQLHLNSKALKFLQYQQRWGLFLLFLDTQSTSVRLLHHVLTLDTEPLTYQTIRLDTRSQSVLHFRQLAPKINTPTLPDTHLRHYYQQLMLARLRHQRGFDALQVACYQRGGTIAQLPTWTMPTVPQLPLLSVPYLVWHAHVFFALRQQSGRLAGSQLEALIWAQLRPLLARRACLQARGTLIQQLITTMITMLSEQQVINWQKTEWQVNSDQLRWRKH

SEQ ID NO: 91 - DNA, 2256 bp, Lactobacillus plantarum
atggaattagatgccgttggtaaggcaattgtacagtatcacttagtcccactcgttcatcaggctaatttaggactagaggtcaccatgcaccgggtggacgcccatggtcacttagcgacgacagcacacccgcaagcgtttggatcagcgcaacaaaatcatcagttacgtccgggcttttccgcaagtgctttaaagtttactacgccggtgcgtcgtgacattcctgcattgatggcgtatctgaagggcttgaataccgcagcacggcggtcactcgatgcggacgaacgactttggccactgtcgagtacgcctgtgttgccggatgatctaacgaacgtaccactggctgatgttgatcaagtcagctatcagcgtcgtcgcgacttagctcgtaagtatgagttacagcgattaatgacgactggtagtcacgtgaatatgagcttgaatgaagctttattcacccgtttatatactgagactttccatcagcagtatcacagttatgttgactttcgcaatgcaatttatctgaaagtcgctcagggattggtgcgcatgaactggctgattcagtatttatttggcgcttcaccacgcctagccgttacggatactacgagtcgtccacagcgcagtagtgttcaacatcccgatggtcgctacagtcaagtgacgggagactatacgtcaattgatcgctacgtggccaagttgacggcggctgttcgtcaacagcagttgttgtctgtcaatgattttgacgggccagttcggcttcggagtaatgggcagctagctatgatggcccggcagggggtctattatcttgaataccggggcttggatctcgatccaactagtccagtcggggtggacgcgaacgcggtggcatttgttcgtttgttggcgagttatttcgtaatgatgccggcacttccagctaagatggtatcccaagtcaacgctcaagctgaccaattgacccgtcaagttttgggtgaaaatccaacgacggctagtgctcaggccgtgccggctgttcaagttttagatgcacttgctgattttgttaaaacctatggcctaccaaatgaagatgccgtgttactcaaacagttgaagtcgtgggtcactgatccaaagaagacgctgagtgcgcagattgccatgcaagccgatccgttagcatgggcactcgaacgggctgcacgctatcaggaatcgagcaatgaacgtccgtttgaacttgcgggctttaccgcgctagatctatcgagccagcaactagcccagcaggccttgacgcggggagtgcaggtggacgttgttgacccacacgctaacattttacgattgactaagttaggacggtcgcaattagttgtgaatgggagcggaacggatttaaatccacaggcgctaacgaccgtactgacacataaagcagcggccaaacaaattctggctgagcacggggttccggtgccggcttcacagacatatcatacagctaatcagttgattgctgattatgatcggtacgttcaagctggtgggatcgtattaaaagcggcggatgagtcgcacaaagtaattgtctttcggattatgcccgaacgcggactgtttgaacaagtcgtccggcaactattcgagcaaacgtccgcggtaatggccgaggaagtggtagtcgcatcaagttatcgctttttggttatcgatagtcgtgtgcaagcaatcgtcgaacgaattccagccaatattgttggtgatggtcgctcaacggtcaagacgttacttgatcgcaaaaatggtcgagcgttgcgcgggaccgcttttaagtggcctcaatcagcgctacagttaggaacgatcgaacggtatcgcctggactcatatcacttgaccttagattctgtggtcagccggggaactcagatcttattacgagaggatgcgacttttggtaacggggcggacgtgctagacgcgacggctgatatgcatcaatcctatgtgcaggcggtggaaaagttggtagcagacttacacttggcggtcgctggggtcgacgtgatgattcccaatctctatgccgaattagtgccagagcatcctgaaatggcggtatacttgggtattcatgcggcgccgtacttgtatccgcacttgttcccaatgtttggtactgcccaaccagtggcggggcagttgttggatgcattgtttaaaaatgaagattaa

SEQ ID NO: 92 - protein, 751 aa, Lactobacillus plantarum
MELDAVGKAIVQYHLVPLVHQANLGLEVTMHRVDAHGHLATTAHPQAFGSAQQNHQLRPGFSASALKFTTPVRRDIPALMAYLKGLNTAARRSLDADERLWPLSSTPVLPDDLTNVPLADVDQVSYQRRRDLARKYELQRLMTTGSHVNMSLNEALFTRLYTETFHQQYHSYVDFRNAIYLKVAQGLVRMNWLIQYLFGASPRLAVTDTTSRPQRSSVQHPDGRYSQVTGDYTSIDRYVAKLTAAVRQQQLLSVNDFDGPVRLRSNGQLAMMARQGVYYLEYRGLDLDPTSPVGVDANAVAFVRLLASYFVMMPALPAKMVSQVNAQADQLTRQVLGENPTTASAQAVPAVQVLDALADFVKTYGLPNEDAVLLKQLKSWVTDPKKTLSAQIAMQADPLAWALERAARYQESSNERPFELAGFTALDLSSQQLAQQALTRGVQVDVVDPHANILRLTKLGRSQLVVNGSGTDLNPQALTTVLTHKAAAKQILAEHGVPVPASQTYHTANQLIADYDRYVQAGGIVLKAADESHKVIVFRIMPERGLFEQVVRQLFEQTSAVMAEEVVVASSYRFLVIDSRVQAIVERIPANIVGDGRSTVKTLLDRKNGRALRGTAFKWPQSALQLGTIERYRLDSYHLTLDSVVSRGTQILLREDATFGNGADVLDATADMHQSYVQAVEKLVADLHLAVAGVDVMIPNLYAELVPEHPEMAVYLGIHAAPYLYPHLFPMFGTAQPVAGQLLDALFKNED

SEQ ID NO: 93 - DNA, 1029 bp, Lactobacillus plantarum
atggcagacgtacaattagactggaacaacttggggtttgaatatcggaatcttccgtaccgttatcgtgcgtattggaaagatggtgcttggtacaaaaaagaattaacgggagatgcaactttacatattagtgaaggctcaacagcattacactatggtcaacaagactttgaaggcttaaaagcttaccgcactaaagatggtagtgttcaattattccggccagatcgtaatgcagcccgaatgcagaccagctgtgaacggttattaatgccacaagtgccgacagacatgtttgtggacgctgttaaacaggttgttaaggctaatcaagattacgtgccaccgtatggaactggtgcgactttatacttacggccattgatgatcggggttgggggtaacattggtgttcatccagctcaggaatacatcttcacggtctttgccatgccagttggtagttatttcaaaggcgggatgacgccaaccaactttacgacgtccgaatatgatcgtgcggcccataaaggaaccggggcttataaagttggtgggaattatgcggctagcttattcccaggtcaagaagctcatgccaacggtttctccgactgtgtttatcttgacccggttgaacatcgtaagattgaagaagtaggttcagcgaacttcttcgggattactaaggatggcacttttgtgacacccaagtcaccatcaattctacctgccgttacgaaatattcattactctacttggcagaacataaatttgggatgaagactgaacaaggcgacgtctacattgatgatttagaccggtttgctgaagctggggcttgtgggacagctgcggttatttcaccaatcggtggcttggaacatcaaggcaagttacacgtgttctacagtgaaacggaagttggtccggtaacgaaaaaattatatgatgaattaactggaattcaatttggcgatcgggaagcgcctgaaggttgggtccagaaagttgaattagactaa

SEQ ID NO: 94 - protein, 342 aa, Lactobacillus plantarum
MADVQLDWNNLGFEYRNLPYRYRAYWKDGAWYKKELTGDATLHISEGSTALHYGQQDFEGLKAYRTKDGSVQLFRPDRNAARMQTSCERLLMPQVPTDMFVDAVKQVVKANQDYVPPYGTGATLYLRPLMIGVGGNIGVHPAQEYIFTVFAMPVGSYFKGGMTPTNFTTSEYDRAAHKGTGAYKVGGNYAASLFPGQEAHANGFSDCVYLDPVEHRKIEEVGSANFFGITKDGTFVTPKSPSILPAVTKYSLLYLAEHKFGMKTEQGDVYIDDLDRFAEAGACGTAAVISPIGGLEHQGKLHVFYSETEVGPVTKKLYDELTGIQFGDREAPEGWVQKVELD

SEQ ID NO: 95 - DNA, 321 bp, Lactobacillus plantarum
atggcaactgcaactttaagcgacgaagaaattcgcgaacgtatcaagacgggtaaacacatgttattctttacggcggactggtgcccggattgcgcttttattaaaccagtaatgccccaaatcgaagcgaagtacgatcaatatgactggatcacggttgatcgtgacgccaacattgaaattgcccaagacatgggtgtgatggggattcctagtttcgttgggatcgaagatggtcaagaaattggtcgatatgttgacaaattccgtaagacccaaaagcaagttgaagatttcttagatacacttgaaaagtag

SEQ ID NO: 96 - protein, 106 aa, Lactobacillus plantarum
MATATLSDEEIRERIKTGKHMLFFTADWCPDCAFIKPVMPQIEAKYDQYDWITVDRDANIEIAQDMGVMGIPSFVGIEDGQEIGRYVDKFRKTQKQVEDFLDTLEK

SEQ ID NO: 97 - DNA, 1254 bp, Lactobacillus plantarum
atggcagaaacggcacagttagaagctgctgtacaggcattaagcgatattgtaaaaatgaatacggtaaataaccatgagcaattggtggcggattatttggtcacacttttaaagcaacatggtattgaagcacaatcaatcgagtacgctcccgggcgggtgaacttggtggccgaaattggtgatggtcacggcccagtcgtcgcgttggatggtcatgaagatacggtggcgttgggtgatgcagataaatggcacacggacccgttggcagcaactatcaaagataatcggttgtatggtcgcggtgtgacggacatgaaggctggactaatggctgaagtcttcgcgatgattgcattgcacgatcaggacgccccactccatggcacggtgaggttactcgcaactgtgggagaagaagtcgaccatttaggcgctgaacaattgacggaactcggttacgccgatgatattcaaacgttgatctgtgcggaaccaagcggtgcggacaaacaacttttactgaccaagtcgattcaagccatgttaggtgttgacggcgatacggcgcaacggatggcggatgcgaatccgacgaccgaacaacacttcatcgaattagcgcataagggttcactgacttatacgattaaagcgcaaggggtggcggcccacagttcgatgccagcgattggtcaaaacgccatcgatatgttgatgacttactatcagaaacaaactgcctattttgacagtttcaagactattgttaatcccgtattgggcccaaccgtgcctgtcgtgacgttaattagtggtggcgaacaggtcaacaccgtcccagccagtgccgaaatgtcagtaaaaattcggacgattccagaattacggaatgaccgcttgattaaggatttggaagccatcattgctgaatgcaatgcagatggtgccaatctaacgatggacatcgcaagttcgttctacccagtgcatacgccggaagatagccagttggtccagttggcgaagaaggttggggaacaagttttacagcaacggctcccgtactttggtgctcctggtgggacggacgcatcgtcttatattgtaaagagtcctgatatgcaagtgattgtcttcggacccggcaatattacagcgcaccaagttaatgaatatgttgatttggatatgtatgggcgcttcatcgagatttatcaaaaaatgattacggaattgctggcttaa

SEQ ID NO: 98 - protein, 417 aa, Lactobacillus plantarum
MAETAQLEAAVQALSDIVKMNTVNNHEQLVADYLVTLLKQHGIEAQSIEYAPGRVNLVAEIGDGHGPVVALDGHEDTVALGDADKWHTDPLAATIKDNRLYGRGVTDMKAGLMAEVFAMIALHDQDAPLHGTVRLLATVGEEVDHLGAEQLTELGYADDIQTLICAEPSGADKQLLLTKSIQAMLGVDGDTAQRMADANPTTEQHFIELAHKGSLTYTIKAQGVAAHSSMPAIGQNAIDMLMTYYQKQTAYFDSFKTIVNPVLGPTVPVVTLISGGEQVNTVPASAEMSVKIRTIPELRNDRLIKDLEAIIAECNADGANLTMDIASSFYPVHTPEDSQLVQLAKKVGEQVLQQRLPYFGAPGGTDASSYIVKSPDMQVIVFGPGNITAHQVNEYVDLDMYGRFIEIYQKMITELLA

SEQ ID NO: 99 - DNA, 2592 bp, Lactobacillus plantarum
atggtaggtgcagcgatgacagcagcttattttaaaacaattatgcgtgagatttggtcgtcgaaagcccgttttgcttcaatattactgatcattttcttaggggttgctttttatacgggaattcgggcgactggtccggatatgtcacaggcagctaatgattattatgcgaagcaaaagctggcgaccaatagtgtccagtcgacaatggggctgactaaggccgatacacgagtactgaatcaacaccgctcacagctcacttaccaagcgacacgctacgctgatgttaatcagttgaacaatagtcaagtcgtgcgggtgatggtgttaccgacaacccaacgactgaatcggttacggatcgttaagggacgattaccgcggcatgccaatgagatcgtgttagacgcgcaagcacagcgattgcagccgaagctaaaagtcggttcaacgtatcgaattagcagtacggctaagcgcaatgcacagttcacgcggcgaacattcaaggtcgtggggtttgtcaattcaccaacatatgttgaaaatacgaatcgtggcgtcaccaacgttggtaaagggactttggactacttggtctatgtccgtccgcaggtgattaagtccagtgtgataacgcgcattgacgtgcaattcaaaaatttgcggggcgtcaccccctacacggcgaagtatcggcgcttgaatcgcgagaacaccgcgcaactcaaacgctggttaaaaccacaagcgcgtaagcggcagcaggcattacaagcccaggcccaggctaagctgaaaccattgcgacaggcgacccagcaacttgctagtcaagtgccagcgggaacggcacaactagtcaagttacaaagccaattaaaacgcgcgaaggcccaggtcgcggccatcacaatgccgacttatttgtacactgaccgtacggataatccgggttacacagaatatcacgaaaatacgcaacgagtcgtggcactgtcgactgtctttccgctgttctttattgcgattgccgcgttaatttgtctaacgacgatgacgcggatggttgaagaattgcggctacagatggggacgttaaaggccctcgggtatacgaataccgcggtcggtagcgagtttatgatttatggtggtttagccgcgctgattgggaccgcgctaggtgtcctgttcggcgtcaattttttcccgcggtttatcgcgcaggcctatggtagtatgtataatttgcccgcaatcaacgttcaatacatttggatggacattggtatcgccttagccattgcgttgttgtgcacgttggggacggcactggtcgtgctccgcgtggatttaaacagtttacccgcgcaactcttacagccacgatcacctaaggccggtaagactttgctattagaacgctggcaatggctatggcatcggctgagttttaatcataaaatcacacttcgtaatctatttcggtataagcaacggttgctgatgaccgtgctcggtattgcgggctgcatggcaatgatgattacggggtttggcttaaaggattccattggtgatattagcgtcaagcaatttaacgaattgtggcactacgatgctgtggtgacgcgtagtgggaacgaaacggaccaacaacggcaagcactcagtcgtggtcaactttaccaggctagtttgaaattacaggccaagcaggtgacggtcaaacagtccggggtagcagaacagacggctacgctcggtataccggcaccccaccaatcgctaagcaagttcgtggtattacggcaccgacaaagtcatcaggccattcatattggtgatcgcggtgcggtcatcgatgaaaaattagctaagttatatggcgttcaggcgggcgatgatttaacgatcaagttggccgggcaaaccaccaagcggattcacatcagtgcggtggctgaaaattacgtcaatcactttatctatatgagtccgacttattatcgacgtgtcttcaagcaggcaccagtatataacacgaactatgtccggtttaagcaggcaacgaaaaagcaagaaaatgcttatgcggaccggctattgaaacaggcgggggttcagaacgtgacactgatgagtacagagaaagccactaattttaaaatgctggatagcatgaacttagtcgtattgatctttgtcatctcggcgggggcactagcgctagtagtgctctataacttaacgaatattaatgtttctgaacggatccgggaattgtcgacaatcaaagtgttgggcttttacgatggtgaagtgacgatgtatattttccgtgaaaatctgatattgacggttttaggcattattgccggttgtttcttgggcaactggttgcacgcatatatcttgcaaacggctgaaacgaacgcgttaatgttttcaccaacgattcatccgttgagttacgtttacgcggcattattgaccctggcctttagtttattagtcatgggaatgatgcatcgtaagttaaagcgagtcaatatgctggatgcactgaaatctgtcgattaa

SEQ ID NO: 100 - protein, 863 aa, Lactobacillus plantarum
MVGAAMTAAYFKTIMREIWSSKARFASILLIIFLGVAFYTGIRATGPDMSQAANDYYAKQKLATNSVQSTMGLTKADTRVLNQHRSQLTYQATRYADVNQLNNSQVVRVMVLPTTQRLNRLRIVKGRLPRHANEIVLDAQAQRLQPKLKVGSTYRISSTAKRNAQFTRRTFKVVGFVNSPTYVENTNRGVTNVGKGTLDYLVYVRPQVIKSSVITRIDVQFKNLRGVTPYTAKYRRLNRENTAQLKRWLKPQARKRQQALQAQAQAKLKPLRQATQQLASQVPAGTAQLVKLQSQLKRAKAQVAAITMPTYLYTDRTDNPGYTEYHENTQRVVALSTVFPLFFIAIAALICLTTMTRMVEELRLQMGTLKALGYTNTAVGSEFMIYGGLAALIGTALGVLFGVNFFPRFIAQAYGSMYNLPAINVQYIWMDIGIALAIALLCTLGTALVVLRVDLNSLPAQLLQPRSPKAGKTLLLERWQWLWHRLSFNHKITLRNLFRYKQRLLMTVLGIAGCMAMMITGFGLKDSIGDISVKQFNELWHYDAVVTRSGNETDQQRQALSRGQLYQASLKLQAKQVTVKQSGVAEQTATLGIPAPHQSLSKFVVLRHRQSHQAIHIGDRGAVIDEKLAKLYGVQAGDDLTIKLAGQTTKRIHISAVAENYVNHFIYMSPTYYRRVFKQAPVYNTNYVRFKQATKKQENAYADRLLKQAGVQNVTLMSTEKATNFKMLDSMNLVVLIFVISAGALALVVLYNLTNINVSERIRELSTIKVLGFYDGEVTMYIFRENLILTVLGIIAGCFLGNWLHAYILQTAETNALMFSPTIHPLSYVYAALLTLAFSLLVMGMMHRKLKRVNMLDALKSVD

SEQ ID NO: 101 - DNA, 1206 bp, Lactobacillus plantarum; identical to SEQ ID NO: 13

SEQ ID NO: 102 - protein, 401 aa, Lactobacillus plantarum
MTPETEQLLRRWYMGQLIVLFGAAFIQLFTFDGGVFFPVGGMQLLIWGLLAWWPAAEEDQAQWRRLRHVNYYVQTVLQFTLLPILLANLVAWLSQLSWLDEQGLIAVGMAYLMVAFVPVAVVVTKPIESVIGRIAVLITAIFSGVVSAQQTFLILPNLQAPSVFEMVSDTGILGALGFVIAVGVLLRGWGLTGPSWRFNRQAQTSLVVGLIVVGTAFSLWNAFSAGGSWATTFTHWDFQLRSATWKMFLSGLEPGIAEEWLYRFAVLTLLLQAFRHRRHQLDLAVWLSGGLFGMWHITNVFAGQPLSATVEQIIFAATLGWFLASTYLYSGSILLPMVIHAAIDILSMMASGSQTMVKPDAFEWQTIGATVIIFVGITIYFLTGSRRQVIQAHVNQRLSVQ

SEQ ID NO: 103 - DNA, 534 bp, Lactobacillus plantarum
gtgccagtggtcattagctcattaattcccgtagttgcgccggttaaacgccaagtgatcaatgtgacgacccttgcaaatacggttcacttatatgggttactaggatttaagcagcaaattataccggcaacagatcaaattgttgcgccctttgctggggttatcacagccgtggcagccaatcaacggttaattggctttcgagcggccaatggtctggtgggctggctacgaattggacaattaaccagtgctttggaatcgcctacatttaagttcaacgttaaaccgggtgactgggtagttgctgggcagatgttggttgaagtgatttcactactgacacaacggctccaaccagttaaaacgacggtggtattgacgattagacatgcggttgttcgtgttcgagaacggttgctggcagcaagtaatcaagtcgatccgatgggaactgttatctcaggtattacgaccagtatggctggtaatcatcgggtcgcaacgatcgggccaccccaaggtaattaa

SEQ ID NO: 104 - protein, 177 aa, Lactobacillus plantarum
MPVVISSLIPVVAPVKRQVINVTTLANTVHLYGLLGFKQQIIPATDQIVAPFAGVITAVAANQRLIGFRAANGLVGWLRIGQLTSALESPTFKFNVKPGDWVVAGQMLVEVISLLTQRLQPVKTTVVLTIRHAVVRVRERLLAASNQVDPMGTVISGITTSMAGNHRVATIGPPQGN

SEQ ID NO: 105 - DNA, 1131 bp, Lactobacillus plantarum
atgacatggtctaattggcgaatttcgccgttcgtaacgagtattttctttatactaggtgttctaacactttactgggtcctctttaattggattacgacgtggtttcatgcccgtcacattaacattgacgatgataccgtcaatgcttggcacggtgtgatttatatgttggtctttgtgtttgtcatgcaactgtcggtcgtcggtaaagcagatagttgggagtttgttaactttcacttgattgccgtcgtgttctgctcgttttttttgaatattcgaatgccgtattattcattgttacctgtggtcatagtgtacatggttttcgaccagtcgattttttactgggaatcgtggagttacgccgtcgtgttcgtgctatttttctggagcatgaattatctacggctgtgggtacctaagcatcggtatccttggctatattactatggcgcggtcgctttttacggcggaatcttgtggggcctgatcaagcttaaatattcgctggattgggacaatactttacaagaatatggctacttaatgatttttgcaggattattgtacgcctacgttaatatgctgacccaagatagtgagattaaattacggttggcccagtttgcgagtcacgacgctttgactgagactgagaactttgccgcttacacggaacatatcaaatatttattcgatgatagtgccaagaacaatctcaacttatcgatgatgatgttcgatattgatcactttaagcacgttaatgacacgtacgggcaccttgcaggggaccgcgttttgcaagaagttgccgccacggtcacaacggtcttggccgccaatgacgagaaggtcaagctgtatcgcaccggtggtgaagaattcaatgtcctgtttcccggttatgatctggctagtaccaaagtgattgtccgtcaggtctttgaagcagtcaatcatctcgttgttaagtatgaagacgaggaaatcaatgtgtcgatttcggttggtgtctcgacactgcatcaagccgatggtagtccgattgatttgtacaaccgtgttgatcagaacctctatttttcaaagcggcacgggcggatgcgtgttacggttgaatag

SEQ ID NO: 106 - protein, 376 aa, Lactobacillus plantarum
MTWSNWRISPFVTSIFFILGVLTLYWVLFNWITTWFHARHINIDDDTVNAWHGVIYMLVFVFVMQLSVVGKADSWEFVNFHLIAVVFCSFFLNIRMPYYSLLPVVIVYMVFDQSIFYWESWSYAVVFVLFFWSMNYLRLWVPKHRYPWLYYYGAVAFYGGILWGLIKLKYSLDWDNTLQEYGYLMIFAGLLYAYVNMLTQDSEIKLRLAQFASHDALTETENFAAYTEHIKYLFDDSAKNNLNLSMMMFDIDHFKHVNDTYGHLAGDRVLQEVAATVTTVLAANDEKVKLYRTGGEEFNVLFPGYDLASTKVIVRQVFEAVNHLVVKYEDEEINVSISVGVSTLHQADGSPIDLYNRVDQNLYFSKRHGRMRVTVE

SEQ ID NO: 107 - DNA, 1485 bp, Lactobacillus plantarum
atggcaacaaaagataatgaaaagattacattgatggcgctagtcatgatgatctttacgaccgttttcggatttgccaatagtacggtggcctattatttaatgggttacagctcgattctattttacctagtcgcagccgtactgttcttcatcccgttcgcgctaatgatggcggagttcggggcagcggttaagtctgatagtagcgggatgtacaagtggctggaagtgagtgtgaatgcgaaatttgcgttcgtgggcacgttcatgtggtttgcgtcgtacattatttggttagtctcaacgtcagctaaagtctggattccgtttacgaccatcttctttgggagcgatcaaacgcagcgctttgcgatgtttggtctgaatgcgacgcagatgattgggattttgtcctgtctatggatggtgctagtgacgttcgtttccatcaaagggatgaaaggcattgtgcgggtcacgagtttaggcggcctggcggtgaccagtttgacggcaatcctgttagtggtttcgggggtcgttttagccttgaatcacggacaattcgcacaaccgttacaacatgtgatgacgtcaccaaatccaagttatcagcatccagtcgggttactcgggtttgccgttttcgccatttttgcttacggtgggctagaagttctcggtgggatggttgataagaccaagaaccccgaaaagaccttcccacgcggaattattatttctgccatcgttattaccttaggctatggtctgggaatcttctgctgggggattagtacgaactggcaagccgttttgtcgaatccaacgactaacctcggtaatattagttacgtcatgatgcaaaacttgggctatgttttagggcaagcgcttggtttgagtacagcggccgctaagacaatgggactgtggtttgcacggtacaccggcttagggatgttcctcgcttacagtggggccttcttcaccttaacttattcaccattgaagacgcttatcttgggaacacctaaggaactgtggccgaagaaatttacgaagctcaacaaagctggtatgccaagttatgccatgatggttcaatgtgccatcgtgattgtgatcattttggtggcgtcctttgcaacggcagacgcgtcagccttttacaatgtgttgaccttgatggcgaacgtttcgatgacgttaccatacctcttcttgctatacgcgtttccgaagtttaaggaaaaccagaacattgttaagccttttgaagtgtacaagtcattgacttggacgaaaattattagttgggtcgtgttcattgtcgtcttaggtgcgaatgttttcacgttgatccagccaatcttggaaactgggcagattcaaaatacgatttggatgctagttggaccaattgtcttcggtgtggccgggattatttggtatcaagttcgagaacggcatgtcaattaa

SEQ ID NO: 108 - protein, 494 aa, Lactobacillus plantarum
MATKDNEKITLMALVMMIFTTVFGFANSTVAYYLMGYSSILFYLVAAVLFFIPFALMMAEFGAAVKSDSSGMYKWLEVSVNAKFAFVGTFMWFASYIIWLVSTSAKVWIPFTTIFFGSDQTQRFAMFGLNATQMIGILSCLWMVLVTFVSIKGMKGIVRVTSLGGLAVTSLTAILLVVSGVVLALNHGQFAQPLQHVMTSPNPSYQHPVGLLGFAVFAIFAYGGLEVLGGMVDKTKNPEKTFPRGIIISAIVITLGYGLGIFCWGISTNWQAVLSNPTTNLGNISYVMMQNLGYVLGQALGLSTAAAKTMGLWFARYTGLGMFLAYSGAFFTLTYSPLKTLILGTPKELWPKKFTKLNKAGMPSYAMMVQCAIVIVIILVASFATADASAFYNVLTLMANVSMTLPYLFLLYAFPKFKENQNIVKPFEVYKSLTWTKIISWVVFIVVLGANVFTLIQPILETGQIQNTIWMLVGPIVFGVAGIIWYQVRERHVN

SEQ ID NO: 109 - DNA, 6099 bp, Lactobacillus plantarum
atgcaaagacgacgcttacaacgagcacaattaacagaaaaacgaacttataaaatgtataaaaaaggacgcctgtggttaattgccgggttgagtactttcactttgggtgctagtctgctaccgatgacggggcgggcagacacgactagtacgcctgctgagaaacaggggacgaggacagagacaactggcaaccaaatcacattggctagtaagtcggttggtagtagttcgatggccaatgatggcgaagaaaaaactaataacagtcaggtagagacgagtagtgaagctagtaatgttactgcatcgactgaagctaagtcaacggaatcaacgactcagactgtggttgattccaccgtgactagtacggctactgaaacaacacgtgcaaacggtgctactaatcagaccagcaaaatgtccatagtcgatacgacgtctaacaacacagaacagaatcaagcagtaggtggtacaacagacagtacggcaagtaccgcaactattgaggatcaagctaaggccgccaacagagcaaccactgatggcaagataaatacggctacagtagctacgaaaacaactacaactgccagttatgcaaccgcggatattagtacgaataccattcgcagtgcgcaaaagctagcacgagctactgtggccaccgttgcgacagtaaattcagcgactaagacgtatgatggtaagatagatacaccaaatcgctatacgattacgctaactgacggaactaaggcgccctcagattgggctgtaacgagtactgcgaatgtttatacggttactgacttaacggacgttgatacgtccaagtttggatcgagtgttggaacatacacattggcactctcaacggctggaattactaagctagctgaagctaatagtagcgcggatataacggctgctaacgtggtgacaggaacactaacaatcaagcaagctccggtaccgactgcgataattaccattggttcagctagtattgactatggggatgctaaaccaagtacgtatacaattacggtgccgagtcagtatgcagttcccagcacctggacgttagctagttcggctactgatggaacgactaatacttatatgattgcaagttctagtggcgatgttatagttcccacagcaacccaatctggaacgtatcagcttgtgttgtcagatcaaggcttgacagctttacaacaggctaatcctaatgctgctattactgctgatacgattattgctggtagtttagttattgcggcacatgacattattacgatgggtgcgacgacaattgtcgttaataaaacgactagtacggttccggtgacggtcaatagtcgtactattgtggttccaacaggttggacaattcgttacgatgatattcagactgatgcgattgtgtatgacgtccccgtttccgatacgacatattcggaagcggttaatactgctgtggttgataaatacaccattacattgactgatgatacgatagaaacattagctaaccttaacagcagtacgacttttaatagtacgacggttggtaagggcgtagtgcttgtcaaggctagtgccgcagttgccatctcacctgcaaactatggcgcgcaggctagtgccgaaactccggtaacagggctgacaatttcacatgcccgaacaaagggaattgatttagcatatggtcaggcgctgtatttgatcttgccgcttattaatatgaatccatcaggaatgactgtggctaatcttactgattatgttattattccatctggttttaaggttgctactaatagtgaaggagctattaacatagcgactgatccaagtagtgtgttaacgtctgctattgaagcaatgatgacgaaaaatgatgtgacctatcaggggttaaaggtgacccaactgacagactacaggggtcgccaaacatttaaaattcattttgataaaaccactgtttatgacggtggtgcatttgcaacgctaaaatatgcattattaccggtcattgctgttcaaaacactggggtgactagtggtttaattggtaatcaagtttcaagcccggattcggcggtggtttatgttactgatgattctaatgaaaataatggtagttattcgttgaatttgcaaaattatactaatattgacagtgtcgctgatgcattaggaattgcggatgctgtcacgattggtagtggtttcacaagttacctatatcattacacgctatcggccaaaacgattaccgatacttatagtttagtaggaaacgatggcacgtcattaggcgaagtaacttttacgggcgacagtggtaagacgtatgtaccgatgactaaattacccatgacaattacacaaaatggcgtgacgtattatttgaacactagtgcagtttcgttaactcagacatattctggtgatagtaattcaaattacacagttacttaccagcgctacgtcacaacgacgactgatactgcggccaagataacgattgcaccagcttcaaaagtctatgataacaacgccacgactgatccaagtcgctatacggtatacttgccaactgaatatacggccccaagcgattggactgctgatagcgcggcgacggctgtggatgggacgacggcgtaccaagtcagtaccgactaccttaacaccactgcaatcgatcaaaacgtgggcacttacgctgtcacgctgaatagcgccgggatggcagccttatccgctgctaatccagatttcttgattgcaggcgatgtgaatgttggtgggactctgacgattactcaacgtccagtgacgattactttgccggatacgattctgtgggccaatggtcaggaacaaaatattacgccggtcattactggtgttgttgcggtgcaaagtttggattacacgttaacgtcagggttaactgatccggacacgacaaccattacggccacgctgacgaatgccgctgctaatagtaattataaattgacgaattcacctagtggtcagttgacggtgggcgccgtaacggttgtctatcagtatgggtaccgcgacaaagcggggacgctacacgtggtaacaacggctaatggaacggcgacgcacgggactgatgttaccgctaaggactatttgagctacaccacgagtgatacgactgctacgcatgccaaaactggttatacgttacaaccagaaagtaccggttaccaagccgatggcactctagcggacgttggtgggcaggtcgtgtacacctatttagcgaacaccgaaaagattgcggtcgtttacgtcgaccaagataagaacaacgtgattttaaaacagattcccctcagtgggagctttggcacacccacgaattatacgacagcgcaggacattgcggcgtatgaaaaattaggctacgtgttagcttcggataaggtcccagcgccgcttgagtttgatcaggatactgaacagacctactacgtatacctgaaacatggcaccatcacggcgacggttgatcagccaggtaacgtggccgttagtgatttgatgaagaccagtcagcgaacgattcattacgtttatgctgataacacacccacggacttagcggatgtgcttcaaacggtcacgtatacgcgcacggcaacgggggatgcggtggatagaacggtcctttcgtacggtaattggacgaccaatgtgaatagctatccggccattgagtcgccgaccattactggttacacggcggatcaaacaaccatcgcggcggctgtacccgctagcatgggcgagactacggaaacaacggtccgatacagcgttaattctgaaacgatccgggttcaatttgtcgatggaactacggataaccaagtcttaagttatattgatttgaatgggaaatacggtgatgctgccgactatacggtcactgctgatatcgcgaagtatgcaaaattaggctatgaaccagttaactcggacttgcctgatcagctgatttataagcagaatacccaagtttatacggttacactagcgcatcgtcacgtgacggtcagcgttgatcatccgggccaacctggtcaggccatcgatgctgattatccagccggtcctaaatatccggcaggcactggtcgtgattcgttggaacaaacagtgactcggacgattacgtatcaatatgcgtcaggtgaatcagcggctgaaacggttaaccagtcggtcacgttcaatcgcacggcaactttcgacatggcaacgggtaagcagctgacttacggtgactggacagtggcacctggtcagtcagcactattggccgcggtcacgtcaccaacgattacaggttatcaagccagtgttacagaagtcgaagcagcgtcggtcactagtcacgataagccgcacttgattgcaatcacgtacacggccaaatcacagaccgcaaccgttgcgtttgtggatgtaacgagtggtaaaacactacctacgacggtagtaactggtgcttatggcactacgaatagttattcgcccgtttcccaaattgctgcgtatgaaaaactgggctatcgattagtttcgaataatgttccgacgactggtatcacctttgatcaaaatgacgtcattaagtcatacacggtcaagctagcgcatcaaatgacgacggtcacgccaactaagcctgggcaaccaggtcaaccagttgatcccgctcatccagaagggcccaagtacccagctggtactgggcttaaagatttaacaaccagcgttcagcgagtcattacctatgtttacaatgatggtcaaactgcggcgccaaccgtcacgcaaacggtcagttttgagcgcaaggcgacctttgatcaagtgacaaaggtggtgacgtatacggattggcgtacacctgaatcagcgttgacgggggcatacgcagtcgttgaatcgccaataattgctggctacaccccgaatgcaacccgtgttgctagtgtaactgtcagtgccaaagatactgagtcgcgacaaacggttacttaccaagcaaatctggaaacggcgacggtgacttatgtcgatgccacgacgggccaccgactgggtacaagcgtgacgttaaccggacgattcggtacgcaagcggattatcaaccaacgacaatgattgcgcagtatacccaggcaggctatgtcttgatggggagtgattatccggcaacgggtgttacttttaatcaggcgggcgtcgttcagaagtatacggtgtacttggctcataacaaaatcgtgattacggcaccagatcagctcaccaaaacgatcacgcaaacggttcactatcaggatcaggctgggcacacgcttcaagctgatacgatccgggcgctgacgttcacgcgttctgggatgaaagatgcggtgactggtgtggcaacgtatcgtgattgggcaccgaccgggttgaactttacagccgtgtctgcgccaacgattgcgaaataccatgcgttgacggcgaccactcaggccgtggcaatcacggctgctagtgctgatgatgtccaaacgctaacatatgcgctggacgtcccaacaccgacgaaaccggtcaaactgactaagccagccaaaccgactaagccgacaacatcggacgatttaatcaagccaacgacgaaaccaatcacggctgctaaaccaacgcaactcactaagccagcaacggttgtgaaggattttcaagccacaactggcaaccagacgccagctaaatcgacaaggacgttggtatcgagtcgcattaaggctgtcaaaacagctccggcatcagcaatcatcaagccgggaagtaaagtaacggagccggctcacaaggctcaagcagatacaacgagtcgattgccacagactggtgaaacgcggtggtctgaaatggctgctgaaacactagggctaacactagcaacattattgctgggctttggtggcttgaagcgtaagcggcatgaaaagtaa

SEQ ID NO: 110 - protein, 2032 aa, Lactobacillus plantarum
MQRRRLQRAQLTEKRTYKMYKKGRLWLIAGLSTFTLGASLLPMTGRADTTSTPAEKQGTRTETTGNQITLASKSVGSSSMANDGEEKTNNSQVETSSEASNVTASTEAKSTESTTQTVVDSTVTSTATETTRANGATNQTSKMSIVDTTSNNTEQNQAVGGTTDSTASTATIEDQAKAANRATTDGKINTATVATKTTTTASYATADISTNTIRSAQKLARATVATVATVNSATKTYDGKIDTPNRYTITLTDGTKAPSDWAVTSTANVYTVTDLTDVDTSKFGSSVGTYTLALSTAGITKLAEANSSADITAANVVTGTLTIKQAPVPTAIITIGSASIDYGDAKPSTYTITVPSQYAVPSTWTLASSATDGTTNTYMIASSSGDVIVPTATQSGTYQLVLSDQGLTALQQANPNAAITADTIIAGSLVIAAHDIITMGATTIVVNKTTSTVPVTVNSRTIVVPTGWTIRYDDIQTDAIVYDVPVSDTTYSEAVNTAVVDKYTITLTDDTIETLANLNSSTTFNSTTVGKGVVLVKASAAVAISPANYGAQASAETPVTGLTISHARTKGIDLAYGQALYLILPLINMNPSGMTVANLTDYVIIPSGFKVATNSEGAINIATDPSSVLTSAIEAMMTKNDVTYQGLKVTQLTDYRGRQTFKIHFDKTTVYDGGAFATLKYALLPVIAVQNTGVTSGLIGNQVSSPDSAVVYVTDDSNENNGSYSLNLQNYTNIDSVADALGIADAVTIGSGFTSYLYHYTLSAKTITDTYSLVGNDGTSLGEVTFTGDSGKTYVPMTKLPMTITQNGVTYYLNTSAVSLTQTYSGDSNSNYTVTYQRYVTTTTDTAAKITIAPASKVYDNNATTDPSRYTVYLPTEYTAPSDWTADSAATAVDGTTAYQVSTDYLNTTAIDQNVGTYAVTLNSAGMAALSAANPDFLIAGDVNVGGTLTITQRPVTITLPDTILWANGQEQNITPVITGVVAVQSLDYTLTSGLTDPDTTTITATLTNAAANSNYKLTNSPSGQLTVGAVTVVYQYGYRDKAGTLHVVTTANGTATHGTDVTAKDYLSYTTSDTTATHAKTGYTLQPESTGYQADGTLADVGGQVVYTYLANTEKIAVVYVDQDKNNVILKQIPLSGSFGTPTNYTTAQDIAAYEKLGYVLASDKVPAPLEFDQDTEQTYYVYLKHGTITATVDQPGNVAVSDLMKTSQRTIHYVYADNTPTDLADVLQTVTYTRTATGDAVDRTVLSYGNWTTNVNSYPAIESPTITGYTADQTTIAAAVPASMGETTETTVRYSVNSETIRVQFVDGTTDNQVLSYIDLNGKYGDAADYTVTADIAKYAKLGYEPVNSDLPDQLIYKQNTQVYTVTLAHRHVTVSVDHPGQPGQAIDADYPAGPKYPAGTGRDSLEQTVTRTITYQYASGESAAETVNQSVTFNRTATFDMATGKQLTYGDWTVAPGQSALLAAVTSPTITGYQASVTEVEAASVTSHDKPHLIAITYTAKSQTATVAFVDVTSGKTLPTTVVTGAYGTTNSYSPVSQIAAYEKLGYRLVSNNVPTTGITFDQNDVIKSYTVKLAHQMTTVTPTKPGQPGQPVDPAHPEGPKYPAGTGLKDLTTSVQRVITYVYNDGQTAAPTVTQTVSFERKATFDQVTKVVTYTDWRTPESALTGAYAVVESPIIAGYTPNATRVASVTVSAKDTESRQTVTYQANLETATVTYVDATTGHRLGTSVTLTGRFGTQADYQPTTMIAQYTQAGYVLMGSDYPATGVTFNQAGVVQKYTVYLAHNKIVITAPDQLTKTITQTVHYQDQAGHTLQADTIRALTFTRSGMKDAVTGVATYRDWAPTGLNFTAVSAPTIAKYHALTATTQAVAITAASADDVQTLTYALDVPTPTKPVKLTKPAKPTKPTTSDDLIKPTTKPITAAKPTQLTKPATVVKDFQATTGNQTPAKSTRTLVSSRIKAVKTAPASAIIKPGSKVTEPAHKAQADTTSRLPQTGETRWSEMAAETLGLTLATLLLGFGGLKRKRHEK

SEQ ID NO: 111 - DNA, 915 bp, Lactobacillus plantarum
atggatttaaagcaaagcgatggttggcgatacttagctgggtggagcttcattctattaatggtggcgagtgccacattgcaacatgatgcgaaaatcattttacccgaaatcggtgctctgacagccgggacgtgggtttatcgtaagacggcgtggactcggcaacccttaaagttattcttagtaccatctggaactgcaattattggcttcttagtcaatcaactaccttggtcgcacgccctcaaagtgcttgtcggtctattactgatgctattattattgaaggggttaaaatcgaatttggcgccagcctttgctactggcttactgccaattatcattaatgcaacgcactggacctttatcgtagccatctttttctggactatttgcctgatgattggggcttggattcaacgaccgcgatcaatctcacgggtaaccgaagcttctgctagtcgctggcaaatgctcggctttatcagcctagtttttgtctgggtgggtattgtttggctagcgggacagccccagatggccgcaatcccacccgtgatcgtcgttttctttgaagcggctcaacagtctgaatatacggtaacgaccgcacttaagcagtggcttgcattgtcggctgctgctagtattggggtcggcattcacctattgattgcttcgtggctattaacgacggtcattgccttaccacttgtgtatttgtggttacgggcgcttaacttacaattgccagcagcgtatgcctttccactattagccttagtgttaccagccaatatgtttaacaaactaccgacatccgccggcttagcggccgctttcttcctaggatcgttactcatctaccatcagatcttgggttgggtacgcatggcggtgactgaaagctag

SEQ ID NO: 112 - protein, 304 aa, Lactobacillus plantarum
MDLKQSDGWRYLAGWSFILLMVASATLQHDAKIILPEIGALTAGTWVYRKTAWTRQPLKLFLVPSGTAIIGFLVNQLPWSHALKVLVGLLLMLLLLKGLKSNLAPAFATGLLPIIINATHWTFIVAIFFWTICLMIGAWIQRPRSISRVTEASASRWQMLGFISLVFVWVGIVWLAGQPQMAAIPPVIVVFFEAAQQSEYTVTTALKQWLALSAAASIGVGIHLLIASWLLTTVIALPLVYLWLRALNLQLPAAYAFPLLALVLPANMFNKLPTSAGLAAAFFLGSLLIYHQILGWVRMAVTES

SEQ ID NO: 113 - DNA, 930 bp, Lactobacillus plantarum
atgaagtatcggttaataggggtaggtgcgagtctagtcgtcgcagtcatgttaacagggtgtcaagcgaaggctacgacattggtcaagtcagatgctggccaggtcacacaagcggaagtatttaaacaaattgaaaaccaagcgacgacgcaacaggctgttcaagaattgactctaaacaaagtccttaatcaacgatatcatgtttcacaagctgaagtaactgctaaattaaaagcattcaaacggcaggcgggcgcaaattatcacatgattttagaacgtaatcatgtcactgaaccgcgtttaaaatcgcaaatcaaagcgaatttattgatggagaaagccgttagtgctaagtatccagtgactaaagcgcaactaaaaaaagcccgagcagcttatatgccaatgacaacggttcaacacattgcgacgaccaatgagaagcaagcgcaaaaaattattgctgaactgaatgcgggtgctagctttgattcgcaagtgcgaaaatatcagaataatcgacaagcgcacacaactgctgggaaattagcgcagtttgacagttataatcaaactctagcaccagcaattgtacaggccacagctaaactacgagtgggacactatgtcacgaaaccggtcaaaacagttatggcaactgccgacacgaaagacaaaccaacttatgaaattatcaacgttgtcagtcgtcgatctaagactgcggctgtaactgatgatagcggtaagcagattgatgtgacaaattatttgcgtgaaaaaatccagcaacagcggatgatggacaagcagacgcaagttgcgacgattcggtcagttttcaaagcggcccacgttaaggtagttgatgcccatttcgcaccagcgtttaatgattatttaaccacccaaaatagctaa

SEQ ID NO: 114 - protein, 309 aa, Lactobacillus plantarum
MKYRLIGVGASLVVAVMLTGCQAKATTLVKSDAGQVTQAEVFKQIENQATTQQAVQELTLNKVLNQRYHVSQAEVTAKLKAFKRQAGANYHMILERNHVTEPRLKSQIKANLLMEKAVSAKYPVTKAQLKKARAAYMPMTTVQHIATTNEKQAQKIIAELNAGASFDSQVRKYQNNRQAHTTAGKLAQFDSYNQTLAPAIVQATAKLRVGHYVTKPVKTVMATADTKDKPTYEIINVVSRRSKTAAVTDDSGKQIDVTNYLREKIQQQRMMDKQTQVATIRSVFKAAHVKVVDAHFAPAFNDYLTTQNS

SEQ ID NO: 115 - DNA, 1410 bp, Lactobacillus plantarum
atggcaatgttatacggtaaacacaatcatgaagctgaagaatacttggaaccagtctttggtgcgccttctgaacaacatgatcttcctaagtatcggttaccaaagcattcattatcccctcgagaagccgatcgcttagttcgtgatgaattattagatgaaggcaattcacgactgaacctggcaactttttgtcagacctatatggaacccgaagccgttgaattgatgaaggatacgctggctaagaatgccatcgacaaatctgagtacccccgcacggccgagattgaaaatcggtgtgtgaacattattgccaatctgtggcacgcacctgatgacgaacactttacgggtacctctacgattggctcctctgaagcttgtatgttaggcggtttagcaatgaaattcgcctggcgtaaacgcgctcaagcggcaggtttagatctgaatgcccatcgacctaacctcgttatttcggctggctatcaagtttgctgggaaaagttttgtgtctactgggacgttgacatgcacgtggtcccaatggatgagcaacacatggcccttgacgttaaccacgtcttagactacgtggacgaatacacaattggtatcgtcggtatcatgggcatcacttataccggtcaatatgacgacctagccgcactcgataaggtcgttactcactacaatcatcagcatcccaaattaccagtctacattcacgtcgacgcagcgtcaggtggcttctataccccatttattgagccgcaactcatctgggacttccggttggctaacgtcgtttcgatcaacgcctccgggcacaagtacggtttagtttatcccggggtcggctgggtcgtttggcgtgatcgtcagtttttaccgccagaattagtcttcaaagttagttatttaggtggggagttgccgacaatggcgatcaacttctcacatagtgcagcccagctcattggacaatactataatttcattcgctttggtatggacggttaccgcgagattcaaacaaagactcacgatgttgcccgctacctggcagccgctctggataaagttggtgagtttaagatgatcaataacggacaccaactccccctgatttgttaccaactagcctcgcgcgaagatcgtgaatggaccctttatgatttatcggatcgcctattaatgaacggttggcaagtaccaacgtatcctttacctgctaatctggaacaacaagtcatccaacgaatcgtcgttcgggctgactttggcatgaatatggcccacgatttcatggatgacctgaccaaggctgtccatgacttaaaccacgcccacattgtctatcatcatgacgcggcacctaagaaatacggattcacacactga

SEQ ID NO: 116 - protein, 469 aa, Lactobacillus plantarum
MAMLYGKHNHEAEEYLEPVFGAPSEQHDLPKYRLPKHSLSPREADRLVRDELLDEGNSRLNLATFCQTYMEPEAVELMKDTLAKNAIDKSEYPRTAEIENRCVNIIANLWHAPDDEHFTGTSTIGSSEACMLGGLAMKFAWRKRAQAAGLDLNAHRPNLVISAGYQVCWEKFCVYWDVDMHVVPMDEQHMALDVNHVLDYVDEYTIGIVGIMGITYTGQYDDLAALDKVVTHYNHQHPKLPVYIHVDAASGGFYTPFIEPQLIWDFRLANVVSINASGHKYGLVYPGVGWVVWRDRQFLPPELVFKVSYLGGELPTMAINFSHSAAQLIGQYYNFIRFGMDGYREIQTKTHDVARYLAAALDKVGEFKMINNGHQLPLICYQLASREDREWTLYDLSDRLLMNGWQVPTYPLPANLEQQVIQRIVVRADFGMNMAHDFMDDLTKAVHDLNHAHIVYHHDAAPKKYGFTH

SEQ ID NO: 117 - DNA, 1113 bp, Lactobacillus plantarum
ttacatgtggatagcaaagcttggtgtgtagtaagctgtgctcttaattgaaacagtttcaccataagttggtgaagcgatgtattggccaccgcctaagtaaatcccaacgtggtaaggtgctgaatcagaaccccagaaaatcaagtcgccagcttgggcttgactgaatgatacgtgagtaccaatcgtagcttgagcatatgtggtccgaccgattgaacgaccaaccttagcaaaggcagcttgcgtgaatgcagagcagtccatttgttcgtaaggtgtgccaatgaaggtcttagcagcagcaattactgaactgtatgatgcagttgaactagtcgttgatgtactagttgatgctgaagcactgtaggccgttgcagtagcagtcgttgttgatgaactagtcgtgttacttgcagttgagctagcttgtgagctcgttgaagtagcagctgattgcgtactagttgcagtcgtgctagtagacgatgctactggagttgagctttgtgatgcttgtgaagctgaactttggctagctgagctttgtgaagttgctgaactagttacagcacttgagctagcttgtgaagctactgaactcgttgaagcacttgaagtgacactgcttgcactagcagttgaagtgctacttgattgagcagctactgagctacttgatactgcttggctcgtactacttgcactagtagttgatgagctggtagcagcagcgctagtagcttgtgaactagttgaagttgcggcactagttgacttagcactgcttgcactagcagtggtcgtcttagtcgtaaccttttcaccagttttaacgccaggaatattgatggttttaccagcaataattaagttagggttgcttaagccattagcttcagcaatcttgtcaacagaaacatggtacttttgtgcgtatgcccaaacagtatcccctgctttgattgtcattgaatcggcattggcaactgcttgactagtaactaacataccagctaaagcagcagttcctaacatcacttgtttaatatttactttcactttaataatgatcactccaaaaagaatgtcacgtatgtacttcaa

SEQ ID NO: 118 - protein, 370 aa, Lactobacillus plantarum
MKYIRDILFGVIIIKVKVNIKQVMLGTAALAGMLVTSQAVANADSMTIKAGDTVWAYAQKYHVSVDKIAEANGLSNPNLIIAGKTINIPGVKTGEKVTTKTTTASASSAKSTSAATSTSSQATSAAATSSSTTSASSTSQAVSSSSVAAQSSSTSTASASSVTSSASTSSVASQASSSAVTSSATSQSSASQSSASQASQSSTPVASSTSTTATSTQSAATSTSSQASSTASNTTSSSTTTATATAYSASASTSTSTTSSTASYSSVIAAAKTFIGTPYEQMDCSAFTQAAFAKVGRSIGRTTYAQATIGTHVSFSQAQAGDLIFWGSDSAPYHVGIYLGGGQYIASPTYGETVSIKSTAYYTPSFAIHM

SEQ ID NO: 119 - DNA, 345 bp, Lactobacillus plantarum
ctatttagatattcgttggcgatgatgcgttcgcggtaatcctaatagccatcgaatatcattacccgtaaagttcaacttcaagaattcgccactttgtccctgttgatacatggtcggtaatacctggtcttgcaacttgatcacttgctgtgcgatctcttcgtcaaataaatattgccgtgtcgcggcagcaatttcaccgaccgtaaaaatctgagcagcgttagcaaatatctgctctttgctcagcgccaactgggcttgtttaaactgggcgtaggactgatccaagacttggcctgcttgagtttccaatgcggttactgtggtcatgatgcgcac

SEQ ID NO: 120 - protein, 114 aa, Lactobacillus plantarum
MRIMTTVTALETQAGQVLDQSYAQFKQAQLALSKEQIFANAAQIFTVGEIAAATRQYLFDEEIAQQVIKLQDQVLPTMYQQGQSGEFLKLNFTGNDIRWLLGLPRTHHRQRISK

SEQ ID NO: 121 - DNA, 372 bp, Lactobacillus plantarum
ttgacgaatacagacaatcgttattatcaaccaaccgacatcaaagatgcgcttcaaacaatccaaaaattatttaatacttataccgatgccccattaacacccgaattaatggcctaccatcaaaaattagttaatcagttagctactaatttattaccactagcacaacaacaacatgacaaattacggatcacccaaattaattcaatgatggccgttatgcaagattggctaaaattaaggctgaatggtcaagtcttcggcggcaaaatgcaacacttcaagtttgtcagcaaccaaaaagcacagtacaaacgacgagtgcataaaattcgtggcaatcaaaatcatcgtgctagtcgccattga

SEQ ID NO: 122 - protein, 123 aa, Lactobacillus plantarum
MTNTDNRYYQPTDIKDALQTIQKLFNTYTDAPLTPELMAYHQKLVNQLATNLLPLAQQQHDKLRITQINSMMAVMQDWLKLRLNGQVFGGKMQHFKFVSNQKAQYKRRVHKIRGNQNHRASRH

SEQ ID NO: 123 - DNA, 960 bp, Lactobacillus plantarum
atggcttacacaaataatcaactacacgttatttacggcgacgggagtttaggactacagggggctaatttccactacctctttagctacgaacgtggcggacttgaatcactcgtcgtcaacgataaagagtggctctatcgtacacccacgcccatcttttggcgggcgacaaccgataatgatcacggtagcggcttttcagtcaaatccgcacagtggtacgcggccgataagttctcaacttgtcaagatatcgaattgacggttgacgaccaaccagtcacaccgttaccaatcgcgccactcaataacaaatacacggatcacgaaatcgccacgaaagtctcactggcttaccacttcgttaccacgaccgttcctagtaccatcgtcacagtgacttatacggtgacagcagacggtcagatcaatatcgccacccattatagcggtcagtctgatttgccagagctacccgcatttggtctgcggtttatcataccaactaccgcgaccggcttcgactataccggtttgtccggtgagacttatcctgaccggctggccggcgcaacgcacgggcgattccacgttgacagtctgccagtcacaccatacttggtcccacaagaatgcggcatgcacatgcaaactgaacaagtgacagtaacgcgatcaacaacacaaaataacgctgaccacgacaacacaccgttcagtttgacatttagccaagccgatgcaccattcgccttcagctgccttccctataccgccgctgaactagaaaacgcaacgcacatggaagaattaccattagcacggcgaacggtcttatcaatctacggtgccgttcgtggggtcggtggcattgatagttggggaacagacgtagaatccccatatcatatccccgctgatcaagacattgacttcagctttaatattcatttctaa

SEQ ID NO: 124 - protein, 319 aa, Lactobacillus plantarum
MAYTNNQLHVIYGDGSLGLQGANFHYLFSYERGGLESLVVNDKEWLYRTPTPIFWRATTDNDHGSGFSVKSAQWYAADKFSTCQDIELTVDDQPVTPLPIAPLNNKYTDHEIATKVSLAYHFVTTTVPSTIVTVTYTVTADGQINIATHYSGQSDLPELPAFGLRFIIPTTATGFDYTGLSGETYPDRLAGATHGRFHVDSLPVTPYLVPQECGMHMQTEQVTVTRSTTQNNADHDNTPFSLTFSQADAPFAFSCLPYTAAELENATHMEELPLARRTVLSIYGAVRGVGGIDSWGTDVESPYHIPADQDIDFSFNIHF

SEQ ID NO: 125 - DNA, 924 bp, Lactobacillus plantarum
atgaggaggtctaaaatgacatatttaattggcgttgactgtggtggcacgcacatcgttggtcaaacttggacgacagcccccgagcatctagtccaaagcgttacgggtggccctggtaacgttgtcctagactactctgctgccgttactaacttaaccactgtcttagaccagctcactgccgcaattccagctagtcagcttgggttgattttaatcggaattgctggcattgaaactgctggccgggctgatcaggtccaacaaaccatcacccaacgttaccacgctaatacccaggtcataagcgatgcaaaactggccctactgaacggtcttgcaggagcagacggcgccttagtgattgccggcacgggctcggtcgtttatggccgccaagccggaaaatttctgcgcgttggcggctggggttacgttttaggtgacgaaggcagtgcctatgacattagcaagcgggcacttaaacaggttctgacccagactgataacggtcaaactagtcaactaacagctcccctattggcacaacttaaagttaccgatattgctgccgccgtccagaaattttacgctcaagatcgacaaactaacgctcaattagcacagttaatcgccaaactggccgagcaacaaaattctgaagccatcacggtattagtcacgtcagcccaagcactggcacaacaagtcgttaccttatatcagcggtttgcagagtcctggccacaacgggtcgccctctctggttccgttttacaacacaatcgcctggtccgcgacacgttaacgacgacagtgcaccagtcaataccaacaattgcttttaacgatattacaactaacaacgcccacgccgtcatctattggcaccggtggactcaggaggaaattaattcatga

SEQ ID NO: 126 - protein, 307 aa, Lactobacillus plantarum
MRRSKMTYLIGVDCGGTHIVGQTWTTAPEHLVQSVTGGPGNVVLDYSAAVTNLTTVLDQLTAAIPASQLGLILIGIAGIETAGRADQVQQTITQRYHANTQVISDAKLALLNGLAGADGALVIAGTGSVVYGRQAGKFLRVGGWGYVLGDEGSAYDISKRALKQVLTQTDNGQTSQLTAPLLAQLKVTDIAAAVQKFYAQDRQTNAQLAQLIAKLAEQQNSEAITVLVTSAQALAQQVVTLYQRFAESWPQRVALSGSVLQHNRLVRDTLTTTVHQSIPTIAFNDITTNNAHAVIYWHRWTQEEINS

SEQ ID NO: 127 - DNA, 438 bp, Lactobacillus plantarum
atgattaggttatatacacaatcaagctgtcattcttctagagttgcacggcaatggttggaagcacatgggattgagttcaaggagaaaaattttagtgttgattcgcccacggtgcaagatctaaaacgtattttgagtttaaccgaacatggtgtagacgatattatctcagctcgatctaaagactatcctgaaattgcgcctaagttacccgaaatgccattgaatgaggcacttaaattgttgtgtgatcatccgaagttgttacgtcggcctatcatcattagtgatagtaaaattcaagttggctttaatgaagatgatattcgccaatttattccacgaccagttcggcgactaaagttcaatgcattgttgagtcgtctagatggtaatacaggggatcacattattaataaaaggatggtcgagtag

SEQ ID NO: 128 - protein, 145 aa, Lactobacillus plantarum
MIRLYTQSSCHSSRVARQWLEAHGIEFKEKNFSVDSPTVQDLKRILSLTEHGVDDIISARSKDYPEIAPKLPEMPLNEALKLLCDHPKLLRRPIIISDSKIQVGFNEDDIRQFIPRPVRRLKFNALLSRLDGNTGDHIINKRMVE

SEQ ID NO: 129 - DNA, 912 bp, Lactobacillus plantarum
gatcttcgcgatttaatggccattcctgacaactatcacgtgctcttctttcaaggcgggggcacgctacagttcacagctgcgccactaaatctggcgcctcatcatcgtatcgggttgcttgacagcggtcactgggcacaacgcgccgccgatgaagctaaacgggtcggtactaaagtcacgatactggggagtagcgctgccaaccattttaaccaactgccaacggtcgtccagcccatcgatcaatccctcgattatattcatcttacaactaataatactattgaaggaaccatgatgacgcgcctgccagttacgggtcaagtaccactggtagccgacatgtcatcaaactttttaggtgaaccttaccaagtcagcgattttgggctcatctttgctggtgctcagaagaatctgggtcccgctggtttgacaatcgtcattgtccgtgatgatttaattggtcaagtcgccaacctgccaagcatgctggattaccagctattcgcggctaaagattcgatgttcaacacgccgcctgtttttgctatttacgccgcgggtctcgtactcaagtggctaaaggcccaaggcgggctcagcacaatgactgctcgcaatcacgctaaagccgccttactctatgatttcttagaccagtcacaactatttactaatccagtcaagaccagcgaccgttcgaccatgaacgttccattcgtcacaggtcaggccgacctcgatgccgcagtcattcaaggcgcccgtgagcacgggttattaaacctaaagggtcaccgcttagttggcggtatgcgtgccagcctctataacgccatgccgttagccggtgttcaggcattagttgactatctagccgcttttgaagcacaccatcgttaa

SEQ ID NO: 130 - protein, 304 aa, Lactobacillus plantarum
QDLRDLMAIPDNYHVLFFQGGGTLQFTAAPLNLAPHHRIGLLDSGHWAQRAADEAKRVGTKVTILGSSAANHFNQLPTVVQPIDQSLDYIHLTTNNTIEGTMMTRLPVTGQVPLVADMSSNFLGEPYQVSDFGLIFAGAQKNLGPAGLTIVIVRDDLIGQVANLPSMLDYQLFAAKDSMFNTPPVFAIYAAGLVLKWLKAQGGLSTMTARNHAKAALLYDFLDQSQLFTNPVKTSDRSTMNVPFVTGQADLDAAVIQGAREHGLLNLKGHRLVGGMRASLYNAMPLAGVQALVDYLAAFEAHHR

SEQ ID NO: 131 - DNA, 380 bp, Lactobacillus plantarum
ggatcgaagggaccattacgccggctagtggcacaattgatcgcccgattggccgggtggctgacagtcctcggcgagtggtcaccacggcgggccaacgcgccattacgacgtatcaagtggaggcggaccaattgcagcataacgtgagtcggttacggttggaacttgtgactggacggacgcatcaaattcgggtccatctaacgacgcttgggcaccccttattaggtgatgcgctgtatggcggtaacttggggtggattcaacggcaagccttacacgccgctagtttacagttctttgaccccttttcggaacagactttacactttgaggcggcattgccagctgatctgcaagccttgaatcacgactaa

SEQ ID NO: 132 - protein, 126 aa, Lactobacillus plantarum
WIEGTITPASGTIDRPIGRVADSPRRVVTTAGQRAITTYQVEADQLQHNVSRLRLELVTGRTHQIRVHLTTLGHPLLGDALYGGNLGWIQRQALHAASLQFFDPFSEQTLHFEAALPADLQALNHD

SEQ ID NO: 133 - DNA, 372 bp, Lactobacillus plantarum
atgcaagtttttggacaatttattgcaacagtcggttggctaggattggcactagtcgccagcgaactaggtgcgacgttaatccattggctcggtcagtgggtcggatttcgattaattggtgctcgaattgtccggattaccggttttcgacttcaattaagtcgggttcgtggtcattggaaattagaacgaccgctgacgcgtcatccacatatcgtggcagcaccctcggcggatgccaaacggttcaatcacgccatttattgttttggcggtggcctgttcaacttactgacggtcatgctcagtttaataactctgaatcaatttaagtttagtttcgatttatggttgtttgcgttcattatt

SEQ ID NO: 134 - protein, 124 aa, Lactobacillus plantarum
MQVFGQFIATVGWLGLALVASELGATLIHWLGQWVGFRLIGARIVRITGFRLQLSRVRGHWKLERPLTRHPHIVAAPSADAKRFNHAIYCFGGGLFNLLTVMLSLITLNQFKFSFDLWLFAFII

SEQ ID NO: 135 - DNA, 894 bp, Lactobacillus plantarum
gatccattgtcgactttgttgccgcgccgtaatcaaacagtgcatctaaagtatcgttctgcacagacgacggcggagctacgcaagacgctacgtcaagcacggtatttacaggccggtactcagaataccgccacgccggtctttcaaaatcgacagcagcgaggtgatgcgacaacgtacggtcgtatcagtaccagccaagacggccggatatggacgaaactacccattagttatccgcatgtgcaattgtcacggccgagtgtctggtacgcgaatggccgcttgacgttgatagatgggaaagaccgttactggacgactaattttaaagattggcaacatcaacggttgaactttaacggggctgattttaagcaaggtcgggttcaggccgtctttccaggtacgactcgttcagcggttgttgtggttcgcggcattgatcgccaaagcagtcgcgccaaactctattatggacagctcacgaagactggacgggtcaaagcttggcacgcgttacaactaggaaagctcccagcgcgccaagtcgctggaatgagcttgattgatcaacacttatacctgtttcttcagcgcggtacgcagttggccatttatcgtgccaatcggttgacgcgtccggtcaggttggttggtcgcgttaagctaaatcatgcgcagtcacaacgagtgaccgcggtgaatttgataccgaccaccaagcatcgctaccggttaatatttgacttgacgacagctgaaaaagttcagaaacagccacgttatcggttacttgatcggcgatttaaagcagtggggcagcagcatctattggtcactgattatctctggagccaatttcaaattagtctacgtgggagtgagtga

SEQ ID NO: 136 - protein, 297 aa, Lactobacillus plantarum
DPLSTLLPRRNQTVHLKYRSAQTTAELRKTLRQARYLQAGTQNTATPVFQNRQQRGDATTYGRISTSQDGRIWTKLPISYPHVQLSRPSVWYANGRLTLIDGKDRYWTTNFKDWQHQRLNFNGADFKQGRVQAVFPGTTRSAVVVVRGIDRQSSRAKLYYGQLTKTGRVKAWHALQLGKLPARQVAGMSLIDQHLYLFLQRGTQLAIYRANRLTRPVRLVGRVKLNHAQSQRVTAVNLIPTTKHRYRLIFDLTTAEKVQKQPRYRLLDRRFKAVGQQHLLVTDYLWSQFQISLRGSE

SEQ ID NO: 137 - DNA, 330 bp, Lactobacillus plantarum
atgatgagacgtaggggagcaagtatgcagcagcaccgtaatgtgctctatctgattatcttcggaatctacttagcctcagtcacactacagacgacgacctttaacgagatgataccgcatcgagtgggcgttttgattgaattagcgactttggccgcattactgggcctcgtggtttgcttagataccttgacccccggccaaattattggagaagtcagtttacttgtactggtgactgtcgtgacactcacatcgggtgcgcattatttgatgccgacaatcatgttggtgattgcagcccgggaagtttcgtttcggcagatc

SEQ ID NO: 138 - protein, 110 aa, Lactobacillus plantarum
MMRRRGASMQQHRNVLYLIIFGIYLASVTLQTTTFNEMIPHRVGVLIELATLAALLGLVVCLDTLTPGQIIGEVSLLVLVTVVTLTSGAHYLMPTIMLVIAAREVSFRQI

SEQ ID NO: 139 - DNA, 1033 bp, Lactobacillus plantarum
atgagtaatcatcaaatccgcttgtccttatcaatcatcaccagttgcttgttggcaactctgattatcggcccgttagtcgccctgattggtcaaacactagtcgggcaatcgccaagccagctatggtcacaactgacgcagccaaccaaccgtgtgagcattcaacacagtctgttcctcagtgggggcacggtcgtcgggacaaccctgctagccacccctttggcatggatcatgacgcacacccgtttaacaaagctcgcctggttgcattggctcttgttagtgccattcatgacaccaccatatattaacgcgatgggctggttatatttctttcaaccacacggattactggctcagcttaatccgagttggcaccaccaatttcagtggctattttcaccgttcgggatggtcattatcatgagtctgcatttgtatcccgtggcatacttaggcttacgcgcagccctcatgcaattcaaccagcgctggcttcaagcggccgaagttcatggggtcaacacctggcaacgactagtgcgaatcacattaccaatcatgttagtcccatacttagctgtatggattttagtctttaccaaaaccttggctgaatttggaacgccagccacctttggtcggagcatccacttcgaagttctgacgactacgattcaaagggacctcagtcagtggcccttagatttccaaaacggggtactcaccggcaccctcctactgaccattgccctgattgcctggggtatccagcaatggttgttacgccggccagctgttaagttcaccggacaacggtcagcgtcacaatatcggcagcttggagtgacaacattagcaggcactttcgtcaccctagtcatcagtattgctattgtcctgccattcagtgccatcgtgctccaatcgctactcaaacaacgcagtcttggttggagtccgtctaatttgacacttgtacactatatagacctcttacgctttgatagtcctgcctggcaggcca

SEQ ID NO: 140 - protein, 344 aa, Lactobacillus plantarum
MSNHQIRLSLSIITSCLLATLIIGPLVALIGQTLVGQSPSQLWSQLTQPTNRVSIQHSLFLSGGTVVGTTLLATPLAWIMTHTRLTKLAWLHWLLLVPFMTPPYINAMGWLYFFQPHGLLAQLNPSWHHQFQWLFSPFGMVIIMSLHLYPVAYLGLRAALMQFNQRWLQAAEVHGVNTWQRLVRITLPIMLVPYLAVWILVFTKTLAEFGTPATFGRSIHFEVLTTTIQRDLSQWPLDFQNGVLTGTLLLTIALIAWGIQQWLLRRPAVKFTGQRSASQYRQLGVTTLAGTFVTLVISIAIVLPFSAIVLQSLLKQRSLGWSPSNLTLVHYIDLLRFDSPAWQA

SEQ ID NO: 141 - DNA, 838 bp, Lactobacillus plantarum
cggttttgaagccaaaactggcattaaagtcaaaagttttgacggcacgaccgggaaaattttaagtaaggtcaaggccgagcaaggcaatccccaagctgatgtgctgattttagcttcaatggccgctggcgtcgatttacaaaagaatggccagctattaacctatcagccttctcaagctaaacacctgaataaacaatttaaagatactagccaccagttgatcaattacagtgcttcggcagtcggcatcacctacaatacgcggcacatcaaatcggcaccgacagactggtctgacttgacaaccgctccgtatcgcaatcaagtgaccattccggacccccaaacctctggttctagcttggacttcattaacgcttatcaaatgaaacacggtacgcaactacttaaagcccttcaagaaaacggtgccgatatcgggggtgctaacaaggaagtactcgatgcagtcatcactggccaaaaaatcgccgtctttggtggggtcgattacatgagtctaacagctattaaaaaaggcgaaaaaattggtttcgtttatcctaagagtgggactttggtcaatccacgaccggcgatgattttgaaggctagtcgtcatcaagccgccgccaaacaatttattgactatctcttatcagctaaagttcaaagacagattcaaaaaagtaacttaattccaggtaccacgagcactttgaccgatccacgcaatggcgaagccatcaaagcctacacggtcaattggaccagtgccaacgcggccctgaccaaaaacgttgtcgcattcaatcaggtctttagccaatga

SEQ ID NO: 142 - protein, 278 aa, Lactobacillus plantarum
GFEAKTGIKVKSFDGTTGKILSKVKAEQGNPQADVLILASMAAGVDLQKNGQLLTYQPSQAKHLNKQFKDTSHQLINYSASAVGITYNTRHIKSAPTDWSDLTTAPYRNQVTIPDPQTSGSSLDFINAYQMKHGTQLLKALQENGADIGGANKEVLDAVITGQKIAVFGGVDYMSLTAIKKGEKIGFVYPKSGTLVNPRPAMILKASRHQAAAKQFIDYLLSAKVQRQIQKSNLIPGTTSTLTDPRNGEAIKAYTVNWTSANAALTKNVVAFNQVFSQ

SEQ ID NO: 143 - DNA, 166 bp, Lactobacillus plantarum
gatcgcacgtaatgggcaagtcaccgtggcttttgatgcgcagcacgatgccatcatggaattcgatttaccagtcaattaccaacgggagtttcccgagacggtggcagtcttagacgatggtcagtatcagaccatgatgttgatggacgaactctccgtctga

SEQ ID NO: 144 - protein, 54 aa, Lactobacillus plantarum
IARNGQVTVAFDAQHDAIMEFDLPVNYQREFPETVAVLDDGQYQTMMLMDELSV

SEQ ID NO: 145 - DNA, 504 bp, Lactobacillus plantarum
gtgtgtctaatggcgaaaacagcagtgtgcattgtcgatcaacaacgttaccaagttgtggacggtatgcgattagaagaattggaaactagtttgcggcaaatgattttaaaagattttccgcaggcccataatagcagtttcatttgtagtgagcatctcgtacattatcgcttagcaaagatggatgcgatgatcgagaacgattatcaacaaaatgataaggtcaatgcgcaattatctaagattctcgctaaccacacgtatcgggtcgtcgatgttaatagcgagctggaaagttcattgacatttggtcaacgggtcgcggatggggtcgcacggttcggggggagctgggcgtttatcatttcgtttgtcgtggtgatgctcgtgtggatgttgctcaacgtcttaccaatttttagccatcattttgacccttatccctttattttattaaatttatttttaagcatggtcgcagcaatccaggcaccattgatc

SEQ ID NO: 146 - protein, 168 aa, Lactobacillus plantarum
VCLMAKTAVCIVDQQRYQVVDGMRLEELETSLRQMILKDFPQAHNSSFICSEHLVHYRLAKMDAMIENDYQQNDKVNAQLSKILANHTYRVVDVNSELESSLTFGQRVADGVARFGGSWAFIISFVVVMLVWMLLNVLPIFSHHFDPYPFILLNLFLSMVAAIQAPLI

SEQ ID NO: 147 - DNA, 1965 bp, Lactobacillus plantarum
gatcaagtcagctatcagcgtcgtcgcgacttagctcgtaagtatgagttacagcgattaatgacgactggtagtcacgtgaatatgagcttgaatgaagctttattcacccgtttatatactgagactttccatcagcagtatcacagttatgttgactttcgcaatgcaatttatctgaaagtcgctcagggattggtgcgcatgaactggctgattcagtatttatttggcgcttcaccacgcctagccgttacggatactacgagtcgtccacagcgcagtagtgttcaacatcccgatggtcgctacagtcaagtgacgggagactatacgtcaattgatcgctacgtggccaagttgacggcggctgttcgtcaacagcagttgttgtctgtcaatgattttgacgggccagttcggcttcggagtaatgggcagctagctatgatggcccggcagggggtctattatcttgaataccggggcttggatctcgatccaactagtccagtcggggtggacgcgaacgcggtggcatttgttcgtttgttggcgagttatttcgtaatgatgccggcacttccagctaagatggtatcccaagtcaacgctcaagctgaccaattgacccgtcaagttttgggtgaaaatccaacgacggctagtgctcaggccgtgccggctgttcaagttttagatgcacttgctgattttgttaaaacctatggcctaccaaatgaagatgccgtgttactcaaacagttgaagtcgtgggtcactgatccaaagaagacgctgagtgcgcagattgccatgcaagccgatccgttagcatgggcactcgaacgggctgcacgctatcaggaatcgagcaatgaacgtccgtttgaacttgcgggctttaccgcgctagatctatcgagccagcaactagcccagcaggccttgacgcggggagtgcaggtggacgttgttgacccacacgctaacattttacgattgactaagttaggacggtcgcaattagttgtgaatgggagcggaacggatttaaatccacaggcgctaacgaccgtactgacacataaagcagcggccaaacaaattctggctgagcacggggttccggtgccggcttcacagacatatcatacagctaatcagttgattgctgattatgatcggtacgttcaagctggtgggatcgtattaaaagcggcggatgagtcgcacaaagtaattgtctttcggattatgcccgaacgcggactgtttgaacaagtcgtccggcaactattcgagcaaacgtccgcggtaatggccgaggaagtggtagtcgcatcaagttatcgctttttggttatcgatagtcgtgtgcaagcaatcgtcgaacgaattccagccaatattgttggtgatggtcgctcaacggtcaagacgttacttgatcgcaaaaatggtcgagcgttgcgcgggaccgcttttaagtggcctcaatcagcgctacagttaggaacgatcgaacggtatcgcctggactcatatcacttgaccttagattctgtggtcagccggggaactcagatcttattacgagaggatgcgacttttggtaacggggcggacgtgctagacgcgacggctgatatgcatcaatcctatgtgcaggcggtggaaaagttggtagcagacttacacttggcggtcgctggggtcgacgtgatgattcccaatctctatgccgaattagtgccagagcatcctgaaatggcggtatacttgggtattcatgcggcgccgtacttgtatccgcacttgttcccaatgtttggtactgcccaaccagtggcggggcagttgttggatgcattgtttaaaaatgaagattaaaaacaagaaagctggccttgtccagcttttttgatagctcataattaagtgtcatgattgaaaacacccatt

SEQ ID NO: 148 - protein, 630 aa, Lactobacillus plantarum
DQVSYQRRRDLARKYELQRLMTTGSHVNMSLNEALFTRLYTETFHQQYHSYVDFRNAIYLKVAQGLVRMNWLIQYLFGASPRLAVTDTTSRPQRSSVQHPDGRYSQVTGDYTSIDRYVAKLTAAVRQQQLLSVNDFDGPVRLRSNGQLAMMARQGVYYLEYRGLDLDPTSPVGVDANAVAFVRLLASYFVMMPALPAKMVSQVNAQADQLTRQVLGENPTTASAQAVPAVQVLDALADFVKTYGLPNEDAVLLKQLKSWVTDPKKTLSAQIAMQADPLAWALERAARYQESSNERPFELAGFTALDLSSQQLAQQALTRGVQVDVVDPHANILRLTKLGRSQLVVNGSGTDLNPQALTTVLTHKAAAKQILAEHGVPVPASQTYHTANQLIADYDRYVQAGGIVLKAADESHKVIVFRIMPERGLFEQVVRQLFEQTSAVMAEEVVVASSYRFLVIDSRVQAIVERIPANIVGDGRSTVKTLLDRKNGRALRGTAFKWPQSALQLGTIERYRLDSYHLTLDSVVSRGTQILLREDATFGNGADVLDATADMHQSYVQAVEKLVADLHLAVAGVDVMIPNLYAELVPEHPEMAVYLGIHAAPYLYPHLFPMFGTAQPVAGQLLDALFKNED

SEQ ID NO: 149 - DNA, 2017 bp, Lactobacillus plantarum
cgttggtaaagggactttggactacttggtctatgtccgtccgcaggtgattaagtccagtgtgataacgcgcattgacgtgcaattcaaaaatttgcggggcgtcaccccctacacggcgaagtatcggcgcttgaatcgcgagaacaccgcgcaactcaaacgctggttaaaaccacaagcgcgtaagcggcagcaggcattacaagcccaggcccaggctaagctgaaaccattgcgacaggcgacccagcaacttgctagtcaagtgccagcgggaacggcacaactagtcaagttacaaagccaattaaaacgcgcgaaggcccaggtcgcggccatcacaatgccgacttatttgtacactgaccgtacggataatccgggttacacagaatatcacgaaaatacgcaacgagtcgtggcactgtcgactgtctttccgctgttctttattgcgattgccgcgttaatttgtctaacgacgatgacgcggatggttgaagaattgcggctacagatggggacgttaaaggccctcgggtatacgaataccgcggtcggtagcgagtttatgatttatggtggtttagccgcgctgattgggaccgcgctaggtgtcctgttcggcgtcaattttttcccgcggtttatcgcgcaggcctatggtagtatgtataatttgcccgcaatcaacgttcaatacatttggatggacattggtatcgccttagccattgcgttgttgtgcacgttggggacggcactggtcgtgctccgcgtggatttaaacagtttacccgcgcaactcttacagccacgatcacctaaggccggtaagactttgctattagaacgctggcaatggctatggcatcggctgagttttaatcataaaatcacacttcgtaatctatttcggtataagcaacggttgctgatgaccgtgctcggtattgcgggctgcatggcaatgatgattacggggtttggcttaaaggattccattggtgatattagcgtcaagcaatttaacgaattgtggcactacgatgctgtggtgacgcgtagtgggaacgaaacggaccaacaacggcaagcactcagtcgtggtcaactttaccaggctagtttgaaattacaggccaagcaggtgacggtcaaacagtccggggtagcagaacagacggctacgctcggtataccggcaccccaccaatcgctaagcaagttcgtggtattacggcaccgacaaagtcatcaggccattcatattggtgatcgcggtgcggtcatcgatgaaaaattagctaagttatatggcgttcaggcgggcgatgatttaacgatcaagttggccgggcaaaccaccaagcggattcacatcagtgcggtggctgaaaattacgtcaatcactttatctatatgagtccgacttattatcgacgtgtcttcaagcaggcaccagtatataacacgaactatgtccggtttaagcaggcaacgaaaaagcaagaaaatgcttatgcggaccggctattgaaacaggcgggggttcagaacgtgacactgatgagtacagagaaagccactaattttaaaatgctggatagcatgaacttagtcgtattgatctttgtcatctcggcgggggcactagcgctagtagtgctctataacttaacgaatattaatgtttctgaacggatccgggaattgtcgacaatcaaagtgttgggcttttacgatggtgaagtgacgatgtatattttccgtgaaaatctgatattgacggttttaggcattattgccggttgtttcttgggcaactggttgcacgcatatatcttgcaaacggctgaaacgaacgcgttaatgttttcaccaacgattcatccgttgagttacgtttacgcggcattattgaccctggcctttagtttattagtcatgggaatgatgcatcgtaagttaaagcgagtcaatatgctggatgcactgaaatctgtcgattaa

SEQ ID NO: 150 - protein, 671 aa, Lactobacillus plantarum
VGKGTLDYLVYVRPQVIKSSVITRIDVQFKNLRGVTPYTAKYRRLNRENTAQLKRWLKPQARKRQQALQAQAQAKLKPLRQATQQLASQVPAGTAQLVKLQSQLKRAKAQVAAITMPTYLYTDRTDNPGYTEYHENTQRVVALSTVFPLFFIAIAALICLTTMTRMVEELRLQMGTLKALGYTNTAVGSEFMIYGGLAALIGTALGVLFGVNFFPRFIAQAYGSMYNLPAINVQYIWMDIGIALAIALLCTLGTALVVLRVDLNSLPAQLLQPRSPKAGKTLLLERWQWLWHRLSFNHKITLRNLFRYKQRLLMTVLGIAGCMAMMITGFGLKDSIGDISVKQFNELWHYDAVVTRSGNETDQQRQALSRGQLYQASLKLQAKQVTVKQSGVAEQTATLGIPAPHQSLSKFVVLRHRQSHQAIHIGDRGAVIDEKLAKLYGVQAGDDLTIKLAGQTTKRIHISAVAENYVNHFIYMSPTYYRRVFKQAPVYNTNYVRFKQATKKQENAYADRLLKQAGVQNVTLMSTEKATNFKMLDSMNLVVLIFVISAGALALVVLYNLTNINVSERIRELSTIKVLGFYDGEVTMYIFRENLILTVLGIIAGCFLGNWLHAYILQTAETNALMFSPTIHPLSYVYAALLTLAFSLLVMGMMHRKLKRVNMLDALKSVD

SEQ ID NO: 151 - DNA, 626 bp, Lactobacillus plantarum
aatattcgctggattgggacaatactttacaagaatatggctacttaatgatttttgcaggattattgtacgcctacgttaatatgctgacccaagatagtgagattaaattacggttggcccagtttgcgagtcacgacgctttgactgagactgagaactttgccgcttacacggaacatatcaaatatttattcgatgatagtgccaagaacaatctcaacttatcgatgatgatgttcgatattgatcactttaagcacgttaatgacacgtacgggcaccttgcaggggaccgcgttttgcaagaagttgccgccacggtcacaacggtcttggccgccaatgacgagaaggtcaagctgtatcgcaccggtggtgaagaattcaatgtcctgtttcccggttatgatctggctagtaccaaagtgattgtccgtcaggtctttgaagcagtcaatcatctcgttgttaagtatgaagacgaggaaatcaatgtgtcgatttcggttggtgtctcgacactgcatcaagccgatggtagtccgattgatttgtacaaccgtgttgatcagaacctctatttttcaaagcggcacgggcggatgcgtgttacggttgaatag

SEQ ID NO: 152 - protein, 207 aa, Lactobacillus plantarum
YSLDWDNTLQEYGYLMIFAGLLYAYVNMLTQDSEIKLRLAQFASHDALTETENFAAYTEHIKYLFDDSAKNNLNLSMMMFDIDHFKHVNDTYGHLAGDRVLQEVAATVTTVLAANDEKVKLYRTGGEEFNVLFPGYDLASTKVIVRQVFEAVNHLVVKYEDEEINVSISVGVSTLHQADGSPIDLYNRVDQNLYFSKRHGRMRVTVE

SEQ ID NO: 153 - DNA, 358 bp, Lactobacillus plantarum
atggcaacaaaagataatgaaaagattacattgatggcgctagtcatgatgatctttacgaccgttttcggatttgccaatagtacggtggcctattatttaatgggttacagctcgattctattttacctagtcgcagccgtactgttcttcatcccgttcgcgctaatgatggcggagttcggggcagcggttaagtctgatagtagcgggatgtacaagtggctggaagtgagtgtgaatgcgaaatttgcgttcgtgggcacgttcatgtggtttgcgtcgtacattatttggttagtctcaacgtcagctaaagtctggattccgtttacgaccatcttctttgggagcgatc

SEQ ID NO: 154 - protein, 119 aa, Lactobacillus plantarum
MATKDNEKITLMALVMMIFTTVFGFANSTVAYYLMGYSSILFYLVAAVLFFIPFALMMAEFGAAVKSDSSGMYKWLEVSVNAKFAFVGTFMWFASYIIWLVSTSAKVWIPFTTIFFGSD

SEQ ID NO: 155 - DNA, 5250 bp, Lactobacillus plantarum
ggatcgagtgttggaacatacacattggcactctcaacggctggaattactaagctagctgaagctaatagtagcgcggatataacggctgctaacgtggtgacaggaacactaacaatcaagcaagctccggtaccgactgcgataattaccattggttcagctagtattgactatggggatgctaaaccaagtacgtatacaattacggtgccgagtcagtatgcagttcccagcacctggacgttagctagttcggctactgatggaacgactaatacttatatgattgcaagttctagtggcgatgttatagttcccacagcaacccaatctggaacgtatcagcttgtgttgtcagatcaaggcttgacagctttacaacaggctaatcctaatgctgctattactgctgatacgattattgctggtagtttagttattgcggcacatgacattattacgatgggtgcgacgacaattgtcgttaataaaacgactagtacggttccggtgacggtcaatagtcgtactattgtggttccaacaggttggacaattcgttacgatgatattcagactgatgcgattgtgtatgacgtccccgtttccgatacgacatattcggaagcggttaatactgctgtggttgataaatacaccattacattgactgatgatacgatagaaacattagctaaccttaacagcagtacgacttttaatagtacgacggttggtaagggcgtagtgcttgtcaaggctagtgccgcagttgccatctcacctgcaaactatggcgcgcaggctagtgccgaaactccggtaacagggctgacaatttcacatgcccgaacaaagggaattgatttagcatatggtcaggcgctgtatttgatcttgccgcttattaatatgaatccatcaggaatgactgtggctaatcttactgattatgttattattccatctggttttaaggttgctactaatagtgaaggagctattaacatagcgactgatccaagtagtgtgttaacgtctgctattgaagcaatgatgacgaaaaatgatgtgacctatcaggggttaaaggtgacccaactgacagactacaggggtcgccaaacatttaaaattcattttgataaaaccactgtttatgacggtggtgcatttgcaacgctaaaatatgcattattaccggtcattgctgttcaaaacactggggtgactagtggtttaattggtaatcaagtttcaagcccggattcggcggtggtttatgttactgatgattctaatgaaaataatggtagttattcgttgaatttgcaaaattatactaatattgacagtgtcgctgatgcattaggaattgcggatgctgtcacgattggtagtggtttcacaagttacctatatcattacacgctatcggccaaaacgattaccgatacttatagtttagtaggaaacgatggcacgtcattaggcgaagtaacttttacgggcgacagtggtaagacgtatgtaccgatgactaaattacccatgacaattacacaaaatggcgtgacgtattatttgaacactagtgcagtttcgttaactcagacatattctggtgatagtaattcaaattacacagttacttaccagcgctacgtcacaacgacgactgatactgcggccaagataacgattgcaccagcttcaaaagtctatgataacaacgccacgactgatccaagtcgctatacggtatacttgccaactgaatatacggccccaagcgattggactgctgatagcgcggcgacggctgtggatgggacgacggcgtaccaagtcagtaccgactaccttaacaccactgcaatcgatcaaaacgtgggcacttacgctgtcacgctgaatagcgccgggatggcagccttatccgctgctaatccagatttcttgattgcaggcgatgtgaatgttggtgggactctgacgattactcaacgtccagtgacgattactttgccggatacgattctgtgggccaatggtcaggaacaaaatattacgccggtcattactggtgttgttgcggtgcaaagtttggattacacgttaacgtcagggttaactgatccggacacgacaaccattacggccacgctgacgaatgccgctgctaatagtaattataaattgacgaattcacctagtggtcagttgacggtgggcgccgtaacggttgtctatcagtatgggtaccgcgacaaagcggggacgctacacgtggtaacaacggctaatggaacggcgacgcacgggactgatgttaccgctaaggactatttgagctacaccacgagtgatacgactgctacgcatgccaaaactggttatacgttacaaccagaaagtaccggttaccaagccgatggcactctagcggacgttggtgggcaggtcgtgtacacctatttagcgaacaccgaaaagattgcggtcgtttacgtcgaccaagataagaacaacgtgattttaaaacagattcccctcagtgggagctttggcacacccacgaattatacgacagcgcaggacattgcggcgtatgaaaaattaggctacgtgttagcttcggataaggtcccagcgccgcttgagtttgatcaggatactgaacagacctactacgtatacctgaaacatggcaccatcacggcgacggttgatcagccaggtaacgtggccgttagtgatttgatgaagaccagtcagcgaacgattcattacgtttatgctgataacacacccacggacttagcggatgtgcttcaaacggtcacgtatacgcgcacggcaacgggggatgcggtggatagaacggtcctttcgtacggtaattggacgaccaatgtgaatagctatccggccattgagtcgccgaccattactggttacacggcggatcaaacaaccatcgcggcggctgtacccgctagcatgggcgagactacggaaacaacggtccgatacagcgttaattctgaaacgatccgggttcaatttgtcgatggaactacggataaccaagtcttaagttatattgatttgaatgggaaatacggtgatgctgccgactatacggtcactgctgatatcgcgaagtatgcaaaattaggctatgaaccagttaactcggacttgcctgatcagctgatttataagcagaatacccaagtttatacggttacactagcgcatcgtcacgtgacggtcagcgttgatcatccgggccaacctggtcaggccatcgatgctgattatccagccggtcctaaatatccggcaggcactggtcgtgattcgttggaacaaacagtgactcggacgattacgtatcaatatgcgtcaggtgaatcagcggctgaaacggttaaccagtcggtcacgttcaatcgcacggcaactttcgacatggcaacgggtaagcagctgacttacggtgactggacagtggcacctggtcagtcagcactattggccgcggtcacgtcaccaacgattacaggttatcaagccagtgttacagaagtcgaagcagcgtcggtcactagtcacgataagccgcacttgattgcaatcacgtacacggccaaatcacagaccgcaaccgttgcgtttgtggatgtaacgagtggtaaaacactacctacgacggtagtaactggtgcttatggcactacgaatagttattcgcccgtttcccaaattgctgcgtatgaaaaactgggctatcgattagtttcgaataatgttccgacgactggtatcacctttgatcaaaatgacgtcattaagtcatacacggtcaagctagcgcatcaaatgacgacggtcacgccaactaagcctgggcaaccaggtcaaccagttgatcccgctcatccagaagggcccaagtacccagctggtactgggcttaaagatttaacaaccagcgttcagcgagtcattacctatgtttacaatgatggtcaaactgcggcgccaaccgtcacgcaaacggtcagttttgagcgcaaggcgacctttgatcaagtgacaaaggtggtgacgtatacggattggcgtacacctgaatcagcgttgacgggggcatacgcagtcgttgaatcgccaataattgctggctacaccccgaatgcaacccgtgttgctagtgtaactgtcagtgccaaagatactgagtcgcgacaaacggttacttaccaagcaaatctggaaacggcgacggtgacttatgtcgatgccacgacgggccaccgactgggtacaagcgtgacgttaaccggacgattcggtacgcaagcggattatcaaccaacgacaatgattgcgcagtatacccaggcaggctatgtcttgatggggagtgattatccggcaacgggtgttacttttaatcaggcgggcgtcgttcagaagtatacggtgtacttggctcataacaaaatcgtgattacggcaccagatcagctcaccaaaacgatcacgcaaacggttcactatcaggatcaggctgggcacacgcttcaagctgatacgatccgggcgctgacgttcacgcgttctgggatgaaagatgcggtgactggtgtggcaacgtatcgtgattgggcaccgaccgggttgaactttacagccgtgtctgcgccaacgattgcgaaataccatgcgttgacggcgaccactcaggccgtggcaatcacggctgctagtgctgatgatgtccaaacgctaacatatgcgctggacgtcccaacaccgacgaaaccggtcaaactgactaagccagccaaaccgactaagccgacaacatcggacgatttaatcaagccaacgacgaaaccaatcacggctgctaaaccaacgcaactcactaagccagcaacggttgtgaaggattttcaagccacaactggcaaccagacgccagctaaatcgacaaggacgttggtatcgagtcgcattaaggctgtcaaaacagctccggcatcagcaatcatcaagccgggaagtaaagtaacggagccggctcacaaggctcaagcagatacaacgagtcgattgccacagactggtgaaacgcggtggtctgaaatggctgctgaaacactagggctaacactagcaacattattgctgggctttggtggcttgaagcgtaagcggcatgaaaagtaa

SEQ ID NO: 156 - protein, 1749 aa, Lactobacillus plantarum
GSSVGTYTLALSTAGITKLAEANSSADITAANVVTGTLTIKQAPVPTAIITIGSASIDYGDAKPSTYTITVPSQYAVPSTWTLASSATDGTTNTYMIASSSGDVIVPTATQSGTYQLVLSDQGLTALQQANPNAAITADTIIAGSLVIAAHDIITMGATTIVVNKTTSTVPVTVNSRTIVVPTGWTIRYDDIQTDAIVYDVPVSDTTYSEAVNTAVVDKYTITLTDDTIETLANLNSSTTFNSTTVGKGVVLVKASAAVAISPANYGAQASAETPVTGLTISHARTKGIDLAYGQALYLILPLINMNPSGMTVANLTDYVIIPSGFKVATNSEGAINIATDPSSVLTSAIEAMMTKNDVTYQGLKVTQLTDYRGRQTFKIHFDKTTVYDGGAFATLKYALLPVIAVQNTGVTSGLIGNQVSSPDSAVVYVTDDSNENNGSYSLNLQNYTNIDSVADALGIADAVTIGSGFTSYLYHYTLSAKTITDTYSLVGNDGTSLGEVTFTGDSGKTYVPMTKLPMTITQNGVTYYLNTSAVSLTQTYSGDSNSNYTVTYQRYVTTTTDTAAKITIAPASKVYDNNATTDPSRYTVYLPTEYTAPSDWTADSAATAVDGTTAYQVSTDYLNTTAIDQNVGTYAVTLNSAGMAALSAANPDFLIAGDVNVGGTLTITQRPVTITLPDTILWANGQEQNITPVITGVVAVQSLDYTLTSGLTDPDTTTITATLTNAAANSNYKLTNSPSGQLTVGAVTVVYQYGYRDKAGTLHVVTTANGTATHGTDVTAKDYLSYTTSDTTATHAKTGYTLQPESTGYQADGTLADVGGQVVYTYLANTEKIAVVYVDQDKNNVILKQIPLSGSFGTPTNYTTAQDIAAYEKLGYVLASDKVPAPLEFDQDTEQTYYVYLKHGTITATVDQPGNVAVSDLMKTSQRTIHYVYADNTPTDLADVLQTVTYTRTATGDAVDRTVLSYGNWTTNVNSYPAIESPTITGYTADQTTIAAAVPASMGETTETTVRYSVNSETIRVQFVDGTTDNQVLSYIDLNGKYGDAADYTVTADIAKYAKLGYEPVNSDLPDQLIYKQNTQVYTVTLAHRHVTVSVDHPGQPGQAIDADYPAGPKYPAGTGRDSLEQTVTRTITYQYASGESAAETVNQSVTFNRTATFDMATGKQLTYGDWTVAPGQSALLAAVTSPTITGYQASVTEVEAASVTSHDKPHLIAITYTAKSQTATVAFVDVTSGKTLPTTVVTGAYGTTNSYSPVSQIAAYEKLGYRLVSNNVPTTGITFDQNDVIKSYTVKLAHQMTTVTPTKPGQPGQPVDPAHPEGPKYPAGTGLKDLTTSVQRVITYVYNDGQTAAPTVTQTVSFERKATFDQVTKVVTYTDWRTPESALTGAYAVVESPIIAGYTPNATRVASVTVSAKDTESRQTVTYQANLETATVTYVDATTGHRLGTSVTLTGRFGTQADYQPTTMIAQYTQAGYVLMGSDYPATGVTFNQAGVVQKYTVYLAHNKIVITAPDQLTKTITQTVHYQDQAGHTLQADTIRALTFTRSGMKDAVTGVATYRDWAPTGLNFTAVSAPTIAKYHALTATTQAVAITAASADDVQTLTYALDVPTPTKPVKLTKPAKPTKPTTSDDLIKPTTKPITAAKPTQLTKPATVVKDFQATTGNQTPAKSTRTLVSSRIKAVKTAPASAIIKPGSKVTEPAHKAQADTTSRLPQTGETRWSEMAAETLGLTLATLLLGFGGLKRKRHEK

SEQ ID NO: 157 - DNA, 879 bp, Lactobacillus plantarum
atggatttaaagcaaagcgatggttggcgatacttagctgggtggagcttcattctattaatggtggcgagtgccacattgcaacatgatgcgaaaatcattttacccgaaatcggtgctctgacagccgggacgtgggtttatcgtaagacggcgtggactcggcaacccttaaagttattcttagtaccatctggaactgcaattattggcttcttagtcaatcaactaccttggtcgcacgccctcaaagtgcttgtcggtctattactgatgctattattattgaaggggttaaaatcgaatttggcgccagcctttgctactggcttactgccaattatcattaatgcaacgcactggacctttatcgtagccatctttttctggactatttgcctgatgattggggcttggattcaacgaccgcgatcaatctcacgggtaaccgaagcttctgctagtcgctggcaaatgctcggctttatcagcctagtttttgtctgggtgggtattgtttggctagcgggacagccccagatggccgcaatcccacccgtgatcgtcgttttctttgaagcggctcaacagtctgaatatacggtaacgaccgcacttaagcagtggcttgcattgtcggctgctgctagtattggggtcggcattcacctattgattgcttcgtggctattaacgacggtcattgccttaccacttgtgtatttgtggttacgggcgcttaacttacaattgccagcagcgtatgcctttccactattagccttagtgttaccagccaatatgtttaacaaactaccgacatccgccggcttagcggccgctttcttcctaggatcgttactcatctaccatcagatc

SEQ ID NO: 158 - protein, 586 aa, Lactobacillus plantarum
MDLKQSDGWRYLAGWSFILLMVASATLQHDAKIILPEIGALTAGTWVYRKTAWTRQPLKLFLVPSGTAIIGFLVNQLPWSHALKVLVGLLLMLLLLKGLKSNLAPAFATGLLPIIINATHWTFIVAIFFWTICLMIGAWIQRPRSISRVTEASASRWQMLGFISLVFVWVGIVWLAGQPQMAAIPPVIVMDLKQSDGWRYLAGWSFILLMVASATLQHDAKIILPEIGALTAGTWVYRKTAWTRQPLKLFLVPSGTAIIGFLVNQLPWSHALKVLVGLLLMLLLLKGLKSNLAPAFATGLLPIIINATHWTFIVAIFFWTICLMIGAWIQRPRSISRVTEASASRWQMLGFISLVFVWVGIVWLAGQPQMAAIPPVIVVFFEAAQQSEYTVTTALKQWLALSAAASIGVGIHLLIASWLLTTVIALPLVYLWLRALNLQLPAAYAFPLLALVLPANMFNKLPTSAGLAAAFFLGSLLIYHQIVFFEAAQQSEYTVTTALKQWLALSAAASIGVGIHLLIASWLLTTVIALPLVYLWLRALNLQLPAAYAFPLLALVLPANMFNKLPTSAGLAAAFFLGSLLIYHQI

SEQ ID NO: 159 - DNA, 195 bp, Lactobacillus plantarum
ttgacgaatacagacaatcgttattatcaaccaaccgacatcaaagatgcgcttcaaacaatccaaaaattatttaatacttataccgatgccccattaacacccgaattaatggcctaccatcaaaaattagttaatcagttagctactaatttattaccactagcacaacaacaacatgacaaattacggatc

SEQ ID NO: 160 - protein, 65 aa, Lactobacillus plantarum
LTNTDNRYYQPTDIKDALQTIQKLFNTYTDAPLTPELMAYHQKLVNQLATNLLPLAQQQHDKLRI

SEQ ID NO: 161 - DNA, 61 bp, Lactobacillus plantarum
aagactatcctgaaattgcgcctaagttacccgaaatgccattgaatgaggcacttaaatt

SEQ ID NO: 162 - protein, 19 aa, Lactobacillus plantarum
DYPEIAPKLPEMPLNEALK

SEQ ID NO: 163 - DNA, 2119 bp, Lactobacillus plantarum
aagtgttcaatcttcattcgacgctaccccgttttaatagtcgaacgcgactcactgccggaattttagacagttcagctattaggtcggcttgttgtggctgctgtaggtcgtcgacgtcaatgatggtgtaagcaatctggtgcttagcggcgttagccatggtagtgatgttgaggttggccgctgctagtttggccgtgatttgactcaccatgttaggcacattttcgtgaatgactgtaaagcggtaagccgcgttgaacgggacgtttaagtctggcagattgatggccgcatgaacgttaccggtttccaaataagtcatgatagtgcgcgcagcttgtgtggcaccgttgatttcagcctcgatagtcgagccgccgatatggggtgtcacggtaaccgcggattggttggcaagctggggttcgccaaaatcggtgtagtagtgggcaacttgtcccgtggctaacgcattcatgacggcagtattgtcaacgatgcccagccgtgaataattaaataattgaacgcctgttggcatggcggctaacgcatctttattaatcagatgaagtgtgtcggcatttttaggaacgtggacggtgacaaaatcagcttgtttgactgcatctggcagcgtggccgctcgctggacttgtttagcaatgttccaagcggcatctgcagatagataggggtcgtaaccaattacattcatgcctagactcaatgctgcattggcaacgagagcgccaacatggccgagtccgatgacggccaaggtcttacccgttaattcaataccattaaattgcgtcttgtcgtgttctgtgcgttgagaaatatcagcttcggtatgctgggccgaataggtcgttgcagctattagattacgggatgccataatgagcaggccgatgatgagttccttaacggcattagcgttacttcccggggtgttgaaaactgcagtcccgttggcggttgcctgatcgataggaatgttgttaacgccggcaccagcgcgcacaatgactttcaacgatgacggtaatgtctcggtatgtaggttgaccgagcgaattaagtaagcatccggatgctcagattgattgagcgtgtaatcagcagtaaacgtgttgaggccggctggggcgatggcgttataggttttaacttgatacataaatatcctccttgattaacgatggtgtgcttcaaaagcggctagatagtcaactaatgcctgaacaccggctaacggcatggcgttatagaggctggcacgcataccgccaactaagcggtgaccctttaggtttaataacccgtgctcacgggcgccttgaatgactgcggcatcgaggtcggcctgacctgtgacgaatggaacgttcatggtcgaacggtcgctggtcttgactggattagtaaatagttgtgactggtctaagaaatcatagagtaaggcggctttagcgtgattgcgagcagtcattgtgctgagcccgccttgggcctttagccacttgagtacgagacccgcggcgtaaatagcaaaaacaggcggcgtgttgaacatcgaatctttagccgcgaatagctggtaatccagcatgcttggcaggttggcgacttgaccaattaaatcatcacggacaatgacgattgtcaaaccagcgggacccagattcttctgagcaccagcaaagatgagcccaaaatcgctgacttggtaaggttcacctaaaaagtttgatgacatgtcggctaccagtggtacttgacccgtaactggcaggcgcgtcatcatggttccttcaatagtattattagttgtaagatgaatataatcgagggattgatcgatgggctggacgaccgttggcagttggttaaaatggttggcagcgctactccccagtatcgtgactttagtaccgacccgtttagcttcatcggcggcgcgttgtgcccagtgaccgctgtcaagcaacccgatacgatgatgaggcgccagatttagtggcgcagctgtgaactgtagcgtgcccccgccttgaaagaagagcacgtgatagttgtcaggaatggccattaaatcgcgaagatc

SEQ ID NO: 164 - DNA, 2806 bp, Lactobacillus plantarum
ggatcgaagggaccattacgccggctagtggcacaattgatcgcccgattggccgggtggctgacagtcctcggcgagtggtcaccacggcgggccaacgcgccattacgacgtatcaagtggaggcggaccaattgcagcataacgtgagtcggttacggttggaacttgtgactggacggacgcatcaaattcgggtccatctaacgacgcttgggcaccccttattaggtgatgcgctgtatggcggtaacttggggtggattcaacggcaagccttacacgccgctagtttacagttctttgaccccttttcggaacagactttacactttgaggcggcattgccagctgatctgcaagccttgaatcacgactaagttcagtttaggaccagtgaccatccggatgaatggttaataaaaatcggcactgctagaatgcgttgaacattctggcagtgccgatttttggttggcaaccgtctaaaatcaagctaacgtttagcgggtaagttgcaggcccgcgttatcctgaaggttgcggtgactattgaatgaggcgctagtgcttacgactgactaagtggcgcgttatcagccgttagcgtcgcaaaagttgctgtaattgctgtaatggtgcggttaacgtgagaaaataaatatgctcgactccgttactgcgcatatcaatgaccccagcttcacgcattaagcgtagatgatgtgaaacggcggggcgtgaaatattgaccaaggcggtaatatcagtaacgttgagaccattatcactgttccccagagcaatgataatctgacgccgaattttttcggcgaagatattaattagtgtggcactttcaataagtgccgtttcggattgttgaaaattagccatggtgactgcgtcctttcaaattcaatttacacttaaggactaactccccccatcggcagtacgctgaattagttgttaagtgttggtatgacagctcgattgttcaagtcctggtgaaatagttgcagccaagaaaactttaagtctacaagttgtcagttagactaattgttattggaacgttcgtacttatgtataattaaagcaagtcgttaatcgattgtcaaatctttttgttaaaagtaagtactattagcgggtttaacaattgctgttcgtttgataaccatatttttatgttgaaatgcgttaaaataaaaatagcttttgaatgaatgatgggagtggaaaatgtgaaagtcttaggaatattaggtgcgcatcgcgctgatggcgtgactgcccagctactgcaatccgtcttaaagggggccgcggccagcgctgacacggaactagtcaacctcaacgattatgagttgcgaccagatcacgatagtcaaccgaatgctgacttagacgcgctggaagcaaaattaatggcggcggatgtctgggtattagctgcaccaacctatttggggagcttatcgggggtaatgaaaaacttctgtgactgttttcgggggcggatcgcacggtttaattccgtgggtgaagcagtacctgatcgctttaagaacaagcattatgtgacgatcacggattgttacgcgggtggtattgaaaattatttgaccggcgtgactgacgcaacgtttaaaacacttgataaatttttgacgatgggtggtctcatcaaattacgggagattgtcgtaactaaaacgtggggtatgcaaaccatcacagctgctaagcaagcagaatgtgaacgggtcggcgcgcgggctgcacataaaaaggaaagggatgacagtacggtgaaacggtatattcaattattcttcatgattgcggtgatggcactactaacaatgggaatcgaagcggggattcaacaattgattccgctgaacaatttttgggcctactacggcgtctttgtcgtcgtcttttatgttcttttagcaatgattttacatttcttcactgttgttaaacaccggcgtcgttaagggatacggcatgtgcattcagcaacaacgacattaaaattcaattgattagcaagctgggcttggcgtcttaatcgccggaggtcagcttgttttgctataataagaacaattacgaagtaccagcgattatttcagtgtccacgcgtataattaatatggtatcgtcgaattagaaaatgaggacacttggtactggttgccaggcgtatgaaccataattaaaacatgttaaattagatgaatttttatttggcagcatgctgtgattggtttcggttttggcgcatccaagctgccggtttttgaatctaactttgtcaaaaactaatcatgggtcatgcccaaaaacgtgttattgatttcaaaattaattttgttaaataaaggctgtcaatcaaggtacgaggaggaatagcatgcaagtttttggacaatttattgcaacagtcggttggctaggattggcactagtcgccagcgaactaggtgcgacgttaatccattggctcggtcagtgggtcggatttcgattaattggtgctcgaattgtccggattaccggttttcgacttcaattaagtcgggttcgtggtcattggaaattagaacgaccgctgacgcgtcatccacatatcgtggcagcaccctcggcggatgccaaacggttcaatcacgccatttattgttttggcggtggcctgttcaacttactgacggtcatgctcagtttaataactctgaatcaatttaagtttagtttcgatttatggttgtttgcgttcattatt

SEQ ID NO: 165 - DNA, 1839 bp, Lactobacillus plantarum
gatccattgtcgactttgttgccgcgccgtaatcaaacagtgcatctaaagtatcgttctgcacagacgacggcggagctacgcaagacgctacgtcaagcacggtatttacaggccggtactcagaataccgccacgccggtctttcaaaatcgacagcagcgaggtgatgcgacaacgtacggtcgtatcagtaccagccaagacggccggatatggacgaaactacccattagttatccgcatgtgcaattgtcacggccgagtgtctggtacgcgaatggccgcttgacgttgatagatgggaaagaccgttactggacgactaattttaaagattggcaacatcaacggttgaactttaacggggctgattttaagcaaggtcgggttcaggccgtctttccaggtacgactcgttcagcggttgttgtggttcgcggcattgatcgccaaagcagtcgcgccaaactctattatggacagctcacgaagactggacgggtcaaagcttggcacgcgttacaactaggaaagctcccagcgcgccaagtcgctggaatgagcttgattgatcaacacttatacctgtttcttcagcgcggtacgcagttggccatttatcgtgccaatcggttgacgcgtccggtcaggttggttggtcgcgttaagctaaatcatgcgcagtcacaacgagtgaccgcggtgaatttgataccgaccaccaagcatcgctaccggttaatatttgacttgacgacagctgaaaaagttcagaaacagccacgttatcggttacttgatcggcgatttaaagcagtggggcagcagcatctattggtcactgattatctctggagccaatttcaaattagtctacgtgggagtgagtgaggcacatgaaggtacagccaaaggaacgctttagtctagcgtggcggtggttgccgctcgaattgctgatcattatgctaagcgtcggccttggatgggcgggcaatcgatggctacctaagccggtgtatcaagcatctgttgatattcagattgcgcaaacgccgcgttcagggctgtcaacagcccgtctaaaacgtcagcgacgccaggatatcaaagctatcacgcagttcaacgtgatgccacaccagagtgcagtgctgactcaagccagcacttatgcctatgcgcattatggcatttggcaaccgattcaggaactgagtgagtcggtccaagcggcaccagttgcgcggcgaccggtcttacgggtgacagcaacgagtagttcacggcaagtggcccagcagaatgctcaggcgttcaatgtggcgattaaagctaatctgacgggcttaaaaaattatcgagtgaagacagttaaacgtaccgtaacgcgtgagacgaacgtgattcgcggggcgctttggaagttaatattagttgttgggggcggcttggcgttgctgagtccgtacctcgtgaaatatggtcagggttgggggcggcacgatgatgagacgtaggggagcaagtatgcagcagcaccgtaatgtgctctatctgattatcttcggaatctacttagcctcagtcacactacagacgacgacctttaacgagatgataccgcatcgagtgggcgttttgattgaattagcgactttggccgcattactgggcctcgtggtttgcttagataccttgacccccggccaaattattggagaagtcagtttacttgtactggtgactgtcgtgacactcacatcgggtgcgcattatttgatgccgacaatcatgttggtgattgcagcccgggaagtttcgtttcggcagatc

SEQ ID NO: 166 - DNA, 3239 bp, Lactobacillus plantarum
gatcatcgagggtcgcttttttaaagtacataattctagctcctctcttgaacagcgattgactgccgaagcagtaatcgacttttaaaagtatacagaatttttagaaaaatgaaactattttctaccgattcttaggagattttcagtgacgattttcaagtaaaatcttgcgcgcttctcgggccatgtcatgttgcgtattataagcagcgaccgcactcggtgcatattggtaagtattagtctgcaaagtttgttcatagaattcgaagtagggtaagtgggtattttgcgcaatcgtgatagcacgctgccaatcgaaatctacggcgcggtaatcaacatgctggaggccagtctcatcaattgtcagaagcaagtagtttgctcgtggttggcgtaggtggggattaatggcagtcggaagaccaactgtcccggcgtttagaatcaactgtcccgtggttgcgtagcgcatgatgggctggtgtgtgtgcgcgtaatgacgatatcgacatcgccttcagcggcttggtcaaagttggcctgactggcagttggtgcgagcgcgtgtccactagcgacggtcggtaaaacgtgttggagacggatggttaacgggccgacgtgtttgatgactgtcattggtaagttcagtaaccgttcaaagtgtgtcgaactcagctggcgccgatcaaaagcggtgagaaccgttgccataatttgtttgggtttagtaaacgtgttgggattggctgccattactttttggtagttttcctcgtgatttccgagaacgtaggcggtggggtgaacgcggtctagtaaggtgagacagcgctccgattctggcccgcgaacggtcatgtcgcctactgtccagtattcgtcaacgtgttgtttctgggcgtctgctaagactgcttcgagtgcagtagcgttaccgtgaacgtcagataggactgcaatttttaccatgttgagatgcttcctttctactatgtaatctaaactctggtgagatgttttattctggtaaacgtgggttttaaaaatttatgaatgtaaccactatgaaaagccatcgtaacttttatctgttataatgggtatgaaacgaagtaatatatagcacgtatattcaagccaattcgtttcgtgccgtggttatccttgcgataatcacggtttttattttaccatagtgccttatgattagtttggttggtgtcatttaattgtatcagtacatggaatgcgggtgggttaccacacatgtaattgcccagagttcaaggtgacttacatatggtgtgatattgtttaaactgaatggattgcttctaaatatagtggttaaactaattgtttgtgaaagaggttttggataaatgatgacaccttacaaaaaagttaacgtaacggatgcgcctcgactaaagctggcaattaatttgactagtgtcttgttgtcttttcaatgttatttagtaatatgattcgagccagcaccattgatattgggcatgtagcttggctaaaatggtcattttagcaattgggactgttgcgattcatgggtgcgggttgtactggggaatttttacctagcatagcaactagtgataacgttgcgatactgtctgatagaagatacattgcggggtatgactatttatcgctaaaaattgggaaattagtatccacgataattgagttggagtatcaaaaggccatcaacctgcgtatatgcttcgcggttgacggcctttagtttagtgctgaagtcaaagtagctatgtgaatggatctcatctcaaaacaagctatacaccaaataaatattcaacaccgtaataattccggccagtccatatcccacccacgttaccgccgggcgattaacgtgggccttcatcaggctggcgtcatttgtcagcgcaactagtggcaacaaggtaaaggggagggcgatgctgagtgccacttgcgcgtaaatgatcaagttttcaaaggcagcgtcactaaagccgactaacataccgataatcagaatcggaattagcgtgacagcgcgagtaagcagccggcgttgccatagcggtaagcggatgtgtaaataaccttccatgacgatctgaccagctaaggtgctagtgatggatgaaattaggccggtaattagtagggctaatgcaaataaccagctcatgaccgggctagcaagggcgccaaccacggtggtactttttaacccatcgaagacggcctgcagactcgctaacgcgttggtgtgaccgaagaaaagcgtcccaccaaggacgagcaaaagtgcgttgatgagaaaagccgcaatcaagtgcactgttgagtcccaattggcgaagcgcagtgcttctgtgacttgggctggattatgataatcataacgccggctttgtgcaagcgatgagtgtaagtataagttatgtggcatgatggttgcgcccaagattccgagactgaggactagtgcggtatgatttttgacgatcaatggtgttggaactaagccgagcaacacgttgccaaattgaacgtgggcccgtcccacctcgataccaaaaataatgccgacggtcaaaatagcgacgaggacgattacttcgacacgccgaatgccaaatctcaaaaacaacaagacaactaagacgtccgcaatcgtcagtaaaattcctgcaagtagtggtaagccgaataacatttttaaggcgattgcggttccaattacgcccgtcatatcagtcgccatcatcgcgacttcgtttaaaatccacgcggcgtaacgcacgggccggggcagcttgctagcgatggcttgtgctaagtcttgccgggctacaacgcctagcctgattgccaggccttgcatgaacatggcgacaatgatggctaatgcgagcacggctaaaagccggtactgaaactgaccgccaccggctagggatgtcaaccagttgccgggatccatatagccgactgccactagtgcaccgggaccactataggctaaaaatttgcgccaaaaggccgtctgatgaacgtcgggaaccgcgacgctctgattgatttcttcaagactttgatgacgatgtgattgcatggttcgacgcctcctgcttttggatagcgaggtcagccagaaattggctgaagccggtatccttattcattggtggtactaagtcaaaccgatc

SEQ ID NO: 167 - DNA, 1866 bp, Lactobacillus plantarum
tggcctgccaggcaggactatcaaagcgtaagaggtctatatagtgtacaagtgtcaaattagacggactccaaccaagactgcgttgtttgagtagcgattggagcacgatggcactgaatggcaggacaatagcaatactgatgactagggtgacgaaagtgcctgctaatgttgtcactccaagctgccgatattgtgacgctgaccgttgtccggtgaacttaacagctggccggcgtaacaaccattgctggataccccaggcaatcagggcaatggtcagtaggagggtgccggtgagtaccccgttttggaaatctaagggccactgactgaggtccctttgaatcgtagtcgtcagaacttcgaagtggatgctccgaccaaaggtggctggcgttccaaattcagccaaggttttggtaaagactaaaatccatacagctaagtatgggactaacatgattggtaatgtgattcgcactagtcgttgccaggtgttgaccccatgaacttcggccgcttgaagccagcgctggttgaattgcatgagggctgcgcgtaagcctaagtatgccacgggatacaaatgcagactcatgataatgaccatcccgaacggtgaaaatagccactgaaattggtggtgccaactcggattaagctgagccagtaatccgtgtggttgaaagaaatataaccagcccatcgcgttaatatatggtggtgtcatgaatggcactaacaagagccaatgcaaccaggcgagctttgttaaacgggtgtgcgtcatgatccatgccaaaggggtggctagcagggttgtcccgacgaccgtgcccccactgaggaacagactgtgttgaatgctcacacggttggttggctgcgtcagttgtgaccatagctggcttggcgattgcccgactagtgtttgaccaatcagggcgactaacgggccgataatcagagttgccaacaagcaactggtgatgattgataaggacaagcggatttgatgattactcattggctaaagacctgattgaatgcgacaacgtttttggtcagggccgcgttggcactggtccaattgaccgtgtaggctttgatggcttcgccattgcgtggatcggtcaaagtgctcgtggtacctggaattaagttacttttttgaatctgtctttgaactttagctgataagagatagtcaataaattgtttggcggcggcttgatgacgactagccttcaaaatcatcgccggtcgtggattgaccaaagtcccactcttaggataaacgaaaccaattttttcgccttttttaatagctgttagactcatgtaatcgaccccaccaaagacggcgatttttggccagtgatgactgcatcgagtacttccttgttagcacccccgatatcggcaccgttttcttgaagggctttaagtagttgcgtaccgtgtttcatttgataagcgttaatgaagtccaagctagaaccagaggtttgggggtccggaatggtcacttgattgcgatacggagcggttgtcaagtcagaccagtctgtcggtgccgatttgatgtgccgcgtattgtaggtgatgccgactgccgaagcactgtaattgatcaactggtggctagtatctttaaattgtttattcaggtgtttagcttgagaaggctgataggttaatagctggccattcttttgtaaatcgacgccagcggccattgaagctaaaatcagcacatcagcttggggattgccttgctcggccttgaccttacttaaaattttcccggtcgtgccgtcaaaacttttgactttaatgccagttttggcttcaaaaccg

SEQ ID NO: 168 - DNA, 889 bp, Lactobacillus plantarum
gatcgcacgtaatgggcaagtcaccgtggcttttgatgcgcagcacgatgccatcatggaattcgatttaccagtcaattaccaacgggagtttcccgagacggtggcagtcttagacgatggtcagtatcagaccatgatgttgatggacgaactctccgtctgaacgcagcgattattcaatgagctgaatacgaggaaactcgtcacttcaggccggcgaataatcgctttttgattggtggtctttaactgggcagatgcgccttaaatttgtataattaaatagatggtgtgaggtgtggttttgcgatgttaggttccggttgctaatcaagggggccgcattgttcacgtaattctcacgccgggatcagagaaaggtgtgtgtctaatggcgaaaacagcagtgtgcattgtcgatcaacaacgttaccaagttgtggacggtatgcgattagaagaattggaaactagtttgcggcaaatgattttaaaagattttccgcaggcccataatagcagtttcatttgtagtgagcatctcgtacattatcgcttagcaaagatggatgcgatgatcgagaacgattatcaacaaaatgataaggtcaatgcgcaattatctaagattctcgctaaccacacgtatcgggtcgtcgatgttaatagcgagctggaaagttcattgacatttggtcaacgggtcgcggatggggtcgcacggttcggggggagctgggcgtttatcatttcgtttgtcgtggtgatgctcgtgtggatgttgctcaacgtcttaccaatttttagccatcattttgacccttatccctttattttattaaatttatttttaagcatggtcgcagcaatccaggcaccattgatc

SEQ ID NO: 169 - DNA, 2054 bp, Lactobacillus plantarum
gatcgcatttctgatgttgaaagtcggatgtacgaaaagccccaaaatattgtagcttacttggaagataacccaaccaaacctttcctagattgtgaatatatgcatgacatggggaattctctgggcggtatgcaatcatataatgatttgatcgacaagtatccgatgtatcaaggtggctttatttgggactttattgatcaagccctcttcgttcatgacccaattaccgaccaagacgtgctccggtatggcggtgatttcgacgaacgccactccgattatgaattctccggtgacggcttaatgtttgccgaccggacaccaaaaccagcaatgcaagaggtgaaatattattatggcttacacaaataatcaactacacgttatttacggcgacgggagtttaggactacagggggctaatttccactacctctttagctacgaacgtggcggacttgaatcactcgtcgtcaacgataaagagtggctctatcgtacacccacgcccatcttttggcgggcgacaaccgataatgatcacggtagcggcttttcagtcaaatccgcacagtggtacgcggccgataagttctcaacttgtcaagatatcgaattgacggttgacgaccaaccagtcacaccgttaccaatcgcgccactcaataacaaatacacggatcacgaaatcgccacgaaagtctcactggcttaccacttcgttaccacgaccgttcctagtaccatcgtcacagtgacttatacggtgacagcagacggtcagatcaatatcgccacccattatagcggtcagtctgatttgccagagctacccgcatttggtctgcggtttatcataccaactaccgcgaccggcttcgactataccggtttgtccggtgagacttatcctgaccggctggccggcgcaacgcacgggcgattccacgttgacagtctgccagtcacaccatacttggtcccacaagaatgcggcatgcacatgcaaactgaacaagtgacagtaacgcgatcaacaacacaaaataacgctgaccacgacaacacaccgttcagtttgacatttagccaagccgatgcaccattcgccttcagctgccttccctataccgccgctgaactagaaaacgcaacgcacatggaagaattaccattagcacggcgaacggtcttatcaatctacggtgccgttcgtggggtcggtggcattgatagttggggaacagacgtagaatccccatatcatatccccgctgatcaagacattgacttcagctttaatattcatttctaaaagttattttgatttcaaaagaacgctccggcgagttatttgccagagcgttcttttagattaacgatgattaagttttaatatgtttaatggctgagcttagtccttagccttgaagtaatgtacctgcgccgtaaagtcactagtttgaacgggagtcgtgtagagtcctagttgcatcaattcatcgccaccgtacattttgtttgtgtcggtctcaacgtaggtctgctgagggtctaatcccgctaacttcgtaatatgcggttctggttgaactgcacctaaaataacgaaggtgaacagcaaggcttctttctgatccggactaacaaacatccacgccaccgtattagattcaaacgggctctctaatcgataaaaggtcccgtattgaactaactcacggtgctgcttatagaaggcaacctgtcttttaacggcctgcttgtccgcatcacttagttgggccgcgtccagttcatagcccaaggtaccactcattgccacagcaccacgcatcttcatcgacgtcgaccgtcctaacaattcatctgggctcgtcccaacatgggcggtaattgcagaaattggataaacgagtgaagtcccatattgaattttgagccgttcaatcgggtcattattatctgatggccaactctgtggcatataatacattaaaccagcatcaaagcggccaccaccgccagagcagccttcaaataagatc

SEQ ID NO: 170 - DNA, 2341 bp, Lactobacillus plantarum
gatctcgttccggttttttatggtgtaagactgatatgaggtgtaattttgattgctgaatactacattattattaatgaactggcaggatctggtcacggtaaggtcgtttgggaaaccgtcaagccgattctagaacaacgacagattcgatttgaatatcgaatttctgaatatgccggccacacaattcggctcgcaaatgagtacgttaaaaccattcaacgacgaccaaacgtgaccccggtcattctggtcattggtggtgatggcacactgaacgaggccttgaatggtattatgcaggtcccacaagctgaaccgatcccgctcgcctacattcctggaggttcgggcaacgactttgctcgcggtctgggtatggcgactgatccagcaattgcacttgcacaagtactcaacaatatgcggccccgttcgttaaatgttggttatttccatgaaaccttgaaaaacgaacaccggtatttcgtcaacaacgttggtttaggatttgacgctcaaatcgttgatgacacaaaccgtagcaaaaagaagggccgtctgggtcgttgggcttatctcagtaacatgctggccgcatattcccaacaggaaggcttcccgctaaccgtacacgttaaccggaagcgagactattataagcgggctttcctttgtacagtctcgaacattccatactttggtggcggagttaaaattctgcctcaggctaatctgcacgataatcagctcgaattgatcgttgtcgaagagcctcactggtggattatcctctggttgttcgtcttactgctactgggtggccgtcatcttaagtcgcgtttcgttcaccattatcgcaacgctaacttgcacttgttggttaactctgttgaaattggtcagatggatggtcaaattattgggaatcgtaattacgacctctacttgtccacccatccctacccattctggatcgacactagtatccatgaccaccactaactctactgctaaaacaatacaaattagcgataaaaagtcgttgcgaagatgctcgcaacgacttttttataggaccaagattggcaattcatactacttgagcgccatccacttaccggccagcaatgctaatgaccacttaattgactccccacccactcggctaataaaaaagactgctgaccatctcaatatggtcacacagtcagtcttgattaatcttagtcctcagaagcgttgattgcttccagaagcatgtcaagttgttcacatttacgagataagtgactcacccgttctttcaaatgcggataaatggtcacaacccgtgagcgatcctgttgccgaacggctagcgtccttaaatgatcgagttcggtattagcctcttgatattcttccagaatctcaaaacgattactcactgatagcacttccttatcacaatagatggtccaatttttcgaattgaaaaacgacaaacataatataccacaattattggtaagtttaaatcataaaattcaaaaaaatttaaataaaaatacccctaacacttaacaatatcgtgttaggggtatttgcaattaaaatcggcttacttaaccgtacctgcaaaacttggcatgaagtaagaactgattgtctgaacttgaacgttttggccttcagttggagccgcaacgtattgattatttccaatatagatggcatcgtgataagtgctaccacgactaccccagaataaaatatctccaggttgagcatccgcaacggaatgcgtcgtcacataagattcctgtgcaaccgtactatgtggcaaggtaatcccagctgcatgttggtaaacataggaaactaaaccagaacaatccattcccgaaagactcgcaccaccccaaacataagggatgttggcactagccaacttcagcgcaaggccggttacagaaccagtagctaaatcactcgttgaactagtagtggtactgctactattagtcgttgcggtactacttgcggtattgcttgcactgctagtggctgctgaagaactactctgactggtcgtgcttgaactagtcgctgccgagctactctggctcgctgtactcgtagttgatgctgcactagcagctgtacttgatgcttgactagccgtgctagtagcagtcgtactgctagtactagttgcactcgcagttgaagcggtactttgactagtcgttgttgaactagcagctgatgaggacgcgctactcgttactgaggcactcgctgcaacactcttcgactgactggtagcagtcgaagaactagcattacttgctga

SEQ ID NO: 171 - DNA, 2477 bp, Lactobacillus plantarum
tttagtgcttcggtttgtgattcatcataagtaatgttgctagctcgaccgacatgctcgatagcatgatccacggcacgttcagacaatttttcatcattatcggcggctaacaaccgttgtaaatgttccgcaacgcgccactcggcttcatagaggccgcgcaagtagatacgattctcgtcacccacgattttattttccttcgccaagacgaccaactgatcagccagcttcgaaggatcaatttgaacattgcgagcattctcaagtagttgcaacgtttgattcagcaacggttgagccgtggtatacgtatcaccgttttccacgcacagctcattaatctcagtcaatagggccgcctttaaccggtcatccgcttggggatcaattttaagttgggccgcaatttgatccgcttttttgaacccgacgcctttgatatcctcaaccaaacagtacggattcgcttgaatcttagccaaagtgtcaccttgatagcgtgcatagatggccgcagctaaccgactcccaaaaccgtacgcattaagtccgatgatagtttgctccaaaccgttattgaccgtgagcgtgtcgatcaaagtcgtctgaacgctggcccgaagtccgattggttttaaaaccttcggatcagctaaaatttgatcaatggcgtcttcgcccaaagtatcgacgattcgctcagcagtccgcttacccaacccaggaaaatcactaccagatagataggcaatcaagcctgcgcgtgtcgtgggcgtttcattttgataattatccgcttgaaactgaaccccgtatttgggatgcgtgaccgttttaccggtaaaacgatacgttgtctcctcctgaatatccgcaaaatttccagtaacaacgatctcatcttccgaccaatcaatgttctgttcagttactttgactaataagactttataaaaactatcggcactgctaaaaaaaacggcagcgactttaccgacgataaagtgtgtgaccggttcccgaccaatatcgtcggaaaataaatgtgcttgttcgtccgtcacggaaatcctcctaaattagtgattttgattatctgcttgtgcggctaacattttttcaatatccgttagctgttgctgatgacttttaaaatattgactgtccaatttctgcgcgcgttcaaaccaggtggcatacggttttcccagtaccattgcggtcagtccaaggttaaaagcagctcgcccagattgcgaatcaatcttatatgcgcgttgatagtaatccgatgcttgctgattattgcctaatgcaagtaatatgtctgctactaacaaattagcgtcaagttgttgtggacgtgcctcttgagccgtaatggcccagactagggcatgttggtaatcgtgttgggccattaaagcttgtgcactcattaaataagcatcctgttttagttgatcatccgtaatttgttgataatatggtaacgctttgtcataagtttgagcttgataatagacatttcccaacgcataacgcaggtagtcggctgcgggttgttggtggtcaaactttcccaacgccttcattgctaacgcctctgcctgttcaaaatcttgaccagccgttaataataccactagttcataataagcatggtagtcctcgggctgtgcatcaatcttaccaatgagttgatggactagtgcttgctgtttagctttgctaggacgtgatgtttttgccatccgaaccacttccttctatattaaatcgtatcagttgcatcgggccgccgtgataggtatgaggtgtcattccaatctaatagcttaaaatgttgaccacggtcacgcgtctctaaaattgttgttgacgtattcgacaagccaccccgttgccgtaacgtcgccagtgtcgctcccagtaatgagttgaccaaggcattcaaggccgcaccatgactaacgatcaaaacattgtcatcgcgacgtggatttgcttgaacaatttgcttaatagcgggagtcatccgcttcagcagttgttgaaagctctccccctgaatcgctgtcgggtcatactgatccggatgatttcgaaacgcgtcgaattcggccggatacgtagcttcaacatccgtgaaggccattccttccatcttaccgagattgaactcccgtaaacgactcagtaccgttatgggtaactctgattgtgtcaaatcattacgtagtgtcatcgctgtatcacgcgcccgttttaacggactgacatagatatgactaaaccgaatatcctggagcgccgctgccaattcatgaatttcttgataactagtcggtaataatggtgaatctccctgagagccttgatagcgcccctcaagattccactctgttttcccatggcgaacaaataatagttttgtcaattgcttacttccttactctagtgtcttgatt

SEQ ID NO: 172 - DNA, 1965 bp, Lactobacillus plantarum
aatgggtgttttcaatcatgacacttaattatgagctatcaaaaaagctggacaaggccagctttcttgtttttaatcttcatttttaaacaatgcatccaacaactgccccgccactggttgggcagtaccaaacattgggaacaagtgcggatacaagtacggcgccgcatgaatacccaagtataccgccatttcaggatgctctggcactaattcggcatagagattgggaatcatcacgtcgaccccagcgaccgccaagtgtaagtctgctaccaacttttccaccgcctgcacataggattgatgcatatcagccgtcgcgtctagcacgtccgccccgttaccaaaagtcgcatcctctcgtaataagatctgagttccccggctgaccacagaatctaaggtcaagtgatatgagtccaggcgataccgttcgatcgttcctaactgtagcgctgattgaggccacttaaaagcggtcccgcgcaacgctcgaccatttttgcgatcaagtaacgtcttgaccgttgagcgaccatcaccaacaatattggctggaattcgttcgacgattgcttgcacacgactatcgataaccaaaaagcgataacttgatgcgactaccacttcctcggccattaccgcggacgtttgctcgaatagttgccggacgacttgttcaaacagtccgcgttcgggcataatccgaaagacaattactttgtgcgactcatccgccgcttttaatacgatcccaccagcttgaacgtaccgatcataatcagcaatcaactgattagctgtatgatatgtctgtgaagccggcaccggaaccccgtgctcagccagaatttgtttggccgctgctttatgtgtcagtacggtcgttagcgcctgtggatttaaatccgttccgctcccattcacaactaattgcgaccgtcctaacttagtcaatcgtaaaatgttagcgtgtgggtcaacaacgtccacctgcactccccgcgtcaaggcctgctgggctagttgctggctcgatagatctagcgcggtaaagcccgcaagttcaaacggacgttcattgctcgattcctgatagcgtgcagcccgttcgagtgcccatgctaacggatcggcttgcatggcaatctgcgcactcagcgtcttctttggatcagtgacccacgacttcaactgtttgagtaacacggcatcttcatttggtaggccataggttttaacaaaatcagcaagtgcatctaaaacttgaacagccggcacggcctgagcactagccgtcgttggattttcacccaaaacttgacgggtcaattggtcagcttgagcgttgacttgggataccatcttagctggaagtgccggcatcattacgaaataactcgccaacaaacgaacaaatgccaccgcgttcgcgtccaccccgactggactagttggatcgagatccaagccccggtattcaagataatagaccccctgccgggccatcatagctagctgcccattactccgaagccgaactggcccgtcaaaatcattgacagacaacaactgctgttgacgaacagccgccgtcaacttggccacgtagcgatcaattgacgtatagtctcccgtcacttgactgtagcgaccatcgggatgttgaacactactgcgctgtggacgactcgtagtatccgtaacggctaggcgtggtgaagcgccaaataaatactgaatcagccagttcatgcgcaccaatccctgagcgactttcagataaattgcattgcgaaagtcaacataactgtgatactgctgatggaaagtctcagtatataaacgggtgaataaagcttcattcaagctcatattcacgtgactaccagtcgtcattaatcgctgtaactcatacttacgagctaagtcgcgacgacgctgatagctgacttgatc

SEQ ID NO: 173 - DNA, 3582 bp, Lactobacillus plantarum
ggatcacgaagatttttcgagttacttgcaacggtttgggcatcacgaataactgaataacgcacaaccgtcaccacaatcattattaagttagatacaccgagattcagtcgccttgctcaaaaaaagtaatagtcatttcaagaatcaattcttgaaacgactattactttttttgaaaccttttccggcacaattaaattcacacttattgacgtttccgttaagccagcaattccgtaatcattttttgataaatctcgatgaagcgcccatacatatccaaatcaacatattcattaacttggtgcgctgtaatattgccgggtccgaagacaatcacttgcatatcaggactctttacaatataagacgatgcgtccgtcccaccaggagcaccaaagtacgggagccgttgctgtaaaacttgttccccaaccttcttcgccaactggaccaactggctatcttccggcgtatgcactgggtagaacgaacttgcgatgtccatcgttagattggcaccatctgcattgcattcagcaatgatggcttccaaatccttaatcaagcggtcattccgtaattctggaatcgtccgaatttttactgacatttcggcactggctgggacggtgttgacctgttcgccaccactaattaacgtcacgacaggcacggttgggcccaatacgggattaacaatagtcttgaaactgtcaaaataggcagtttgtttctgatagtaagtcatcaacatatcgatggcgttttgaccaatcgctggcatcgaactgtgggccgccaccccttgcgctttaatcgtataagtcagtgaacccttatgcgctaattcgatgaagtgttgttcggtcgtcggattcgcatccgccatccgttgcgccgtatcgccgtcaacacctaacatggcttgaatcgacttggtcagtaaaagttgtttgtccgcaccgcttggttccgcacagatcaacgtttgaatatcatcggcgtaaccgagttccgtcaattgttcagcgcctaaatggtcgacttcttctcccacagttgcgagtaacctcaccgtgccatggagtggggcgtcctgatcgtgcaatgcaatcatcgcgaagacttcagccattagtccagccttcatgtccgtcacaccgcgaccatacaaccgattatctttgatagttgctgccaacgggtccgtgtgccatttatctgcatcacccaacgccaccgtatcttcatgaccatccaacgcgacgactgggccgtgaccatcaccaatttcggccaccaagttcacccgcccgggagcgtactcgattgattgtgcttcaataccatgttgctttaaaagtgtgaccaaataatccgccaccaattgctcatggttatttaccgtattcatttttacaatatcgcttaatgcctgtacagcagcttctaactgtgccgtttctgccatgccattcatcccctagtcatccttaattaaactgttccaatcaatttaattataccacttcggctaccgttggccgttgtcagacacgtttaatcgacagatttcagtgcatccagcatattgactcgctttaacttacgatgcatcattcccatgactaataaactaaaggccagggtcaataatgccgcgtaaacgtaactcaacggatgaatcgttggtgaaaacattaacgcgttcgtttcagccgtttgcaagatatatgcgtgcaaccagttgcccaagaaacaaccggcaataatgcctaaaaccgtcaatatcagattttcacggaaaatatacatcgtcacttcaccatcgtaaaagcccaacactttgattgtcgacaattcccggatccgttcagaaacattaatattcgttaagttatagagcactactagcgctagtgcccccgccgagatgacaaagatcaatacgactaagttcatgctatccagcattttaaaattagtggctttctctgtactcatcagtgtcacgttctgaacccccgcctgtttcaatagccggtccgcataagcattttcttgctttttcgttgcctgcttaaaccggacatagttcgtgttatatactggtgcctgcttgaagacacgtcgataataagtcggactcatatagataaagtgattgacgtaattttcagccaccgcactgatgtgaatccgcttggtggtttgcccggccaacttgatcgttaaatcatcgcccgcctgaacgccatataacttagctaatttttcatcgatgaccgcaccgcgatcaccaatatgaatggcctgatgactttgtcggtgccgtaataccacgaacttgcttagcgattggtggggtgccggtataccgagcgtagccgtctgttctgctaccccggactgtttgaccgtcacctgcttggcctgtaatttcaaactagcctggtaaagttgaccacgactgagtgcttgccgttgttggtccgtttcgttcccactacgcgtcaccacagcatcgtagtgccacaattcgttaaattgcttgacgctaatatcaccaatggaatcctttaagccaaaccccgtaatcatcattgccatgcagcccgcaataccgagcacggtcatcagcaaccgttgcttataccgaaatagattacgaagtgtgattttatgattaaaactcagccgatgccatagccattgccagcgttctaatagcaaagtcttaccggccttaggtgatcgtggctgtaagagttgcgcgggtaaactgtttaaatccacgcggagcacgaccagtgccgtccccaacgtgcacaacaacgcaatggctaaggcgataccaatgtccatccaaatgtattgaacgttgattgcgggcaaattatacatactaccataggcctgcgcgataaaccgcgggaaaaaattgacgccgaacaggacacctagcgcggtcccaatcagcgcggctaaaccaccataaatcataaactcgctaccgaccgcggtattcgtatacccgagggcctttaacgtccccatctgtagccgcaattcttcaaccatccgcgtcatcgtcgttagacaaattaacgcggcaatcgcaataaagaacagcggaaagacagtcgacagtgccacgactcgttgcgtattttcgtgatattctgtgtaacccggattatccgtacggtcagtgtacaaataagtcggcattgtgatggccgcgacctgggccttcgcgcgttttaattggctttgtaactgactagttgtgccgttcccgctggcacttgactagcaagttgctgggtcgcctgtcgcaatggtttcagcttagcctgggcctgggcttgtaatgcctgctgccgcttacgcgcttgtggttttaaccagcgtttgagttgcgcggtgttctcgcgattcaagcgccgatacttcgccgtgtagggggtgacgccccgcaaatttttgaattgcacgtcaatgcgcgttatcacactggacttaatcacctgcggacggacatagaccaagtagtccaaagtccctttaccaacg

SEQ ID NO: 174 - DNA, 1965 bp, Lactobacillus plantarum
gatccgccgctgtcattggtcgtaatatcaaccgttccgtttcaaactgagtcaaattgccatctccaaattattattgattcgttaattataataatctactggcaaatgtaaattatttcgttccactctcaatgatttggcaaagtcacctgtgccagcatcgcagttcctaccgcttcaaatcgggcgcagttctaaatccaattctcgcaaattttaaccacaataaaagggccggtaaccaaccgtccctttctctgatcgacttgcaaaccccaagcttgccattgtgtatgtgcctcgtcatggacccagcccatcccgagaggtataaatgcttgtcaaacgttatgattcagctaactgagctacttatcactatcaataattgcttattaacaatgtcctgacaaagtaaatcgatccggctcccaacctaaatccgctgacaaagcgtatgtattgcttcataactttgtctttagtatagcgagccagcattgcgtttagataacacgaagattacaaaaatgttacaactggtctaaaccaactaaaagttagcctggtattgcttaatcgtctctggctacggacacccagtttatctttaacgaacaaagtcgcccactatggtcttaacagtcggcctttattgaactgaaagccgttgattgacatgtgcttgaataacttgtcgccgagaaccggtcaagaaataaatcgttatgccaacaaaaataatgacggtagcaccgattgtttgccattcgaacgcatccggcttaaccattgtctggctacctgatgccatcatgctcaaaatatcaatagcagcatggatcaccatcggcagcaagatactacctgagtacaggtacgtcgaggctaaaaaccagcctagtgtcgctgcaaaaatgatttgctcaaccgtggctgacaagggttggcccgcaaaaacgtttgtaatatgccacattccaaataggccaccgcttagccacactgccaagtcgagttggtgacgccgatgccgaaaagcttgtaataacaaggttaaaacggcaaaacgatacaaccattcctctgcgattcccggttctaacccactcaaaaacattttccaagtcgctgaccgtagctggaagtcccaatgtgtgaacgttgtcgcccatgaaccacccgcactaaaggcattccatagactaaaagccgttcccaccacgatcagcccaaccactaaactagtttgggcctgacgattaaaccgccacgatgggcccgtcaatccccatccccgcagtaagaccccaacagcaatcacaaagcccagggcgcctaaaataccagtatcactgaccatctcgaatactgatggtgcttgcagattcggtaaaatcaaaaaagtctgctgcgcactgacgacaccactaaaaatagccgtaattaggaccgcaatccggccaatcacagattcgatcggtttagtgaccaccactgccaccggtacgaatgcgaccattaaataagccatccccacagcaatcaatccctgctcgtctaaccatgacagctgacttaaccaagccacgaggttcgccagtaaaatcggcaagagtgtgaactgcagtactgtttggacataataattaacatgtcgcaaacgccgccactgtgcttggtcctcctcggcagctggccaccaggctaacagtccccatatcagcaactgcataccaccaactgggaaaaacacaccaccatcaaacgtaaatagttgaataaaggccgcgccaaataacacgatgagctgccccatgtaccagcgtcgtaataattgttcggtttccggcgtcataaaaaaatcccccaatctgatatgcattaatcataccagattggggacgctcacacgtgattttgtttagatttatcggattccacgactaactaataaagatc

SEQ ID NO: 175 - DNA, 1777 bp, Lactobacillus plantarum
cctcctagtacggcgaccttgtgaaccagtactgttcatgtgtccagtactgcccctaactgcgttattaaaacgcacacctcagccactactgcccattcaatgggctgcgtcgaagcgtgacgtctagacacaccccgtatctaggacgtcgattgcttattgtcaaacaattaattaccttggggtggcccgatcgttgcgacccgatgattaccagccatactggtcgtaatacctgagataacagttcccatcggatcgacttgattacttgctgccagcaaccgttctcgaacacgaacaaccgcatgtctaatcgtcaataccaccgtcgttttaactggttggagccgttgtgtcagtagtgaaatcacttcaaccaacatctgcccagcaactacccagtcacccggtttaacgttgaacttaaatgtaggcgattccaaagcactggttaattgtccaattcgtagccagcccaccagaccattggccgctcgaaagccaattaaccgttgattggctgccacggctgtgataaccccagcaaagggcgcaacaatttgatctgttgccggtataatttgctgcttaaatcctagtaacccatataagtgaaccgtatttgcaagggtcgtcacattgatcacttggcgtttaaccggcgcaactacgggaattaatgagctaatgaccactggcacacttctcacttccttactttataaattggcaaccattcgtatttaaataaccttaacaaacgtaacgtaaaaaacttgttttctttaatatgtaagtcaattagtttaagtcattgactgttatctttacaacataaaccataccataattaaactagaatgccagcataattaacggtttcgattaaggtttacatctgttagcgttaattcgtaaactcaattaacgaacgatgctactaaatagctgaaattggccaccataaccccaatgtcgcactcaagcaatcttgtagatttgacatcatgtttccccaatacgtcaatcaggcaatgacggttaggcctacaatttcagtcagaccgattgcgagcaaaaaagagcttccggcttttattccgggagctcaacaagtcaacgttatttatttttaatctcagccactattcaaccgtaacacgcatccgcccgtgccgctttgaaaaatagaggttctgatcaacacggttgtacaaatcaatcggactaccatcggcttgatgcagtgtcgagacaccaaccgaaatcgacacattgatttcctcgtcttcatacttaacaacgagatgattgactgcttcaaagacctgacggacaatcactttggtactagccagatcataaccgggaaacaggacattgaattcttcaccaccggtgcgatacagcttgaccttctcgtcattggcggccaagaccgttgtgaccgtggcggcaacttcttgcaaaacgcggtcccctgcaaggtgcccgtacgtgtcattaacgtgcttaaagtgatcaatatcgaacatcatcatcgataagttgagattgttcttggcactatcatcgaataaatatttgatatgttccgtgtaagcggcaaagttctcagtctcagtcaaagcgtcgtgactcgcaaactgggccaaccgtaatttaatctcactatcttgggtcagcatattaacgtaggcgtacaataatcctgcaaaaatcattaagtagccatattcttgtaaagtattgtcccaatccagcgaatatt

SEQ ID NO: 176 - DNA, 810 bp, Lactobacillus plantarum
gatcgctcccaaagaagatggtcgtaaacggaatccagactttagctgacgttgagactaaccaaataatgtacgacgcaaaccacatgaacgtgcccacgaacgcaaatttcgcattcacactcacttccagccacttgtacatcccgctactatcagacttaaccgctgccccgaactccgccatcattagcgcgaacgggatgaagaacagtacggctgcgactaggtaaaatagaatcgagctgtaacccattaaataataggccaccgtactattggcaaatccgaaaacggtcgtaaagatcatcatgactagcgccatcaatgtaatcttttcattatcttttgttgccattattgcactccctcacttactcctttaatacttttgtaaagctattccaatttaagattaccgaacgaaggcaataaagcgctttagttttcctaacaatattatgattttttcatgtgatagtcagcgcaattattcatctaaacaattgccgcaacagttattgacaaaaactatttcataacggaaacgttcatttaataacatagtaacttttctttaacatagctgacacataattggcgtattctacacttatcagcttagggaaaatataaaacggggttatacatatcatgaaaaaatttaactttaaaaccatgttgctattagttttggctagttgtgtcttcggggtcgtcgttaacgtgactactagtcttggaccacaaaccgcaatcaccgcccaggcctccaagaagctcagccaagcccagcaaattgctaagatc

SEQ ID NO: 177 - DNA, 3846 bp, Lactobacillus plantarum
gatcaaaggtgataccagtcgtcggaacattattcgaaactaatcgatagcccagtttttcatacgcagcaatttgggaaacgggcgaataactattcgtagtgccataagcaccagttactaccgtcgtaggtagtgttttaccactcgttacatccacaaacgcaacggttgcggtctgtgatttggccgtgtacgtgattgcaatcaagtgcggcttatcgtgactagtgaccgacgctgcttcgacttctgtaacactggcttgataacctgtaatcgttggtgacgtgaccgcggccaatagtgctgactgaccaggtgccactgtccagtcaccgtaagtcagctgcttacccgttgccatgtcgaaagttgccgtgcgattgaacgtgaccgactggttaaccgtttcagccgctgattcacctgacgcatattgatacgtaatcgtccgagtcactgtttgttccaacgaatcacgaccagtgcctgccggatatttaggaccggctggataatcagcatcgatggcctgaccaggttggcccggatgatcaacgctgaccgtcacgtgacgatgcgctagtgtaaccgtataaacttgggtattctgcttataaatcagctgatcaggcaagtccgagttaactggttcatagcctaattttgcatacttcgcgatatcagcagtgaccgtatagtcggcagcatcaccgtatttcccattcaaatcaatataacttaagacttggttatccgtagttccatcgacaaattgaacccggatcgtttcagaattaacgctgtatcggaccgttgtttccgtagtctcgcccatgctagcgggtacagccgccgcgatggttgtttgatccgccgtgtaaccagtaatggtcggcgactcaatggccggatagctattcacattggtcgtccaattaccgtacgaaaggaccgttctatccaccgcatcccccgttgccgtgcgcgtatacgtgaccgtttgaagcacatccgctaagtccgtgggtgtgttatcagcataaacgtaatgaatcgttcgctgactggtcttcatcaaatcactaacggccacgttacctggctgatcaaccgtcgccgtgatggtgccatgtttcaggtatacgtagtaggtctgttcagtatcctgatcaaactcaagcggcgctgggaccttatccgaagctaacacgtagcctaatttttcatacgccgcaatgtcctgcgctgtcgataattcgtgggtgtgccaaagctcccactgaggggaatctgttttaaaatcacgttgttcttatcttggtcgacgtaaacgaccgcaatcttttcggtgttcgctaaataggtgtacacgacctgcccaccaacgtccgctagagtgccatcggcttggtaaccggtactttctggttgtaacgtataaccagttttggcatgcgtagcagtcgtatcactcgtggtgtagctcaaatagtccttagcggtaacatcagtcccgtgcgtcgccgttccattagccgttgttaccacgtgtagcgtccccgctttgtcgcggtacccatactgatagacaaccgttacggcgcccaccgtcaactgaccactaggtgaattcgtcaatttataattactattagcagcggcattcgtcagcgtggccgtaatggttgtcgtgtccggatcagttaaccctgacgttaacgtgtaatccaaactttgcaccgcaacaacaccagtaatgaccggcgtaatattttgttcctgaccattggcccacagaatcgtatccggcaaagtaatcgtcactggacgttgagtaatcgtcagagtcccaccaacattcacatcgcctgcaatcaagaaatctggattagcagcggataaggctgccatcccggcgctattcagcgtgacagcgtaagtgcccacgttttgatcgattgcagtggtgttaaggtagtcggtactgacttggtacgccgtcgtcccatccacagccgtcgccgcgctatcagcagtccaatcgcttggggccgtatattcagttggcaagtataccgtatagcgacttggatcagtcgtggcgttgttatcatagacttttgaagctggtgcaatcgttatcttggccgcagtatcagtcgtcgttgtgacgtagcgctggtaagtaactgtgtaatttgaattactatcaccagaatatgtctgagttaacgaaactgcactagtgttcaaataatacgtcacgccattttgtgtaattgtcatgggtaatttagtcatcggtacatacgtcttaccactgtcgcccgtaaaagttacttcgcctaatgacgtgccatcgtttcctactaaactataagtatcggtaatcgttttggccgatagcgtgtaatgatataggtaacttgtgaaaccactaccaatcgtgacagcatccgcaattcctaatgcatcagcgacactgtcaatattagtataattttgcaaattcaacgaataactaccattattttcattagaatcatcagtaacataaaccaccgccgaatccgggcttgaaacttgattaccaattaaaccactagtcaccccagtgttttgaacagcaatgaccggtaataatgcatattttagcgttgcaaatgcaccaccgtcataaacagtggttttatcaaaatgaattttaaatgtttggcgacccctgtagtctgtcagttgggtcacctttaacccctgataggtcacatcatttttcgtcatcattgcttcaatagcagacgttaacacactacttggatcagtcgctatgttaatagctccttcactattagtagcaaccttaaaaccagatggaataataacataatcagtaagattagccacagtcattcctgatggattcatattaataagcggcaagatcaaatacagcgcctgaccatatgctaaatcaattccctttgttcgggcatgtgaaattgtcagccctgttaccggagtttcggcactagcctgcgcgccatagtttgcaggtgagatggcaactgcggcactagccttgacaagcactacgcccttaccaaccgtcgtactattaaaagtcgtactgctgttaaggttagctaatgtttctatcgtatcatcagtcaatgtaatggtgtatttatcaaccacagcagtattaaccgcttccgaatatgtcgtatcggaaacggggacgtcatacacaatcgcatcagtctgaatatcatcgtaacgaattgtccaacctgttggaaccacaatagtacgactattgaccgtcaccggaaccgtactagtcgttttattaacgacaattgtcgtcgcacccatcgtaataatgtcatgtgccgcaataactaaactaccagcaataatcgtatcagcagtaatagcagcattaggattagcctgttgtaaagctgtcaagccttgatctgacaacacaagctgatacgttccagattgggttgctgtgggaactataacatcgccactagaacttgcaatcatataagtattagtcgttccatcagtagccgaactagctaacgtccaggtgctgggaactgcatactgactcggcaccgtaattgtatacgtacttggtttagcatccccatagtcaatactagctgaaccaatggtaattatcgcagtcggtaccggagcttgcttgattgttagtgttcctgtcaccacgttagcagccgttatatccgcgctactattagcttcagctagcttagtaattccagccgttgagagtgccaatgtgtatgttccaacactcgatcc

SEQ ID NO: 178 - DNA, 1466 bp, Lactobacillus plantarum
gatctgatggtagatgagtaacgatcctaggaagaaagcggccgctaagccggcggatgtcggtagtttgttaaacatattggctggtaacactaaggctaatagtggaaaggcatacgctgctggcaattgtaagttaagcgcccgtaaccacaaatacacaagtggtaaggcaatgaccgtcgttaatagccacgaagcaatcaataggtgaatgccgaccccaatactagcagcagccgacaatgcaagccactgcttaagtgcggtcgttaccgtatattcagactgttgagccgcttcaaagaaaacgacgatcacgggtgggattgcggccatctggggctgtcccgctagccaaacaatacccacccagacaaaaactaggctgataaagccgagcatttgccagcgactagcagaagcttcggttacccgtgagattgatcgcggtcgttgaatccaagccccaatcatcaggcaaatagtccagaaaaagatggctacgataaaggtccagtgcgttgcattaatgataattggcagtaagccagtagcaaaggctggcgccaaattcgattttaaccccttcaataataatagcatcagtaatagaccgacaagcactttgagggcgtgcgaccaaggtagttgattgactaagaagccaataattgcagttccagatggtactaagaataactttaagggttgccgagtccacgccgtcttacgataaacccacgtcccggctgtcagagcaccgatttcgggtaaaatgattttcgcatcatgttgcaatgtggcactcgccaccattaatagaatgaagctccacccagctaagtatcgccaaccatcgctttgctttaaatccatttcgacaaaatattcctttcttatattcgattagatatatttatgttatcaagtgtcaacgactagcattgtaccataaatacaatcgtctcctcaaataatcctgaaaacacatcaacagttttcaggattattttcaatcactttcctgacctataaatcaaaattcgactgagtaaagtcttacccagtcgaattcagtcgtcgcgccaacgcagtcgcaacgttatcaacaccgcagtcagaaaattatgcattttgcataagccttttgataactaaacggttatgctatttatcgtactagtaattggttaaccagcctacgtacactagccgttacggcctaacatcaaaaatatgaagatggcttctgctactcgtaaccaccaccgatatcggtgccagcgtaataccgaccaatggtgttttagccactgattggcatagtgcgcaatcagtggcgccccgataatcataaaaccctcggtccaaaaatcgacgcgggcgggccaagtcgtgttatcaaagccagcaccaatggtcgcaactacgagcaagatgtagaggccccacaaactgatttg

SEQ ID NO: 179 - DNA, 16876 bp, Lactobacillus plantarum
aatggcagatgaacggccacttaacttggtacaatttgtcgttggctattttgatattggaactttaggtgaaatatcagagtgccttctccactttgccaaaacgttcagttgttactacggcgattcagaataacgagctatcacaatatcattttcagcctaacagttgattgacaagattactgtctataagaaacaacgacaaagccatcaaaaactgagtacgcacatgtccgccattcgaatactgtcccgagcacgtttcaccaatatggcattaaagcgtggaagaccagtatttaagacgtggtcttcggattaaatctgtgtccaccgcgttccagcaattggcagaaatgcctggaagtcggcttaggacgaactaactaagcaagtttacgctaactcgaactgtttccagcgaacctaagctggctgattttgaactcaaaaaatggcatgttttaatcatagttaatgctagttgttttcgatggttaccagtgtccaaatatgctgatttaagcgtcaagtggactgggattgttgttgatgagcccgctgaaaatggcgttaggtcgactaataacacgtctgctggtgtgatttgcggtgcagtcgtgattgagattgggtcacagtccaataacgatgtaacggtatcaggcgacagttctattgtaatatactaacgcgtaatttgaatgaataagacccataatgatgaaacagataaaagaccgactaacaagcaatcgcaccgcttgttagtcggtcttagtgttcattgattaaagctttttaccagattttgacccgcttatctggtgccaaatacatggcatcgtcgggttgaatgtcgaaggttgtgtagaaatcatctaagttcttaggttgtacgtttgctcggagcttagctggagcgtggacgtcgattgaaagtaagagttgcatgtattcagtcgtggccttcatacgccaaaccatggcccagttagtgaagaacgcactcaaatcaacgtcatcttcgcccttagcagcttcttccgcgcaacttaagcctcccgcatcggcgatgttttcggatacggtcaacgtcccgttaactttcgcaccagcaaagtctaagccatcgaattcactgatcatggacttggcaagttccttgaagtgggcagaatcttcctcggtccaccaattatgcaagttaccgaattcatcaaatagggcaccattgttgtcaaaagcgtgcgagatttcatgagcaataactgcaccaataccaccatagttagcgctagaagattgttctaagctgtagaaaggtgcttgtaggatagcagctggaaagacaataatgttcataaatgggtggtagtaagcattgacggtgtccgcactcatttcccagcgggtccgatcagtggctttgccccactttgagaacatgtcttggcgtgccaaacggttaaagtggaggacattgctcaatacgttgccaccttgttcgggcgtgtgcgtcttgaatttcgtgtaaatcgtttctagtttatcagggtaaccgacttgaatgccgagtttatcaagcttggtcacggctttagcacgcgtatctgcactgagccaagtgttcacttgcaatcggcgtttgtagacggcaatcatcttttcgaccatctgatgaacgtccgctttggccgcttcaccaaaatacttgtggccgtaatagagtccaacgacttgatcaaaagttccggtagctagatagtaggcactcttagcctggttccgcggttctttttgaccagacaatgcccgtgaataagtcgtggctaatacccgcatttcatcactgaggtaaccactgtaccgttgaacgagtttagctttcatccagttcttcatcagaccgaagttatcaggattgactacttcgttaaagtgatcgaagaaggcgggttctggcaagatgacgaggtcgggattaccatcgatgacggagtaggtgatggcggctaagtctaaataacgactagtgttaacaaaatcgttgaatttgcgaggattatacatcttactgtaatcagccgactcttcagcggatttgatccacggaacgatcaaacgatcgaattgcagcgtgtcatcaacgattttttgtgcttcgtccttttcgtaaccagttttttgcaacagttcggtcatcattttagcgtaaattgctagtaacttagggccggcctggttgccttcgtcgtagtatgtcttgtctggcaagatagtcccaggtgcttgggcaaataaggcgttgaccttggtatttttcatatcagcgtcgacatccaagctgaatggtaatgggaggccgtcgtagatccaatcgggcatccgttgttgtaagtcagcaaagtcgcttaaactatcaatttgcttgagacacggcagaattggttccgcaccattggcattgcgttggtcgaaatctttagctaagcggtaaaacttaatgaattcggttagctgaggatcgtcgggggtaactgttcccgccgccattgcatcaaagtcatgcattaacgttttttcaatggcatcaactaagtccataaagcccccggttgaagaatggtcatccggaatgacggcggtttgagccactcgccgttgacggcatcgtagagatcttgttttactgctgcttgattaatggttgccattaaatccactccctaataaattttctaatactaagttaattataccttagattattctcagtggtatcatctggtaccgactcagtaaaaaatgatactaatcatcttaatagtcctgaatcagtagctgatgggttgtgaatgttaatgtaaaggtcaagattacttctgaattatcggcatgcaggagctgttagtgcctgtaaatcaccttatgaggcagcgtgatggttgacgtaaaagcccccggatgactaacagtgatttcgctagtcatccgggggaagggataatttaattagtagtcattattcagttaataatagctggtcggcatcgttaatttcgatgaccttgccaggcttttgcgtaattaggccttgacgctcgaataaggcgagcttacggctgatgctttcaggggtggtgccgaggaacgtcgccaggtcttttttctttaatggtagtttaaacgtgtcggttttgagcgctgccgctgtttcagtgatatagttcgctaaacgggcttcgacggattcagtggccgtgctggttgtttgacgttccagttgactgagacgtttgccaaagacgtttaagacgttgatgctgatactgggatatttttgcatcaagtcttgaaagtctgaacggcgaatactgcaaacatccgtcggtactaaggcttcaccgaatgaggttcgccgttgattttcaaataaggcagcttcgccatcgatatcaccagtttgtaataggtatagtaactgctcgcgaccattggcagctagctgatagactttaacctgcccattggcaacaatcatcagtgaatccagtggatcgtcagcaccaaataaagtgctgccggctggatagtggtggtgacgaacgatcgattcgatagtgtctaattgttcactaccgagttcgctaaaaattgggactaactgcacacattcatgttctgccataaggcccacctcctgaataaattcaccctaactataacagattttgcttggtagatagggtttgacgttttaaatttcctactgttttaataactatattgtagcgtaaaacttttttgaatttcttgacggccgtcaagttctggtcaatcaaatccggttatgatagaaccatcaaatgaacgacacggaggtaattgattatgaaaattattatgcaattaggaacgttgacttgcccatcatgcatgaccaagatcgaaaaggccgttgctaaccatgacggcgttgagaatgttaaggtgctattcaatgccagcaaggtcaaggctaactttgatcctgaagtgacgaatgccgatgatttggcacaagttgttaccggacttggttacgaagttgaaaacgtcaaggttaagtaggaggaataaacgatgagtgaactaacgatcgacgaacaatacgcagcggaattaaaacagagtgacattgatcaccatgtcccaacagcgggcgcaatgacgaatcacattttatctaacttaatggttgcttacgttaagttgacccaagtgaagtggtacgttaaaggaccacaatcattggcattgcggacagcatatcaacgtttgttagatcagaatgtccgtcagtttgctgaattaggcgaattactcctagatgaaaaccagaagccatcttccacaacggctgaattaaccaagtattccatgttggaagaaaatggggctttcaagtatcaatctgttgatgaactcgtagccgcaacaatcaaggattttgatacggaaaacctattcgttgaccgggctatcaagcttgctgaaaaagaaaaccggccggcactggcagcttggttagttgcttatcgtggcagtaacaatcgcaatattcgggaattacaagcttatcttggtaacgatgctcgaactggtttggatgaagaagatgaggatgacgacgattaggtcgttgtcgctgaagaattaaacgctctggatagcgattgtgtgggcaaactatccagggcgtttttgctattacaacgggcctgcgctatactaacgacaattaaattatatgggagggccccgagatggcgattagcgacattgtgatgtggacatttgtctttattattgcgactgggttcgtaacggtgatttcaacggcggtactgttgaagcgagcaaaacgagcgcccaaggatgccgagcagtcccacagtgatgacgacgatgactggaaaaaagatgattggaaccatgatgactggaaaaagggcgattggaaagattgactcgataagttgcctgacagatacggtgcttaactgagtcaactggcactaaaaagacggtataaccaactgagattggcgacaccgtcttttttggcgttatttggtttttagcagtagatccaaggagtgttcaagattctgaacatcagccgtggtcatcgcatctacacaattattaatattacgggcgatgacgagtttcatggccttgtcgatacttgatttcactgctgaaagctgaaccaagacatcttcgcaaggacgattttcgtccatcatacgcaaaacgccgtccagttgaccagccgagcgttttagccgggtcttgatctgcttgctagtgacatattcttcagtagttgccatgattcatgatgcccctttcgtaattaagcaagtgcttttttgagttcggcttcaaaatcaggcttaggacgtgctccgaccatgtgactgacgacttggccatctttcttaatcaggaaggttggaattccttgaacttggaattgactggccgtagcttggtcatggtcaatattcaatgaaacgaagttgacctggtccttgtaagctgggtcttcagaaagggacttaagaattgggtccatcatgcgacatggtgggcaccagtctgcgcgaaaatcaataacggcgataccagtatctgtttctttggcaaaatcttggtcatgtatttctttaatcattttagttgactcctttagtaagtatttgtcactaaacaatatacccccataggtatacacgagcaagtattctgcttctggccgtgggggatttaaaaaggactgatgtattgggtaattagataataacgatttgttgtggaagctagcgtcacctgagaatcttctagttgtgatcgcgagcattaacgttgaagatctcatgttgacgcgacgtgttatactagcgcttcataattatacaacccagggtatctggtagaattatgttgaattattagaaaccgcttatttaagcaaaataaagcgtttacataaacagctttatctggtatcttcaaattatcatagatgctcggtaccatacgaggaggatttacagatgacaggttcatgggaagcacgctacgccgctgaattttttggcacgctgatcctagtactactaggtaatggtgctgtcgctaacgcattcctgaagaacacgactggtaacgatgatccaggactcgccaatggaggatggctcctggttgcatcgggatatggacttggggtcatgctcccagccatgatgtttggctcgatttctggtaaccatttaaaccccgcgattacgattgggcaagccgtgattggaatctttccatgggcgcacgttgcgccttacttaatctggcaatttctaggagcaattgcaggccaatgcttgattttggcactgtactggccccattatcggcaaacgactgataatgaggcagtcttagggacgtttgcaactagtgatcatgcgaacagtcagttaaacggttttgttacggaaatggtaggaaccgcagtcctgatttttggtgccatgggattatatcgcgggatgttttttcatcaaaatatcgatatcgccaatattggcgttggacttttgattgctgccatggttatttcgttaggcggtccaaccggtccggccctaaatccggcccgtgacttgggaccacggctagtacacgcgttattcccagtaccgaacaagggtagttctcactgggaatatagttgggttcccgtggttgccccaattgttggtgccgttattggaatttggatttataagatcttttttggtttataatcgcaattaattaaccgagcgtatttatgaaattattattggtctatcatttttgattgcaatatgataaaacggtccgccgactagctggggaagactagtcggcggaccgttattttttgctaggcctgtaaccaagttggggagacttgattaagctgtggctgcttgaatggttttagttggttggtgcttgtcgaacttggtttggaaattgattaaccgcatagcgttgaagataacgactaagatactagcttcatggacaaacatcccactggccatgtagatataaccgaagattagcccaatcagtaggaaggccacggtggcaatggcgatgaagatgttttcacgagtgttcaagacagttttcttggcgagaccgtgggcgtgtactaatgcggggaagcttgattgcatcaagacgacatcggatgtgtcgatggcaacgtcagtcccactacccattgcgattccgatatcggcgttagcgattgaaggactgtcgttgatgccatctccaataaaggcgaccgtattaccggctgcttttagtttcttaacgtattcgactttttcttctggcaagaggttggcgtgaacttcatctaggttcaattcgttggcaacggcttgagcggtgagttcattgtctccagttaacataacgagtttcttgattccttgggcttttaatgcggcgagggaatccttcacgcctggacgaatcgtatcggcaatcccgaagatgagttgaacttgtccgtcgactgccatgataacggttgattgaccaccagcttgtaaaccattgagatctttgagttgggtaggatttagtttgatattatgagccgtcaacattttttgattaccaatgacaacttcttgcttaccgacttgtgcacagattccttggcccttaacggtttcagtgtcatctaggacgggagcgacccccgctgactgctggtcggcataactgacgattgcttggcctaacggatggtcggaaacgccctcaatagcggcagcgagagctagttgattatccgcattgtttgtgtaagtgtgcatggttgtaacggcagtgttaccttccgttaaagtaccggtcttatcgaagaccagggtatcgactttggcaaaagtatcgacgacttcaccacccttgatcaggataccacgcttggcaccattcccgattccggcaacgtttgacactggggcaccgataactaaggcacctgggcaaccgagaaccagtaccgtgattgctaagcggaagtcacgggagaaggcgaagactaagactgctaaaacgaggacggcaggcgtataatattgagcaaagcggtcgatgaacttttcagctttggacttggtatcttgagcttcttcaaccaattcaataattttggcaaaagtcgtgtcgtcgccaacctgagtcgctttgatcttcagatagccattttcaaccattgttccggagtaaaccgagtccttgagttgcttgttgatttggcgtgcttcacccgtaattgaggcttcgttaaggtagccgttaccttcaacaacaatcccgtcaacgggaacctgactcccagttttaactaggacaacgtcaccttcgtccacatcatcaacgtcgacttcttcggtgccatcatcagtaactaaagttgcggtagttggtgacatgtcagtcaagcctttgattgcggtccgcgtcttctgcaaggtcttgctttcgagataggagccaaacaagaagaggaaagtaacgattgcggattcgttgaattcaccaataatgaatgccccaatcactgcgatactaactaacagttcaatactgaaaactttgttccgaagtgcgctccaagcacgaaccgcaatcggaatgacggcaataattgaagcaactgctaaaatgacttggtaaccgagcgtaaattgtaagagatatttactgagcatcccaaggacaatcagaatccccgtaactaatgtaatgtgattcgtatgtttagtgagaaataattggaatttcataatttgcaactccttatttgatttgatgaccctagtatagtgagttcagcttgatggcaacttgataggcgtcaagttttgaaaaataacagtattaattctggattgctggcagtgattatgattaaaacatgccattttttaaattcaaaagcagccagcttagacccactggaaacagttcgggttagcgtaaacctgcttagttcgttcgtcctacgccgacctccaggcatttctgccaattgccggaacgtggtgggcacagatttgagccgaaaaaacacgtctcaaatactggcctttcactaaccaagcaaaggacgcttgctaagtgaaatttcaccactgggcattgtcagaaacgccttccggtcgggacagtattcgaatggcagacggagatgtacccaacttttattggactcatcatttttctttataaacagtaattttgtcgagcaactgtcaggtagaaggtaacattttgatagctcactatttcgaaatcactatagtaataatcgaacgtctcgacaaagtggttaatgtaaccttaaagtctcgtttaaaattccattatcaaaatagtcaacgaccaattgtaccaagttaagtggccgttcatctgccattcgagcggcttcccgactggaggggctggatggcaatgctcagtagccaaattattcttagtcggaagtgcctttcttccggcttagaataagactcgtatttgagatgacgcggtttgtgcgttagctcaaatcgacgtcggctgcgttccagcaattgtcatccagccccggaggtcggaatagaacgctcaccggctgacgaacagtaagccaactaaatggcatgttttaatcatgtttcatgacaagttaacggaagaataggtaggatgatcaagtttgagtaaacaagaggtataggagggaaaaacgtggattatcaaataacgcaggccaatcagcccttgatgccggcatactttcaggggcgttgggcggtcaagcaaattgcggatcgagacgtgatgtatagcactaatttgggtgctgaaatcgattttcaagtcactgatgctagttttgtgcggttgacattcttgccgttggcgtatgagctaccgagttgggtggcgatccagattgatggcttgccttttcaacggcaggcggtgacgaatgaccccctatggttgacactggatggtcggcctcacgtggtacgggtagttttgagtggtaatactgatgaggaccgtgtgtgggatggcaatcaaggttttacagtgaaagccctgacgacagatggtgagttacagccagttcggttaggccgacacagtgtgacgtggattggggattcactgacggccggctgctgggtcatgggtaagaaccctgccgaagattatcgcgcggaagccaactatgcggcggttgcgagtgacttgttgaatgcacggaatgtccgcattgcttatagtgccgttggtctcagtaaaccaggaactggtggcgtaccggtactgccggaggttttaacagccgtggatagtaaaacgacgtggcaaccagtacccacggatttagttgtgattaatgttggtactaatgatcgtcatacggatgatacgacgttcacggtagttttgcgccgcttccttaatcaggtgcagacgctttacccgaatagccggcttgctgtaatgatcccgtttaatcagaattttgcgtcgattattcgcgcggtcgtggctgaatttatgcaattgcaactcattgaaacggctacttggcaactcagtaccacggacggggtgcatttagatttagctggctcccggatggcgggcgaattaacagcacaagcgttgcggacacagtatccagatgtttttaaatcgtgatcaacccatcttataaaaatcccgttttactgtagcaatgttctgctgcagtgaaacgggattttttggtggcactactagattttaaaaataaacgttaacgctggcggtgtagtgattgcgactgtaaataccgggccgcagcgagaacttcttcgggaatatccacatgggcgatgaagccctgaccacggggaccataaccggtgggggtatccactaggcgttgaaattcaccgagtaagagttcagtaaaataagcgggttgccagccagctactattgtgtttggcgtaaattgcattggttgtagccggtttaattgcaaggtcgccggcgcatagcgaataaagttggcattggaccaattggctgagaacgttagggtagtccgccgcgcgagtaacgggtcttgtacgagtaggacgtggcgccaatcggtgggggctaacgcgcgggcatgcgtggcattgctaccggtattggtagaagtcttgtcgaggcggatactaccttgatagccagtctttcgaacaagcgtcgccatcaactcagcctcactagcttgagattttggcaggtttaagttttgccgcaaatacttagtggcgtggccgacgccgcccgcaataatgagtagtggtaattgatagtcttgggctaactggcccgctgcagtggctgttgctgggaggctgttgccacaaaggaccaggccatcgaggtgctcaggcaaggcggtggtgtgtgtcaaccaagccagacattgattgaaagcggtcagttcgtccaattacttggcctccttagtcccaaagtgagccgctgcggccccaaagttgaaccgttggaagtgaacattttgaaagcctgcttgctgaaacatcgttgctaattgttgatagcttgcaaagtgacgagtggtttcctgtaaataggaatattcctgatattgatgggcgaataagcgcccgaatagtgggactacttttgtgaaataccactgccagactggtttaatcaatggctgatcgggttgggacgtctctaagcagaccaagcgagcccccggttttaggacgcgataaatttcagttaaagcttgggctttatcaggcaagttgcggagtccgaagccaatcgtgacgaggtcaaaggtgttgtctttaaacggaagatgcatcgcattaccgcggcgcaaccagacccgatcggcaacctgttgctgggtcactttttgttgtgccagttttaacattggggctgaaaagtctaatccgatgacttcaccgggtgcctgtaattctttagccagcgcgatcgtccaatcacccgtaccacaacagaggtcaagaacatgggcgttagacgctaaatggatttgggccatcgtttgcttgcgccaatgacgatgcgttccgagactgatgatgttattcatccgatcatagttcggcgcgatggtgtcgaacaaaccttggacattatgtaaataacgatttgccataaatgatacctcctgatagttgctttacttactactatagcagatgttagttgccagttaccgtgaaaaggaatgataatcaatataaagctacgatattgatttcgttgctgttaatattgttcgtgaataaaccgttaaactaaagttaaaataacataaaggcagtctcgattggtatttattaagtaccaatcaagactgccttttttgacagcgcgttcctaatcgaagccttaacgtcaaaacgagtacgctagtaatagcttaaaaatcatcatctgcaatttgagctaacaggtcacgtgatggaatgaagaacgcttgtccggatagtaaggtggagaaagttagtaaaaagtcactttggtcgaccatgttttgcagcatgcctttggtaaccgtccagtgacgggcataaccaatgaagtacgtccctgtgttaccagctgctggattggagtacgggacattcatccgaacgattttttgttcgacaccatcaatttccaattttgaagcaacgttgtgagcattcttaaacttgtcttcgtcttctaactcgaagtcgctgaatttttcacggcccacggctttttcctgatcctcggtgtgcatatgttcccacacgggcatatcatgttgccatttctgtgcaaaggcgtaagaaccgttgatgaactggggatcttcgtcaccaactaaggcataatcagcggcatcttctacggccggcgcttccgtaccgtcgataaagccaatgatggcccggccttcgaagtaacgaaagcccttcgtctcatcgaggacggtcgtgattggcgctaaaacccgctggaactgcgtttggcattcgtagacgaccgcttcgttgcttgcgcgaatgtggaagaagaggtcgccgggtgttgctggcatagtgtatttgggtccacttaaggtggtgtaagtttctaattctttaggtttgggtgcgccaggaaacaggtaatcccaagcggcactactgatgccgatgctaacttttaattgggtgccagtttcaggtttggcgtcccggatgcgtaacgagcgaatgatggcttgtgaccgatctgcaaattcttgaaagacttcacggtcgtgttgctgatcctgacgatttaattgaagaacggtaaattgaacgtgttcaccgacatccttccagacatcttgggcgcgatttggattgattggcatggtatatttcctccataaattgtttgatattgttagcgtacatggtggtacgggtagcgtcaaataaaggcaaacggcgggggtgaataatgacgacattcgccaccatcagggacaggcggccaaactaccgtggtgtgtatcatgataactaaacggattagtcagttggaattgttggtgcttgtaagacattaggaattaattgaataaatagccggtaagtctcaattctgctgagtgttggctgcgtggtcgcaagttgccaagctaactgatgaagacagttgccgagataagttgccataaaatgccgttgcttaggatggatggctaagctagttagcagctgttgggcaaaaccaacttctaatgggatgacccgatcgcgacactgtgcccatgacacgagtaaggggagatccagatgcgcgcgaagcgtcgtgacgattgcaccagcaatggtcgcgtaagatgtggtagttaagtcatttaacgcttgtcctaactgttgtaaggcaattaagtattcaatcacaatgatatcttcaatatcctggtgatggcgataaaaagtggcacgtccaacgtgagcatccagacaaagttgtttgacagttaactcgttgaagatcttttgagccttgaaattcgcttgcagggcagtaatcaatttacgatgagtttgatgagttcgctgatcaatatgcaattgattgttcataattaattttgtcatatgtcaatgatacaccaacttgtgatatgtctcaaaacgtcccttttttagcatgtgaaagcgtctataatagctgtcacagttagatatagctgaccaattaaggagattgtcgactaatgaaattaattaccgttgaagagcattttgaatcggcagctgtgactgccgcaatgcgtcaagctgttggtaacgctgcgttaccaacagttagtccagcgctacgccagtacatgcgggacaatttaccgagtctgcaattatgcaagacacccagcatgaacgcctggcatttatggctcaatcgggcattgatatgcaggtgttatcttatggtaattcatcaccacagaacttatcgccggaacaagcagttccattctcacaattagctaatgatgaattagcgaaagcggtggcggctcatcctgatcgttatgctgggttagccgttctaccagttggtgatccgcaagctgcggtcgctgaattaaaccgagcggtgacgacgctgggattacggggtgtgttgctgaaaggaaattatcagaataaattttttgatgagccattcttcttaccaatctttgaagcagctgcgacgcttgatgtccccgtgtattttcacccctcatttattccgcaagcagtaacgagtcattattttgaaagtaaccaatggtctgacgttgtcacggggatcttatcatctgccggttatggttggcatatggatgttgggattcaagtgatacggatgattgctagtggtatttttgataaattgcctggcttgaaactcatttcaggacactggggcgaactagtcccgctatttttggaacgtttggatgatgagttaacaacatatacagacttgcagtatccatttagtacttattatcggcataatgtgtacgtgacgccaagtggtattttgagtgcacctcagttgcaatttatgttagcggagatgggggcggatcacctgatgtattcgatcgattacccgtataagcagccggagacgagtggaagtttcctggatatcgccgacttgaccgatgaacaacgggctcaaatcgcgtttggcacggcaaccacgttatttaaattataggaggaacgaaataatgtcattaaacgcacaacaaatgcaggcaacccgggatgaattacaagctaactttgcgttgactggtcttacgaaagcccaggtagctgatgatttagcgattagtgcaactaagctcgatcacttatttgacttaacacaacaatctttgaacgatccgtggatcttacgtaattatttgattgaaaaggtggaagcaggcgggaagacgccggtgccatttacggcattaagtggcgactggcatcgtcattggtttttgaatagcgttgtcattgatcgccgcgaaatgtcggccggtgattgctaaaatgattggaggaaatcgtcaatgagtaacggaactgatatggcactaattagtgaactagtggcccgtgagcgcttatttcgtgcccggcataaccctgaaatccgagattgctattacgccgacgcgactgtggcaactagttggcaacaaggaccactaagtacatttattggcgcagaatcgaaggaagttgatccgcgctttgttattgtggggagcgtcagtacgcctgtcgttcatctgaatggcgataaggcatacgtggaactaccaaccacgacgcatatgcgcatgatggtcaatggtactttagcggaattagaatcatatcggcgactgatttaccgcgtcgaacgccgtgatactaagtggaagattagtcgattgacctcaattaacgaaagtgataatttacgaccagtcattgtgggacaggatttacacgtgatacctcaagattttaacggtttacggtcctcgtatcaattcctagcctatgtccgacaagctgccggtggtcaaattagtcaggatttattagggacggatcgaccagaagaagttgagcgattgtacaaggaaacgaatgcgtggttgcgagctggagtatagttgaaggtaaaataaaaaaagcgatatcatgtaaaatcaaatacttttagcgtcttgtaaccagaaacgaaaataatttagtacattcgttaaaataacagaccgttactttgataaatataaataaatgatgatccaaaaaataaggtgtgttgcaatcgaaagttgattgcaacacaccttatttttgacgtctattaaaatggaccctaccggatttgaaccgacgacctcctgcatgcgaagcaggcgctctcccaactgagctaagggcccaatacacttttagtataccgcaaaatgctagtcgtgcaagtctagtttggtcatcaatggcgactagcacgatgattttgattgccacgaattttatgcactcgtcgtttgtactgtgctttttggttgctgacaaacttgaagtgttgcattttgccgccgaagacttgaccattcagccttaattttagccaatcttgcataacggccatcattgaattaatttgggtg

SEQ ID NO: 180 - DNA, 1972 bp, Lactobacillus plantarum
ctttcctagattgtgaatatatgcatgacatggggaattctctgggcggtatgcaatcatataatgatttgatcgacaagtatccgatgtatcaaggtggctttatttgggactttattgatcaagccctcttcgttcatgacccaattaccgaccaagacgtgctccggtatggcggtgatttcgacgaacgccactccgattatgaattctccggtgacggcttaatgtttgccgaccggacaccaaaaccagcaatgcaagaggtgaaatattattatggcttacacaaataatcaactacacgttatttacggcgacgggagtttaggactacagggggctaatttccactacctctttagctacgaacgtggcggacttgaatcactcgtcgtcaacgataaagagtggctctatcgtacacccacgcccatcttttggcgggcgacaaccgataatgatcacggtagcggcttttcagtcaaatccgcacagtggtacgcggccgataagttctcaacttgtcaagatatcgaattgacggttgacgaccaaccagtcacaccgttaccaatcgcgccactcaataacaaatacacggatcacgaaatcgccacgaaagtctcactggcttaccacttcgttaccacgaccgttcctagtaccatcgtcacagtgacttatacggtgacagcagacggtcagatcaatatcgccacccattatagcggtcagtctgatttgccagagctacccgcatttggtctgcggtttatcataccaactaccgcgaccggcttcgactataccggtttgtccggtgagacttatcctgaccggctggccggcgcaacgcacgggcgattccacgttgacagtctgccagtcacaccatacttggtcccacaagaatgcggcatgcacatgcaaactgaacaagtgacagtaacgcgatcaacaacacaaaataacgctgaccacgacaacacaccgttcagtttgacatttagccaagccgatgcaccattcgccttcagctgccttccctataccgccgctgaactagaaaacgcaacgcacatggaagaattaccattagcacggcgaacggtcttatcaatctacggtgccgttcgtggggtcggtggcattgatagttggggaacagacgtagaatccccatatcatatccccgctgatcaagacattgacttcagctttaatattcatttctaaaagttattttgatttcaaaagaacgctccggcgagttatttgccagagcgttcttttagattaacgatgattaagttttaatatgtttaatggctgagcttagtccttagccttgaagtaatgtacctgcgccgtaaagtcactagtttgaacgggagtcgtgtagagtcctagttgcatcaattcatcgccaccgtacattttgtttgtgtcggtctcaacgtaggtctgctgagggtctaatcccgctaacttcgtaatatgcggttctggttgaactgcacctaaaataacgaaggtgaacagcaaggcttctttctgatccggactaacaaacatccacgccaccgtattagattcaaacgggctctctaatcgataaaaggtcccgtattgaactaactcacggtgctgcttatagaaggcaacctgtcttttaacggcctgcttgtccgcatcacttagttgggccgcgtccagttcatagcccaaggtaccactcattgccacagcaccacgcatcttcatcgacgtcgaccgtcctaacaattcatctgggctcgtcccaacatgggcggtaattgcagaaattggataaacgagtgaagtcccatattgaattttgagccgttcaatcgggtcattattatctgatggccaactctgtggcatataatacattaaaccagcatcaaagcggccaccaccgccagagcagccttcaaataagatc

SEQ ID NO: 181 - DNA, 1402 bp, Lactobacillus plantarum
tgatggtggacgttaaaccaactaacgaaaaactcgtgattcgcgcgaaacacatgatcgaactggccaccggcgtttctgccgatgaagccagcgaactattcgctgcagctcatcaaaacgttaagacagcgattgtcatggacttggctggcgtctctgtcagcgacgccgagcagcgtttacagcgcgcacacggtgtcgttcgtgacgcactcgcactgcaatgaggaggtctaaaatgacatatttaattggcgttgactgtggtggcacgcacatcgttggtcaaacttggacgacagcccccgagcatctagtccaaagcgttacgggtggccctggtaacgttgtcctagactactctgctgccgttactaacttaaccactgtcttagaccagctcactgccgcaattccagctagtcagcttgggttgattttaatcggaattgctggcattgaaactgctggccgggctgatcaggtccaacaaaccatcacccaacgttaccacgctaatacccaggtcataagcgatgcaaaactggccctactgaacggtcttgcaggagcagacggcgccttagtgattgccggcacgggctcggtcgtttatggccgccaagccggaaaatttctgcgcgttggcggctggggttacgttttaggtgacgaaggcagtgcctatgacattagcaagcgggcacttaaacaggttctgacccagactgataacggtcaaactagtcaactaacagctcccctattggcacaacttaaagttaccgatattgctgccgccgtccagaaattttacgctcaagatcgacaaactaacgctcaattagcacagttaatcgccaaactggccgagcaacaaaattctgaagccatcacggtattagtcacgtcagcccaagcactggcacaacaagtcgttaccttatatcagcggtttgcagagtcctggccacaacgggtcgccctctctggttccgttttacaacacaatcgcctggtccgcgacacgttaacgacgacagtgcaccagtcaataccaacaattgcttttaacgatattacaactaacaacgcccacgccgtcatctattggcaccggtggactcaggaggaaattaattcatgacaatgcgtcaactaggtttatccatttatcccgatcatagcgactttgaagaaaacgccgcttatctcaaattaggccaacactacggcttcactcgaatttttatgagcatgctggaaatatctggtacggtcgccgaaaccaaagctaagtaccagaaaatcattgacgttggaaatcagttgggttaccaaacgattttggacgtttcaccacggatttttaaacaactaggaatttcctataaagatc

SEQ ID NO: 182 - DNA, 61 bp, Lactobacillus plantarum
aatttaagtgcctcattcaatggcatttcgggtaacttaggcgcaatttcaggatagtctt

SEQ ID NO: 183 - DNA, 706 bp, Lactobacillus plantarum
agcgtccccaatctggtatgattaatgcatatcagattgggggatttttttaggccgactgttaagaccatagtgggcgactttgttcgttaaagataaactgggtgtccgtagccagagacgattaagcaataccaggctaacttttagttggtttagaccagttgtaacatttttgtaatcttcgtgttatctaaacgcaatgctggctcgctatactaaagacaaagttatgaagcaatacatacgctttgtcagcggatttaggttgggagccggatcgatttactttgtcaggacattgttaataagcaattattgatagtgataagtagctcagttagctgaatcataacgtttgacaagcatttatacctctcgggatgggctgggtccatgacgaggcacatacacaatggcaagcttggggtttgcaagtcgatcagagaaagggacggttggttaccggcccttttattgtggttaaaatttgcgagaattggatttagaactgcgcccgatttgaagcggtaggaactgcgatgctggcacaggtgactttgccaaatcattgagagtggaacgaaataatttacatttgccagtagattattataattaacgaatcaataataatttggagatggcaatttgactcagtttgaaacggaacggttgatattacgaccaatgacagcggcggatc

SEQ ID NO: 184 - DNA, 104 bp, Lactobacillus plantarum; identical to SEQ ID NO: 12

SEQ ID NO: 185 - DNA, 111 bp, Lactobacillus plantarum
gtcgacggatctttattagttagtcgtggaatccgataaatctaaacaaaatcacgtgtgagcgtccccaatctggtatgattaatgcatatcagattgggggattttttt

SEQ ID NO: 186 - DNA, 655 bp, Lactobacillus plantarum; identical to SEQ ID NO: 14

SEQ ID NO: 187 - DNA, 650 bp, Lactobacillus plantarum
aggccgactgttaagaccatagtgggcgactttgttcgttaaagataaactgggtgtccgtagccagagacgattaagcaataccaggctaacttttagttggtttagaccagttgtaacatttttgtaatcttcgtgttatctaaacgcaatgctggctcgctatactaaagacaaagttatgaagcaatacatacgctttgtcagcggatttaggttgggagccggatcgatttactttgtcaggacattgttaataagcaattattgatagtgataagtagctcagttagctgaatcataacgtttgacaagcatttatacctctcgggatgggctgggtccatgacgaggcacatacacaatggcaagcttggggtttgcaagtcgatcagagaaagggacggttggttaccggcccttttattgtggttaaaatttgcgagaattggatttagaactgcgcccgatttgaagcggtaggaactgcgatgctggcacaggtgactttgccaaatcattgagagtggaacgaaataatttacatttgccagtagattattataattaacgaatcaataataatttggagatggcaatttgactcagtttgaaacggaacggttgatattacgaccaatgacagcggc

SEQ ID NO: 188 - DNA, 1263 bp, Lactobacillus plantarum
atggctaataaatcattaatcaaagtcgcagtaaccgcactagtagctggtttaatcggtggtggtgttgcttacggcggtattaattatttccaaaacaataacatcgcaacgtcatcgaccagtgtaccaactggttctaataaatcggggtcaacgtcaacgacgaacgttaaggtcaatgtcagttcacaggcgaccaaggtgtttgaaaataacaaggcggccgttgtatcggtcattaatctccaaaagaagagctcttcaagcagttggagtggtattttgggtggcgatgactcgtctggcagtgatagttcatccagttcagattctagctctagtaagctggaagagtacagtgaaggttctgggttgatctataagaagagcggtgacgcggcttatattgtaacgaataatcacgtggtgagtggttcaagtgccattcgagtgattatgagtgatgggactaagttgtcagctaaaattgtcggaaccgattctgtgactgacttggccgttctgaaaatcaattcttctaaagtaacgaagacggctagctttggtaactcagataatatcaaggttggtgaaacggccttagccattgggtcaccgatgggctctaattacgcaacgaccttgacgcaaggaatcatttcagcgaagaagcggaccgtggcgacgacgaatacatctggtcaaacgacggggtacgcgacggttatccaaacagatacggcaattaactctggaaactctggtggtccgttgttcaacattgctggacaagttatcgggatcaactcgatgaagttggcctcagataattctgggactagtgtcgaagggatgggctttgcaattccaagtaatgaagttgtgaagatcatcaatgaattggttcaaaagggtgaagtcgttcggccggctttaggggttgcaacctatgacctatccaatatttcttctagtgatcagaagtctgttcttaagttaccaaccagtgtgacgaagggtgtcgtcatcatgaagacgtactcaggttcaccggctaaagctgctgggttaacgaagtacgatgtgattacggagctaggtggcaagaaagtgaccagcttagccacgttacggagtgccctgtatgcccattcggttaatgataccgtgacggtgaaatactaccataacggaaaactcaagacagccaacatgaagttgacggaaactaccaaaacgttaactaaacaaagtaactaa

SEQ ID NO: 189 - protein, 420 aa, Lactobacillus plantarum
MANKSLIKVAVTALVAGLIGGGVAYGGINYFQNNNIATSSTSVPTGSNKSGSTSTTNVKVNVSSQATKVFENNKAAVVSVINLQKKSSSSSWSGILGGDDSSGSDSSSSSDSSSSKLEEYSEGSGLIYKKSGDAAYIVTNNHVVSGSSAIRVIMSDGTKLSAKIVGTDSVTDLAVLKINSSKVTKTASFGNSDNIKVGETALAIGSPMGSNYATTLTQGIISAKKRTVATTNTSGQTTGYATVIQTDTAINSGNSGGPLFNIAGQVIGINSMKLASDNSGTSVEGMGFAIPSNEVVKIINELVQKGEVVRPALGVATYDLSNISSSDQKSVLKLPTSVTKGVVIMKTYSGSPAKAAGLTKYDVITELGGKKVTSLATLRSALYAHSVNDTVTVKYYHNGKLKTANMKLTETTKTLTKQSN

SEQ ID NO: 190 - DNA, 285 bp, Lactobacillus plantarum
gtgttaaaaccattaggagatcgcgttatcttgcaacaacaagaagaagaagaacaaacaattggcggtattgtcattgccaataacgctaaggaaaagccccaaagcggtaaggttgttgccgtcaatgacggtcgtgttttagataacgggacaaaagttgaccccagcgtgaaggtcggcgatcaagtattattcgataagtatgccggtaccgaagtcaagtatcaaggtgctaagtatttggtattgcacgaaaaagatatcgttgcaatcgaagactaa

SEQ ID NO: 191 - protein, 94 aa, Lactobacillus plantarum
MLKPLGDRVILQQQEEEEQTIGGIVIANNAKEKPQSGKVVAVNDGRVLDNGTKVDPSVKVGDQVLFDKYAGTEVKYQGAKYLVLHEKDIVAIED

SEQ ID NO: 192 - DNA, 1626 bp, Lactobacillus plantarum
atggctaaagaattaaagttctctgaagatgcacgttcagcgatgctaaaaggtgtcgatcaattagctgacacagttaagtcaacgttaggtcctaagggtcgcaacgttgttttggaacaatcatatggttcaccaacaattactaatgatggtgtaacgattgctaaggcgatcgaattagacgatcatttcgaaaacatgggtgctaagttagtttctgaagttgcttcaaagactaatgacatcgctggtgatgggacgactactgcaacggtcttaacacaatcaatcgttaatgaaggtatgaagaacgttacggccggtgctaaccctgttggcattcgtcgtgggattgaagaagctactaagacggcggttgactcattacacgctatggcacacgaagttaagacgcaagaagatattgcgcaaatcgcttctgtatcttcagcaagtgaagaaactggtaaattgattgccgaagccatggaaaaagttggtcatgacggtgttatcacgattgaagaatcacgtggtgttgatactagcttagacgttgttgaagggatgcaattcgaccgcggctacttatcacaatacatggttactgataatgataagatggaagcggatcttgacaatccatatatcttaattactgataagaagatttcaaacattcaagatatcttaccactattacaatccatcgttgaacaaggcaagccattgttgatcattgctgatgacatttctggtgaagctttaccaaccttagtcttgaacaagatgcgtgggacgtttaacgttgtcgccgttaaggcacccggttttggtgatcggcgtaaggaacaattacaagatatcgctatcttaactggcgggacggttatcactgacgaccttggccttgaattgaaggacacgaccatcgatcaattaggtcaagccaacaaagttacggttactaaggataacaccaccattgttgaaggcgctggttccaaggatgctatctcagaacgggttgaatttatccgtaaccaaatcggtgaaacaacttctgactttgacaaagaaaagttacaagaacgtttagctaaattagctggtggggttgccgttgttcgtgtcggtgccgctactgaaactgaattgaaggaacgtaaataccggattgaagatgctttgaacgcaactcgggccgccgttgaagaaggctttgttgctggtggtggtactgctttgattaacgttatcaaagatgttgctgcattgaaggaaactggtgacgttcaaactgggatcaacattgttaaacgtgctttggaagaaccagttcgccaaatcgctgaaaatgctggtttagaaggctctgttatcgttgaaaagatgaaggaacaaaagccaggtgttggtttcaacgccgcaactgatgaatgggttgacatgatcaaagctggtatcgtggacccaactaaggtaacgcgttctgctttacaaaatgccgcttctgtttcagcccttctcttaacgactgaagccgttgtcgctgaaaaacctgaagaaaatgcaccagctgcaccagccgcaccaaacccaggtatgggcggtatgatgtaa

SEQ ID NO: 193 - protein, 539 aa, Lactobacillus plantarum
MAKELKFSEDARSAMLKGVDQLADTVKSTLGPKGRNVVLEQSYGSPTITNDGVTIAKAIELDDHFENMGAKLVSEVASKTNDIAGDGTTTATVLTSIVNEGMKNVTAGANPVGIRRGIEEATKTAVDSLHAMAHEVKTQEDIAQIASVSSASEETGKLIAEAMEKVGHDGVITIEESRGVDTSLDVVEGMQDRGYLSQYMVTDNDKMEADLDNPYILITDKKISNIQDILPLLQSIVEQGKPLLIIADDISGEALPTLVLNKMRGTFNVVAVKAPGFGDRRKEQLQDIAILTGGTVITDDLGLELKDTTIDQLGQANKVTVTKDNTTIVEGAGSKDAISERVEFIRNQIGETTSDFDKEKLQERLAKLAGGVAVVRVGAATETELKERKYRIEDALNATRAAVEEGFVAGGGTALINVIKDVAALKETGDVQTGINIVKRALEEPVRQIAENAGLEGSVIVEKMKEQKPGVGFNAATDEWVDMIKAGIVDPTKVTRSALQNAASVSALLLTTEAVVAEKPEENAPAAPAAPNPGMGGMM

SEQ ID NO: 194 - DNA, 591 bp, Lactobacillus plantarum
atgtatccagttcctacagttattgaacagtcatcacgtggcgaacgtgcttatgacatctattcacgactattaaaggaccgtatcattatgttatccggtcccattgaagataacatggcaaacgccattattgcccaactactcttcttggatgcccaagattcaggtaaggacatctatctctatatcaactcaccaggtggtgtcgttactgccggcttagcaatctacgatacgatgaacttcatcaaatctgatgttcaaaccatcgttatggggatggctgcttccatggccagcgtcttagcttcatctggtactaagggcaagcgttttgctttacctaactctgaaatcttgattcaccaaccatctggtggtgctcaaggtcaacaaacggaaattgaaattgttgcggaagaaatcttgaagactcgtaaaaagatcaaccagattttagctgacaactcgggacaatccgttgaaaagttgaaccatgatactgaacgtgataactacttaagcgcacaagaggctaaagactacggtttgatcgatgatattatggaaaacaacaaattaaaataa

SEQ ID NO: 195 - protein, 196 aa, Lactobacillus plantarum
MYPVPTVIEQSSRGERAYDIYSRLLKDRIIMLSGPIEDNMANAIIAQLLFLDAQDSGKDIYLYINSPGGVVTAGLAIYDTMNFIKSDVQTIVMGMAASMASVLASSGTKGKRFALPNSEILIHQPSGGAQGQQTEIEIVAEEILKTRKKINQILADNSGQSVEKLNHDTERDNYLSAQEAKDYGLIDDIMENNKLK

SEQ ID NO: 196 - DNA, 1173 bp, Lactobacillus plantarum
atgttagacaaaatcatttataaaaacttatttagtaaagcgttcgatattactattgaagtcacttattgggatgggcaaattgaacggtatggtaccggcatgccagctgttaaagttcgattaaataaagaaatcccaattaagctattaactaatcagccaacattggttttaggtgaagcatacatgaatggggatattgaagtagacgggagcattcaggaattaattgcctctgcttaccgccaaaaagacagttttttgacacataattcatttttgaaacacttgcccaaaatatcacattccgaaaaaagcagcacaaaagatattcaaagtcattatgatatcggcaatgatttttataaactatggttagatgataccatgacctactcttgtgcgtactttgaacatgacgatgatactttaaaacaggcacaactcaataaagtgagacatattttaaataagctggcaacccagcctggtaaaagattattggatgttgggagtggttggggaacattattatttatggccgcggatgagtttgggttagatgcaacgggtattactttaagtcaagaacagtatgattatacacaagcgcaaatcaagcagcgtcatttggaggaaaaagtgcatgtgcagttaaaggactatcgagaagtcactggccaatttgattatgtcacctcggtaggtatgtttgaacatgttggtaaagaaaatctagggttgtactttaataaaattcaagcgttcttagttccaggaggccgagctttaattcatggcattacaggtcaacatgaaggtgccggcgttgatccatttattaaccaatatattttcccagggggctatatcccaaatgttgctgagaatctcaaacatattatggctgctaagttacaattttcagacattgaacccttgcggcgccattaccaaaagacgttagaaatctggtatcacaattatcagcaggtcgaacaacaggtcgtcaagaattatggggaacgatttgaccgcatgtggcaattatatttacaggcatgtgcagctgcttttgaggccggaaatatcgatgttattcaatatctattagtgaaagcgccgagtggaactggccttccgatgactcgccattatatttatgattga

SEQ ID NO: 197 - protein, 390 aa, Lactobacillus plantarum
MLDKIIYKNLFSKAFDITIEVTYWDGQIERYGTGMPAVKVRLNKEIPIKLLTNQPTLVLGEAYMNGDIEVDGSIQELIASAYRQKDSFLTHNSFLKHLPKISHSEKSSTKDIQSHYDIGNDFYKLWLDDTMTYSCAYFEHDDDTLKQAQLNKVRHILNKLATQPGKRLLDVGSGWGTLLFMAADEFGLDATGITLSQEQYDYTQAQIKQRHLEEKVHVQLKDYREVTGQFDYVTSVGMFEHVGKENLGLYFNKIQAFLVPGGRALIHGITGQHEGAGVDPFINQYIFPGGYIPNVAENLKHIMAAKLQFSDIEPLRRHYQKTLEIWYHNYQQVEQQVVKNYGERFDRMWQLYLQACAAAFEAGNIDVIQYLLVKAPSGTGLPMTRHYIYD

SEQ ID NO: 198 - DNA, 2268 bp, Lactobacillus plantarum
atgcccaaacaacctacctggactgcccaggatgtcctggacatggttcaaaagtatatgaatagtgatcacgtcgcgttagttaaacgggcgtgtgattttgcaacttatgtgcataaggatcagtatcgccaatctggtgagccgtatattatgcatccgattcaagttgctggtatcttagctgaattgaagatggaccctgaaaccgtcgcttcgggtttcttacacgacgttgtggaagatactggtgttactttaggagacgttgaagaactgtttggtcatgacgtggccgttattgttgacggggtcaccaagctgggtaagattcggtacaagtccaacaaagaacagcttgctgaaaatcaccgtaaattactgttggcgatgtctaaagatattcgagtcatgattgtcaaattagctgatcgcttgcataatatgcggacattgcagcatctgcggcccgataaacagcggcgaattgcaaatgaaacgttggaaatttacgcccccattgccgatcgattagggatcagcacgattaaatgggaactagaagatatttcactacgttatttgaatcctcaacagtattatcgcattgtccacttgatgaattcgcggcgtgaggaccgtgaaaagtacatcgagattgccattcaagacattcaaaaggcgctccatgatctggaactaccagaagctgaaatttatggtcgtccgaagcatatctattcaatttataagaagatgcgggacaaacacaaacagtttagccaactttacgatctgctggcaattcgggtggtcgtggattcaatcaaggactgttatgcagttttaggtgcgattcacacacaatggaagcccatgccggggcgttttaaagattatattgcgatgcccaaggccaatatgtatcaatctttgcataccacggtggtcggtcctgaaggtaagcccctcgaaatacagatccggacgtttgaaatgcaccgggtcgctgaatacggggtcgcagcacactgggcgtataaggaaggtaaacgcgacgaggtccaagagactcagtcgggcaacaagttgaacttagtcaaagaaatcattgagctacaggatgaaagtaaggacgctgccgactttatggagggcgtcaagggcgacctctttagtgaccgggtctatgcttttacgcccaagggtgacgtgacagaattaccaaagggcgctggaccactggatatggcatattcgatccatacggaagtgggtaaccatacgactggtgcgaaagtcaatggcaagatcgttccattggattaccaaatcaaaaatggtgatatcgtggatattttaacgtccactagttcaactggtcctagccgtgattggcagaaattagtctatacgcggcgggcccgtaataaaatcaaacagttcttccgcaatgctgaccgtgaggaaaacatcattacgggtcgtgatttgcttgagaagcagctacgtgatttagagtttaatccaaaagaaatcatgactaaggacaaggtgacggcggtcgctcaaaagatgcactacggtagtgaggatgatttgttcgcggccttggggtttggtgacgtccaaccggtagggattgctaaccggttaacgagtgatgttcgtaaacagcgcgaggctaatcggcagcgtgaacgtgaggaggccattttggcagactctacggaagcgccagcgaagaagaaatcgaaagatcatcataatgaggatcaggagaagcaggatcggaagcggcaaaaggtctcatcttctggtggggtgattattcaaggcgtcgacaacttactcgtacgtctaagtcattgctgttctccaattccgggtgatgagattgttggttatattacgaaggggcgcggtgtttcggttcaccgtgttgattgtccgaacgttaagagcgcagaagcaaatggtgaacggttgattgatgttcagtgggagaatcccgagggtgaccgaacgaactacaattctgatttggaaattcaaggttataaccgtaatggcatgctcaacgatgtgttgaaagttatcaataatcacacgaaatttttgaccaatgtcaacggtaaggtcgatcacaacaagatggtcattattagtgtttcgttgggggttcgcaacttggaacatctccaacgaatcattgacagtctgaaaaatgttcaggatctttacgttgtcgaacggaaaatgttttag

SEQ ID NO: 199 - protein, 755 aa, Lactobacillus plantarum
MPKQPTWTAQDVLDMVQKYMNSDHVALVKRACDFATYVHKDQYRQSGEPYIMHPIQVAGILAELKMDPETVASGFLHDVVEDTGVTLGDVEELFGHDVAVIVDGVTKLGKIRYKSNKEQLAENHRKLLLAMSKDIRVMIVKLADRLHNMRTLQHLRPDKQRRIANETLEIYAPIADRLGISTIKWELEDISLRYLNPQQYYRIVHLMNSRREDREKYIEIAIQDIQKALHDLELPEAEIYGRPKHIYSIYKKMRDKHKQFSQLYDLLAIRVVVDSIKDCYAVLGAIHTQWKPMPGRFKDYIAMPKANMYQSLHTTVVGPEGKPLEIQIRTFEMHRVAEYGVAAHWAYKEGKRDEVQETQSGNKLNLVKEIIELQDESKDAADFMEGVKGDLFSDRVYAFTPKGDVTELPKGAGPLDMAYSIHTEVGNHTTGAKVNGKIVPLDYQIKNGDIVDILTSTSSTGPSRDWQKLVYTRRARNKIKQFFRNADREENIITGRDLLEKQLRDLEFNPKEIMTKDKVTAVAQKMHYGSEDDLFAALGFGDVQPVGIANRLTSDVRKQREANRQREREEAILADSTEAPAKKKSKDHHNEDQEKQDRKRQKVSSSGGVIIQGVDNLLVRLSHCCSPIPGDEIVGYITKGRGVSVHRVDCPNVKSAEANGERLIDVQWENPEGDRTNYNSDLEIQGYNRNGMLNDVLKVINNHTKFLTNVNGKVDHNKMVIISVSLGVRNLEHLQRIIDSLKNVQDLYVVERKMF

SEQ ID NO: 200 - DNA, 1044 bp, Lactobacillus plantarum
atgatcacgttaactgaacgacaaagcctaattttaaaggccattgtccgtgactataccgagggcggtaatccagttggatccaaatcgctagttcaagaactaccgatcaaggtcagttcagcgacgattcgtaatgaaatggcccgactagaagatttaggattaatcgtcaaaacgcatttgtcttcagggcgaattccatcgattaaggggtatcggtactatgttgaccatatcctaaagcctgaaaaggtggatggcaaagacttgaaggtgattcaacattcattaggcggtgaatttcacaagatcgatgagatcgttgctcagtcggcggatatcttgtcgcaactgacaagttacacgacctttacattgcgacctgaacttaaagatagtcggttgagtggtttcagactcgttccgttggggaatcatcaagtaatggcgattctagtgacgaataatggtgacgttgaaaaccagacgtttactattcctagtgacattaccggcgatgagctggaaccggtcgttcgtttcattgacgatcaactggttggcctgccgttacaagacgtcctccgccaattaacgcatgagattccgttaaaacttgcacagtatttgcaagatccagatggtttcttagatatttttggcagtgtgttgtccaaggcagcttccgagcgcttttatgttggtggtaagttgaatttgttcaactatacggaccagcagagccctaaagagttacagtcattgtactcgttactcgaccaaacggaccggttagctaacgtgattggtccacccggtcaacggattcaagtccgaatcggtaatgagatcaccaacgatttgttgaagaactacagtttaattaccgcgacttacgatgttgatcaacacggacaaggtgtgattgctttgctcgggccgaccgccatgccgtattcacggatgattggactgatgggtgcgttccaacgagaattagcccgcaaattattagattattaccggtactttgacgagtga

SEQ ID NO: 201 - protein, 347 aa, Lactobacillus plantarum
MITLTERQSLILKAIVRDYTEGGNPVGSKSLVQELPIKVSSATIRNEMARLEDLGLIVKTHLSSGRIPSIKGYRYYVDHILKPEKVDGKDLKVIQHSLGGEFHKIDEIVAQSADILSQLTSYTTFTLRPELKDSRLSGFRLVPLGNHQVMAILVTNNGDVENQTFTIPSDITGDELEPVVRFIDDQLVGLPLQDVLRQLTHEIPLKLAQYLQDPDGFLDIFGSVLSKAASERFYVGGKLNLFNYTDQQSPKELQSLYSLLDQTDRLANVIGPPGQRIQVRIGNEITNDLLKNYSLITATYDVDQHGQGVIALLGPTAMPYSRMIGLMGAFQRELARKLLDYYRYFDE

SEQ ID NO: 202 - DNA, 1194 bp, Lactobacillus plantarum
atgctagaaaaaaccttttaccacacccttctaagccactcattcaatatgcccgtcacagtcaactactgggatggaagtagtgaaacttatggtgaaggcacaccagaagtcacggtgacttttaaagaagccattccaatgcgtgaaattaccaagaacgcttcaattgcccttggtgaagcttatatggatggcaagattgaaattgatggcagtattcaaaaattaattgaatcggcctatgaatcggcagaaagtttcttcaacaattctaagttcaagaagttcatgcctaaacaatctcactctgaaaagaagagtcaacaagacatccaaagccattacgatgtgggtaacgacttctacaagatgtggcttgatccaaccatgacctattcttgtgcttacttcaaacatgacactgatacattagaagaagcccagattcataaggttcatcacatcattcaaaagctcaacccacaacctggcaagaccttactagacattggttgcggttggggtacgttgatgttgactgccgctaaagaatacggcttaaaagtcgtcggggtcacgttatcacaagaacaatataacctagttgctcaacgcatcaaggatgaaggcctcagtgatgttgctgaagtccggttacaagattaccgtgaacttggcaacgaaactttcgactacattaccagtgttgggatgttcgaacacgtcggtaaggacaacttagcaatgtactttgaacgcgttaaccactatcttaaagctgacggggttgccttattgcacggcatcacccggcaacaaggtggcgccactaacggttggttagataagtacattttcccaggtggctacgttcctgggatgacagaaaacttacaacacattgttgacgccggcttacaagtcgctgacgttgaaaccctccgtcgccattaccaacggacgactgaaatctgggataaaaactttaacgctaagcgcgctgccatcgaagaaaagatgggcgtgcgcttcactcgcatgtgggatctctacctacaagcctgtgccgcttccttccagtctggtaacattgacgtcatgcagtacctcgtaactaaaggtgcttcatcacgaaccttaccaatgacccggaaatacatgtatgcggataaccgaatcaataaagcttaa

SEQ ID NO: 203 - protein, 397 aa, Lactobacillus plantarum
MLEKTFYHTLLSHSFNMPVTVNYWDGSSETYGEGTPEVTVTFKEAIPMREITKNASIALGEAYMDGKIEIDGSIQKLIESAYESAESFFNNSKFKKFMPKQSHSEKKSQQDIQSHYDVGNDFYKMWLDPTMTYSCAYFKHDTDTLEEAQIHKVHHIIQKLNPQPGKTLLDIGCGWGTLMLTAAKEYGLKVVGVTLSQEQYNLVAQRIKDEGLSDVAEVRLQDYRELGNETFDYITSVGMFEHVGKDNLAMYFERVNHYLKADGVALLHGITRQQGGATNGWLDKYIFPGGYVPGMTENLQHIVDAGLQVADVETLRRHYQRTTEIWDKNFNAKRAAIEEKMGVRFTRMWDLYLQACAASFQSGNIDVMQYLVTKGASSRTLPMTRKYMYADNRINKA